(19) United States
(12) Patent Application Publication
(10) Pub. No.: US 2016/0201132 A1
(43) Pub. Date: Jul. 14, 2016

(54) GENE EXPRESSION BIOMARKERS OF LAQUINIMOD RESPONSIVENESS

(71) Applicant: Teva Pharmaceutical Industries Ltd., Petach Tikva (IL)

(72) Inventors: Liat Hayardeny, Tel Aviv (IL); Anat Achiron, Tel Aviv (IL); Michael Gurevich, Rechovot (IL)

(73) Assignee: Teva Pharmaceutical Industries Ltd., Petach Tikva (IL)

(21) Appl. No.: 14/914,523

(22) PCT Filed: Sep. 12, 2014
(86) PCT No.: PCT/US2014/055502

§ 371 Date: Feb. 25, 2016

Related U.S. Application Data
(60) Provisional application No. 61/972,782, filed on Mar. 31, 2014.
(60) Provisional application No. 61/877,210, filed on Sep. 12, 2013.

(51) Int. Cl. C12Q 1/68; A61K 45/06; A61K 9/00; G01N 33/68; A61K 31/4704

(52) U.S. Cl. CPC C12Q 1/6883; G01N 33/6896; A61K 31/4704; A61K 9/0053; A61K 45/06; C12Q 2600/106; C12Q 2600/158; G01N 2800/285; G01N 2800/52

(57) ABSTRACT

This invention provides a method of predicting clinical responsiveness to laquinimod therapy in a subject afflicted with multiple sclerosis or presenting a clinically isolated syndrome comprising evaluating expression of a biomarker in the subject. This invention also provides a method of treating said subject comprising determining whether the subject is a laquinimod-responder by evaluating expression of a biomarker. Also provided is laquinimod or a pharmaceutical composition comprising laquinimod for use in treating said subject, and a therapeutic package for use in dispensing to said subject, wherein the subject has been identified as a laquinimod-responder or expression of a biomarker in the subject is up-regulated or suppressed.

Throughout this application, various publications are referred to by first author and year of publication. Full citations for these publications are presented in a References section immediately before the claims. Disclosures of the publications cited in the References section in their entireties are hereby incorporated by reference into this application in order to more fully describe the state of the art as of the date of the invention described herein.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Various MS disease stages and/or types are described in Multiple Sclerosis Therapeutics (Duntiz, 1999). Among them, relapsing-remitting multiple sclerosis (RRMS) is the most common form at the time of initial diagnosis. Many subjects with RRMS have an initial relapsing-remitting course for 5-15 years, which then advances into the secondary progressive MS (SPMS) disease course. There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and Fingolimod (Gilenya®). Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Laquinimod (LAQ)

Laquinimod (TV-5600) is a novel synthetic compound with high oral bioavailability which has been suggested as an oral formulation for the treatment of Multiple Sclerosis (MS) (Polman, 2005; Sandberg-Wollheim, 2005; Comi et al 2008). Laquinimod and its sodium salt form are described, for example, in U.S. Pat. No. 6,077,851. The mechanism of action of laquinimod is not fully understood.

Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2002; Brück, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

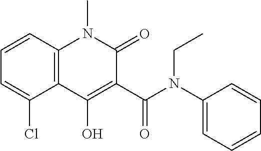 [SMILES: CCN(C(=O)c1c(O)c2c(Cl)cccc2n(C)c1=O)c1ccccc1]

IUPAC: 5-chloro-N-ethyl-4-hydroxy-1-methyl-2-oxo-N-phenyl-1,2-dihydroquinoline-3-carboxamide

As MS therapeutic options grow, the ability to identify subjects who will respond more favorably to therapy and specifically to laquinimod has become increasingly significant.

### SUMMARY OF THE INVENTION

The subject invention provides a method of predicting clinical responsiveness to laquinimod therapy in subject afflicted with multiple sclerosis or presenting a clinically isolated syndrome, the method comprising evaluating expression of a biomarker in the subject, so as to thereby predict clinical responsiveness to laquinimod, wherein the biomarker is a gene associated with inflammatory response, a gene associated with cellular movement, a gene associated with cell signaling, a gene associated with cell development, a gene associated with hematological system, or a combination thereof.

The subject invention provides a method of treating a subject afflicted with multiple sclerosis or presenting a clinically isolated syndrome with laquinimod, comprising the steps of: a) determining whether the subject is a laquinimod responder by evaluating expression of a biomarker in the subject, and b) administering to the subject an amount of laquinimod effective to treat the subject only if the subject is identified as a laquinimod responder, so as to thereby treat the subject, wherein the biomarker is a gene associated with inflammatory response, a gene associated with cellular movement, a gene associated with cell signaling, a gene associated with cell development, a gene associated with hematological system, or a combination thereof.

The subject invention also provides a method for treating a subject afflicted with multiple sclerosis or presenting a clinically isolated syndrome comprising the steps of: a) administering to the subject a therapeutically effective amount of laquinimod, b) determining whether the subject is a laquinimod responder by evaluating expression of a biomarker in the subject; and c) administering to the subject an amount of laquinimod effective to treat the subject only if the subject is identified as a laquinimod responder, or modifying the administration of laquinimod to the subject if the subject is not identified as a laquinimod responder, so as to thereby treat the subject, wherein the biomarker is a gene associated with inflammatory response, a gene associated with cellular movement, a gene associated with cell signaling a gene associated with cell development, a gene associated with hematological system, or a combination thereof.

The subject invention also provides laquinimod for use in treating a subject afflicted with multiple sclerosis or presenting a clinically isolated syndrome, wherein the subject has been identified as a laquinimod responder.

The subject invention also provides a pharmaceutical composition comprising an amount of laquinimod for use in treating a subject afflicted with multiple sclerosis or presenting a clinically isolated syndrome, wherein the subject has been identified as a laquinimod responder.

The subject invention also provides laquinimod for use in treating a subject afflicted with multiple sclerosis or presenting a clinically isolated syndrome, wherein expression of a biomarker in the subject is up-regulated and the biomarker is a gene associated with inflammatory response, a gene associated with cellular movement, a gene associated with cell signaling, a gene associated with cell development, a gene associated with hematological system, or a combination thereof.

The subject invention also provides a pharmaceutical composition comprising an amount of laquinimod for use in treating a subject afflicted with multiple sclerosis or presenting a clinically isolated syndrome, wherein expression of a biomarker in the subject is up-regulated and the biomarker is a gene associated with inflammatory response, a gene associated with cellular movement, a gene associated with cell signaling, a gene associated with cell development, a gene associated with hematological system, or a combination thereof.

The subject invention also provides laquinimod for use in treating a subject afflicted with multiple sclerosis or presenting a clinically isolated syndrome, wherein expression of a biomarker in the subject is suppressed and the biomarker is a gene associated with inflammatory response, a gene associated with cellular movement, a gene associated with cell signaling, a gene associated with cell development, a gene associated with hematological system, or a combination thereof.

The subject invention also provides a pharmaceutical composition comprising an amount of laquinimod for use in treating a subject afflicted with multiple sclerosis or presenting a clinically isolated syndrome, wherein expression of a biomarker in the subject is suppressed and the biomarker is a gene associated with inflammatory response, a gene associated with cellular movement, a gene associated with cell signaling, a gene associated with cell development, a gene associated with hematological system, or a combination thereof.

The subject invention also provides a therapeutic package for dispensing to, or for use in dispensing to, a subject identified as a laquinimod responder afflicted with multiple sclerosis or presenting a clinically isolated syndrome, which comprises: a) one or more unit doses, each such unit dose comprising an amount of laquinimod, and b) a finished pharmaceutical container therefor, said container containing said unit dose or unit doses, said container further containing or comprising labeling directing the use of said package in the treatment of said subject.

The subject invention also provides a therapeutic package for dispensing to, or for use in dispensing to, a subject afflicted with multiple sclerosis or presenting a clinically isolated syndrome, which comprises: a) one or more unit doses, each such unit dose comprising an amount of laquinimod, and b) a finished pharmaceutical container therefor, said container containing said unit dose or unit doses, said container further containing or comprising labeling directing the use of said package in the treatment of said subject, wherein expression of a biomarker in the subject is suppressed or up-regulated and the biomarker is a gene associated with inflammatory response, a gene associated with cellular movement, a gene associated with cell signaling, a gene associated with cell development, a gene associated with hematological system, or a combination thereof.

### BRIEF DESCRIPTION OF THE DRAWINGS

FIG. 1: Source of variation in data set (point 4) before (FIG. 1A) and after (FIG. 1B) normalization.

FIG. 2: PCA analysis. FIG. 2A—PCA analysis based on 43 genes passed FDR criteria after 6 month of treatment. FIG. 2B PCA analysis based on 1564 genes passed FDR in combined 6 and 24 months treatment data. Red dots represent patients at baseline, blue after treatment.

FIG. 3: TGFB canonical pathway. Green color represents suppressed genes from combined 6 and 24 month LAQ related gene expression signature overlaid with TGFB.

FIG. 4: Immunomodulatory effects of TGFB in MS. Paradoxical effects TGFB in multiple sclerosis is shown (according to Mirshafiey et al., 2009).

FIG. 5: Expression of TGFB1 in PBMCs of RRMS patients following treatment with LAQ. Protein samples (30 mg) were prepared from phenol/ethanol fractions of PBMCs derived from patients after 6 month of LAQ treatment (+) and compared to PBMCs samples of the same patients before treatment (−) (FIGS. 5A and 5C). From each sample 30 ug was separated on 10% SDS-PAGE. Blots were incubated with Rabbit a-TGFb1 (1:250) and mouse Tubulin (1:500). The signal intensities of a protein band and its surrounding background were scanned for each protein and quantified by using Quantity One 4.6.9 software (Bio-Rad Laboratories, Hercules, Calif.). The resultant background-subtracted values of protein expression were normalized to those of Tubulin and then plotted as the relative protein levels for each patient with or without LAQ treatment (FIGS. 5B and 5D).

FIG. 6: Expression of PAI-1 (Serpine 1) in PBMCs of patients following treatment with LAQ. Protein samples were prepared from phenol/ethanol fractions of PBMCs derived from patients after 6 month of LAQ treatment (+) and compare to PBMCs samples of the same patients before treatment (−) (A). From each sample 30 mg was separated on 10% SDS-PAGE. Blots were incubated with Rabbit a-PAI-1 (1:500) and mouse Tubulin (1:500). The signal intensities of a protein band and its surrounding background were scanned for each protein and quantified by using Quantity One 4.6.9 software. The resultant background-subtracted values of protein expression were normalized to those of Tubulin and then plotted as the relative protein levels for each patient with or without LAQ treatment (B).

FIG. 7: Profiles of PTCRA, TGFB1 and TGFB1 related genes PF4 and CSGP5 under LAQ treatment.

FIG. 8: FIG. 8A and FIG. 8B show proposed mechanism of LAQ effect on PBMC of RRMS patients. FIG. 8C shows LAQ down-regulates leukocyte extravasation in RRMS patients. LAQ can inhibit infiltration of inflammatory cells to the CNS by direct suppression of genes associated with leukocyte extravasation or via suppression of TGFb superfamily and inflammatory cytokines.

FIG. 9: FIG. 9A shows LAQ treatment for six months in RRMS patients down-regulates multiple genes associated with TGFb and NFkB signaling, pro inflammatory cytokines, cell adhesion and migration. Shaded color represents down regulated genes. FIG. 9B shows LAQ effects in RRMS patients after six months of treatment demonstrate down-regulation of multiple genes associated with TGFb and NFkB signaling, pro inflammatory cytokines, cell adhesion and migration. Grey color represents down regulated genes and white color depicts genes with no change in their expression level.

FIG. 10: Expression of TGFb, ITGB1 and CXCR1 in RRMS patients treated with LAQ. Protein extracts were prepared from PBMCs samples derived from RRMS patients before treatment (black bars) and compared to PBMCs samples of the same patients after six months of LAQ treatment (white bars). The signal intensity of a protein bands were quantified by Quantity One 4.6.9 software. The resultant background-subtracted values of protein expression were normalized to those of Tubulin and then calculated as the relative protein levels for each patient before or after LAQ treatment. The blot images in A, B and C are representative of two out of five analyzed patients showing down regulation of TGFb, ITGB1 and CXCR1, respectively as also quantified by densitometry of bands analyzed from five patients. Data are presented as mean±SEM. Statistically significant differences are marked in graphs (n=5, paired one-tailed t-test).

### DETAILED DESCRIPTION OF THE INVENTION

The subject invention provides a method of predicting clinical responsiveness to laquinimod therapy in a subject afflicted with multiple sclerosis or presenting a clinically isolated syndrome, the method comprising evaluating expression of a biomarker in the subject, so as to thereby predict clinical responsiveness to laquinimod, wherein the biomarker is a gene associated with inflammatory response, a gene associated with cellular movement, a gene associated with cell signaling, a gene associated with cell development, a gene associated with hematological system, or a combination thereof.

In one embodiment of the present invention, the method further comprises predicting positive clinical responsiveness to laquinimod if the biomarker is up-regulated in the subject. In another embodiment, the subject is naïve to laquinimod.

In another embodiment of the present invention, the method further comprises predicting positive clinical responsiveness to laquinimod if the biomarker suppressed in the subject. In another embodiment, the subject has previously received periodic laquinimod administration. In another embodiment, the expression of the biomarker is suppressed in comparison to expression of said biomarker of the patient at baseline.

In one embodiment, the subject has received periodic laquinimod administration for at least one month. In another embodiment, the subject has received periodic laquinimod administration for at least 6 months. In another embodiment, the subject has received periodic laquinimod administration for at least 12 months. In another embodiment, the subject has received periodic laquinimod administration for at least 24 months.

In one embodiment, the gene associated with inflammatory response is a gene associated with or involved in TGFb signaling, IL-12 signaling, the pathway of adhesion of phagocytes, chemotaxis of neutrophils, transmigration of leukocytes, caveolar mediated endocytosis, clathrin mediated endocytosis, and/or leukocyte extravasation signaling.

In another embodiment, the gene associated with cellular movement is a gene associated with or involved in adhesion and migration of phagocytes, chemotaxis of neutrophils, transmigration of leukocytes, invasion of cells, adhesion of cells, and/or leukocyte extravasation signaling.

In another embodiment, the gene associated with cell signaling is a gene associated with or involved in the pathway of adhesion of cells and/or neurotransmission.

In another embodiment, the gene associated with cell development is a gene associated with or involved in the pathway of G protein coupled receptor signaling, arachidonic acid metabolism and/or TGFβ signaling.

In another embodiment, the gene associated with hematological system is a gene associated with or involved in the pathway of aggregation of blood platelets, activation of blood platelets, aggregation of blood cells, coagulation of blood, intrinsic prothrombin activation pathway and/or coagulation system.

In another embodiment, the gene is TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1 or a combination thereof.

In one embodiment of any one of the methods, uses, pharmaceutical composition or packages described herein, the gene is ITGB1/3/5, CXCL5/7, BMP6, ITGA2/8, ITGB1/3/4/5/6, ITGBL1, MMP16/24/26/28, ADAM12/18/22, IL-5/20/22, IL-9/36, TNFRSF11A/B, TGβ, LTBP4, MEK1/2, Smad2/3/4, PAI-1, SELP, ITFA8, ITGB1/3/5, CXCL5/7, BMP6, ITGA2/8, ITGB1/3/4/5/6, ITGBL1, MMP16/24/26/28, ADAM12/18/22, IL-5/13/20/22, IL-9/11/36, TNFRSF11A/B, TGβ, LTBP4, MEK1/2, Smad1/2/3/4/5/6/8, PAI-1, CCL19, IKKg, LTBP1, Alpha tubulin, BMP4/7, MIS, TCF2, IL5R, IL13R, IL20R, ITGB2, NKTR, TEF, CLSTN2, LUC7L2, FABP7, TPTE, FSTL1, SF3B1, LIMS1, PDE5A, XPNPEP1, C5orf4, SPANXB1, SPANXB2, SPANXF1, KRT20, TBC1D1, GRHL2, C5orf4, SEPT6, KIAA1199, SSX2IP, TPM1, CDC14B, USP47, MMRN1, CTNNAL1, SMOX, ALOX12, GLRA3, CA2, GUCY1B3, RFPL1, CLEC1B, GNG11, TSPAN32, RGS10, CALD1, PRKAR2B, CYP4F11, CLCA3P, CELSR3, CDC14B, TPM1, SEPT6, PRKG1, MAX, CCDC93, ARMCX6, LOC653354, TUBB2B, HIST1H2AJ, MFAP3L, LIMS1, GNB5, GPRASP1, SRRT, C1orf116, FBXO7, PPM1A, GUCY1B3, CTDSPL, GNAS, IGF2BP3, TPM1, HIST1H2BK, DLG4, WDR48, CALD1, LOC157627, GNB5, ZNF415, ASAP2, PSD3, GNAS, POPDC3, NRGN, ABLIM3, XYLT1, PTGIS, ARHGEF10, PDGFA, PGRMC1, HIST1H2AC, GNAS, CLDN5, MFAP3L, PGRMC1, MYST3, CAPRIN1, CALD1, FBXW7, DNM3, CD84, PRPF4B, RBM25, WASF3, GRAP2, SPARC, TAL1, NENF, XK, GP1BA, HLA-E, CA5A, LYVE1, MARCH6, NAT8B, TRIM58, RET, SDPR, TBXA2R, TMED10, APBA2, MYL9, POU1F1, H2BFS, HIST1H2BK, FAM12B, VCL, GSPT1, ALDOB, LOC150776, SMPD4, SLC37A1, SPARC, GNAS, TAS2R4, CALM3, POM121, POM121C, GRIK2, GREM1, TNNC2, EPS15L2, ENDOD1, RGS6, SF3B1, TMSB15A, ZBTB20, FUT9, ATP9A, MAX, HIST1H2AI, BAT2D1, ABL1, SNCA, GFI1B, CTSA, SNX13, RPA1, FLNA, XPNPEP1, KIF2A, ZBTB33, PSMD11, UBE2N, FOLR1, TSC22D1, PCNP, CELSR3, ACSBG1, RNF11, SEMA3E, MARCH2, PCDH24, SUPT5H, HLA-E, EGF, HLA-C, FLNA, CDK2AP1, LEPROT, SH3TC2, TUBA4A, MTMR1, TF, PRKD1, NAPIL1, DAB2, FUCA1, HIP1, THPO, MAP1B, PARVB, GP1BB, SEPT5, GJA4, PTGS1, GUCY1A3, HIST1H2AG, GNAS, LRBA, HYAL3, GP6, IGHG1, CYP2A13, CDC14B, MAX, KDM2A, CALD1, GNAZ, C19orf22, ARHGAP6, RHOC, RBX1, GP1BB, SEPT5, PRDX6, PRB4, FLNA, HIST1H2BF, RHBDF2, NUP205, SYT1, EGFL8, PPT2, TUBB1, TMC6, FLJ11292, NAP1L1, ALDH1A3, CSNK1E, PRUNE, COL4A3, ZNF221, ILF3, CABP5, RPA1, ARF1, HIST1H2BI, PTGS1, PRKAA1, GNB5, HIST2H4A, HIST2H4B, CYB5R3, TNS1, DCT, GMPR, ABI3BP, GNAS, SASH1, AAK1, XPO6, CTSL2, QSER1, MAP1LC3B, TBX6, CABP2, MRE11A, MAPRE2, TMC6, BDKRB2, MGLL, HRASLS, WHAMML1, WHAMML2, CLU, STC1, C6orf54, PABPN1, PDLIM1, CLU, PHF20, UBL4A, RNF115, HGD, RASGRP2, PNN, SAPS3, SFI1, GOLGA2, HIST2H2BE, SGEF, HGD, DUS1L, MPP1, HLA-E, GRB14, MMD, ZFHX4, CSNK1G2, HIST1H2BE, MPDZ, B2M, TBXA2R, CTDSPL, SNCA, CD99, POLS, MPL, HIST1H3F, SFRS8, NR5A2, ZMYM2, C6orf10, TMEM40, RNF43, PRUNE, MSH6, PLCB4, PARVB, TOX3, PKNOX1, RUFY1, SNCA, C10orfB1, PDGFA, ASMT, HMGB1, CCDC90A, PROS1, hCG_1757335, RAP1B, MTSS1, GNRHR, LRRN3, MCM3AP, PLOD2, NAP1L1, PLOD2, HOXD13 CASKIN2, MFAP5, PITX2, SNCA, MYLK, PBX1, PRDX6, H3F3A, H3F3B, LOC440926, TMEM158, TRIM58, FSTL1, SNCA, TNS1, ATP1B1, C5orf4, LRP12, CTNNAL1, GEM, KIAA1466, ALDH1A2, MAP4K3, SNCA, RAB6B, PSD3, RIPK2, RAMP3, CALD1, CYP2E1, PSD3, PDLIM7, COBLL1, FUT3, SMOX, TGM2, LRRC50, CST6, OR7A17, C6orf145, DLEU2, DLEU2L, CPT2, HGF, TNS1, SPRY1, PLOD2, CD80, KYNU, BCAT1, NHLH1, AHCTF1, HOXA10, MTMR3, VAC14, CLCF1, FGF5, TAL1, SAMD14, ELL2, CHN1, SLC7A1, GRK5, PARD3, VPS37B, CYP2B6, CYP2B7P1, MALL, ALX4, SOX15, KRT5, ESPL1, STARD8, PSD3, KIAA0195, MYO9B, HIP1R, LOC100294412, EFNB1, ERN1, RHD, MFAP3L, PLA1A, POFUT2, C8orf39, CRYBB2, CYP4A11, PVRL2, CLCNKB, MRAS, NFIB, FKSG2, SLC11A2, FZR1, ZNF550, GLP1R, SLC19A1, RTN2, PAPOLA, STC1, GK, EXOSC6, RAPSN, HFE, EHD2, RIOK3, UBE2I, C15orf2, DMD, PRLH, MAP2K2, TP63, DACH1, PPP5C, SLC26A1, NUDT7, KCNJ12, ENTPD7, SLC26A1, PRRG3, RGS6, ZBED2, FICD, ARHGAP1, ARHGDIA, SDHB, AMHR2, ABCA4, TCF20, BGN, CASP7, LPAR4, GNA12, CYP2W1, RAX, C4A, C4B, LOC100292046, LOC100294156, PXN, ESR2, MYL10, EFS, TFF3, SRPK1, LOC441601, BIRC5, CCT8L2, PPAP2B, CMA1, APOA2, KDELR2, ASCL3, RUNX1, BUB1, SLC6A8, HNRNPC, HNRNPCL1, LOC440563, LOC649330, RIBC2, CLIC4, RAB17, SCML2, SPINLW1, ANK1, EDA2R, HTR4, CDC42EP4, KANK2, ANK1, SYN1, DUX3, DUX4, FRG2C, HPX-2, LOC100134409, LOC652119, LOC653543, LOC653544, LOC653545, LOC728410, PKNOX2, MLLT4, APOA2, PENK, GNAT1, FURIN, SEMA6A, EGFL6, HRH1, TSPAN1, DBC1, TRPC7, GPR52, HAMP, PRSS2, GPR107, FLJ11292, FLJ20184, B4GALT1, NKX3-1, ASIP, EFCAB6, GPR20, CA5A, PLK4, TAAR5, SRPX2, CNTD2, AZGP1, TIMP3, RGS6, ADARB1, DYNC1I1, C10orf10, PDIA2, PITX3, HOXC13, LPAR3, CTRC, CTSL2, MUC8, AQP5, UGT1A1, UGT1A10, UGT1A4, UGT1A6, UGT1A8, UGT1A9, KCNQ2, CYP2A13, ZNF155, KIAA0892, ATP2A2, FGF5, FGF18, FUT2, SHROOM2, PRSS3, CREB3L1, MGAT2, PLCE1, MLXIPL, OR10H3, ABCB11, CD84, ARHGEF4, ORC1L, PCIF1, CD177, C1orf116, IFT122, C11orf20, DUSP13, C6orf208, PLA2G5, PRAMEF1, PRAMEF2, CYP4F8, KCNA1, MFAP4, SLC4A3, IL1RAPL1, SERPINE1, ZCCHC14, POLR3G, C16orf68, FLJ14100, SMCHD1, ASCL1, FOXA2, SLC23A2, KLK13, MTSS1L, DNMT3L, RREB1, DNMBP, PKLR, C1orf106, CCDC134, MTSS1, CCDC40, HOXB1, SCNN1B, SEMA4G, RAPGEFL1, MAGEL2, PLSCR2, CHD2, PLCD1, C1orf116, CHRNA2, MBP, CDC42BPA, MYF6, PI15, LOC440895, SBF1, MAST1, GLT8D2, ERBB3, LOH3CR2A, AMH, HR, RDH8, PAWR, DRD3, CCT8, PRELP, SPOCK3, EPS8L3, NXN, SEMA4G, P2RY1, AVL9, TEK, MOGAT2, KLK7, MT1E, MT1H, MT1M, CLDN18, RHBDF2, SIX1, INPP5A, KCNMB3, MAP2K5, GPD1, LPO, LOC729143, MPRIP, WNT7A, RARG, CDH7, MBNL2, RASGRP2, RBMY2FP, MASP1, CASR, EGR4, APOC2, HECW1, HOXB3, IRF5, NNMT, AOC2, ESRRG, LPIN1, ACOT11, CCDC33, MBD2, ZNF323, NTRK2, TMEM151B, GPLD1, LENEP, HNF1B, NXPH3, ALDH1A3, PHF20L1, CKM, PARD6B, CRYGB, HAB1, LARGE, RAB40C, MPL, CHIT1, METTL10, DUS4L, PNLIPRP1, ELL, ST8SIA5, GRIN2B, MC4R, RTDR1, HDAC6, KCNJ13, CPSF1, SPANXC, CNOT4, LAMA2, SLC1A6, ABCA2, KLK11, GFRA3, CYP3A4, SLC1A3, ATP2B2, APBB2, VPS45, GHRHR, HOXD4, PRPH, ADCY2, LEFTY2, CYP1B1, PCP4, C8B, RANBP3, PDE6H, TRIM1S, VGLL1, TRIM3, CRKL, ADH7, PSG3, GPR153, MFAP2, FGF13, NAPA, ALDH3A1, MCM10, TLE4, ITPR3, CCDC87, C9orf7, ACTC1, OBSL1, MAP2, CRYM, RNF122, SST, HLA-DRB6, SLC22A17, HSPG2, HIP1, GRIK2, UNKL, GPR144, KIR3DX1, NARFL, UCP3, PLXNA2, BTN1A1, ERCC4, CIITA, EGFR, KRT33A, CLTB, B3GALT5, AP3M2, GJC1, MYO3A, ARHGAP1, PPP2R3A, CLIC4, C20orf195, SIGLEC8, GPRC5A, CACNB1, MYL10, PRLR, OR2S2, NCR2, CHAF1B, EYA3, CDS1, FBXL18, ACTL6B, ZNF821, C16orf71, HBBP1, PLXNA1, CDC45L, MTCP1, PLCB4, PLVAP, PROX1, CYP3A43, IGHG1, RECQL5, IDUA, DLGAP4, PLXNB1, HSD17B14, FOXP3, C19orf26, EPB41L1, RBBP9, GJB4, UPK1B, CYP19A1, LOC55908, CLDN18, C2orf72, NTRK3, NRXN2, SPDEF, IGH@, IGHD, IGHG1, IGHM, LOC100289944, VSIG6, ACRV1, PHLDB1, SORBS1, HAPLN2, FABP3, EFS, ACVR1B, CHST3, UGT2A1, UGT2A2, TAF1, MT4, MFAP3, ETV5, UBQLN3, TBX10, GJB1, ABO, SPINK5, ATAD4, CDH11, CARD14, ALPP, ALPPL2, CBL, LRP4, CDKL2, SSX3, DSG2, SLC45A2, LAMA4, WFDC8, HTR7, EFNB3, TUBB2B, OR7E19P, PMS2L4, ASAP3, FRZB, PDLIM4, PVT1, TFR2, AHI1, TAF4, ADAMTSL2, CLDN4, KIR2DL1, KIR2DL2, KIR2DL3, KIR2DL5A, KIR2DL5B, KIR2DS1, KIR2DS2, KIR2DS4, KIR2DS5, KIR3DL2, KIR3DL3, KIR3DP1, LOC727787, RAPGEF5, CRMP1, LDB3, F11, USP46, IBSP, SLC9A3, FLRT3, TRIM17, FGF17, CAMK1G, GLYR1, CSH1, NTF3, ABHD6, TRIM15, OR52A1, FGFR2, ORAI2, C17orf53, GLP1R, SLIT1, TP63, DDR1, CFTR, DIO2, LETM1, ACSM5, ACTA1, NPR1, KCND3, POPDC3, DNAH3, SPDEF, CLEC4M, SLC30A3, NAGLU, AAK1, DHX34, NNAT, AKAP9, ICMT, FAM189A1, C10orf81, MYOZ1, PKNOX2, MGC31957, PRDM11, RET, IGHG1, XPNPEP2, NTRK2, SLC25A10, NR1I2, GRM8, OR3A3, GIPR, PAH, PACRG, CLN8, ZNF215, TRIO, TTLL5, GRM1, PRKG1, HHLA1, LAMA3, SLC37A4, HOXC11, SLCO5A1, CA10, RRBP1, SOD3, NTRK3, CYR61, STRA6, SLC6A11, CNOT4, ATN1, BCAP29, NOVA2, RELN, LAMC2, RAD51, PRSS7, DCBLD2, TACR2, RAB11B, OR2J2, VSNL1, IFNA17, DPYSL4, MGC2889, RRBP1, POLQ, OR1A2, PURA, AIF1, CBS, NECAB2, PRKCE, NOX1, IHH, EXO1, GPRIN2, PDX1, GPR12, FAM188A, HS3ST3B1, ASCL1, ZNF484, CSH1, BCAN, DDN, DUOX2, MORN1, SLC39A2, CLCN7, RUNX2, TTYH1, ZNF280B, PAX3, LZTS1, SLC8A2, HAB1, KIF1A, ARL4D, UGT2B15, NACA2, THRB, C6orf15, GPR176, WSCD1, PLXNB3, CADM3, HAP1, CYP1A2, SPAM1, IL22RA1, CDC2L5, IRX5, PPFIA2, KDELR3, CEACAM7, KCMF1, DUOX1, CDC27, HIST2H2AA3, CAV3, APOA4, NPR3, PRG3, TBC1D22B, TUSC3, RIMS2, CYP4F12, TBXA2R, HBEGF, PSG9, PYGO1, RASGRF1, SCN2A, KLHL1, DTNB, GREM1, SNCG, C22orf24, PALM, COBLL1, DNPEP, MNS1, NFATC4, DLC1, HSPC072, MCAM, CA12, CSHL1, RPAIN, COL5A2, UGT1A8, UGT1A9, IGH@, IGHA1, IGHG1, IGHG2, IGHG3, IGHM, LOC100126583, LOC100290036, LOC100290320, LOC100293211, LOC652494, ACSM5, ALOX12P2, ERBB4, CLDN16, CIB2, GALR3, MSMB, FABP7, ATXN3, KCNJ5, TRDN, CYP3A43, BAZ2A, ACCN4, SILV, DGCR14, SEMA6C, DIO2, PTHLH, LEP, PDZRN3, RGSL1, GJA4, SLC22A6, RASGRF1, MAPRE2, PVRL1, AKAP1, POMP, SOX21, DNAH9, HOXC5, SERHL2, KIAA0485, ITSN1, B4GALT1, NEK2, NUPR1, CCDC93, EPO, CRABP2, TYRO3, GOLGA2, SEMA3F, BFSP2, NCAM1, FOLH1, SSX2, TMPRSS4, DCN, LPHN3, POU4F3, CEACAM5, BCL3, EXTL3, CCNA1, DDR2, PAX8, SOX5, POU3F1, PEX16, NUP62, SIGLEC11, ALDOB, GPC3, IGFALS, WDR25, FGF1, OSR2, ARID1A, GYPA, KLK13, PARVB, LILRB5, RIMS2, C19orf21, HOXD1, PRSS3, FLT1, ATP6V1C1, LOX, CRYBB3, CA12, PRKG2, MASP1, LOC728395, LOC728403, TSPY1, PDCD1, GGTLC1, AQP8, KRT16, AICDA, BRD8, C1orf95, OR3A2, PFKFB2, FRZB, PAK3, MEIS2, ZSCAN2, MYH7, VWA1, LSAMP, SRC, UGT1 A1, UGT1A10, UGT1A3, UGT1A4, UGT1A5, UGT1A6, UGT1A7, UGT1A8, UGT1A9, DIO1, TADA3L, NFASC, CALCRL, NBLA00301, MAB21L1, FBXO42, COL10A1, CFB, SNX7, FOXN1, SRY, HLF, CLCA3P, DAZ1, DAZ2, DAZ3, DAZ4, GPR3, TMPRSS11E, EMID1, KCNMB2, MUC5AC, SORT1, HIF3A, MAPK4, TCP11L1, ZZEF1, DCAF7, DMWD, CLCA2, VAC14, CSPG5, STMN2, MLLT4, GALNT14, FGF12, MFAP5, SUMO3, HTR3A, GDF5, TSSK1B, CYP2A7P1, MARK1, ATP1B2, TBX6, PAX8, IL1R1, RALYL, OR2B2, TAAR3, C12orf32, IGHG1, LOC642131, DICER1, GLRA3, PPARD, HSPA4L, WNT2, VIPR2, CYP2C9, SRPX2, IGSF1, ALPK3, TFPI, KCNS3, MARCH8, FRMD4B, TACR3, FIGF, PDCD6, TNN, SPANXB1, SPANXB2, SPANXF1, RHBDD3, SPP2, PDE10A, ZNF224, FGL1, PGAM2, CADM4, APOBEC2, SLC9A5, GNAT1, ARHGEF16, SMARCA2, DNAH9, RBM26, WNT2B, KCNK2, NPBWR2, SP2, TMPRSS11D, DENND2A, TNIP3, STC1, DOCK6, ADAM5P, SYDE1, TNPO2, LRTM1, USH1C, PDE12, SRCAP, OR10J1, OR2H2, KCNJ8, RP11-257K9.7, DOCK5, TPD52L1, PAEP, GGA2, PHLDA3, HES2, MLL, CHRNA6, CIB2, PTPRF, TM7SF4, DAZ1, DAZ2, DAZ3, DAZ4, ALX1, OR2F1, OR2F2, PLAT, HGC6.3, WNT11, PGK2, SNAI2, COL4A6, PRUNE2, ANKS1B, LOC81691, FERMT2, TIMP3, CST8, CAPN6, IDUA, GPR32, AKR1B10, GRHL2, FBXO24, HSF4, IGHG1, HCN2, LRP12, ARHGEF15, UGT1A1, UGT1A10, UGT1A7, UGT1A8, GUCA2A, ITIH1, EGFR, UGT1A1, UGT1A10, UGT1A3, UGT1A4, UGT1A5, UGT1A6, UGT1A7, UGT1A8, UGT1A9, MYOG, TMSB15A, TLX1, EDNRA, LOC100289791, MDFI, ZER1, MYH15, CDH20, GPR63, LOC440345, LOC440354, LOC595101, LOC641298, SMG1, HOXC10, KRTAP1-1, ARSD, CPLX3, LMAN1 L, IFNA4, ABCC1, SEMA3E, MRE11A, C1QL1, LIPF, TRIM9, BBOX1, LRRC17, WNT2B, CYP3A4, SI, ANO3, OBSL1, CHRD, MSX2, PSG1, FAM107A, LRRC37B2, ANKLE2, PAX2, UNC5B, ADCYAP1R1, HFE, SYT1, GJC2, LOC100293871, FGF8, ACRV1, NRXN1, GDPD2, RGS4, CELA2A, IFNW1, MLNR, RNF17, LAD1, GLRA2, RASL12, MAGOH2, C6orf54, ZNF214, IKBKG, AP4E1, ZNRF4, OSBPL10, C1orf175, TTC4, PCDHB3, ADRBK1, ITSN1, XAGE1A, XAGE1B, XAGE1C, XAGE1D, XAGE1E, CDH22, FARP2, MYT1, TNC, MUC5AC, SLC6A15, PP14571, SMR3A, SMR3B, RXRG, SNX1, GLP1R, C6orf155, ATP1A2, TFAP4, PNPLA2, DIRAS3, ANO2, TACSTD2, MCM3AP, IL13RA2, TRIM10, RTEL1, PRRX2, TSHB, TIMELESS, FMO1, KIF18A, KIAA1199, CALB2, MFAP3L, PTGER3, EPAS1, SQSTM1, TSPY1, CPM, DLGAP1, CYP4F11, TLX3, PCDHA10, TAOK2, ERC1, TBX2, KALRN, DICER1, PAPPA, KIF5A, DNAJC22, OTUB1, KIAA1644, SEZ6L2, PCNXL2, HMHB1, ERG, SNTB2, GJA5, AGTR2, GJA3, GCK, LRRC61, CNTF, ZFP91, ZFP91-CNTF, PDLIM4, MPPED2, IFNA10, ACTN2, VGLL1, GJA9, LDLR, ANK2, COL1A1, TIMP3, OTOF, AGXT, GLI2, TRMT61A, FOXD2, TMEM212, DENND2A, B3GALT1, SPAG11A, PRDM4, TF, ELF5, GSC2, EPB41 L4B, GYG2, LYZL6, DCHS2, OBP2A, OBP2B, ANGPTL3, MYH11, NES, SLC17A1, RBM15B, CSH1, HTR5A, CYP3A7, HTR2A, KCNV2, TOX3, CLOCK, MAGEA6, FAM12A, COL4A3, S1PR2, NAT8, ACE2, SLC22A6, SLC13A2, MYH4, APBB2, RAP1QGAP, SHOX2, SLCO1A2, ETV1, MAGEA12, PLA2G6, ADRA1A, SYT5, GPR161, SEMA3F, CYP3A43, HOMER2, KCNJ5, PPL, COL17A1, CSHL1, C9orf116, PARK2, UGT2B15, CDK6, FAM174B, CELA2A, CELA2B, SPDEF, EPB41, GAB1, SMR3A, PDE60, COL5A1, ABCA6, DMD, CYLC2, CIDEA, RAG2, HIST1H2BN, FMO6P, MAOA, ANKRD53, HAPLN1, MT1M, EHD2, GAD2, CRISP2, CSN2, SULT1C2, PCDHGA3, SSX3, FGFR2, GPR161, ATN1, CHD5, A4GALT, MYBPH, CSHL1, NCAPH2, CAPN9, CNGB1, BCAM, DRD5, NR5A2, TEF, ELAVL2, DGKB, HTR7P, RHAG, GH2, COL4A6, BMP7, SOSTDC1, SOX14, TAS2R9, LPHN2, MAP1A, OSGIN2, SLC10A2, FAM13C, EMX1, FLJ40330, CHI3L1, CDH16, SPRR1A, LOX, CALCB, GABBR2, CPB2, RASL11B, CCDC81, RUNX1, CPA1, CLCNKA, CLCNKB, FHL5, THSD7A, TFAP2C, SPAG11B, CAP2, PODNL1, SSX4, SSX4B, G6PC, RPE65, TMEM222, KDR, CHP2, GPR64, TPM2, TCEB3B, E2F5, IL5RA, AOC3, ABCF3, CPN2, ACE, NRP2, INPP5J, SMAD9, FAM155A, GART, PIR, ZNF467, ITSN2, NR1D1, THRA, RP11-35N6.1, LAMB1, EPHB3, PLA2R1, RAPGEF4, DNAJC8, ARSJ, TRIM49, GC, CDH2, ATXN3L, BTF3L1, BICC1, FAM186A, PTPRF, TRPC4, TCL6, CYP4A22, FUT6, MUC1, DKFZP434B2016, LOC643313, LDHA, LOC100131613, TRIM3, MLLT10, DZIP1, ANKRD34C, BUB1, CSPG5, FBLN1, GAD2, CLDN1, CHRNA3, SCN11A, TEX11, IL20RA, AKAP5, KBTBD10, MSTN, TLL2, NACAD, UNC93A, PTGER1, OLAH, NHLH2, SERPINA6, KRT17, KCNMA1, PRKCA, STS, LAMA1, GPR88, ACTN2, TREH, AKAP4, DKK4, PRICKLE3, IRS4, TRPV4, PCDH11Y, APBB2, SLCO2A1, DRD2, MTMR7, ZNF471, TF, NRIP2, ST6GALNAC5, COMT, PAH, LRRC19, PRKAR1B, HPR, PRDM5, NCRNA00120, LOC79999, ITSN2, CACNB2, GPR98, PREX2, FAM182B, LAMA4, ARVCF, HAS2, YOD1, PPP2R3A, COL4A1, RBM12B, GSTA3, FAM66D, OR10H2, PTHLH, ZNF674, KRT19, ACCN2, COL6A1, LOC100288442, LOC100289169, LOC728888, LOC729602, NPIPL2, NPIPL3, PDXDC2, SLC37A1, ATP6V1B1, ABI3BP, HR44, ZNF324B, ZNF584, HOXD13, ADH6, IFNA8, MYOZ2, NFATC4, ADAMTS7, FOXL1, GPR17, SLC18A3, MYH6, BOK, FGA, TEAD4, GRM1, EDNRA, C8orf79, METTL7A, FOLH1, RAD54L, SOX11, CNOT3, NTS, MAPK12, DOCK6, DNAJC6, HS3ST3A1, LOC728395, TSPY1, TSPY3, PTH, LAMB4, ALDOB, FLG, MLANA, UBE2D4, LOC100287483, KRT20, POU1F1, SLCO1B3, CLTA, MECOM, C8orf71, SULT2A1, C6orf10, SLC27A6, PRKD1, SYNPO2L, THPO, GABRR1, CFTR, PPP2R3A, DCBLD2, ANP32A, ANP32C, ANP32D, LOC723972, XYLT1, STAB1, STAB1, SASH1, PID1, FUCA1, SASH1, LRRN3, LRRN3 or a combination thereof.

In another embodiment, the gene is ITGB1/3/5, CXCL5/7, BMP6, ITGA2/8, ITGB1/3/4/5/6, ITGBL1, MMP16/24/26/28, ADAM12/18/22, IL-5/20/22, IL-9/36, TNFRSF11A/B, TGβ, LTBP4, MEK1/2, Smad2/3/4, PAI-1, SELP, ITFA8, ITGB1/3/5, CXCL5/7, BMP6, ITGA2/8, ITGB1/3/4/5/6, ITGBL1, MMP16/24/26/28, ADAM12/18/22, IL-5/13/20/22, IL-9/11/36, TNFRSF11A/B, TGβ, LTBP4, MEK1/2, Smad1/2/3/4/5/6/8, PAI-1, CCL19, IKKg, LTBP1 or a combination thereof. In another embodiment, the gene is SELP, ITFA8, ITGB1/3/5, CXCL5/7, BMP6, ITGA2/8, ITGB1/3/4/5/6, ITGBL1, MMP16/24/26/28, ADAM12/18/22, IL-1/1R/5/8/13/20/22, IL-9/11/12/36, TNFRSF11A/B, IFNA4/8/10/17, TGβ, LTBP4, MEK1/2, Smad1/2/3/4/5/6/8, PAI-1, CCL19, IKKg, LTBP1 or a combination thereof.

In one embodiment of any one of the methods, uses, pharmaceutical composition or packages described herein, the gene is TNFSF4, ITGB1/3/5, CXCL5/7, BMP6, ITGA2/8, ITGB1/3/4/5/6, ITGBL1, MMP16/24/26/28, ADAM12/18/22, IL-1/5/8/20/22, IL-9/12/36, TNFRSF11A/B, IFNA4/8/10/17, TGβ, LTBP4, MEK1/2, TGFβ type 1 receptor, Smad2/3/4, PAI-1, TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1, IL8R (CXCR1/2), Alpha tubulin, BMP2/4/7, MIS, TCF2, LFA-1, VLA-4, IL5R, IL13R, IL20R, ITGB2, IFN gamma, TNF alpha, NKTR, TEF, CLSTN2, LUC7L2, FABP7, TPTE, FSTL1, SF31B1, LIMS1, PDE5A, XPNPEP1, C5orf4, SPANXB1, SPANXB2, SPANXF1, KRT20, TBC1D1, GRHL2, C5orf4, SEPT6, KIAA1199, SSX2IP, TPM1, CDC14B, USP47, MMRN1, CTNNAL1, SMOX, ALOX12, GLRA3, CA2, GUCY1B3, RFPL1, CLEC1B, GNG11, TSPAN32, RGS10, CALD1, PRKAR2B, CYP4F11, CLCA3P, CELSR3, CDC14B, TPM1, SEPT6, PRKG1, MAX, CCDC93, ARMCX6, LOC653354, TUBB2B, HIST1H2AJ, MFAP3L, LIMS1, GNB5, GPRASP1, SRRT, C1orf116, FBXO7, PPM1A, GUCY1B3, CTDSPL, GNAS, IGF2BP3, TPM1, HIST1H2BK, DLG4, WDR48, CALD1, LOC157627, GNB5, ZNF415, ASAP2, PSD3, GNAS, POPDC3, NRGN, ABLIM3, XYLT1, PTGIS, ARHGEF10, PDGFA, PGRMC1, HIST1H2AC, GNAS, CLDN5, MFAP3L, PGRMC1, MYST3, CAPRIN1, CALD1, FBXW7, DNM3, CD84, PRPF4B, RBM25, WASF3, GRAP2, SPARC, TAL1, NENF, XK, GP1BA, HLA-E, CA5A, LYVE1, MARCH6, NAT8B, TRIM58, RET, SDPR, TBXA2R, TMED10, APBA2, MYL9, POU1F1, H2BFS, HIST1H2BK, FAM12B, VCL, GSPT1, ALDOB, LOC150776, SMPD4, SLC37A1, SPARC, GNAS, TAS2R4, CALM3, POM121, POM121C, GRIK2, GREM1, TNNC2, EPS15L2, ENDOD1, RGS6, SF3B1, TMSB15A, ZBTB20, FUT9, ATP9A, MAX, HIST1H2AI, BAT2D1, ABL1, SNCA, GFI1B, CTSA, SNX13, RPA1, FLNA, XPNPEP1, KIF2A, ZBTB33, PSMD11, UBE2N, FOLR1, TSC22D1, PCNP, CELSR3, ACSBG1, RNF11, SEMA3E, MARCH2, PCDH24, SUPT5H, HLA-E, EGF, HLA-C, FLNA, CDK2AP1, LEPROT, SH3TC2, TUBA4A, MTMR1, TF, PRKD1, NAP1L1, DAB2, FUCA1, HIP1, THPO, MAP1B, PARVB, GP1BB, SEPT5, GJA4, PTGS1, GUCY1A3, HIST1H2AG, GNAS, LRBA, HYAL3, GP6, IGHG1, CYP2A13, CDC14B, MAX, KDM2A, CALD1, GNAZ, C19orf22, ARHGAP6, RHOC, RBX1, GP1BB, SEPT5, PRDX6, PRB4, FLNA, HIST1H2BF, RHBDF2, NUP205, SYT1, EGFL8, PPT2, TUBB1, TMC6, FLJ11292, NAP1L1, ALDH1A3, CSNK1E, PRUNE, COL4A3, ZNF221, ILF3, CABP5, RPA1, ARF1, HIST1H2BI, PTGS1, PRKAA1, GNB5, HIST2H4A, HIST2H4B, CYB5R3, TNS1, DCT, GMPR, ABI3BP, GNAS, SASH1, AAK1, XPO6, CTSL2, QSER1, MAP1LC3B, TBX6, CABP2, MRE11A, MAPRE2, TMC6, BDKRB2, MGLL, HRASLS, WHAMML1, WHAMML2, CLU, STC1, C6orf54, PABPN1, PDLIM1, CLU, PHF20, UBL4A, RNF115, HGD, RASGRP2, PNN, SAPS3, SFI1, GOLGA2, HIST2H2BE, SGEF, HGD, DUS1L, MPP1, HLA-E, GRB14, MMD, ZFHX4, CSNK1G2, HIST1H2BE, MPDZ, B2M, TBXA2R, NGFRAP1, CTDSPL, SNCA, CD99, POLS, MPL, HIST1H3F, SFRS8, NR5A2, ZMYM2, C6orf10, TMEM40, RNF43, PRUNE, MSH6, PLCB4, PARVB, TOX3, PKNOX1, RUFY1, SNCA, C10orf81, PDGFA, ASMT, HMGB1, CCDC90A, PROS1, hCG_1757335, RAP1B, MTSS1, GNRHR, LRRN3, MCM3AP, PLOD2, NAPIL1, PLOD2, HOXD13 CASKIN2, MFAP5, PITX2, SNCA, MYLK, PBX1, PRDX6, EIF2AK1, H3F3A, H3F3B, LOC440926, TMEM158, TRIM58, FSTL1, SNCA, TNS1, ATP1B1, C5orf4, LRP12, CTNNAL1, GEM, KIAA1466, ALDH1A2, MAP4K3, SNCA, RAB6B, PSD3, RIPK2, RAMP3, CALD1, CYP2E1, PSD3, PDLIM7, COBLL1, FUT3, SMOX, TGM2, LRRC50, CST6, OR7A17, C6orf145, DLEU2, DLEU2L, CPT2, HGF, TNS1, SPRY1, PLOD2, CD80, KYNU, BCAT1, NHLH1, AHCTF1, HOXA10, MTMR3, VAC14, CLCF1, FGF5, TAL1, SAMD14, ELL2, CHN1, SLC7A1, GRK5, PARD3, VPS37B, CYP2B6, CYP2B7P1, MALL, ALX4, SOX15, KRT5, ESPL1, STARD8, PSD3, KIAA0195, MYO9B, HIP1R, LOC100294412, EFNB1, ERN1, RHD, MFAP3L, PLA1A, POFUT2, C8orf39, CRYBB2, CYP4A11, PVRL2, CLCNKB, MRAS, NFIB, FKSG2, SLC11A2, FZR1, ZNF550, GLP1R, SLC19A1, RTN2, PAPOLA, STC1, GK, EXOSC6, RAPSN, HFE, EHD2, RIOK3, UBE2I, C15orf2, DMD, PRLH, MAP2K2, TP63, DACH1, PPP5C, SLC26A1, NUDT7, KCNJ12, ENTPD7, SLC26A1, PRRG3, RGS6, ZBED2, FICD, ARHGAP1, ARHGDIA, SDHB, AMHR2, ABCA4, TCF20, BGN, CASP7, LPAR4, GNA12, CYP2W1, RAX, C4A, C4B, LOC100292046, LOC100294156, ELAVL4, PXN, ESR2, MYL10, EFS, TFF3, SRPK1, LOC441601, BIRC5, CCT8L2, PPAP2B, CMA1, APOA2, KDELR2, ASCL3, RUNX1, BUB1, SLC6A8, HNRNPC, HNRNPCL1, LOC440563, LOC649330, RIBC2, CLIC4, RAB17, SCML2, SPINLW1, ANK1, EDA2R, HTR4, CDC42EP4, KANK2, ANK1, SYN1, DUX3, DUX4, FRG2C, HPX-2, LOC100134409, LOC652119, LOC653543, LOC653544, LOC653545, LOC728410, PKNOX2, MLLT4, APOA2, PENK, GNAT1, FURIN, SEMA6A, EGFL6, HRH1, TSPAN1, DBC1, TRPC7, MDM2, GPR52, HAMP, PRSS2, GPR107, FLJ11292, FLJ20184, B4GALT1, NKX3-1, ASIP, EFCAB6, GPR20, CA5A, PLK4, TAAR5, SRPX2, CNTD2, AZGP1, TIMP3, RGS6, ADARB1, DYNC1I1, C10orf10, PDIA2, PITX3, HOXC13, LPAR3, CTRC, CTSL2, MUC8, AQP5, UGT1A1, UGT1A10, UGT1A4, UGT1A6, UGT1A8, UGT1A9, KCNQ2, CYP2A13, ZNF155, KIAA0892, ATP2A2, FGF5, FGF18, FUT2, SHROOM2, PRSS3, CREB3L1, MGAT2, PLCE1, MLXIPL, OR10H3, ABCB11, CD84, ARHGEF4, ORC1L, PCIF1, CD177, C1orf116, IFT122, C11orf20, DUSP13, C6orf208, PLA2G5, PRAMEF1, PRAMEF2, CYP4F8, KCNA1, MFAP4, C6, SLC4A3, IL1RAPL1, SERPINE1, ZCCHC14, POLR3G, C16orf8, FLJ14100, SMCHD1, ASCL1, FOXA2, SLC23A2, KLK13, MTSS1L, DNMT3L, RREB1, DNMBP, PKLR, C1orf106, CCDC134, MTSS11, CCDC40, HOXB1, SCNN1B, SEMA4G, RAPGEFL1, MAGEL2, PLSCR2, CHD2, PLCD1, C1orf116, CHRNA2, MBP, CDC42BPA, MYF6, PI15, LOC440895, SBF1, MAST1, GLT8D2, ERBB3, LOH3CR2A, AMH, HR, RDH8, PAWR, DRD3, CCT8, PRELP, SPOCK3, EPS8L3, NXN, SEMA4G, P2RY1, AVL9, TEK, MOGAT2, KLK7, MT1E, MT1H, MT1M, CLDN18, RHBDF2, SIX1, INPP5A, KCNMB3, MAP2K5, GPD1, LPO, LOC729143, MPRIP, WNT7A, RARG, CDH7, MBNL2, RASGRP2, RBMY2FP, MASP1, CASR, EGR4, APOC2, HECW1, HOXB3, IRF5, NNMT, AOC2, ESRRG, LPIN1, ACOT11, CCDC33, MBD2, ZNF323, NTRK2, TMEM151B, GPLD1, LENEP, HNF1B, NXPH3, ALDH1A3, PHF20L1, CKM, PARD6B, CRYGB, HAB1, LARGE, RAB40C, MPL, CHIT1, METTL10, DUS4L, PNLIPRP1, ELL, ST8SIA5, GRIN2B, MC4R, RTDR1, HDAC6, KCNJ13, CPSF1, SPANXC, CNOT4, LAMA2, SLC1A6, ABCA2, KLK11, GFRA3, CYP3A4, SLC1A3, ATP2B2, APBB2, VPS45, GHRHR, HOXD4, PRPH, ADCY2, LEFTY2, CYP1B1, PCP4, C8B, RANBP3, PDE6H, TRIM15, VGLL1, TRIM3, CRKL, ADH7, PSG3, GPR153, MFAP2, FGF13, NAPA, ALDH3A1, MCM10, TLE4, ITPR3, CCDC87, C9orf7, ACTC1, OBSL1, MAP2, CRYM, RNF122, SST, HLA-DRB6, SLC22A17, HSPG2, HIP1, GRIK2, UNKL, GPR144, KIR3DX1, NARFL, UCP3, PLXNA2, BTN1A1, ERCC4, CIITA, EGFR, KRT33A, CLTB, B3GALT5, AP3M2, GJC1, MYO3A, ARHGAP1, PPP2R3A, CLIC4, C20orf195, SIGLEC8, GPRC5A, CACNB1, MYL10, PRLR, OR2S2, NCR2, CHAF1B, EYA3, CDS1, FBXL18, ACTL6B, ZNF821, C16orf71, HBBP1, PLXNA1, CDC45L, MTCP1, PLCB4, PLVAP, PROX1, CYP3A43, IGHG1, RECQL5, IDUA, DLGAP4, PLXNB1, HSD17B14, FOXP3, C19orf26, EPB41L1, RBBP9, GJB4, UPK1B, CYP19A1, LOC55908, CLDN18, C2orf72, NTRK3, NRXN2, SPDEF, IGH@, IGHD, IGHG1, IGHM, LOC100289944, VSIG6, ACRV1, PHLDB1, SORBS1, HAPLN2, FABP3, EFS, ACVR1B, CHST3, UGT2A1, UGT2A2, TAF1, MT4, MFAP3, ETV5, UBQLN3, TBX10, GJB1, ABO, SPINK5, ATAD4, CDH11, CARD14, ALPP, ALPPL2, CBL, LRP4, CDKL2, SSX3, DSG2, SLC45A2, LAMA4, WFDC8, HTR7, EFNB3, TUBB2B, OR7E19P, PMS2L4, ASAP3, FRZB, PDLIM4, PVT1, TFR2, AHI1, TAF4, ADAMTSL2, CLDN4, KIR2DL1, KIR2DL2, KIR2DL3, KIR2DL5A, KIR2DL5B, KIR2DS1, KIR2DS2, KIR2DS3, KIR2DS4, KIR2DS5, KIR3DL2, KIR3DL3, KIR3DP1, LOC727787, RAPGEF5, CRMP1, LDB3, F11, USP46, PTN, IBSP, SLC9A3, FLRT3, TRIM17, FGF17, CAMK1G, GLYR1, CSH1, NTF3, ABHD6, TRIM15, OR52A1, FGFR2, ORAI2, C17orf53, GLP1R, SLIT1, TP63, DDR1, CFTR, DIO2, LETM1, ACSM5, ACTA1, NPR1, KCND3, POPDC3, DNAH3, SPDEF, CLEC4M, SLC30A3, NAGLU, AAK1, DHX34, NNAT, AKAP9, ICMT, FAM189A1, C10orf81, MYOZ1, PKNOX2, MGC31957, PRDM11, RET, IGHG1, XPNPEP2, NTRK2, SLC25A10, NR1I2, GRM8, OR3A3, GIPR, PAN, PACRG, CLN8, ZNF215, TRIO, TTLL5, GRM1, PRKG1, HHLA1, LAMA3, PTN, SLC37A4, HOXC11, SLCO5A1, CA10, RRBP1, SOD3, NTRK3, CYR61, STRA6, SLC6A11, CNOT4, ATN1, BCAP29, NOVA2, RELN, LAMC2, RAD51, PRSS7, DCBLD2, TACR2, RAB11B, OR2J2, VSNL1, IFNA17, DPYSL4, MGC2889, RRBP1, POLQ, OR1A2, PURA, AIF1, CBS, NECAB2, PRKCE, NOX1, INH, EXO1, GPRIN2, PDX1, GPR12, FAM188A, HS3ST3B1, ASCL1, ZNF484, CSH1, BCAN, DDN, DUOX2, MORN1, SLC39A2, CLCN7, RUNX2, TTYH1, ZNF280B, PAX3, LZTS1, SLC8A2, HAB1, KIF1A, ARL4D, UGT2B15, NACA2, THRB, C6orf15, GPR176, WSCD1, PLXNB3, CADM3, HAP1, CYP1A2, SPAM1, IL22RA1, CDC2L5, IRX5, PPFIA2, KDELR3, CEACAM7, KCMF1, DUOX1, CDC27, HIST2H2AA3, CAV3, APOA4, NPR3, PRG3, TBC1D22B, TUSC3, RIMS2, CYP4F12, TBXA2R, HBEGF. PSG9, PYGO1, RASGRF1, SCN2A, KLHL1, DTNB, GREM1, SNCG, C22orf24, PALM, COBLL1, DNPEP, MNS1, NFATC4, DLC1, HSPC072, MCAM, CA12, CSHL1, RPA1N, COL5A2. UGT1A8, UGT1A9, IGH@, IGHA1, IGHG1, IGHG2, IGHG3, IGHM, LOC100126583, LOC100290036, LOC100290320, LOC100293211, LOC652494, TGFB2, ACSM5, ALOX12P2, ERBB4, CLDN16, CIB2, GALR3, MSMB, FABP7, ATXN3, KCNJ5, TRDN, CYP3A43, BAZ2A, ACCN4, SILV, DGCR14, SEMA6C, DIO2, PTHLH, LEP, PDZRN3, RGSL1, GJA4, SLC22A6, RASGRF1, MAPRE2, PVRL1, AKAP1, POMP, SOX21, DNAH9, HOXC5, SERHL2, KIAA0485, ITSN1, B4GALT1, NEK2, NUPR1, CCDC93, EPO, CRABP2, TYRO3, GOLGA2, SEMA3F, BFSP2, NCAM1, FOLH1, SSX2, TMPRSS4, DCN, LPHN3, POU4F3, CEACAM5, BCL3, EXTL3, CCNA1, DDR2, PAX8, SOX5, POU3F1, PEX16, 1L4I1, NUP62, SIGLEC11, ALDOB, GPC3, IGFALS, WDR25, FGF1, OSR2, ARID1A, GYPA, KLK13, PARVB, LILRB5, RIMS2, C19orf21, HOXD1, PRSS3, FLT1, ATP6V1C1, LOX, CRYBB3, CA12, PRKG2, MASP1, LOC728395, LOC728403, TSPY1, PDCD1, GGTLC1, AQP8, IL1F9, KRT16, AICDA, BRD8, C1orf95, OR3A2, PFKFB2, FRZB, PAK3, MEIS2, ZSCAN2, MYH7, VWA1, LSAMP, SRC, UGT1A1, UGT1A10, UGT1A3, UGT1A4, UGT1A5, UGT1A6, UGT1A7, UGT1A8, UGT1A9, DIO1, TADA3L, NFASC, CALCRL, NBLA00301, MAB21L1, FBXO42, COL10A1, CFB, SNX7, FOXN1, SRY, HLF, CLCA3P, DAZ1, DAZ2, DAZ3, DAZ4, GPR3, TMPRSS11E, EMID1, KCNMB2, MUC5AC, SORT1, HIF3A, MAPK4, TCP11L1, ZZEF1, DCAF7, DMWD, CLCA2, VAC14, CSPG5, STMN2, MLLT4, GALNT14, FGF12, MFAP5, SUMO3, HTR3A, GDF5, TSSK1B, CYP2A7P1, MARK1, ATP1B2, TBX6, PAX8, IL1R1, RALYL, OR2B2, TAAR3, C12orf32, IGHG1, LOC642131, DICER1, GLRA3, PPARD, HSPA4L, WNT2, VIPR2, CYP2C9, SRPX2, IGSF1, ALPK3, TFPI, KCNS3, MARCH8, FRMD4B, TACR3, FIGF, PDCD6, TNN, SPANXB1, SPANXB2, SPANXF1, RHBDD3, SPP2, PDE10A, ZNF224, FGL1, PGAM2, CADM4, APOBEC2, SLC9A5, GNAT1, ARHGEF16, SMARCA2, DNAH9, RBM26, WNT2B, KCNK2, NPBWR2, SP2, TMPRSS11D, DENND2A, TNIP3, STC1, DOCK6, ADAM5P, SYDE1, TNPO2, LRTM1, USH1C, PDE12, SRCAP, OR10J1, OR2H2, KCNJ8, RP11-257K9.7, DOCK5, TPD52L1, PAEP, GGA2, PHLDA3, HES2, MLL, PTN, CHRNA6, CIB2, PTPRF, TM7SF4, DAZ1, DAZ2, DAZ3, DAZ4, ALX1, OR2F1, OR2F2, PLAT, HGC6.3, WNT11, PGK2, SNAI2, COL4A6, PRUNE2, ANKS1B, LOC81691, FERMT2, TIMP3, CST8, CAPN6, IDUA, GPR32, AKR1B10, GRHL2, FBXO24, HSF4, IGHG1, HCN2, LRP12, ARHGEF15, UGT1A1, UGT1A10, UGT1A7, UGT1A8, GUCA2A, MDK, ITIH1, EGFR, UGT1A1, UGT1A10, UGT1A3, UGT1A4, UGT1A5, UGT1A6, UGT1A7, UGT1A8, UGT1A9, MYOG, TMSB15A, TLX1, EDNRA, LOC100289791, MDFI, ZER1, MYH15, CDH20, GPR63, LOC440345, LOC440354, LOC595101, LOC641298, SMG1, HOXC10, KRTAP1, ARSD, CPLX3, LMAN1L, IFNA4, ABCC1, SEMA3E, MRE11A, C1QL1, LIPF, TRIM9, BBOX1, LRRC17, WNT2B, CYP3A4, SI, ANO3, OBSL1, CHRD, MSX2, PSG1, FAM107A, LRRC37B2, ANKLE2, PAX2, UNC5B, ADCYAP1R1, HFE, SYT1, GJC2, LOC100293871, FGF8, ACRV1, NRXN1, GDPD2, RGS4, CELA2A, IFNW1, MLNR, RNF17, LAD1, GLRA2, RASL12, MAGOH2, C6orf54, ZNF214, IKBKG, AP4E1, ZNRF4, OSBPL10, C1orf175, TTC4, PCDHB3, ADRBK1, ITSN1, XAGE1A, XAGE1B, XAGE1C, XAGE1D, XAGE1E, CDH22, FARP2, MYT1, TNC, MUC5AC, SLC6A15, PP14571, SMR3A, SMR3B, RXRG, SNX1, GLP1R, C6orf155, ATP1A2, TFAP4, PNPLA2, DIRAS3, ANO2, TACSTD2, MCM3AP, IL13RA2, TRIM10, RTEL1, PRRX2, TSHB, TIMELESS, FMO1, KIF18A, KIAA1199, CALB2, MFAP3L, PTGER3, EPAS1, SQSTM1, TSPY1, CPM, DLGAP1, CYP4F11, TLX3, PCDHA10, TAOK2, ERC1, TBX2, KALRN, DICER1, PAPPA, KIF5A, DNAJC22, OTUB1, KIAA1644, SEZ6L2, PCNXL2, HMHB1, ERG, SNTB2, GJA5, AGTR2, GJA3, GCK, LRRC61, CNTF, ZFP91, ZFP91-CNTF, PDLIM4, MPPED2, IFNA10, ACTN2, VGLL1, GJA9, LDLR, ANK2, COL1A1, TIMP3, OTOF, AGXT, GLI2, TRMT61A, FOXD2, TMEM212, DENND2A, B3GALT1, SPAG11A, PRDM4, TF, ELF5, GSC2, EPB41L4B, GYG2, LYZL6, DCHS2, OBP2A. OBP2B, ANGPTL3, MYH11, NES, SLC17A1, RBM15B, CSH1, HTR5A, CYP3A7, HTR2A, KCNV2, TOX3, CLOCK, MAGEA6, FAM12A, COL4A3, S1PR2, NAT8, ACE2, SLC22A6, SLC13A2, MYH4, APBB2, RAP1GAP, SHOX2, SLCO1A2, ETV1, MAGEA12, PLA2G6, ADRA1A, SYT5, GPR161, SEMA3F, CYP3A43, HOMER2, KCNJ5, PPL, COL17A1, CSHL1, C9orf116, PARK2, UGT2B15, CDK6, FAM174B, CELA2A, CELA2B, SPDEF, EPB41, GAB1, SMR3A, PDE6G, COL5A1, ABCA6, DMD, CYLC2, CIDEA, RAG2, HIST1H2BN, FMO6P, MAOA, ANKRD53, HAPLN1, MT1M, EHD2, GAD2, CRISP2, CSN2, SULT1C2, PCDHGA3, SSX3, FGFR2, GPR161, ATN1, CHD5, A4GALT, MYBPH, CSHL1, NCAPH2, CAPN9, CNGB1, BCAM, DRD5, NR5A2, TEF, ELAVL2, DGKB, HTR7P, RHAG, GH2, COL4A6, BMP7, SOSTDC1, SOX14, TAS2R9, LPHN2, MAP1A, OSGIN2, SLC10A2, FAM13C, EMX1, FLJ40330, CHI3L1, CDH16, SPRR1A, LOX, CALCB, GABBR2, CPB2, RASL11B, CCDC81, RUNX1, CPA1, CLCNKA, CLCNKB, FHL5, THSD7A, TFAP2C, SPAG11B, CAP2, PODNL1, SSX4, SSX4B, G6PC, RPE65, TMEM222, KDR, CHP2, GPR64, TPM2, TCEB3B, E2F5, IL5RA, AOC3, ABCF3, CPN2, ACE, NRP2, INPP5J, SMAD9, FAM155A, GART, PIR, ZNF467, ITSN2, NR1D1, THRA, RP11-35N6.1, LAMB1, EPHB3, PLA2R1, RAPGEF4, DNAJC8, ARSJ, TRIM49, GC, IL12B, CDH2, ATXN3L, BTF3L1, BICC1, FAM186A, PTPRF, TRPC4, TCL6, CYP4A22, FUT6, MUC1, DKFZP434B2016, LOC643313, LDHA, LOC100131613, TRIM3, MLLT10, DZIP1, ANKRD34C, BUB1, CSPG5, FBLN1, GAD2, CLDN1, CHRNA3, SCN11A, TEX11, IL20RA, AKAP5, KBTBD10, MSTN, TLL2, NACAD, UNC93A, PTGER1, OLAH, NHLH2, SERPINA6, KRT17, KCNMA1, PRKCA, STS, LAMA1, GPR88, ACTN2, TREH, AKAP4, DKK4, PRICKLE3, IRS4, TRPV4, PCDH11Y, APBB2, SLCO2A1, DRD2, MTMR7, ZNF471, TF, NRIP2, ST6GALNAC5, COMT, PAH, LRRC19, PRKAR1B, HPR, PRDM5, NCRNA00120, LOC79999, ITSN2, CACNB2, GPR98, PREX2, FAM182B, LAMA4, ARVCF, HAS2, YOD1, PPP2R3A, COL4A1, RBM12B, GSTA3, FAM66D, OR10H2, PTHLH, ZNF674, KRT19, ACCN2, COL6A1, LOC100288442, LOC100289169, LOC728888, LOC729602, NPIPL2, NPIPL3, PDXDC2, SLC37A1, ATP6V1B1, PTN, ABI3BP, HR44, ZNF324B, ZNF584, HOXD13, ADH6, IFNA8, MYOZ2, NFATC4, ADAMTS7, FOXL1, GPR17, SLC18A3, MYH6, BOK, FGA, TEAD4, GRM1, EDNRA, C8orf79, METTL7A, FOLH1, RAD54L, SOX11, CNOT3, NTS, MAPK12, DOCK6, DNAJC6, HS3ST3A1, LOC728395, TSPY1, TSPY3, PTH, LAMB4, ALDOB, FLG, MLANA, UBE2D4, LOC100287483, KRT20, POU1F1, SLCO1B3, CLTA, MECOM, C8orf71, SULT2A1, C6orf10, SLC27A6, PRKD1, SYNPO2L, THPO, GABRR1, CFTR, PPP2R3A, DCBLD2, ANP32A, ANP32C, ANP32D, LOC723972, XYLT1, STAB1, STAB1, SASH1, PID1, FUCA1, SASH1, LRRN3, LRRN3 or any combination thereof.

In another embodiment, the gene is TNFSF4, ITGB1/3/5, CXCL5/7, BMP6, ITGA2/8, ITGB1/3/4/5/6, ITGBL1, MMP16/24/26/28, ADAM12/18/22, IL-1/5/8/20/22, IL-9/12/36, TNFRSF11A/B, IFNA4/8/10/17, TGβ, LTBP4, MEK1/2, TGFβ type 1 receptor, Smad2/3/4, PAI-1, TNFSF4, SELP, ITFA8, ITGB1/3/5, CXCL5/7, a BMP6 gene, ITGA2/8, ITGβ1/3/4/5/6, ITGBL1, MMP6/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1 or any combination thereof. In yet another embodiment, the gene is TNFSF4, ITGB1/3/5, CXCL5/7, BMP6, ITGA2/8, ITGB1/3/4/5/6, ITGBL1, MMP16/24/26/28, ADAM12/18/22, IL-1/5/8/20/22, IL-9/12/36, TNFRSF11A/B, IFNA4/8/10/17, TGβ, LTBP4, MEK1/2, TGFβ type 1 receptor, Smad2/3/4, PAI-1 or any combination thereof.

The subject invention also provides a method of treating a subject afflicted with multiple sclerosis or presenting a clinically isolated syndrome with laquinimod, comprising the steps of: a) determining whether the subject is a laquinimod responder by evaluating expression of a biomarker in the subject, and b) administering to the subject an amount of laquinimod effective to treat the subject only if the subject is identified as a laquinimod responder, so as to thereby treat the subject, wherein the biomarker is a gene associated with inflammatory response, a gene associated with cellular movement, a gene associated with cell signaling, a gene associated with cell development, a gene associated with hematological system, or a combination thereof.

The subject invention also provides a method for treating a subject afflicted with multiple sclerosis or presenting a clinically isolated syndrome comprising the steps of: a) administering to the subject a therapeutically effective amount of laquinimod, b) determining whether the subject is a laquinimod responder by evaluating expression of a biomarker in the subject; and c) administering to the subject an amount of laquinimod effective to treat the subject only if the subject is identified as a laquinimod responder, or modifying the administration of laquinimod to the subject if the subject is not identified as a laquinimod responder, so as to thereby treat the subject, wherein the biomarker is a gene associated with inflammatory response, a gene associated with cellular movement, a gene associated with cell signaling, a gene associated with cell development, a gene associated with hematological system, or a combination thereof.

In one embodiment, the subject is identified as a laquinimod responder if the biomarker is up-regulated in the subject. In another embodiment, the subject is identified as a laquinimod responder if the biomarker is suppressed in the subject.

In one embodiment, the gene associated with inflammatory response is a gene associated with or involved in TGFb signaling, IL-12 signaling, the pathway of adhesion of phagocytes, chemotaxis of neutrophils, transmigration of leukocytes, caveolar mediated endocytosis, clathrin mediated endocytosis, and/or leukocyte extravasation signaling.

In another embodiment, the gene associated with cellular movement is a gene associated with or involved in adhesion and migration of phagocytes, chemotaxis of neutrophils, transmigration of leukocytes, invasion of cells, adhesion of cells, and/or leukocyte extravasation signaling.

In another embodiment, the gene associated with cell signaling is a gene associated with or involved in the pathway of adhesion of cells and/or neurotransmission.

In another embodiment, the gene associated with cell development is a gene associated with or involved in the pathway of G protein coupled receptor signaling, arachidonic acid metabolism and/or TGFβ signaling.

In another embodiment, the gene associated with hematological system is a gene associated with or involved in the pathway of aggregation of blood platelets, activation of blood platelets, aggregation of blood cells, coagulation of blood, intrinsic prothrombin activation pathway and/or coagulation system.

In another embodiment, the gene is TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1 or a combination thereof.

In one embodiment of any one of the methods, uses, pharmaceutical composition or packages described herein, the gene is ITGB1/3/5, CXCL5/7, BMP6, ITGA2/8, ITGB1/3/4/5/6, ITGBL1, MMP16/24/26/28, ADAM12/18/22, IL-5/20/22, IL-9/36, TNFRSF11A/B, TGβ, LTBP4, MEK1/2, Smad2/3/4, PAI-1, SELP, ITFA8, ITGB1/3/5, CXCL5/7, BMP6, ITGA2/8, ITGB1/3/4/5/6, ITGBL1, MMP16/24/26/28, ADAM12/18/22, IL-5/13/20/22, IL-9/11/36, TNFRSF11A/B, TGβ, LTBP4, MEK1/2, Smad1/2/3/4/5/6/8, PAI-1, CCL19, IKKg, LTBP1, Alpha tubulin, BMP4/7, MIS, TCF2, IL5R, IL13R, IL20R, ITGB2, NKTR, TEF, CLSTN2, LUC7L2, FABP7, TPTE, FSTL1, SF3B1, LIMS1, PDE5A, XPNPEP1, C5orf4, SPANXB1, SPANXB2, SPANXF1, KRT20, TBC1D1, GRHL2, C5orf4, SEPT6, KIAA1199, SSX2IP, TPM1, CDC14B, USP47, MMRN1, CTNNAL1, SMOX, ALOX12, GLRA3, CA2, GUCY1B3, RFPL1, CLEC1B, GNG11, TSPAN32, RGS10, CALD1, PRKAR2B, CYP4F11, CLCA3P, CELSR3, CDC14B, TPM1, SEPT6, PRKG1, MAX, CCDC93, ARMCX6, LOC653354, TUBB2B, HIST1H2AJ, MFAP3L, LIMS1, GNB5, GPRASP1, SRRT, C1orf116, FBXO7, PPM1A, GUCY1B3, CTDSPL, GNAS, IGF2BP3, TPM1, HIST1H2BK, DLG4, WDR48, CALD1, LOC157627, GNB5, ZNF415, ASAP2, PSD3, GNAS, POPDC3, NRGN, ABLIM3, XYLT1, PTGIS, ARHGEF10, PDGFA, PGRMC1, HIST1H2AC, GNAS, CLDN5, MFAP3L, PGRMC1, MYST3, CAPRIN1, CALD1, FBXW7, DNM3, CD84, PRPF4B, RBM25, WASF3, GRAP2, SPARC, TAL1, NENF, XK, GP1BA, HLA-E, CA5A, LYVE1, MARCH6, NAT8B, TRIM58, RET, SDPR, TBXA2R, TMED10, APBA2, MYL9, POU1F1, H2BFS, HIST1H2BK, FAM12B, VCL, GSPT1, ALDOB, LOC150776, SMPD4, SLC37A1, SPARC, GNAS, TAS2R4, CALM3, POM121, POM121C, GRIK2, GREM1, TNNC2, EPS15L2, ENDOD1, RGS6, SF3B1, TMSB15A, ZBTB20, FUT9, ATP9A, MAX, HIST1H2AI, BAT2D1, ABL1, SNCA, GFI1B, CTSA, SNX13, RPA1, FLNA, XPNPEP1, KIF2A, ZBTB33, PSMD11, UBE2N, FOLR1, TSC22D1, PCNP, CELSR3, ACSBG1, RNF11, SEMA3E, MARCH2, PCDH24, SUPT5H, HLA-E, EGF, HLA-C, FLNA, CDK2AP1, LEPROT, SH3TC2, TUBA4A, MTMR1, TF, PRKD1, NAP1L1, DAB2, FUCA1, HIP1, THPO, MAP1B, PARVB, GP1BB, SEPT5, GJA4, PTGS1, GUCY1A3, HIST1H2AG, GNAS, LRBA, HYAL3, GP6, IGHG1, CYP2A13, CDC14B, MAX, KDM2A, CALD1, GNAZ, C19orf22, ARHGAP6, RHOC, RBX1, GP1BB, SEPT5, PRDX6, PRB4, FLNA, HIST1H2BF, RHBDF2, NUP205, SYT1, EGFL8, PPT2, TUBB1, TMC6, FLJ11292, NAP1L1, ALDH1A3, CSNK1E, PRUNE, COL4A3, ZNF221, ILF3, CABP5, RPA1, ARF1, HIST1H2BI, PTGS1, PRKAA1, GNB5, HIST2H4A, HIST2H4B, CYB5R3, TNS1, DCT, GMPR, ABI3BP, GNAS, SASH1, AAK1, XPO6, CTSL2, QSER1, MAP1LC3B, TBX6, CABP2, MRE11A, MAPRE2, TMC6, BDKRB2, MGLL, HRASLS, WHAMML1, WHAMML2, CLU, STC1, C6orf54, PABPN1, PDLIM1, CLU, PHF20, UBL4A, RNF115, HGD, RASGRP2, PNN, SAPS3, SFI1, GOLGA2, HIST2H2BE, SGEF, HGD, DUS1L, MPP1, HLA-E, GRB14, MMD, ZFHX4, CSNK1G2, HIST1H2BE, MPDZ, B2M, TBXA2R, CTDSPL, SNCA, CD99, POLS, MPL, HIST1H3F, SFRS8, NR5A2, ZMYM2, C6orf10, TMEM40, RNF43, PRUNE, MSH6, PLCB4, PARVB, TOX3, PKNOX1, RUFY1, SNCA, C10orf81, PDGFA, ASMT, HMGB1, CCDC90A, PROS1, hCG_1757335, RAP1B, MTSS1, GNRHR, LRRN3, MCM3AP, PLOD2, NAPIL1, PLOD2, HOXD13 CASKIN2, MFAP5, PITX2, SNCA, MYLK, PBX1, PRDX6, H3F3A, H3F3B, LOC440926, TMEM158, TRIM58, FSTL1, SNCA, TNS1, ATP1B1, C5orf4, LRP12, CTNNAL1, GEM, KIAA1466, ALDH1A2, MAP4K3, SNCA, RAB6B, PSD3, RIPK2, RAMP3, CALD1, CYP2E1, PSD3, PDLIM7, COBLL1, FUT3, SMOX, TGM2, LRRC50, CST6, OR7A17, C6orf145, DLEU2, DLEU2L, CPT2, HGF, TNS1, SPRY1, PLOD2, CD80, KYNU, BCAT1, NHLH1, AHCTF1, HOXA10, MTMR3, VAC14, CLCF1, FGF5, TAL1, SAMD14, ELL2, CHN1, SLC7A1, GRK5, PARD3, VPS37B, CYP2B6, CYP2B7P1, MALL, ALX4, SOX15, KRT5, ESPL1, STARD8, PSD3, KIAA0195, MYO9B, HIP1R, LOC100294412, EFNB1, ERN1, RHD, MFAP3L, PLA1A, POFUT2, C8orf39, CRYBB2, CYP4A11, PVRL2, CLCNKB, MRAS, NFIB, FKSG2, SLC11A2, FZR1, ZNF550, GLP1R, SLC19A1, RTN2, PAPOLA, STC1, GK, EXOSC6, RAPSN, HFE, EHD2, RIOK3, UBE2I, C15orf2, DMD, PRLH, MAP2K2, TP63, DACH1, PPP5C, SLC26A1, NUDT7, KCNJ12, ENTPD7, SLC26A1, PRRG3, RGS6, ZBED2, FICD, ARHGAP1, ARHGDIA, SDHB, AMHR2, ABCA4, TCF20, BGN, CASP7, LPAR4, GNA12, CYP2W1, RAX, C4A, C4B, LOC100292046, LOC100294156, PXN, ESR2, MYL10, EFS, TFF3, SRPK1, LOC441601, BIRC5, CCT8L2, PPAP2B, CMA1, APOA2, KDELR2, ASCL3, RUNX1, BUB1, SLC6A8, HNRNPC, HNRNPCL1, LOC440563, LOC649330, RIBC2, CLIC4, RAB17, SCML2, SPINLW1, ANK1, EDA2R, HTR4, CDC42EP4, KANK2, ANK1, SYN1, DUX3, DUX4, FRG2C, HPX-2, LOC100134409, LOC652119, LOC653543, LOC653544, LOC653545, LOC728410, PKNOX2, MLLT4, APOA2, PENK, GNAT1, FURIN, SEMA6A, EGFL6, HRH1, TSPAN1, DBC1, TRPC7, GPR52, HAMP, PRSS2, GPR107, FLJ11292, FLJ20184, B4GALT1, NKX3-1, ASIP, EFCAB6, GPR20, CA5A, PLK4, TAAR5, SRPX2, CNTD2, AZGP1, TIMP3, RGS6, ADARB11, DYNC1I1, C10orf10, PDIA2, PITX3, HOXC13, LPAR3, CTRC, CTSL2, MUC8, AQP5, UGT1A1, UGT1A10, UGT1A4, UGT1A6, UGT1A8, UGT1A9, KCNQ2, CYP2A13, ZNF155, KIAA0892, ATP2A2, FGF5, FGF18, FUT2, SHROOM2, PRSS3, CREB3L1, MGAT2, PLCE1, MLXIPL, OR10H3, ABCB11, CD84, ARHGEF4, ORC1L, PCIF1, CD177, C1orf116, IFT122, C11orf20, DUSP13, C6orf208, PLA2G5, PRAMEF1, PRAMEF2, CYP4F8, KCNA1, MFAP4, SLC4A3, IL1RAPL1, SERPINE1, ZCCHC14, POLR3G, C16orf8, FLJ14100, SMCHD1, ASCL1, FOXA2, SLC23A2, KLK13, MTSS1L, DNMT3L, RREB1, DNMBP, PKLR, C1orf106, CCDC134, MTSS1, CCDC40, HOXB1, SCNN1B, SEMA4G, RAPGEFL1, MAGEL2, PLSCR2, CHD2, PLCD1, C1orf116, CHRNA2, MBP, CDC42BPA, MYF6, PI15, LOC440895, SBF1, MAST1, GLT8D2, ERBB3, LOH3CR2A, AMH, HR, RDH8, PAWR, DRD3, CCT8, PRELP, SPOCK3, EPS8L3, NXN, SEMA4G3, P2RY1, AVL9, TEK, MOGAT2, KLK7, MT1E, MT1H, MT1M, CLDN18, RHBDF2, SIX1, INPP5A, KCNMB3, MAP2K5, GPD1, LPO, LOC729143, MPRIP, WNT7A, RARG, CDH7, MBNL2, RASGRP2, RBMY2FP, MASP1, CASR, EGR4, APOC2, HECW1, HOXB3, IRF5, NNMT, AOC2, ESRRG, LPIN1, ACOT11, CCDC33, MBD2, ZNF323, NTRK2, TMEM151B, GPLD1, LENEP, HNF1B, NXPH3, ALDH1A3, PHF20L1, CKM, PARD6B, CRYGB, HAB1, LARGE, RAB40C, MPL, CHIT1, METTL10, DUS4L, PNLIPRP1, ELL, ST8SIA5, GRIN2B, MC4R, RTDR1, HDAC6, KCNJ13, CPSF1, SPANXC, CNOT4, LAMA2, SLC1A6, ABCA2, KLK11, GFRA3, CYP3A4, SLC1A3, ATP2B2, APBB2, VPS45, GHRHR, HOXD4, PRPH, ADCY2, LEFTY2, CYP1B1, PCP4, C8B, RANBP3, PDE6H, TRIM15, VGLL1, TRIM3, CRKL, ADH7, PSG3, GPR153, MFAP2, FGF13, NAPA, ALDH3A1, MCM10, TLE4, ITPR3, CCDC87, C9orf7, ACTC1, OBSL1, MAP2, CRYM, RNF122, SST, HLA-DRB6, SLC22A17, HSPG2, HIP1, GRIK2, UNKL, GPR144, KIR3DX1, NARFL, UCP3, PLXNA2, BTN1A1, ERCC4, CIITA, EGFR, KRT33A, CLTB, B3GALT5, AP3M2, GJC1, MYO3A, ARHGAP1, PPP2R3A, CLIC4, C20orf19S, SIGLEC8, GPRC5A, CACNB1, MYL10, PRLR, OR2S2, NCR2, CHAF1B, EYA3, CDS1, FBXL18, ACTL6B, ZNF821, C16orf71, HBBP1, PLXNA1, CDC45L, MTCP1, PLCB4, PLVAP, PROX1, CYP3A43, IGHG1, RECQL5, IDUA, DLGAP4, PLXNB1, HSD17B14, FOXP3, C19orf26, EPB41L1, RBBP9, GJB4, UPK1B, CYP19A1, LOC55908, CLDN18, C2orf72, NTRK3, NRXN2, SPDEF, IGH@, IGHD, IGHG1, IGHM, LOC100289944, VSIG6, ACRV1, PHLDB1, SORBS1, HAPLN2, FABP3, EFS, ACVR1B, CHST3, UGT2A1, UGT2A2, TAF1, MT4, MFAP3, ETV5, UBQLN3, TBX10, GJB1, ABO, SPINK5, ATAD4, CDH11, CARD14, ALPP, ALPPL2, CBL, LRP4, CDKL2, SSX3, DSG2, SLC45A2, LAMA4, WFDC8, HTR7, EFNB3, TUBB2B, OR7E19P, PMS2L4, ASAP3, FRZB, PDLIM4, PVT1, TFR2, AHI1, TAF4, ADAMTSL2, CLDN4, KIR2DL1, KIR2DL2, KIR2DL3, KIR2DL5A, KIR2DL5B, KIR2DS1, KIR2DS2, KIR2DS4, KIR2DS5, KIR3DL2, KIR3DL3, KIR3 DP1, LOC727787, RAPGEF5, CRMP1, LDB3, F11, USP46, IBSP, SLC9A3, FLRT3, TRIM17, FGF17, CAMK1G, GLYR1, CSH1, NTF3, ABHD6, TRIM15, OR52A1, FGFR2, ORAI2, C17orf53, GLP1R, SLIT1, TP63, DDR1, CFTR, DIO2, LETM1, ACSM5, ACTA1, NPR1, KCND3, POPDC3, DNAH3, SPDEF, CLEC4M, SLC30A3, NAGLU, AAK1, DHX34, NNAT, AKAP9, ICMT, FAM189A1, C10orf81, MYOZ1, PKNOX2, MGC31957, PRDM11, RET, IGHG1, XPNPEP2, NTRK2, SLC25A10, NR1I2, GRM8, OR3A3, GIPR, PAH, PACRG, CLN8, ZNF215, TRIO, TTLL5, GRM1, PRKG1, HHLA1, LAMA3, SLC37A4, HOXC11, SLCO5A1, CA10, RRBP1, SOD3, NTRK3, CYR61, STRA6, SLC6A11, CNOT4, ATN1, BCAP29, NOVA2, RELN, LAMC2, RAD51, PRSS7, DCBLD2, TACR2, RAB11B, OR2J2, VSNL1, IFNA17, DPYSL4, MGC2889, RRBP1, POLQ, OR1A2, PURA, AIF1, CBS, NECAB2, PRKCE, NOX1, IHH, EXO1, GPRIN2, PDX1, GPR12, FAM188A, HS3ST3B1, ASCL1, ZNF484, CSH1, BCAN, DDN, DUOX2, MORN1, SLC39A2, CLCN7, RUNX2, TTYH1, ZNF280B, PAX3, LZTS1, SLC8A2, HAB1, KIF1A, ARL4D, UGT2B15, NACA2, THRB, C6orf15, GPR176, WSCD1, PLXNB3, CADM3, HAP1, CYP1A2, SPAM1, IL22RA1, CDC2L5, IRX5, PPFIA2, KDELR3, CEACAM7, KCMF1, DUOX1, CDC27, HIST2H2AA3, CAV3, APOA4, NPR3, PRG3, TBC1D22B, TUSC3, RIMS2, CYP4F12, TBXA2R, HBEGF, PSG9, PYGO1, RASGRF1, SCN2A, KLHL1, DTNB, GREM1, SNCG, C22orf24, PALM, COBLL1, DNPEP, MNS1, NFATC4, DLC1, HSPC072, MCAM, CA12, CSHL1, RPA1N, COL5A2, UGT1A8, UGT1A9, IGH@, IGHA1, IGHG1, IGHG2, IGHG3, IGHM, LOC100126583, LOC100290036, LOC100290320, LOC100293211, LOC652494, ACSM5, ALOX12P2, ERBB4, CLDN16, CIB2, GALR3, MSMB, FABP7, ATXN3, KCNJ5, TRDN, CYP3A43, BAZ2A, ACCN4, SILV, DGCR14, SEMA6C, DIO2, PTHLH, LEP, PDZRN3, RGSL1, GJA4, SLC22A6, RASGRF1, MAPRE2, PVRL1, AKAP1, POMP, SOX21, DNAH9, HOXC5, SERHL2, KIAA0485, ITSN1, B4GALT1, NEK2, NUPR1, CCDC93, EPO, CRABP2, TYRO3, GOLGA2, SEMA3F, BFSP2, NCAM1, FOLH1, SSX2, TMPRSS4, DCN, LPHN3, POU4F3, CEACAM5, BCL3, EXTL3, CCNA1, DDR2, PAX8, SOX5, POU3F1, PEX16, NUP62, SIGLEC11, ALDOB, GPC3, IGFALS, WDR25, FGF1, OSR2, ARID1A, GYPA, KLK13, PARVB, LILRB5, RIMS2, C19orf21, HOXD1, PRSS3, FLT1, ATP6V1C1, LOX, CRYBB3, CA12, PRKG2, MASP1, LOC728395, LOC728403, TSPY1, PDCD1, GGTLC1, AQP8, KRT16, AICDA, BRD8, C1orf95, OR3A2, PFKFB2, FRZB, PAK3, MEIS2, ZSCAN2, MYH7, VWA1, LSAMP, SRC, UGT1A1, UGT1A10, UGT1A3, UGT1A4, UGT1A5, UGT1A6, UGT1A7, UGT1A8, UGT1A9, DIO1, TADA3L, NFASC, CALCRL, NBLA00301, MAB21L1, FBXO42, COL10A1, CFB, SNX7, FOXN1, SRY, HLF, CLCA3P, DAZ1, DAZ2, DAZ3, DAZ4, GPR3, TMPRSS11E, EMID1, KCNMB2, MUC5AC, SORT1, HIF3A, MAPK4, TCP11L1, ZZEF1, DCAF7, DMWD, CLCA2, VAC14, CSPG5, STMN2, MLLT4, GALNT14, FGF12, MFAP5, SUMO3, HTR3A, GDF5, TSSK1B, CYP2A7P1, MARK1, ATP1B2, TBX6, PAX8, IL1R1, RALYL, OR2B2, TAAR3, C12orf32, IGHG1, LOC642131, DICER1, GLRA3, PPARD, HSPA4L, WNT2, VIPR2, CYP2C9, SRPX2, IGSF1, ALPK3, TFPI, KCNS3, MARCH8, FRMD4B, TACR3, FIGF, PDCD6, TNN, SPANXB1, SPANXB2, SPANXF1, RHBDD3, SPP2, PDE10A, ZNF224, FGL1, PGAM2, CADM4, APOBEC2, SLC9A5, GNAT1, ARHGEF16, SMARCA2, DNAH9, RBM26, WNT2B, KCNK2, NPBWR2, SP2, TMPRSS11D, DENND2A, TNIP3, STC1, DOCK6, ADAM5P, SYDE1, TNPO2, LRTM1, USH1C, PDE12, SRCAP, OR10J1, OR2H2, KCNJ8, RP11-257K9.7, DOCK5, TPD52L1, PAEP, GGA2, PHLDA3, HES2, MLL, CHRNA6, CIB2, PTPRF, TM7SF4, DAZ1, DAZ2, DAZ3, DAZ4, ALX1, OR2F1, OR2F2, PLAT, HGC6.3, WNT11, PGK2, SNAI2, COL4A6, PRUNE2, ANKS1B, LOC81691, FERMT2, TIMP3, CST8, CAPN6, IDUA, GPR32, AKR1B10, GRHL2, FBXO24, HSF4, IGHG1, HCN2, LRP12, ARHGEF115, UGT1A1, UGT1A10, UGT1A7, UGT1A8, GUCA2A, ITIH1, EGFR, UGT1A1, UGT1A10, UGT1A3, UGT1A4, UGT1A5, UGT1A6, UGT1A7, UGT1A8, UGT1A9, MYOG, TMSB15A, TLX1, EDNRA, LOC100289791, MDFI, ZER1, MYH15, CDH20, GPR63, LOC440345, LOC440354, LOC595101, LOC641298, SMG1, HOXC10, KRTAP1-1, ARSD, CPLX3, LMAN1L, IFNA4, ABCC1, SEMA3E, MRE11A, C1QL1, LIPF, TRIM9, BBOX1, LRRC17, WNT2B, CYP3A4, SI, ANO3, OBSL1, CHRD, MSX2, PSG1, FAM107A, LRRC37B2, ANKLE2, PAX2, UNC5B, ADCYAP1R1, HFE, SYT1, GJC2, LOC100293871, FGF8, ACRV1, NRXN1, GDPD2, RGS4, CELA2A, IFNW1, MLNR, RNF17, LAD1, GLRA2, RASL12, MAGOH2, C6orf5S4, ZNF214, IKBKG, AP4E1, ZNRF4, OSBPL10, C1orf175, TTC4, PCDHB3, ADRBK1, ITSN1, XAGE1A, XAGE1B, XAGE1C, XAGE1D, XAGE1E, CDH22, FARP2, MYT1, TNC, MUC5AC, SLC6A15, PP14571, SMR3A, SMR3B, RXRG, SNX1, GLP1R, C6orf155, ATP1A2, TFAP4, PNPLA2, DIRAS3, ANO2, TACSTD2, MCM3AP, IL13RA2, TRIM10, RTEL1, PRRX2, TSHB, TIMELESS, FMO1, KIF18A, KIAA1199, CALB2, MFAP3L, PTGER3, EPAS1, SQSTM1, TSPY1, CPM, DLGAP1, CYP4F11, TLX3, PCDHA10, TAOK2, ERC1, TBX2, KALRN, DICER1, PAPPA, KIF5A, DNAJC22, OTUB1, KIAA1644, SEZ6L2, PCNXL2, HMHB1, ERG, SNTB2, GJA5, AGTR2, GJA3, GCK, LRRC61, CNTF, ZFP91, ZFP91-CNTF, PDLIM4, MPPED2, IFNA10, ACTN2, VGLL1, GJA9, LDLR, ANK2, COL1A1, TIMP3, OTOF, AGXT, GLI2, TRMT61A, FOXD2, TMEM212, DENND2A, B3GALT1, SPAG11A, PRDM4, TF, ELF5, GSC2, EPB41 L4B, GYG2, LYZL6, DCHS2, OBP2A, OBP2B, ANGPTL3, MYH11, NES, SLC17A1, RBM15B, CSH1, HTR5A, CYP3A7, HTR2A, KCNV2, TOX3, CLOCK, MAGEA6, FAM12A, COL4A3, S1PR2, NAT8, ACE2, SLC22A6, SLC13A2, MYH4, APBB2, RAP1GAP, SHOX2, SLCO1A2, ETV1, MAGEA12, PLA2G6, ADRA1A, SYT5, GPR161, SEMA3F, CYP3A43, HOMER2, KCNJ5, PPL, COL17A1, CSHL1, C9orf116, PARK2, UGT2B15, CDK6, FAM174B, CELA2A, CELA2B, SPDEF, EPB41, GAB1, SMR3A, PDE60, COL5A1, ABCA6, DMD, CYLC2, CIDEA, RAG2, HIST1H2BN, FMO6P, MAOA, ANKRD53, HAPLN1, MT1M, EHD2, GAD2, CRISP2, CSN2, SULT1C2, PCDHGA3, SSX3, FGFR2, GPR161, ATN1, CHD5, A4GALT, MYBPH, CSHL1, NCAPH2, CAPN9, CNGB1, BCAM, DRD5, NR5A2, TEF, ELAVL2, DGKB, HTR7P, RHAG, GH2, COL4A6, BMP7, SOSTDC1, SOX14, TAS2R9, LPHN2, MAP1A, OSGIN2, SLC10A2, FAM13C, EMX1, FLJ140330, CHI3L1, CDH16, SPRR1A, LOX, CALCB, GABBR2, CPB2, RASL11B, CCDC81, RUNX1, CPA1, CLCNKA, CLCNKB, FHL5, THSD7A, TFAP2C, SPAG11B, CAP2, PODNL1, SSX4, SSX4B, G6PC, RPE65, TMEM222, KDR, CHP2, GPR64, TPM2, TCEB3B, E2F5, IL5RA, AOC3, ABCF3, CPN2, ACE, NRP2, INPP5J, SMAD9, FAM155A, GART, PIR, ZNF467, ITSN2, NR1D1, THRA, RP11-35N6.1, LAMB1, EPHB3, PLA2R1, RAPGEF4, DNAJC8, ARSJ, TRIM49, GC, CDH2, ATXN3L, BTF3L1, BICC1, FAM186A, PTPRF, TRPC4, TCL6, CYP4A22, FUT6, MUC1, DKFZP434B2016, LOC643313, LDHA, LOC100131613, TRIM3, MLLT10, DZIP1, ANKRD34C, BUB1, CSPG5, FBLN1, GAD2, CLDN1, CHRNA3, SCN11A, TEX11, IL20RA, AKAP5, KBTBD10, MSTN, TLL2, NACAD, UNC93A, PTGER1, OLAH, NHLH2, SERPINA6, KRT17, KCNMA1, PRKCA, STS, LAMA1, GPR88, ACTN2, TREH, AKAP4, DKK4, PRICKLE3, IRS4, TRPV4, PCDH11Y, APBB2, SLCO2A1, DRD2, MTMR7, ZNF471, TF, NRIP2, ST6GALNAC5, COMT, PAH, LRRC19, PRKAR1B, HPR, PRDM5, NCRNA00120, LOC79999, ITSN2, CACNB2, GPR98, PREX2, FAM182B, LAMA4, ARVCF, HAS2, YOD1, PPP2R3A, COL4A1, RBM12B, GSTA3, FAM66D, OR10H2, PTHLH, ZNF674, KRT19, ACCN2, COL6A1, LOC100288442, LOC100289169, LOC728888, LOC729602, NPIPL2, NPIPL3, PDXDC2, SLC37A1, ATP6V1B1, ABI3BP, HR44, ZNF324B, ZNF584, HOXD13, ADH6, IFNA8, MYOZ2, NFATC4, ADAMTS7, FOXL1, GPR17, SLC18A3, MYH6, BOK, FGA, TEAD4, GRM1, EDNRA, C8orf79, METTL7A, FOLH1, RAD54L, SOX11, CNOT3, NTS, MAPK12, DOCK6, DNAJC6, HS3ST3A1, LOC728395, TSPY1, TSPY3, PTH, LAMB4, ALDOB, FLG, MLANA, UBE2D4, LOC100287483, KRT20, POU1F1, SLCO1B3, CLTA, MECOM, C8orf71, SULT2A1, C6orf1, SLC27A6, PRKD1, SYNPO2L, THPO, GABRR1, CFTR, PPP2R3A, DCBLD2, ANP32A, ANP32C, ANP32D, LOC723972, XYLT1, STAB1, STAB1, SASH1, PID1, FUCA1, SASH1, LRRN3, LRRN3 or a combination thereof.

In another embodiment, the gene is ITGB1/3/5, CXCL5/7, BMP6, ITGA2/8, ITGB1/3/4/5/6, ITGBL1, MMP16/24/26/28, ADAM12/18/22, IL-5/20/22, IL-9/36, TNFRSF11A/B, TGβ, LTBP4, MEK1/2, Smad2/3/4, PAI-1, SELP, ITFA8, ITGB1/3/5, CXCL5/7, BMP6, ITGA2/8, ITGB1/3/4/5/6, ITGBL1, MMP16/24/26/28, ADAM12/18/22, IL-5/13/20/22, IL-9/11/36, TNFRSF11A/B, TGβ, LTBP4, MEK1/2, Smad1/2/3/4/5/6/8, PAI-1, CCL19, IKKg, LTBP1 or a combination thereof. In another embodiment, the gene is SELP, ITFA8, ITGB1/3/5, CXCL5/7, BMP6, ITGA2/8, ITGB1/3/4/5/6, ITGBL1, MMP16/24/26/28, ADAM12/18/22, IL-1/1R/5/8/13/20/22, IL-9/11/12/36, TNFRSF11A/B, IFNA4/8/10/17, TGβ, LTBP4, MEK1/2, Smad1/2/3/4/5/6/8, PAI-1, CCL19, IKKg, LTBP1 or a combination thereof.

In one embodiment of any one of the methods, uses, pharmaceutical composition or packages described herein, the gene is TNFSF4, ITGB1/3/5, CXCL5/7, BMP6, ITGA2/8, ITGB1/3/4/5/6, ITGBL1, MMP16/24/26/28, ADAM12/18/22, IL-1/5/8/20/22, IL-9/12/36, TNFRSF11A/B, IFNA4/8/10/17, TGβ, LTBP4, MEK1/2, TGFβ type 1 receptor, Smad2/3/4, PAI-1, TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1, IL8R (CXCR1/2), Alpha tubulin, BMP2/4/7, MIS, TCF2, LFA-1, VLA-4, IL5R, IL13R, IL2OR, ITGB2, IFN gamma, TNF alpha, NKTR, TEF, CLSTN2, LUC7L2, FABP7, TPTE, FSTL1, SF3B1, LIMS1, PDE5A, XPNPEP1, C5orf4, SPANXB1, SPANXB2, SPANXF1, KRT20, TBC1D1, GRHL2, C5orf4, SEPT6, KIAA1199, SSX2IP, TPM1, CDC14B, USP47, MMRN1, CTNNAL1, SMOX, ALOX12, GLRA3, CA2, GUCY1B3, RFPL1, CLEC1B, GNG11, TSPAN32, RGS10, CALD1, PRKAR2B, CYP4F11, CLCA3P, CELSR3, CDC114B, TPM1, SEPT6, PRKG1, MAX, CCDC93, ARMCX6, LOC653354, TUBB2B, HIST1H2AJ, MFAP3L, LIMS1, GNB5, GPRASP1, SRRT, C1orf116, FBXO7, PPM1A, GUCY1B3, CTDSPL, GNAS, IGF2BP3, TPM1, HIST1H2BK, DLG4, WDR48, CALD1, LOC157627, GNB5, ZNF415, ASAP2, PSD3, GNAS, POPDC3, NRGN, ABLIM3, XYLT1, PTGIS, ARHGEF10, PDGFA, PGRMC1, HIST1H2AC, GNAS, CLDN5, MFAP3L, PGRMC1, MYST3, CAPRIN1, CALD1, FBXW7, DNM3, CD84, PRPF4B, RBM25, WASF3, GRAP2, SPARC, TAL1, NENF, XK, GP1BA, HLA-E, CA5A, LYVE1, MARCH6, NAT8B, TRIM58, RET. SDPR, TBXA2R, TMED10, APBA2, MYL9, POU1F1, H2BFS, HIST1H2BK, FAM12B, VCL, GSPT1, ALDOB, LOC150776, SMPD4, SLC37A1, SPARC, GNAS, TAS2R4, CALM3, POM121, POM121C, GRIK2, GREM1, TNNC2, EPS15L2, ENDOD1, RGS6, SF3B1, TMSB15A, ZBTB20, FUT9, ATP9A, MAX, HIST1H2AI, BAT2D1, ABL1, SNCA, GFI1B, CTSA, SNX13, RPA1, FLNA, XPNPEP1, KIF2A, ZBTB33, PSMD11, UBE2N, FOLR1, TSC22D1, PCNP, CELSR3, ACSBG1, RNF11, SEMA3E, MARCH2, PCDH24, SUPT5H, HLA-E, EGF, HLA-C, FLNA, CDK2AP1, LEPROT, SH3TC2, TUBA4A, MTMR1, TF, PRKD1, NAP1L1, DAB2, FUCA1, HIP1, THPO, MAP1B, PARVB, GP1BB, SEPT5, GJA4, PTGS1, GUCY1A3, HIST1H2AG, GNAS, LRBA, HYAL3, GP6, IGHG1, CYP2A13, CDC14B, MAX, KDM2A, CALD1, GNAZ, C19orf22, ARHGAP6, RHOC, RBX1, GP1BB, SEPT5, PRDX6, PRB4, FLNA, HIST1H2BF, RHBDF2, NUP205, SYT1, EGFL8, PPT2, TUBB1, TMC6, FLJ11292, NAP1L1, ALDH1A3, CSNK1E, PRUNE, COL4A3, ZNF221, ILF3, CABP5, RPA1, ARF1, HIST1H2BI, PTGS1, PRKAA1, GNB5, HIST2H4A, HIST2H4B, CYB5R3, TNS1, DCT, GMPR, ABI3BP, GNAS, SASH1, AAK1, XPO6, CTSL2, QSER1, MAP1LC3B, TBX6, CABP2, MRE11A, MAPRE2, TMC6, BDKRB2, MGLL, HRASLS, WHAMML1, WHAMML2, CLU, STC1, C6orf54, PABPN1, PDLIM1, CLU, PHF20, UBL4A, RNF115, HGD, RASGRP2, PNN, SAPS3, SFI1, GOLGA2, HIST2H2BE, SGEF, HGD, DUS1L, MPP1, HLA-E, GRB14, MMD, ZFHX4, CSNK1G2, HIST1H2BE, MPDZ, B2M, TBXA2R, NGFRAP1, CTDSPL, SNCA, CD99, POLS, MPL, HIST1H3F, SFRS8, NR5A2, ZMYM2, C6orf10, TMEM40, RNF43, PRUNE, MSH6, PLCB4, PARVB, TOX3, PKNOX1, RUFY1, SNCA, C10orf81, PDGFA, ASMT, HMGB1, CCDC90A, PROS1, hCG_1757335, RAP1B, MTSS1, GNRHR, LRRN3, MCM3AP, PLOD2, NAP1L1, PLOD2, HOXD13 CASKIN2, MFAP5, PITX2, SNCA, MYLK, PBX1, PRDX6, EIF2AK1, H3F3A, H3F3B, LOC440926, TMEM158, TRIM58, FSTL1, SNCA, TNS1, ATP1B1, C5orf4, LRP12, CTNNAL1, GEM, KIAA1466, ALDH1A2, MAP4K3, SNCA, RAB6B, PSD3, RIPK2, RAMP3, CALD1, CYP2E1, PSD3, PDLIM7, COBLL1, FUT3, SMOX, TGM2, LRRC50, CST6, OR7A17, C6orf145, DLEU2, DLEU2L, CPT2, HGF, TNS1, SPRY1, PLOD2, CD80, KYNU, BCAT1, NHLH1, AHCTF1, HOXA10, MTMR3, VAC14, CLCF1, FGF5, TAL1, SAMD14, ELL2, CHN1, SLC7A1, GRK5, PARD3, VPS37B, CYP2B6, CYP2B7P1, MALL, ALX4, SOX15, KRT5, ESPL1, STARD8, PSD3, KIAA0195, MYO9B, HIP1R, LOC100294412, EFNB1, ERN1, RHD, MFAP3L, PLA1A, POFUT2, C8orf39, CRYBB2, CYP4A11, PVRL2, CLCNKB, MRAS, NFIB, FKSG2, SLC11A2, FZR1, ZNF550, GLP1R, SLC19A1, RTN2, PAPOLA, STC1, GK, EXOSC6, RAPSN, HFE, EHD2, RIOK3, UBE2I, C15orf2, DMD, PRLH, MAP2K2, TP63, DACH1, PPP5C, SLC26A1, NUDT7, KCNJ12, ENTPD7, SLC26A1, PRRG3, RGS6, ZBED2, FICD, ARHGAP1, ARHGDIA, SDHB, AMHR2, ABCA4, TCF20, BGN, CASP7, LPAR4, GNA12, CYP2W1, RAX, C4A, C4B, LOC100292046, LOC100294156, ELAVL4, PXN, ESR2, MYL10, EFS, TFF3, SRPK1, LOC441601, BIRC5, CCT8L2, PPAP2B, CMA1, APOA2, KDELR2, ASCL3, RUNX1, BUB1, SLC6A8, HNRNPC, HNRNPCL1, LOC440563, LOC649330, RIBC2, CLIC4, RAB17, SCML2, SPINLW1, ANK1, EDA2R, HTR4, CDC42EP4, KANK2, ANK1, SYN1, DUX3, DUX4, FRG2C, HPX-2, LOC100134409, LOC652119, LOC653543, LOC653544, LOC653545, LOC728410, PKNOX2, MLLT4, APOA2, PENK, GNAT1, FURIN, SEMA6A, EGFL6, HRH1, TSPAN1, DBC1, TRPC7, MDM2, GPR52, HAMP, PRSS2, GPR107, FLJ11292, FLJ20184, B40ALT1, NKX3-1, ASIP, EFCAB6, GPR20, CA5A, PLK4, TAAR5, SRPX2, CNTD2, AZGP1, TIMP3, RGS6, ADARB1, DYNC1I1, C10orf10, PDIA2, PITX3, HOXC13, LPAR3, CTRC, CTSL2, MUC8, AQP5, UGT1A1, UGT1A10, UGT1A4, UGT1A6, UGT1A8, UGT1A9, KCNQ2, CYP2A13, ZNF155, KIAA0892, ATP2A2, FGF5, FGF18, FUT2, SHROOM2, PRSS3, CREB3L1, MGAT2, PLCE1, MLXIPL, OR10H3, ABCB11, CD84, ARHGEF4, ORC1L, PCIF1, CD177, C1orf116, IFT122, C11orf20, DUSP13, C6orf208, PLA2G5, PRAMEF1, PRAMEF2, CYP4F8, KCNA1, MFAP4, C6, SLC4A3, IL1RAPL1, SERPINE1, ZCCHC14, POLR3G, C16orf8, FLJ14100, SMCHD1, ASCL1, FOXA2, SLC23A2, KLK13, MTSS1L, DNMT3L, RREB1, DNMBP, PKLR, C1orf106, CCDC134, MTSS1, CCDC40, HOXB1, SCNN1B, SEMA4G, RAPGEFL1, MAGEL2, PLSCR2, CHD2, PLCD1, C1orf116, CHRNA2, MBP, CDC42BPA, MYF6, PI15, LOC440895, SBF1, MAST1, GLT8D2, ERBB3, LOH3CR2A, AMH, HR, RDH8, PAWR, DRD3, CCT8, PRELP, SPOCK3, EPS8L3, NXN, SEMA4G, P2RY1, AVL9, TEK, MOGAT2, KLK7, MT1E, MT1H, MT1M, CLDN18, RHBDF2, SIX1, INPP5A, KCNMB3, MAP2K5, GPD1, LPO, LOC729143, MPRIP, WNT7A, RARG, CDH7, MBNL2, RASGRP2, RBMY2FP, MASP1, CASR, EGR4, APOC2, HECW1, HOXB3, IRF5, NNMT, AOC2, ESRRG, LPIN1, ACOT11, CCDC33, MBD2, ZNF323, NTRK2, TMEM151B, GPLD1, LENEP, HNF1B, NXPH3, ALDH1A3, PHF20L1, CKM, PARD6B, CRYGB, HAB1, LARGE, RAB40C, MPL, CHIT1, METTL10, DUS4L, PNLIPRP1, ELL, ST8SIA5, GRIN2B, MC4R, RTDR1, HDAC6, KCNJ13, CPSF1, SPANXC, CNOT4, LAMA2, SLC1A6, ABCA2, KLK11, GFRA3, CYP3A4, SLC1A3, ATP2B2, APBB2, VPS45, GHRHR, HOXD4, PRPH, ADCY2, LEFTY2, CYP1B1, PCP4, C8B, RANBP3, PDE6H, TRIM15, VGLL1, TRIM3, CRKL, ADH7, PSG3, GPR153, MFAP2, FGF13, NAPA, ALDH3A1, MCM10, TLE4, ITPR3, CCDC87, C9orf7, ACTC1, OBSL1, MAP2, CRYM, RNF122, SST, HLA-DRB6, SLC22A17, HSPG2, HIP1, GRIK2, UNKL, GPR144, KIR3DX1, NARFL, UCP3, PLXNA2, BTN1A1, ERCC4, CIITA, EGFR, KRT33A, CLTB, B3GALT5, AP3M2, GJC1, MYO3A, ARHGAP1, PPP2R3A, CLIC4, C20orf195, SIGLEC8, GPRC5A, CACNB1, MYL10, PRLR, OR2S2, NCR2, CHAF1B, EYA3, CDS1, FBXL18, ACTL6B, ZNF821, C16orf71, HBBP1, PLXNA1, CDC45L, MTCP1, PLCB4, PLVAP, PROX1, CYP3A43, IGHG1, RECQL5, IDUA, DLGAP4, PLXNB1, HSD17B14, FOXP3, C19orf26, EPB41L1, RBBP9, GJB4, UPK1B, CYP19A1, LOC55908, CLDN18, C2orf72, NTRK3. NRXN2, SPDEF, IGH@IGHD, IGHG1, IGHM, LOC100289944, VSIG6, ACRV1, PHLDB1, SORBS1, HAPLN2, FABP3, EFS, ACVR1B, CHST3, UGT2A1, UGT2A2, TAF1, MT4, MFAP3, ETV5, UBQLN3, TBX10, GJB1, ABO, SPINK5, ATAD4, CDH11, CARD14, ALPP, ALPPL2, CBL, LRP4, CDKL2, SSX3, DSG2, SLC45A2, LAMA4, WFDC8, HTR7, EFNB3, TUBB2B, OR7E19P, PMS2L4, ASAP3, FRZB, PDLIM4, PVT1, TFR2, AHI1, TAF4, ADAMTSL2, CLDN4, KIR2DL1, KIR2DL2, KIR2DL3, KIR2DL5A, KIR2DL5B, KIR2DS1, KIR2DS2, KIR2DS3, KIR2DS4, KIR2DS5, KIR3DL2, KIR3DL3, KIR3 DP1, LOC727787, RAPGEF5, CRMP1, LDB3, F11, USP46, PTN, IBSP, SLC9A3, FLRT3, TRIM17, FGF17, CAMK1G, GLYR1, CSH1, NTF3, ABHD6, TRIM15, OR52A1, FGFR2, ORAI2, C17orf53, GLP1R, SLIT1, TP63, DDR1, CFTR, DIO2, LETM1, ACSM5, ACTA1, NPR1, KCND3, POPDC3, DNAH3, SPDEF, CLEC4M, SLC30A3, NAGLU, AAK1, DHX34, NNAT, AKAP9, ICMT, FAM189A1, C10orf81, MYOZ1, PKNOX2, MGC31957, PRDM11, RET, IGHG1, XPNPEP2, NTRK2, SLC25A10, NR1I2, GRM8, OR3A3, GIPR, PAH, PACRG, CLN8, ZNF215, TRIO, TTLL5, GRM1, PRKG1, HHLA1, LAMA3, PTN, SLC37A4, HOXC11, SLCO5A1, CA10, RRBP1, SOD3, NTRK3, CYR61, STRA6, SLC6A11, CNOT4, ATN1, BCAP29, NOVA2, RELN, LAMC2, RAD51, PRSS7, DCBLD2, TACR2, RAB11B, OR2J2, VSNL1, IFNA17, DPYSL4, MGC2889, RRBP1, POLQ, OR1A2, PURA, AIF1, CBS, NECAB2, PRKCE, NOX1, IHH, EXO1, GPRIN2, PDX1, GPR12, FAM188A, HS3ST3B1, ASCL1, ZNF484, CSH1, BCAN, DDN, DUOX2, MORN11, SLC39A2, CLCN7, RUNX2, TTYH1, ZNF280B, PAX3, LZTS1, SLC8A2, HAB1, KIF1A, ARL4D, UGT2B15, NACA2, THRB, C6orf15, GPR176, WSCD1, PLXNB3, CADM3, HAP1, CYP1A2, SPAM1, IL22RA1, CDC2L5, IRX5, PPFIA2, KDELR3, CEACAM7, KCMF1, DUOX1, CDC27, HIST2H2AA3, CAV3, APOA4, NPR3, PRG3, TBC1D22B, TUSC3, RIMS2, CYP4F12, TBXA2R, HBEGF, PSG9, PYGO1, RASGRF1, SCN2A, KLHL1, DTNB, GREM1, SNCG, C22orf24, PALM, COBLL1, DNPEP, MNS1, NFATC4, DLC1, HSPC072, MCAM, CA12, CSHL1, RPAIN, COL5A2, UGT1A8, UGT1A9, IGH@, IGHA1, IGHG1, IGHG2, IGHG3, IGHM, LOC100126583, LOC100290036, LOC100290320, LOC100293211, LOC652494, TGFB2, ACSM5, ALOX12P2, ERBB4, CLDN16, CIB2, GALR3, MSMB, FABP7, ATXN3, KCNJ5, TRDN, CYP3A43, BAZ2A, ACCN4, SILV, DGCR14, SEMA6C, DIO2, PTHLH, LEP, PDZRN3, RGSL1, GJA4, SLC22A6, RASGRF1, MAPRE2, PVRL1, AKAP1, POMP, SOX21, DNAH9, HOXC5, SERHL2, KIAA0485, ITSN1, B4GALT1, NEK2, NUPR1, CCDC93, EPO, CRABP2, TYRO3, GOLGA2, SEMA3F, BFSP2, NCAM1, FOLH1, SSX2, TMPRSS4, DCN, LPHN3, POU4F3, CEACAM5, BCL3, EXTL3, CCNA1, DDR2, PAX8, SOX5, POU3F1, PEX16, IL4I1, NUP62, SIGLEC11, ALDOB, GPC3, IGFALS, WDR25, FGF1, OSR2, ARID1A, GYPA, KLK13, PARVB, LILRB5, RIMS2, C19orf21, HOXD1, PRSS3, FLT1, ATP6V1C1, LOX, CRYBB3, CA12, PRKG2, MASP1, LOC728395, LOC728403, TSPY1, PDCD1, GGTLC1, AQP8, IL1F9, KRT16, AICDA, BRD8, C1orf95, OR3A2, PFKFB2, FRZB, PAK3, MEIS2, ZSCAN2, MYH7, VWA1, LSAMP, SRC, UGT1A1, UGT1A10, UGT1A3, UGT1A4, UGT1A5, UGT1A6, UGT1A7, UGT1A8, UGT1A9, DIO1, TADA3L, NFASC, CALCRL, NBLA00301, MAB21L1, FBXO42, COL10A1, CFB, SNX7, FOXN1, SRY, HLF, CLCA3P, DAZ1, DAZ2, DAZ3, DAZ4, GPR3, TMPRSS11E, EMID1, KCNMB2, MUC5AC, SORT1, HIF3A, MAPK4, TCP11L1, ZZEF1, DCAF7, DMWD, CLCA2, VAC14, CSPG5, STMN2, MLLT4, GALNT14, FGF12, MFAP5, SUMO3, HTR3A, GDF5, TSSK1B, CYP2A7P1, MARK1, ATP1B2, TBX6, PAX8, IL1R1, RALYL, OR2B2, TAAR3, C12orf32, IGHG1, LOC642131, DICER1, GLRA3, PPARD, HSPA4L, WNT2, VIPR2, CYP2C9, SRPX2, IGSF1, ALPK3, TFPI, KCNS3, MARCH8, FRMD4B, TACR3, FIGF, PDCD6, TNN, SPANXB1, SPANXB2, SPANXF1, RHBDD3, SPP2, PDE10A, ZNF224, FGL1, PGAM2, CADM4, APOBEC2, SLC9A5, GNAT1, ARHGEF16, SMARCA2, DNAH9, RBM26, WNT2B, KCNK2, NPBWR2, SP2, TMPRSS11D, DENND2A, TNIP3, STC1, DOCK6, ADAM5P, SYDE1, TNPO2, LRTM1, USH1C, PDE12, SRCAP, OR10J1, OR2H2, KCNJ8, RP11-257K9.7, DOCK5, TPD52L1, PAEP, GGA2, PHLDA3, HES2, MLL, PTN, CHRNA6, CIB2, PTPRF, TM7SF4, DAZ1, DAZ2, DAZ3, DAZ4, ALX1, OR2F1, OR2F2, PLAT, HGC6.3, WNT11, PGK2, SNAI2, COL4A6, PRUNE2, ANKS1B, LOC81691, FERMT2, TIMP3, CST8, CAPN6, IDUA, GPR32, AKR1B10, GRHL2, FBXO24, HSF4, IGHG1, HCN2, LRP12, ARHGEF15, UGT1A1, UGT1A10, UGT1A7, UGT1A8, GUCA2A, MDK, ITIH1, EGFR, UGT1A1, UGT1A10, UGT1A3, UGT1A4, UGT1A5, UGT1A6, UGT1A7, UGT1A8, UGT1A9, MYOG, TMSB15A, TLX1, EDNRA, LOC100289791, MDFI, ZER1, MYH15, CDH20, GPR63, LOC440345, LOC440354, LOC595101, LOC641298, SMG1, HOXC10, KRTAP1-1, ARSD, CPLX3, LMAN1L, IFNA4, ABCC1, SEMA3E, MRE11A, C1QL1, LIPF, TRIM9, BBOX1, LRRC17, WNT2B, CYP3A4, SI, ANO3, OBSL1, CHRD, MSX2, PSG1, FAM107A, LRRC37B2, ANKLE2, PAX2, UNC5B, ADCYAP1R1, HFE, SYT1, GJC2, LOC100293871, FGF8, ACRV1, NRXN1, GDPD2, RGS4, CELA2A, IFNW1, MLNR, RNF17, LAD1, GLRA2, RASL12, MAGOH2, C6orf54, ZNF214, IKBKG, AP4E1, ZNRF4, OSBPL10, C1orf175, TTC4, PCDHB3, ADRBK1, ITSN1, XAGE1A, XAGE1B, XAGE1C, XAGE1D, XAGE1E, CDH22, FARP2, MYT1, TNC, MUC5AC, SLC6A15, PP14571, SMR3A, SMR3B, RXRG, SNX1, GLP1R, C6orf155, ATP1A2, TFAP4, PNPLA2, DIRAS3, ANO2, TACSTD2, MCM3AP, IL13RA2, TRIM10, RTEL1, PRRX2, TSHB, TIMELESS, FMO1, KIF18A, KIAA1199, CALB2, MFAP3L, PTGER3, EPAS1, SQSTM1, TSPY1, CPM, DLGAP1, CYP4F11, TLX3, PCDHA10, TAOK2, ERC1, TBX2, KALRN, DICER1, PAPPA, KIF5A, DNAJC22, OTUB1, KIAA1644, SEZ6L2, PCNXL2, HMHB1, ERG, SNTB2, GJA5, AGTR2, GJA3, GCK, LRRC61, CNTF, ZFP91, ZFP91-CNTF, PDLIM4, MPPED2, IFNA10, ACTN2, VGLL1, GJA9, LDLR, ANK2, COL1A1, TIMP3, OTOF, AGXT, GLI2, TRMT61A, FOXD2, TMEM2I2, DENND2A, B3GALT1, SPAG11A, PRDM4, TF, ELF5, GSC2, EPB41L4B, GYG2, LYZL6, DCHS2, OBP2A, OBP2B, ANGPTL3, MYH11, NES, SLC17A1, RBM15B, CSH1, HTR5A, CYP3A7, HTR2A, KCNV2, TOX3, CLOCK, MAGEA6, FAM12A, COL4A3, S1PR2, NAT8, ACE2, SLC22A6, SLC13A2, MYH4, APBB2, RAP1GAP, SHOX2, SLCO1A2, ETV1, MAGEA12, PLA2G6, ADRA1A, SYT5, GPR161, SEMA3F, CYP3A43, HOMER2, KCNJ5, PPL, COL17A1, CSHL1, C9orf116, PARK2, UGT2B15, CDK6, FAM174B, CELA2A, CELA2B, SPDEF, EPB41, GAB1, SMR3A, PDE6G, COL5A1, ABCA6, DMD, CYLC2, CIDEA, RAG2, HIST1H2BN, FMO6P, MAOA, ANKRD53, HAPLN1, MT1M, EHD2, GAD2, CRISP2, CSN2, SULT1C2, PCDHGA3, SSX3, FGFR2, GPR161, ATN1, CHD5, A4GALT, MYBPH, CSHL1, NCAPH2, CAPN9, CNGB1, BCAM, DRD5, NR5A2, TEF, ELAVL2, DGKB, HTR7P, RHAG, GH2, COL4A6, BMP7, SOSTDC1, SOX14, TAS2R9, LPHN2, MAP1A, OSGIN2, SLC10A2, FAM13C, EMX1, FLJ40330, CHI3L1, CDH16, SPRR1A, LOX, CALCB, GABBR2, CPB2, RASL11B, CCDC81, RUNX1, CPA1, CLCNKA, CLCNKB, FHL5, THSD7A, TFAP2C, SPAG11B, CAP2, PODNL1, SSX4, SSX4B, G6PC, RPE65, TMEM222, KDR, CHP2, GPR64, TPM2, TCEB3B, E2F5, IL5RA, AOC3, ABCF3, CPN2, ACE, NRP2, INPP5J, SMAD9, FAM155A, GART, PIR, ZNF467, ITSN2, NRID1, THRA, RP11-35N6.1, LAMB1, EPHB3, PLA2R1, RAPGEF4, DNAJC8, ARSJ, TRIM49, GC, IL12B, CDH2, ATXN3L, BTF3L1, BICC1, FAM186A, PTPRF, TRPC4, TCL6, CYP4A22, FUT6, MUC1, DKFZP434B2016, LOC643313, LDHA, LOC100131613, TRIM3, MLLT10, DZIP1, ANKRD34C, BUB1, CSPG5. FBLN1, GAD2, CLDN1, CHRNA3, SCN11A, TEX11, IL20RA, AKAP5, KBTBD10, MSTN, TLL2, NACAD, UNC93A, PTGER1, OLAH, NHLH2, SERPINA6, KRT17, KCNMA1, PRKCA, STS, LAMA1, GPR88, ACTN2, TREH, AKAP4, DKK4, PRICKLE3, IRS4, TRPV4, PCDH11Y, APBB2, SLCO2A1, DRD2, MTMR7, ZNF471, TF, NRIP2, ST6GALNAC5, COMT, PAH, LRRC19, PRKAR1B, HPR, PRDM5, NCRNA00120, LOC79999, ITSN2, CACNB2, GPR98, PREX2, FAM182B, LAMA4, ARVCF, HAS2, YOD1, PPP2R3A, COL4A1, RBM12B, GSTA3, FAM66D, OR10H2, PTHLH, ZNF674, KRT19, ACCN2, COL6A1, LOC100288442, LOC100289169, LOC728888, LOC729602, NPIPL2, NPIPL3, PDXDC2, SLC37A1, ATP6V1B1, PTN, ABI3BP, HR44, ZNF324B, ZNF584, HOXD13, ADH6, IFNA8, MYOZ2, NFATC4, ADAMTS7, FOXL1, GPR17, SLC18A3, MYH6, BOK, FGA, TEAD4, GRM1, EDNRA, C8orf79, METTL7A, FOLH1, RAD54L, SOX11, CNOT3, NTS, MAPK12, DOCK6, DNAJC6, HS3ST3A1, LOC728395, TSPY1, TSPY3, PTH, LAMB4, ALDOB, FLG, MLANA, UBE2D4, LOC100287483, KRT20, POU1F1, SLCO1B3, CLTA, MECOM, C8orf71, SULT2A1, C6orf10, SLC27A6, PRKD1, SYNPO2L, THPO, GABRR1, CFTR, PPP2R3A, DCBLD2, ANP32A, ANP32C, ANP32D, LOC723972, XYLT1, STAB1, STAB1, SASH1, PID1, FUCA1, SASH1, LRRN3, LRRN3 or any combination thereof.

In another embodiment, the gene is TNFSF4, ITGB1/3/5, CXCL5/7, BMP6, ITGA2/8, ITGβB/3/4/5/6, ITGBL1, MMP16/24/26/28, ADAM12/18/22, IL-1/5/8/20/22, IL-9/12/36, TNFRSF11A/B, IFNA4/8/10/17, TGβ, LTBP4, MEK1/2, TGFβ type 1 receptor, Smad2/3/4, PAI-1, TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type 11 BMPR, smad1/2/3/4/5/6/8, PAI-1, CCL19, IKKg, LTBP1 or any combination thereof. In yet another embodiment, the gene is TNFSF4, ITGB1/3/5, CXCL5/7, BMP6, ITGA2/8, ITGB1/3/4/5/6, ITGBL1, MMP16/24/26/28, ADAM12/18/22, IL-1/5/8/20/22, IL-9/12/36, TNFRSF11A/B, IFNA4/8/10/17, TGβ, LTBP4, MEK1/2, TGFβ type 1 receptor, Smad2/3/4, PAI-1 or any combination thereof.

In one embodiment, laquinimod is administered orally. In another embodiment, laquinimod is administered daily.

In one embodiment, laquinimod is administered at a dose of less than 0.6 mg/day. In another embodiment, laquinimod is administered at a dose of 0.1-40.0 mg/day. In another embodiment, laquinimod is administered at a dose of 0.1-2.5 mg/day. In another embodiment, laquinimod is administered at a dose of 0.25-2.0 mg/day. In another embodiment, laquinimod is administered at a dose of 0.5-1.2 mg/day. In another embodiment, laquinimod is administered at a dose of 0.25 mg/day. In another embodiment, laquinimod is administered at a dose of 0.3 mg/day. In another embodiment, laquinimod is administered at a dose of 0.5 mg/day. In another embodiment, laquinimod is administered at a dose of 0.6 mg/day. In another embodiment, laquinimod is administered at a dose of 1.0 mg/day. In another embodiment, laquinimod is administered at a dose of 1.2 mg/day. In another embodiment, laquinimod is administered at a dose of 1.5 mg/day. In yet another embodiment, laquinimod is administered at a dose of 2.0 mg/day.

In one embodiment, the subject is a naïve subject. In another embodiment, the subject is naïve to laquinimod. In another embodiment, the subject has been previously administered laquinimod.

In another embodiment, the subject has been previously administered a multiple sclerosis drug other than laquinimod.

In an embodiment, the step of evaluating expression of the biomarker comprises normalization of the subject's gene expression. In another embodiment, the step of evaluating expression of the biomarker comprises comparing expression level in the subject relative to a reference value. In another embodiment, the reference value is based on the level of expression of the biomarker in a laquinimod Non-Responder population. In another embodiment, the reference value is based on the level of expression of the biomarker in a healthy control population. In yet another embodiment, the reference value is based on the level of expression of the subject at baseline.

In one embodiment, the subject is identified as a laquinimod responder if expression of the biomarker is higher than a reference value. In yet another embodiment, the subject is identified as a laquinimod responder if expression level of the biomarker is lower than a reference value.

In one embodiment, expression of the biomarker is evaluated in the blood of the subject. In another embodiment, expression of the biomarker is evaluated in the peripheral blood mononuclear cells (PBMCs) of the subject. In another embodiment, expression of the biomarker is evaluated prior to treatment with laquinimod.

In one embodiment, expression of the biomarker is evaluated after beginning treatment with laquinimod. In another embodiment, expression of the biomarker is evaluated one month after beginning treatment with laquinimod. In another embodiment, expression of the biomarker is evaluated 6 months after beginning treatment with laquinimod. In another embodiment, expression of the biomarker is evaluated 12 months after beginning treatment with laquinimod. In another embodiment, expression of the biomarker is evaluated 24 months after beginning treatment with laquinimod.

In one embodiment, if the subject is identified as a laquinimod responder, the subject is thereafter administered a pharmaceutical composition comprising laquinimod and a pharmaceutically acceptable carrier as monotherapy. In another embodiment, if the subject is identified as a laquinimod responder, the subject is thereafter administered a pharmaceutical composition comprising laquinimod and a pharmaceutically acceptable carrier in combination with another multiple sclerosis drug. In another embodiment, if the subject is identified as a laquinimod non-responder, the subject is thereafter administered a multiple sclerosis drug which is not laquinimod.

In one embodiment, the subject is a human patient.

The subject invention also provides laquinimod for use in treating a subject afflicted with multiple sclerosis or presenting a clinically isolated syndrome, wherein the subject has been identified as a laquinimod responder.

The subject invention also provides a pharmaceutical composition comprising an amount of laquinimod for use in treating a subject afflicted with multiple sclerosis or presenting a clinically isolated syndrome, wherein the subject has been identified as a laquinimod responder

The subject invention also provides laquinimod for use in treating a subject afflicted with multiple sclerosis or presenting a clinically isolated syndrome, wherein expression of a biomarker in the subject is up-regulated and the biomarker is a gene associated with inflammatory response, a gene associated with cellular movement, a gene associated with cell signaling, a gene associated with cell development, a gene associated with hematological system, or a combination thereof.

The subject invention also provides a pharmaceutical composition comprising an amount of laquinimod for use in treating a subject afflicted with multiple sclerosis or presenting a clinically isolated syndrome, wherein expression of a biomarker in the subject is up-regulated and the biomarker is a gene associated with inflammatory response, a gene associated with cellular movement, a gene associated with cell signaling, a gene associated with cell development, a gene associated with hematological system, or a combination thereof.

The subject invention also provides laquinimod for use in treating a subject afflicted with multiple sclerosis or presenting a clinically isolated syndrome, wherein expression of a biomarker in the subject is suppressed and the biomarker is a gene associated with inflammatory response, a gene associated with cellular movement, a gene associated with cell signaling, a gene associated with cell development, a gene associated with hematological system, or a combination thereof.

The subject invention also provides a pharmaceutical composition comprising an amount of laquinimod for use in treating a subject afflicted with multiple sclerosis or presenting a clinically isolated syndrome, wherein expression of a biomarker in the subject is suppressed and the biomarker is a gene associated with inflammatory response, a gene associated with cellular movement, a gene associated with cell signaling, a gene associated with cell development, a gene associated with hematological system, or a combination thereof.

The subject invention also provides a therapeutic package for dispensing to, or for use in dispensing to, a subject identified as a laquinimod responder afflicted with multiple sclerosis or presenting a clinically isolated syndrome, which comprises: a) one or more unit doses, each such unit dose comprising an amount of laquinimod, and b) a finished pharmaceutical container therefor, said container containing said unit dose or unit doses, said container further containing or comprising labeling directing the use of said package in the treatment of said subject.

The subject invention also provides a therapeutic package for dispensing to, or for use in dispensing to, a subject afflicted with multiple sclerosis or presenting a clinically isolated syndrome, which comprises: a) one or more unit doses, each such unit dose comprising an amount of laquinimod, and b) a finished pharmaceutical container therefor, said container containing said unit dose or unit doses, said container further containing or comprising labeling directing the use of said package in the treatment of said subject, wherein expression of a biomarker in the subject is suppressed or up-regulated and the biomarker is a gene associated with inflammatory response, a gene associated with cellular movement, a gene associated with cell signaling, a gene associated with cell development, a gene associated with hematological system, or a combination thereof.

For the foregoing embodiments, each embodiment disclosed herein is contemplated as being applicable to each of the other disclosed embodiment. For example, the elements recited in the method embodiments can be used in the use and package embodiments described herein and vice versa.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Patent Application Publication No. 2005/0192315 and PCT International Application Publication No. WO 2005/074899, which are hereby incorporated by reference into this application.

A dosage unit may comprise a single compound or mixtures of compounds thereof. A dosage unit can be prepared for oral dosage forms, such as tablets, capsules, pills, powders, and granules.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents flow-inducing agents, and melting agents.

Specific examples of the techniques, pharmaceutically acceptable carriers and excipients that may be used to formulate oral dosage forms of the present invention are described, e.g., in U.S. Patent Application Publication No. 2005/0192315, PCT International Application Publication Nos. WO 2005/074899, WO 2007/047863, and 2007/146248.

General techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

### TERMS

As used herein, and unless stated otherwise, each of the following terms shall have the definition set forth below.

As used herein, “laquinimod” means laquinimod acid or a pharmaceutically acceptable salt thereof.

As used herein, an “amount” or “dose” of an agent, e.g., laquinimod as measured in milligrams refers to the milligrams of the agent, e.g., laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, a “unit dose”, “unit doses” and “unit dosage form(s)” mean a single drug administration entity/entities.

As used herein, “about” in the context of a numerical value or range means±10% of the numerical value or range recited or claimed.

As used herein, “effective” or “therapeutically effective” when referring to an amount of laquinimod or a therapy regimen using laquinimod refers to the quantity or regimen of laquinimod that is sufficient to yield a desired therapeutic response. Efficacy can be measured by an improvement of a symptom of multiple sclerosis. Such symptoms can include a MRI-monitored multiple sclerosis disease activity, relapse rate, accumulation of physical disability, frequency of relapses, time to confirmed disease progression, time to confirmed relapse, frequency of clinical exacerbation, brain atrophy, neuronal dysfunction, neuronal injury, neuronal degeneration, neuronal apoptosis, risk for confirmed progression, visual function, fatigue, impaired mobility, cognitive impairment, brain volume, abnormalities observed in whole Brain MTR histogram, general health status, functional status, quality of life, and/or symptom severity on work.

As used herein, “clinical responsiveness” is a measure of the degree of a patients' response to an agent, e.g., laquinimod. Positive clinical responsiveness corresponds to a patient who responds favorably to and/or benefits from receiving laquinimod (a laquinimod responder) while negative clinical responsiveness corresponds a patient who responds unfavorably to and/or does not benefit from receiving laquinimod (a laquinimod non-responder).

As used herein, “a gene associated with” a process or a system, e.g., a gene associated with inflammatory response or a gene associated with hematological system, is a gene which plays a role in that process or system. As an example, a gene associated with inflammatory response can be IL-1R, IL-8R, IL-22R, IL-9, TNFRSF4 or RORC.

Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

“Treating” as used herein encompasses, e.g., inducing inhibition, regression, or stasis of a disease or disorder, e.g., Relapsing MS (RMS), or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with MS or RMS includes any clinical or laboratory manifestation associated with MS or RMS and is not limited to what the subject can feel or observe.

As used herein, “a subject afflicted with multiple sclerosis” or “a subject afflicted with relapsing multiple sclerosis” means a subject who has been clinically diagnosed to have multiple sclerosis or relapsing multiple sclerosis (RMS), which includes relapsing-remitting multiple sclerosis (RRMS) and Secondary Progressive multiple sclerosis (SPMS).

As used herein, a subject at “baseline” is as subject prior to administration of laquinimod.

A “patient at risk of developing MS” (i.e. clinically definite MS) as used herein is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

As used herein, a “multiple sclerosis drug” is a drug or an agent intended to treat clinically defined MS, CIS, any form of neurodegenerative or demyelinating diseases, or symptoms of any of the above mentioned diseases. “Multiple sclerosis drugs” may include but are not limited to antibodies, immunosuppressants, anti-inflammatory agents, immunomodulators, cytokines, cytotoxic agents and steroids and may include approved drugs, drugs in clinical trial, or alternative treatments, intended to treat clinically defined MS, CIS or any form of neurodegenerative or demyelinating diseases. “Multiple sclerosis drugs” include but are not limited to Interferon and its derivatives (including BETASERON®, AVONEX® and REBIF®), Mitoxantrone and Natalizumab. Agents approved or in-trial for the treatment of other autoimmune diseases, but used in a MS or CIS patient to treat MS or CIS are also included.

As used herein, a “naïve patient” is a subject that has not been treated with a multiple sclerosis drug as defined herein. Similarly, a patient or subject who is “naïve” to an agent, e.g., laquinimod, is a patient or subject that has not been treated with said agent.

As used herein, “in the blood of the subject” is represented by PBMCs, lymphocytes, monocytes, macrophages, basophils, dendritic cells or other cells derived from the subject's blood.

As used herein a “reference value” is a value or range of values that characterizes a specified population in a defined state of health.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, 0.4 mg/day, 0.5 mg/day etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### High-Through Output Gene Expression Ancillary Study for Phase III Clinical Trial (“ALLEGRO” or MS-LAQ-301) to Assess Effect of Laquinimod on Peripheral Blood Mononuclear Cells in Relapsing-Remitting Multiple Sclerosis

In a previous study by Gurevich et al. (Gurevich et al. 2010), in vitro molecular effects of laquinimod (LAQ) in peripheral blood mononuclear cells (PBMC) of healthy subjects and relapsing-remitting multiple sclerosis (RRMS) patients were characterized by gene expression microarrays. Gurevich et al. demonstrated that LAQ induced suppression of genes related to antigen presentation and corresponding inflammatory pathways. To further elucidate the molecular mechanism/s underlying the therapeutic effect of LAQ following treatment of patients displaying RRMS, the inventors performed gene expression microarray analysis of PBMCs from RRMS patients treated with LAQ as ancillary study to ALLEGRO clinical trial.

### ALLEGRO Clinical Trial

ALLEGRO was a multinational (24 countries), multicenter (approximately 139 sites), randomized, double-blinded, parallel-group, placebo-controlled clinical trial conducted to evaluate the efficacy, safety and tolerability of daily oral administration of laquinimod 0.6 mg in subjects with relapsing remitting multiple sclerosis (RRMS) for a 24 months duration.

One thousand one hundred and six (1106) patients were equally randomized to either laquinimod 0.6 mg or placebo and treated in a double-blind manner and baseline characteristics were balanced between groups. The primary endpoint of the study was the number of confirmed relapses during the double-blind treatment period, which corresponds to the annualized relapse rate (ARR—number of relapses divided by total exposure of all patients). Secondary endpoints included disability as measured by Expanded Disability Status Scale (EDSS) changes confirmed at 3 months, and cumulative number of gadolinium enhancing (GdE) and new/enlarging T2 MRI lesions.

### Study Duration

Screening phase: 1 month.

Double blind treatment phase: 24 months of once-daily oral administration of daily dose of 0.6 mg laquinimod or matching placebo.

Upon blinded variance and power reassessment of the population progression (planned prior to first subject completes the 20 months of treatment), the double blind study duration may be extended to 30 months. This is planned in order to enhance the statistical power to detect the effect of laquinimod on disability accumulation. The recommendation to extend the study duration is based on a pre-defined rule.

### Study Design

Eligible subjects were equally randomized 1:1 into one of the following treatment arms:
1. Laquinimod capsules 0.6 mg: One 0.6 mg laquinimod capsule was administered orally once daily. The 0.6 mg laquinimod capsules contain 0.6 mg of Laquinimod Acid per capsule with meglumine, and were manufactured according to the method disclosed in PCT International Application Publication No. WO/2007/146248, published Dec. 21, 2007 (see, page 10, line 5 to page 11, line 3).
2. Matching placebo for laquinimod arm: one capsule is administered once daily.

Subjects were evaluated at study sites for 12 scheduled visits of the double blind phase at months: −1 (screening), 0 (baseline), 1, 2, 3, 6, 9, 12, 15, 18, 21 and 24 (termination/early discontinuation). In case of the 6 months extended study, subjects were evaluated at study sites at months 27 and 30 (termination/early discontinuation of extended study), in this case month 24 was a regular scheduled visit.

EDSS was assessed every 3 months, MSFC every 6 months, and MRI was performed annually in all patients. A subgroup of patients (n=189) underwent additional MRI scans at months 3 and 6. Subjects successfully completing the study were offered the opportunity to enter into a 1-year open label extension. Patients who discontinued the study underwent a final termination visit and were not further evaluated, except for those who discontinued due to adverse events.

The following assessments were performed at specified time points:
1. Vital signs were measured at each study visit.
2. A physical examination is performed at months −1 (screening), 0 (baseline) 1, 3, 6, 12, 18 and 24 (termination/early discontinuation core study). In case of the 6 months extended study, additional examination was performed at month 30 (termination/early discontinuation of extended study).
3. The following safety clinical laboratory tests were performed:
   a. Complete blood count (CBC) with differential—at all scheduled visits. A reticulocyte count was added to the CBC at months 0 (baseline) and 24/30 (termination/early discontinuation).
   b. Serum chemistry (including electrolytes, liver enzymes, direct and total bilirubin and pancreatic amylase and CPK), and urinalysis—at all scheduled visits.
   c. A rapid urine β-hCG test was performed in women of child-bearing potential at baseline (month 0) and at each scheduled study visit thereafter (at site).
   d. β-hCG in women of child-bearing potential was performed at all scheduled visits.
   e. Starting after visit Month 3 a rapid urine β-hCG test was performed in women of child-bearing potential every 28 (±2) days. The subject was contacted by telephone within 72 hours after the test was scheduled to be performed and asked specific questions regarding the test. In case of suspected pregnancy (positive urine fi-hCG test result), the caller made sure that the study drug has been discontinued and the subject was instructed to arrive at the site as soon as possible with all study drugs.
4. Markers of inflammation (serum conventional C-reactive protein and fibrinogen)—at screening, baseline and all scheduled visits thereafter.
5. During the first 3 months periodical phone calls were placed by the site personnel every two weeks. A list of predefined questions relating to signs/symptoms suggestive of vascular thrombosis was presented to the subjects.
6. ECG was performed at months −1 (screening; additional recording, up to 30 minutes apart is performed if QT_{c} is less than 450 msec), (baseline; three recordings, 15 minutes apart), 1, 2, 3, 6, 12, 18 and 24 (termination/early discontinuation). In case of the 6 months extended study, ECG is performed at month 30 (termination/early discontinuation of the extended study).
7. Chest X-ray is performed at months −1 (screening), (if not performed within 7 months prior to the screening visit).
8. Adverse Events (AEs) are monitored throughout the study.
9. Concomitant medications are monitored throughout the study.
10. Neurological evaluations, including Expanded Disability Status Scale (EDSS), 25 foot walk test/Ambulation Index (AI), Functional systems (FS) are performed at months −1 (screening), 0 (baseline) and every 3 months during the study and the extended study period.
11. MS functional Composite (MSFC) was assessed at months −1 (screening) (three practices for training purposes only), at month 0 (baseline), 6, 12, 18 and 24 (termination/early discontinuation). In case of the 6 months extended study, the last MSFC was performed at months 30 (termination/early discontinuation of the extended study).
12. Subject-reported fatigue was assessed by the Modified Fatigue Impact Scale (MFIS) at months 0, 6, 12, 18, and 24 (termination/early discontinuation). In case of the 6 months extended study, additional MFIS was performed at month 30 (termination/early discontinuation of the extended study).
13. The general health status was assessed by the EuroQoL (EQ5D) questionnaire at month 0 (baseline) and month 24 (termination/early discontinuation of the study). In case of the 6 months extended study, the last EuroQoL (EQ5D) was performed at month 30 (termination/early discontinuation of the extended study) instead of month 24.
14. The general health status was assessed by the Short-Form general health survey (SF-36) subject-reported questionnaire at month 0 (baseline) and every 6 months thereafter, until termination/early discontinuation.
15. The subject underwent 5 assessments of binocular low-contrast visual acuity using the 100%, 2.5% and 1.25% contrast level charts [Sloan letter or Tumbling-E] in each assessment, at months 0 (baseline), 6, 12, 18 and 24 (termination/early discontinuation). In case of extending the study for 6 months, additional binocular low-contrast visual acuity assessment is performed at month 30 (termination/early discontinuation of the extended study).
16. Serum samples were collected from all subjects in order to investigate the potential mechanism of action of laquinimod and additional biomarkers of inflammation and potential biomarkers of MS disease at months: 0, 1, 12 and 24. In case of extending the study for 6 months the last serum sample is performed at month 30 (termination/early discontinuation of the extended study) instead of month 24.
17. The subjects underwent 3 MRI scans at months 0 (baseline), 12 and 24 (termination/early discontinuation). In case of the 6 months extended study, an additional MRI was performed at month 30 (termination/early discontinuation of the extended study).
18. Population PK study (PPK): Blood samples for PPK evaluation were collected from all subjects at months 1, 12 and 24. In case of extending the study for 6 months the last PPK evaluation was performed at month 30 (termination/early discontinuation of the extended study) instead of month 24.
19. Relapses were confirmed/monitored through the study. Since the “in study” relapse definition must be supported by an objective neurological evaluation, a neurological deficit must sustain long enough to eliminate pseudo-relapses. Therefore, in this clinical trial, a relapse was the appearance of one or more new neurological abnormalities or the reappearance of one or more previously observed neurological abnormalities wherein the change in clinical state lasts at least 48 hours and is immediately preceded by an improving neurological state of at least thirty (30) days from onset of previous relapse.
20. The allowed treatment for a relapse was intravenous Methylprednisolone 1 gr/day for up to 5 consecutive days.

### Inclusion/Exclusion Criteria

### Inclusion Criteria

1. Subjects must have a confirmed and documented diagnosis as defined by the Revised McDonald Criteria (Polman, 2005), with relapsing-remitting disease course.
2. Subjects must be ambulatory with converted Kurtzke EDSS score of 0-5.5.
3. Subjects must be in a stable neurological condition and free of corticosteroid treatment [intravenous (iv), intramuscular (im) and/or per os (po)]30 days prior to screening (month −1).
4. Subjects must have experienced one of the following:
   a. At least one documented relapse in the 12 months prior to screening.
   b. At least two documented relapses in the 24 months prior to screening.
   c. One documented relapse between 12 and 24 months prior to screening with at least one documented T1-Gd enhancing lesion in an MRI performed within 12 months prior to screening.
5. Subjects must be between 18 and 55 years of age, inclusive.
6. Subjects must have disease duration of at least 6 months (from the first symptom) prior to screening.
7. Women of child-bearing potential must practice an acceptable method of birth control. Acceptable method of birth control in this study include: surgical sterilization, intrauterine devices, oral contraceptive, contraceptive patch, long-acting injectable contraceptive, partner's vasectomy or double barrier method (condom or diaphragm with spermicide).
8. Subjects must be able to sign and date a written informed consent prior to entering the study.
9. Subjects must be willing and able to comply with the protocol requirements for the duration of the study.

### Exclusion Criteria

1. Subjects with progressive forms of MS.
2. An onset of relapse, unstable neurological condition or any treatment with corticosteroids [(iv), intramuscular (im) and/or per os (po)] or ACTH between months −1 (screening) and 0 (baseline).
3. Use of experimental or investigational drugs, and/or participation in drug clinical studies within the 6 months prior to screening.
4. Use of immunosuppressive including mitoxantrone (Novantrone®) or cytotoxic agents within 6 months prior to screening visit.
5. Previous use of any one of the following: natalizumab (Tysabri®), caldribine, laquinimod.
6. Previous treatment with glatiramer acetate (Copaxone®) Interferon-β (either 1a or 1b) or intravenous immunoglobulin (IVIG) within 2 months prior to screening visit.
7. Systemic corticosteroid treatment of ≧30 consecutive days duration within 2 months prior to screening visit.
8. Previous total body irradiation or total lymphoid irradiation.
9. Previous stem cell treatment, autologous bone marrow transplantation or allogenic bone marrow transplantation.
10. A known history of tuberculosis.
11. Acute infection two weeks prior to baseline visit.
12. Major trauma or surgery two weeks prior to baseline.
13. Use of inhibitors of CYP3A4 within 2 weeks prior to baseline visit (1 month for fluoxetine).
14. Use of amiodarone within 2 years prior to screening visit.
15. Pregnancy or breastfeeding.
16. A ≧3xULN serum elevation of either ALT or AST at screening.
17. Serum direct bilirubin which is ≧2xULN at screening.
18. A QTc interval which is 450 msec (according to machine output) obtained from:
   a. Two ECG recordings at screening visit, or
   b. The mean value calculated from 3 baseline ECO recordings.
19. Subjects with clinically significant or unstable medical or surgical condition that would preclude safe and complete study participation, as determined by medical history, physical examination, ECG, laboratory tests or chest X-ray. Such conditions may include:
   a. A cardiovascular or pulmonary disorder that cannot be well-controlled by standard treatment permitted by the study protocol.
   b. A gastrointestinal disorder that may affect the absorption of study medication.
   c. Renal or metabolic diseases.
   d. Any form of chronic liver disease.
   e. Known human immunodeficiency virus (HIV positive status.
   f. A family history of Long-QT syndrome.
   g. A history of drug and/or alcohol abuse.
   h. Major psychiatric disorder.
20. A known history of sensitivity to Gd.
21. Inability to successfully undergo MRI scanning.
22. Known drug hypersensitivity that would preclude administration of laquinimod, such as hypersensitivity to: mannitol, meglumine or sodium stearyl fumarate.

### Ancillary High-Through Output Gene Expression Study

The goal of this ancillary study was to characterize gene expression changes and corresponding biological mechanisms induced in PBMC of RRMS patients by LAQ treatment. According to ALLEGRO clinical trial inclusion criteria, 25 patients were randomly assigned to receive LAQ (n=13, age 38.8±2.3 years, female/male ratio: 8/5) or placebo (n=12, age 37.2±3.4 years, female/male ratio: 8/4).

Peripheral blood samples were obtained from RRMS patients at baseline before start of LAQ treatment or placebo, after 0, 1, 6 and 24 month of treatment (visit 0, 1, 6 and 7 according to ALLEGRO clinical trial protocol correspondently) for gene microarray analysis.

Briefly, 1) Peripheral blood mononuclear cells (PBMC) were obtained from RRMS patients that participated in ALLEGRO and were treated daily with 0.6 mg LAQ or placebo. PBMC were subjected for gene expression analysis (HU-133A-2-Affymatrix arrays) at baseline and at 1 and 6 months of LAQ treatment; 2) Data was analyzed by Partek Genomics Solution software. Most informative genes (MIGs) were defined as those that differentiated between groups with p<0.01. Gene functional annotation, enrichment and pathway analysis were performed by Ingenuity software. For each time point, genes that changed in placebo group were excluded from further analysis; and 3) Verification of LAQ related mechanism was performed by Western blot.

LAQ was found to induce a differential gene expression of 354 MIGs at 1 month and 1562 MIGs at 6 months of treatment.

This study shows that LAQ down-regulates genes associated with adhesion, migration and chemotaxis of PBMC either directly or via TGFb suppression. These effects were observed after 1 and strengthened after 6 month of LAQ treatment. LAQ also down-regulates PAI-1 suggesting activation of fibrinolysis and possibly subsequent neuroprotection. Both effects can contribute to amelioration of MS clinical symptoms.

### RNA Isolation and Hybridization

PBMC were extracted from 15 ml peripheral blood, separated by Ficoll-Hypaque gradient. Total RNA was extracted using both Trizol (Invitrogen, USA) and Phase-Lock-Gel columns (Eppendorf, Germany) including a DNase digestion step. RNA integrity was assessed by RNA Experion automated electrophoresis system (Bio-Rad Laboratories, Hercules, Calif.).

Probe synthesis using 3 μg total RNA, hybridization, detection, and scanning was performed according to the standard Affymetrix, Inc. USA protocols; cDNA was synthesized using the Two-Cycle cDNA Synthesis Kit (Affymetrix, Inc., USA), and in-vitro transcription performed with the GeneChip IVT Labeling Kit (Affymetrix, Inc., USA). The biotin-labeled IVT-RNA was hybridized to HG-U133A-2 arrays containing 18,400 gene transcripts, each corresponding to 14,500 well-annotated human genes, washed in a GeneChip Fluidics Station 450 (Hewlett Packard, USA, GeneArray-™ scanner G2500A) and scanned according to the manufacturer's protocol (Affymetrix, Inc., USA).

### Data Analysis

Data analysis was performed on Partek Genomics Solution software (www.partek.com; Partek Incorporated, St. Louis, Mo.). Expression values were computed from raw CEL files by applying the Robust Multi-Chip Average (RMA) background correction algorithm. RMA correction included: 1) values background correction; 2) quintile normalization; 3) log 2 transformation; and 4) median polish summarization. The ANOVA, Repeated Measures and correlation analysis implicated in Partek software ware applied to evaluate LAQ effects. Most informative genes MIGs were defined as those that differentiated between experimental groups with p<0.01. All p-values were calculated for False Discovery Rate (FDR) multiple test correction at p=0.05.

Additionally, significance of individual genes was tested by parametric T-test and non parametric Mann-Whitney test using Bootstrapping approach based on repeated permutations of the data with 5% FDR for multiple testing.

Gene functional annotation, enrichment, and pathway analyses to identify the leading biological pathways that operated under LAQ treatment were performed by Ingenuity Pathways Analysis software (www.ingenuity.com). Enrichment was defined as significantly (p<0.05) higher proportion of genes than expected by chance in a given gene set.

### Western Blot Analysis

For verification of key genes on protein level Western blot analysis was performed. Supernatant was collected and protein concentration was determined using a Bradford assay (Pierce, Rockford, Ill., USA) according the manufacturer's guidelines. Equal amounts of protein were suspended in sample buffer and boiled for 5 min. Cell lysates were resolved on 10% SDS-polyacrylamide gel electrophoresis (PAGE). Gels were transferred to a Nitrocellulose membrane (Amersham, Buckinghamshire, UK), blocked with 1% BSA in Tris-buffered saline Tween (TBST) buffer (20 mM Tris, 137 mM NaCl and 0.1% Tween 20) and incubated with primary antibody overnight at 4° C. After washing three times with TBST buffer, blots were incubated with alkaline peroxidase-conjugated secondary antibody. Antibodies were diluted in the blocking solution. The blots were then washed three times with TBST buffer and analyzed by standard chemiluminescence (Supersignal Kit, Pierce, Rockford, Ill., USA) according to the company's protocol.

### Results

According to ancillary study aims, 72 blood samples were collected. The number of samples and corresponding demographical data is presented in Table 1.

TABLE 1
Clinical and demographical data of subjects

| Visit(Month) | 0(0) | 1(1) | 4(6) | 7(24) |
|---|---|---|---|---|
| N of patients | 21 | 23 | 17 | 11 |
| LAQ(N) | 12 | 13 | 12 | 8 |
| Placebo(N) | 9 | 10 | 5 | 3 |
| age | 38.07 ± 2.20 | 36.66 ± 1.93 | 38.17 ± 2.39 | 40.25 ± 2.84 |
| N(%) male | 7/21(33.33%) | 8/23(34.78%) | 7/17(41.18%) | 5/11(45.45%) |
| N(%) female | 14/21(66.67%) | 15/23(65.22%) | 10/17(58.82%) | 6/11(54.55%) |
| LAQ | N(%) male | 5/12(41.67%) | 5/13(38.46%) | 5/12(41.67%) |
| | N(%) Female | 7/12(58.33%) | 8/13(61.54%) | 7/12(58.33%) |
| Placebo | N(%) male | 2/9(22.22%) | 3/10(30.00%) | 2/5(40.00%) |
| | N(%) Female | 7/9(77.78%) | 7/10(70.00%) | 3/5(60.00%) |

### ANOVA Analysis

ANOVA analysis was used to compare PBMC gene expression after 1, 6 or 24 month of LAQ treatment with baseline gene expression.

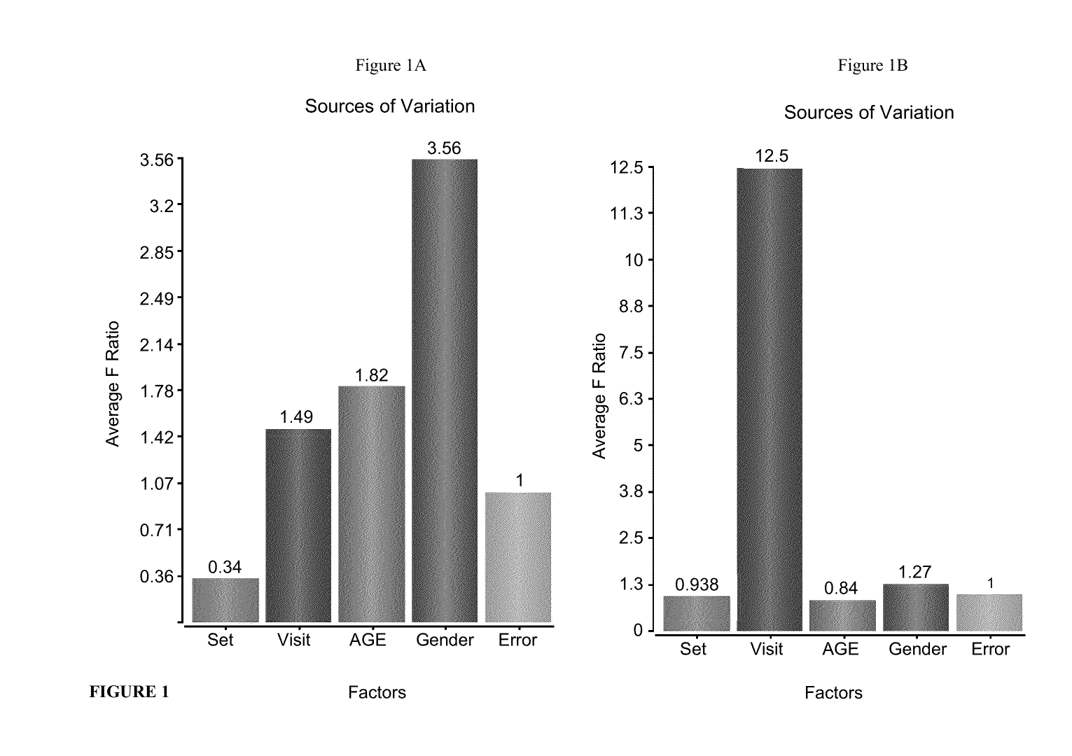

For each time point inventors performed analysis source of variation in dataset. (FIG. 1). Age, gender and batch effects were considered as confounders regarding LAQ or Placebo related changes. For each time point genes associated with Placebo effect were evaluated and excluded from further analysis.

Table 2 below shows number of genes significantly changed by ANOVA test in each time point as compared with baseline.

TABLE 2
Number of LAQ related MIGs according to ANOVA p values.

| | Visits | | | | |
|---|---|---|---|---|---|
| | according to | # of genes | Down | Up- | |
| Time Point | protocol | p < 0.01 | regulated | regulated | FDR |
| 1 month | 1 | 354 | 348 | 6 | No |
| 6 month | 4 | 1562 | 1552 | 10 | 43 |
| 6 and 24 | 4 and 7 | 2922 | 2911 | 11 | 1564 |
| month | | | | | |

Table 3 and Table 4 below shows the main biological pathways and functions affected by LAQ.

TABLE 3
Main biological pathways and functions affected by LAQ

| After one month of treatment | | | | After six months of treatment | | | |
|---|---|---|---|---|---|---|---|
| | | genes | p- | | | genes | p- |
| function | pathway | (#) | value | function | pathway | (#) | value |
| Inflammatory | Adhesion of | 9 | 1.2*10⁻³ | Development | G protein | 73 | 3.1*10⁻⁵ |
| response | phagocytes | | | | Coupled | | |
| | | | | | Receptor | | |
| | | | | | Signaling | | |
| | Chemotaxis | 4 | 6.0*10⁻³ | | Arachidonic | 18 | 2.2*10⁻³ |
| | of Neutrophils | | | | Acid | | |
| | | | | | metabolism | | |
| | Transmigration | 8 | 1.9*10⁻³ | | TGFB | 14 | 4.3*10⁻² |
| | of | | | | signaling | | |
| | leukocytes | | | | | | |
| | Caveolar | 8 | 1.8*10⁻⁴ | Inflammatory | Leukocyte | 29 | 9.4*10⁻³ |
| | mediated | | | response | Extravasation | | |
| | endocytosis | | | | Signaling | | |
| | Clathrin | 12 | 2.1*10⁻⁴ | | Caveolar | 13 | 2.1*10⁻² |
| | mediated | | | | mediated | | |
| | endocytosis | | | | endocytosis | | |
| Hematological | Aggregation | 18 | 3.5*10⁻¹⁰ | Cell | Adhesion of | 119 | 2.4*10⁻⁵ |
| system | of blood | | | signaling | cells | | |
| | platelets | | | | | | |
| | Activation of | 13 | 1.4*10⁻⁸ | | Neuro- | 56 | 2.1*10⁻⁵ |
| | blood | | | | transmission | | |
| | platelets | | | | | | |
| | Aggregation | 19 | 2.6*10⁻⁸ | Hematological | Intrinsic | 6 | 6.2*10⁻² |
| | of blood cells | | | system | prothrombin | | |
| | | | | | activation | | |
| | | | | | pathway | | |
| | Coagulation | 14 | 7.4*10⁻⁷ | | Coagulation | 7 | 7.4*10⁻² |
| | of blood | | | | system | | |

TABLE 4
Main biological pathways and functions affected by LAQ

| After one month of treatment | | | | After six months of treatment | | | |
|---|---|---|---|---|---|---|---|
| | | genes | p- | | | genes | p- |
| function | pathway | (#) | value | function | pathway | (#) | value |
| Inflammatory | TGFb | 10 | 3.2*10⁻³ | Inflammatory | TGFb | 14 | 3.7*10⁻² |
| response | signaling | | | response | signaling | | |
| | | | | | IL-12 | 11 | 3.2*10⁻³ |
| | | | | | signaling | | |
| Cellular | Adhesion & | 9 | 1.2*10⁻³ | Cellular | Invasion of | 120 | 5.6*10⁻⁵ |
| Movement | migration of | | | movement | cells | | |
| | phagocytes | | | | | | |
| | Chemotaxis of | 4 | 6.0*10⁻³ | | Adhesion | 119 | 2.4*10⁻⁵ |
| | neutrophils | | | | of cell | | |
| | Transmigration | 8 | 1.9*10⁻³ | | Leukocyte | 31 | 4*10⁻³ |
| | of leukocytes | | | | extravasation | | |
| | | | | | signaling | | |

The majority of genes that showed significant changes at each time points were down regulated. The functional enrichment analysis of 354 genes affected after 1 month of treatment showed suppression of 50 molecules associated with different mechanisms of inflammatory response (p value from 3.4*10⁻¹⁰ to 1.1*10⁻²). This included for example suppression of adhesion of phagocytes (p=1.2*10⁻³) and chemotaxis of neutrophils (p=6.0*10⁻³) based on suppression of TGFB1, ITGB1, ITGB3, ITGB5 and CXCL5, ITGB1, MMP1, TGFB1 correspondently. The most significant canonical pathways are suppression of Caveolar and Clatrin mediated Endocytosis Signaling (p=1.8*10⁻⁴ and 2.1*10⁻⁴). The interesting findings are suppression of PTCR and CD84 that function in adhesion interaction between T lymphocytes and accessory cells.

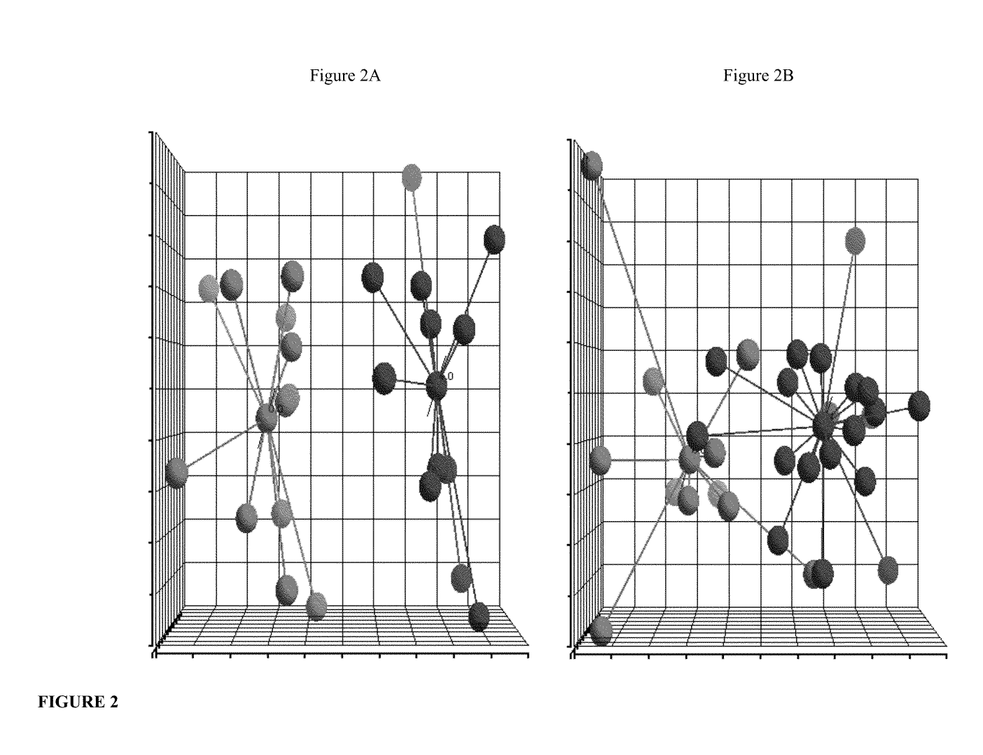

As shown in Table 2 the number of genes significantly affected by LAQ (p<0.01) changed from 354 to 1562 between 1 and 6 months of treatment, and 43 genes passed stringent FDR criteria for 6 months of treatment (FIG. 2A). Total 260 genes out of 1562 were related to suppression of Cellular movement functions (p value of enrichment from 4.6*10⁻⁷ to 5.4*10⁻³). G protein Coupled Receptor Signaling (p=3.1*10⁻⁵), Arachidonic Acid metabolism (p=2.2*10⁻³), Leukocyte Extravasation Signaling (p=9.4*10⁻³), Caveolar mediated endocytosis Signaling (p=2.1*10⁻²), TGF beta Signaling (p=4.3*10⁻²), Adhesion of cells (p=2.4*10⁻⁵), Neurotransmission (p=2.1*10⁻⁵), Intrinsic prothrombin activation pathway (p=6.2*10⁻²) and Coagulation system (p=7.4*10⁻²) were the most significantly down-regulated canonical pathways after 6 months of treatment.

The number of patients involved in analysis at 24 months of treatment was relatively low, thus in order to improve statistical power, the inventors combined data from 6 and 24 months which resulted in evaluation of 2922 genes with p<0.01 and 1564 genes that passed FDR criteria (FIG. 2B). Due to high statistical significance of combined 6 and 24 months LAQ signature the most detailed functional analysis was applied.

### LAQ Down-Regulates Expression of Migration/Adhesion Molecules

Functional analysis of 1564 genes that passed FDR criteria after more than 6 months of treatment showed significant enrichment of down-regulated genes (n=305) related to different kind of cellular movement mechanisms with p values from 1.5*10⁻³ to 4.5*10⁻¹⁴. This included for example suppression of cell migration function (n=233, p=4.5*10⁻¹⁴) and chemotaxis (n=78, p=4.3*10⁻⁵).

LAQ significantly down-regulated a range of Metalloproteinase family members such as MMP1, MMP14, MMP16, MMP24, MMP25, MMP26, MMP28, ADAM12 and ADAM22. Several Integrin and chemokine related genes were down-regulated upon treatment of LAQ: ITGB1, ITGB5, ITGB6, ITGA8, ITGB8, and GPIIB-III3 (fibrinogen receptor), CXCL4, CCL14, CCL18, CCXCR1 (XCR1), CXCL7 (PPBP).

These results are in line with previous studies reporting that LAQ interferes with the migratory capacity of T cells in mice with EAE (Wegner et al., 2010, Jadidi-Niaragh et al., 2011).

### LAQ Down-Regulates Pro-Inflammatory Constituents

In addition to suppression of cell migration ability, treatment of LAQ demonstrated significant down-regulation of IL-1R, IL-8R and IL-22R, IL-9, TNFRSF4, and RORC (RORgamma), all of which are inflammation-related genes that are known to play a role in EAE (Jadidi-Niaragh et al., 2011). Recently, it has been shown that IL-9 is important for T-cell activation and differentiation in autoimmune inflammation of the CNS, and that IL-9−/− mice developed significantly less severe EAE than their WT counterparts (Li et al., 2011). The results show reduced expression of SOCS (suppressor of cytokine signaling), a negative regulator of immune response, which is indirectly regulated by TGFb1 and ICOSLG (inducible T-cell co-stimulator ligand). In correlation with down-regulation of the pro-inflammatory constituents, LAQ treatment significantly reduced the expression of CSF1, CSF2 and CSF3 and indirectly affected FoxP3 expression. ROR (RORgamma) can directly interact with FoxP3. However, the functional consequence of this interaction is not clear because none of the previous studies on LAQ effect described an effect on Treg. Clatrin and Caveolar-mediated Endocytosis pathways are significantly suppressed (p=5.0*10⁻⁴ and p=5.8*10⁻⁴) after 1 month of treatment.

### TGFB1 Related Mechanism

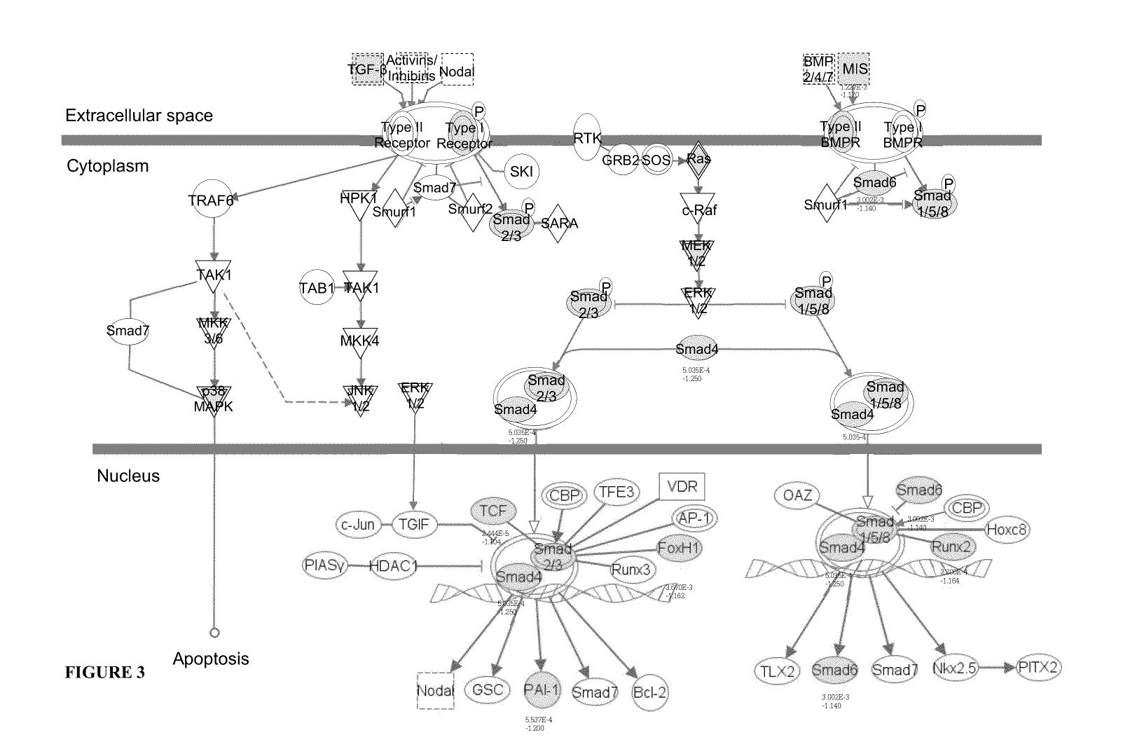

6 months or longer treatment of LAQ induced significant suppression of genes related to the TGFB pathway (p=1.9*10⁻²) (FIG. 3)

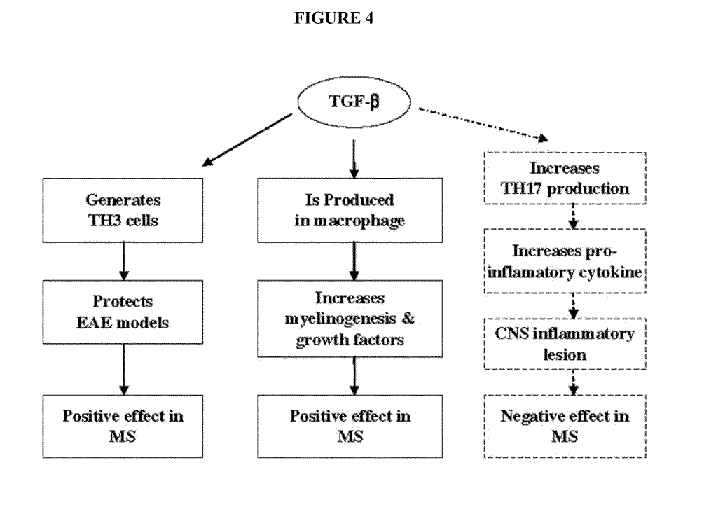

TGFB is a potent regulatory cytokine with diverse effects on hematopoietic cells. The pivotal function of TGFB in the immune system is to maintain tolerance via the regulation of lymphocyte proliferation, differentiation, and survival. Among T cells, CD4+CD25+FOXP3+ T regs contain the main source of TGFB that suppresses immune responses in inflammatory sites. Defects in TGFB1 expression or its signaling in T cells correlate with the onset of several autoimmune diseases. It has been shown previously that besides its anti-inflammatory role, TGFB paradoxically acts as a pro-inflammatory cytokine and induces IL-17-producing pathogenic T helper cells (Th IL-17 cells) during an inflammatory response in which IL-6 is produced (Mirshafiey and Mohsenzadegan, 2009) (FIG. 4). Consistent with the proposed pro-inflammatory role of TGFB our analysis showed down-regulation of several TGFB-related genes and its downstream signaling components including: LTBP1 (latent transforming growth factor beta binding protein 1), Type I receptor, Smad2/3, Smad4, TCF [hepatocyte nuclear factor 4 alpha (HNF4A)] and PAI-1 (FIG. 3).

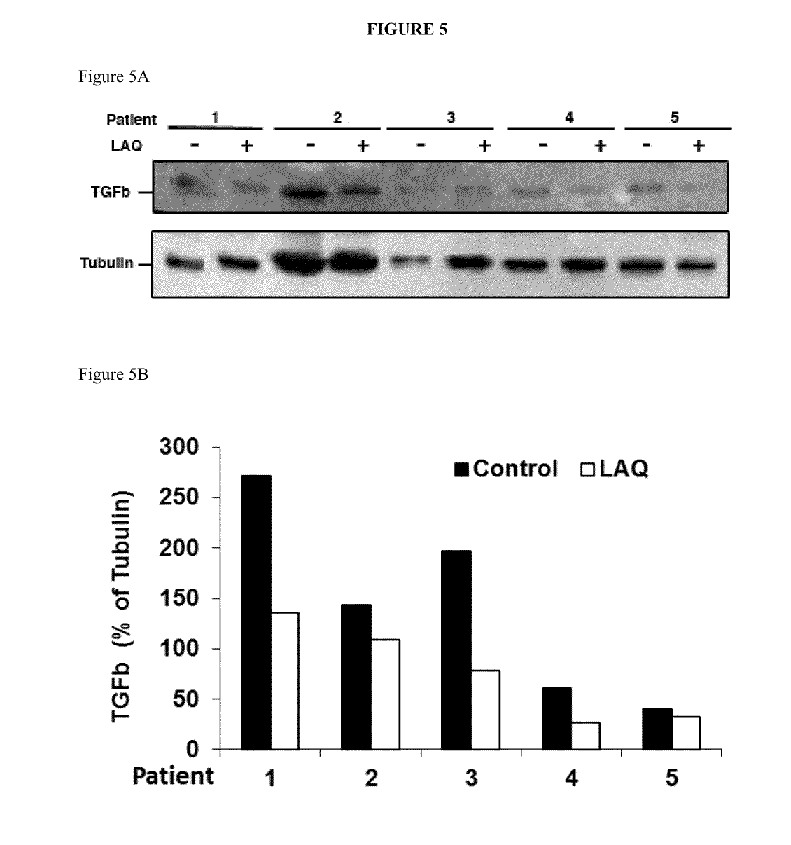

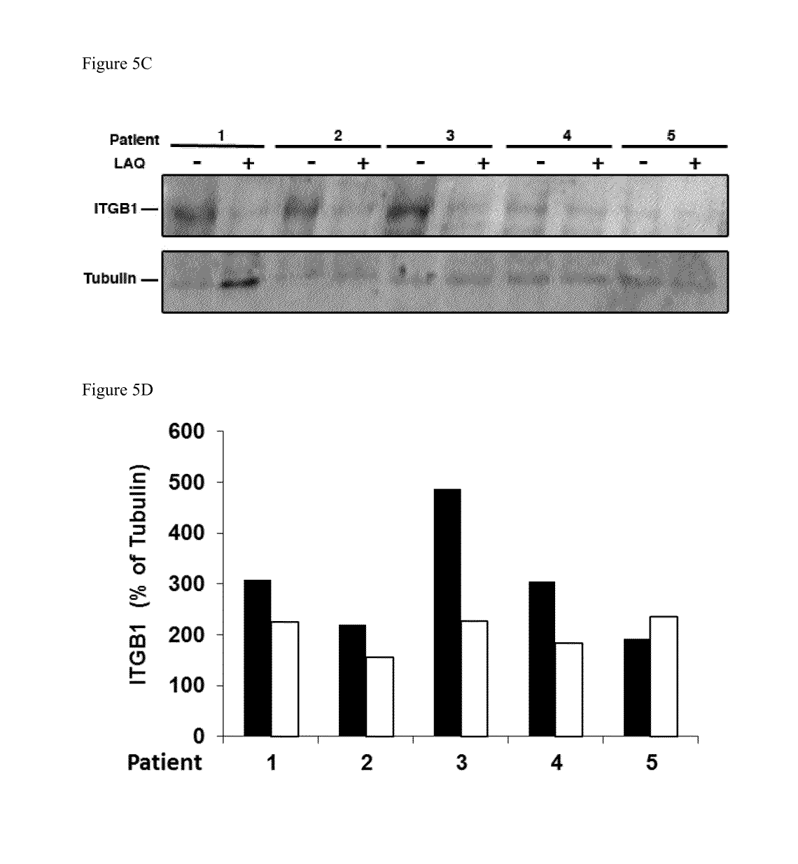

Western blot analyses in four out of five patients who received 6 months of LAQ treatment verified down-regulation of TGFB1 protein level by 20-50%, as shown by quantification of band intensities normalized against Tubulin (FIGS. 5A and 5B).

### LAQ Induced Down-Regulation of Serpine 1 [(Plasminogen Activator Inhibitor 1(PAI-1)] and Other Members of the Coagulation System

While anti-inflammatory properties of LAQ were previously reported (Gurevich et al., 2010, Brück and Wegner, 2011), the current study demonstrated down-regulation of several members of the coagulation pathway including F2 (thrombin), F7 (factor VII), F10 (factor X), FGB (fibrinogen beta-chain), TFPI [(tissue factor pathway inhibitor (lipoprotein-associated coagulation inhibitor)], Serpine 1 [plasminogen activator inhibitor (PAI-1)] and also two other members of the Serpine 1 family (SerpinA3 and SerpinB3). PAI-1 inhibits the serine proteases tissue plasminogen activator (tPA) and uPA/urokinase, thus it is an inhibitor of fibrinolysis, the physiological process that degrades blood clots. Although the tPA-plasmin cascade promotes neurodegeneration in excitotoxin-induced neuronal death, it has been demonstrated to have a protective role in inflammatory conditions with BBB disruption by removing fibrin, which exacerbates axonal injury (Gveric et al., 2003). Moreover, in the mouse model of MS, EAE incidence and clinical severity were reduced in PAI-1−/− mice, where clinical relapses were absent in PAI-1−/− mice and the subsequent reduction in neuroinflammation was coupled with a higher capacity for fibrinolysis in spinal cord samples from PAI-1−/− mice, in association with increased tPA activity (East et al., 2008).

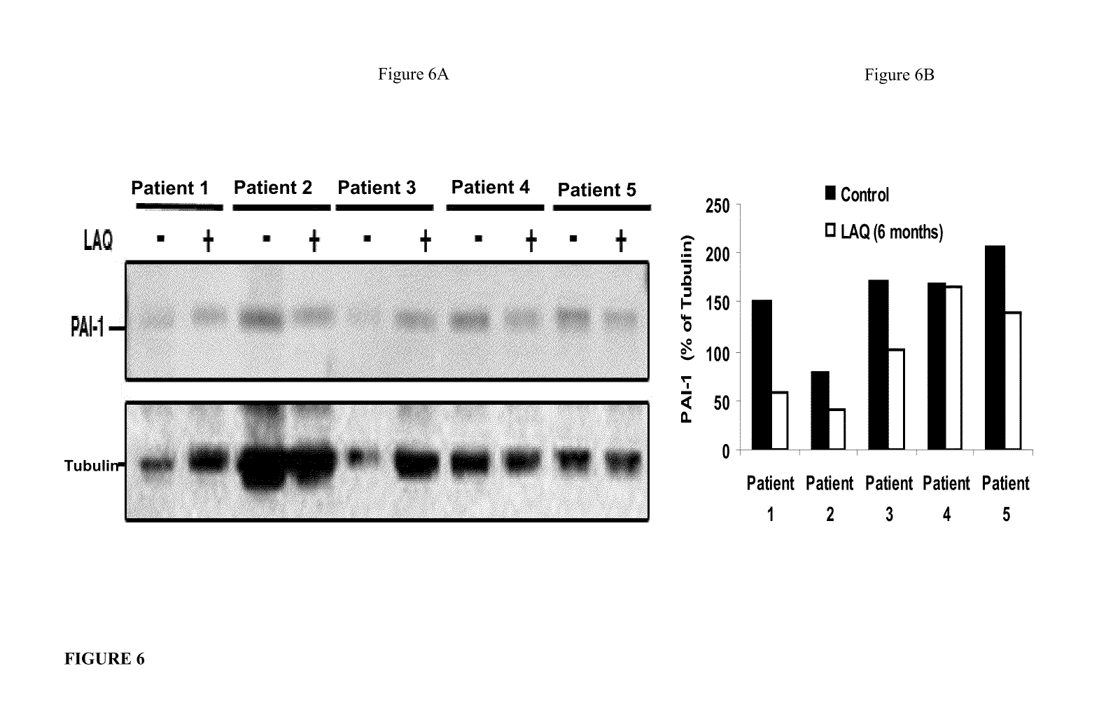

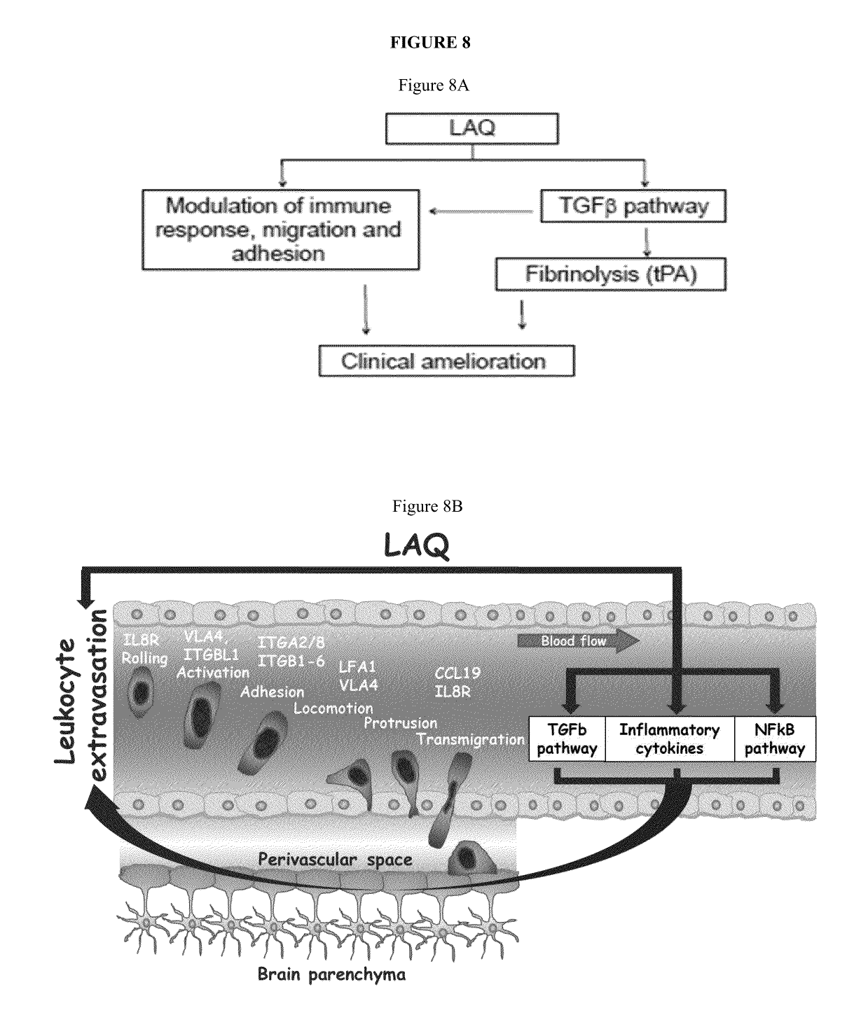

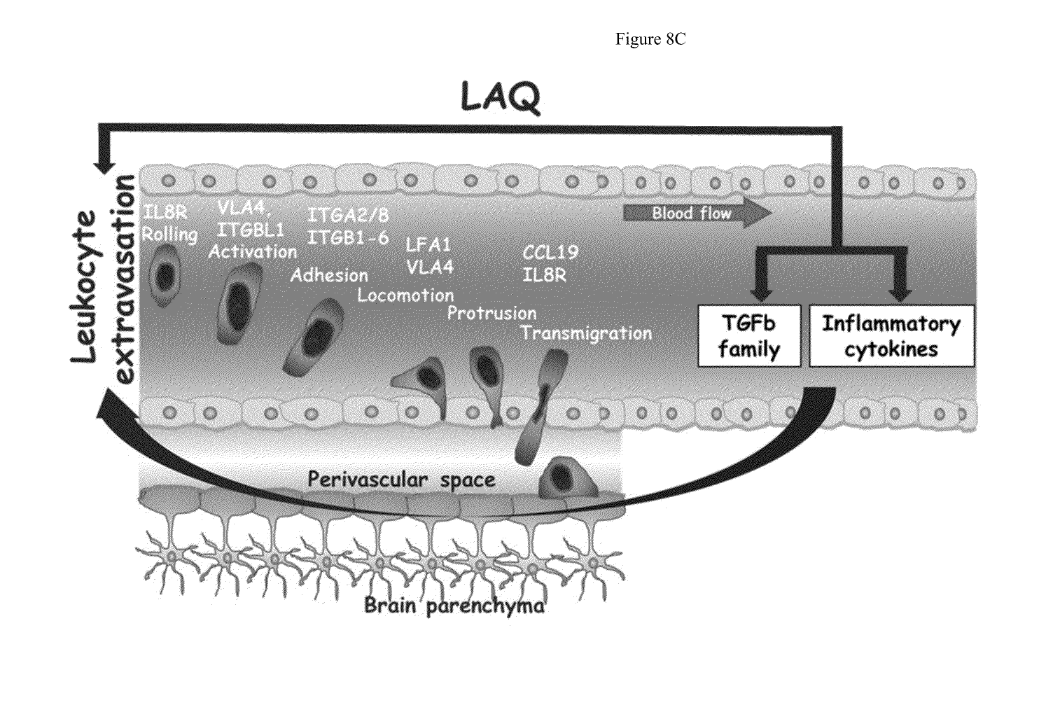

Importantly, consistent with our gene expression results, which shows significant down-regulation of PAI-1, the Western blot analysis shown in FIG. 6 demonstrates reduced expression of PAI-1 protein by 30-50% in four out of five patients after 6 months of LAQ treatment. The quantification of band intensities were normalized against tubulin. Previous gene expression analysis of PBMCs treated in vitro with LAQ, also showed significant down-regulation of PAI-1 (Gurevich et al., 2010). These results suggest positive correlation between LAQ-induced down-regulation of TGFB1 and PAI-1 expression and implicate LAQ in suppression of the neurodegenerative role of PAI-1, as demonstrated by East et al., 2008 and Overic et al., 2003. The proposed mechanism of LAQ effects on PBMS is shown in FIG. 8A and FIG. 8B.

### Correlation and Repeated Measures Analysis.

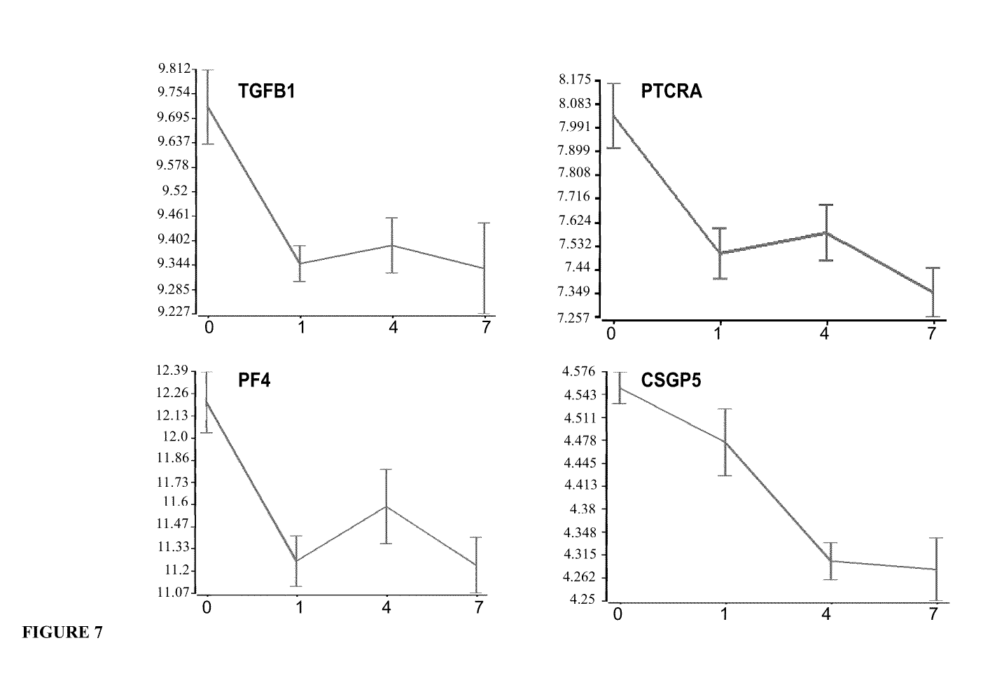

In ANOVA model each patients has to be independent under each condition. However in repeated measures algorithm the independence requirement is removed and each patients can repeatedly tested in different condition and responses from the same patients are correlated. Repeated measures increase statistical power and thus fewer subjects are needed to have adequate power. The inventors applied repeated measures analysis to evaluated effect of LAQ in same patients across all visits (28 microarrays related to 7 patients). First, using this approach the inventors evaluated Placebo effects and excluded placebo related genes from further analysis. The effect of LAQ realized in significant changing of 174 genes that pass FDR criteria with p<0.0004. Functional analysis of this gene list confirmed ANOVA results and among other included PTCRA, ITGB3, ITGA2B, ITGB5, PF4 and TGFB1 genes. The same of those gene profiles demonstrated in FIG. 7

### Summary

In vivo effects of LAQ on PBMC showed down-regulation of genes as shown in Tables 3 and 4, including genes involving cell motility, adhesion, chemotaxis, IL1 and IL8 mediated inflammation, and Clatrin and Caveolar-mediated Endocytosis pathways, etc.

Functional enrichment analysis of most informative genes at 1 month of LAQ treatment demonstrated down-regulation of genes associated with inflammatory response, genes associated with TGFb signaling including TGFb1, TGFb1I1 and LTBP1 (p value range=3.8*10⁻⁴ to 6.7*10⁻³), (see Table 4) and other genes associated with cellular movement and migration (TNFSF4, SELP, ITGA8, ITGB1/3/5, CXCL5/7 and BMP6 genes).

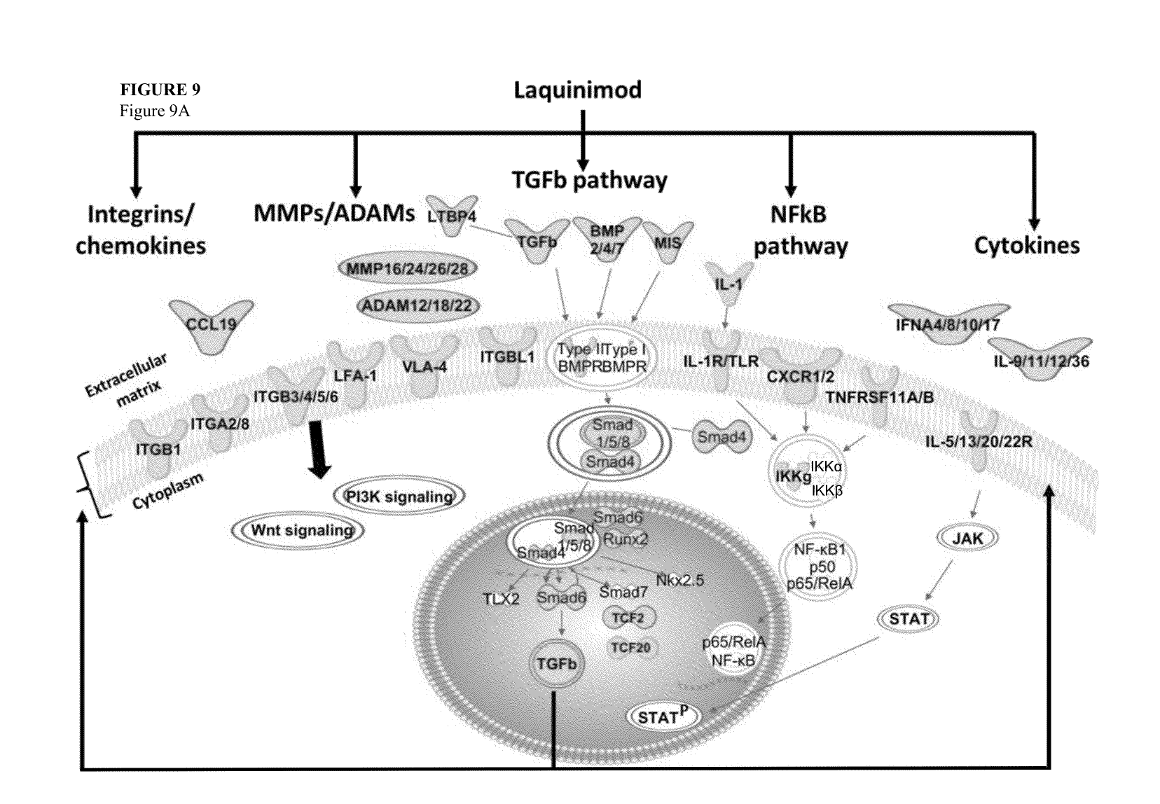

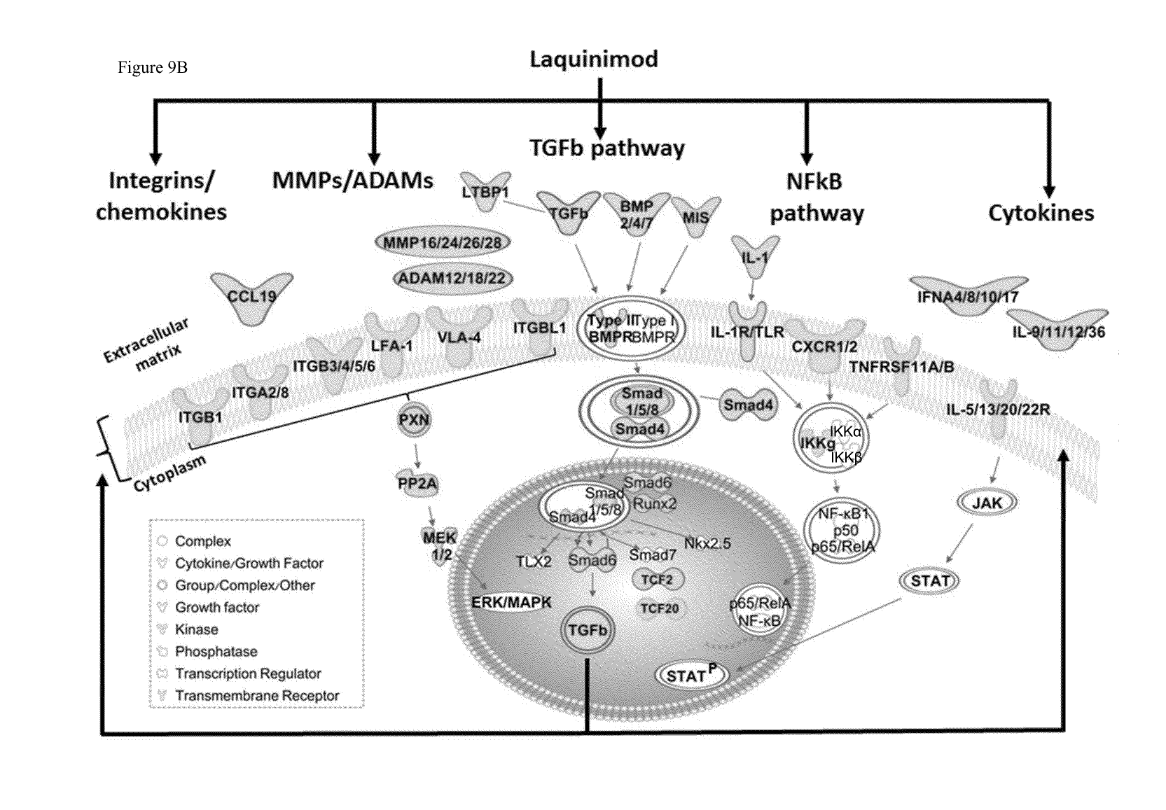

Suppression of inflammation was further strengthened after 6 months of LAQ treatment, where there was suppression of large number of genes associated with adhesion, migration and leukocyte extravasation signaling (ITGA2/8, ITGb1/3/4/5/6, ITGBL1, MMP16/24/26/28 and ADAM12/18/22) accompanied by suppression of IL-1/5/8/13/20/22R, IL-9/11/12/36, TNFRSF11A/B, and IFNA4/8/10/17. Notably, LAQ treatment also down-regulated TGFB expression including its downstream signaling constituents (LTBP4, MEK1/2, TGFB type I receptor and smad2/3/4). Laquinimod treatment down-regulated TGFb expression including its associated signaling constituents (LTBP4, type II BMPR and smad1/4/5/6/8) and the NFkB signaling constituents (IL-1, IL-1R and IKKg) (see, FIG. 9A). Interestingly, the final downstream affected molecule in the TGFb pathway is the ITGB1 constituent of several Integrins that participates in rolling, adhesion, activation and locomotion and thereby regulates cellular movement in concert with CCL19, MMPs and ADAMs. The suppression of TGFB and ITGB1 was confirmed by Western blot (see FIG. 5). The proposed mechanism of Laquinimod effects on PBMC is depicted in FIG. 8A and FIG. 8B. The underlying mechanism of LAQ treatment is characterized by down-regulation of Serpine 1 (Plasminogen activator inhibitor 1, PAI-1), a potent inhibitor of fibrinolysis and the final downstream molecule affected in the TGFB pathway, and other members of the coagulation system. These results suggest that in addition to its ability to modulate cytokines expression and adhesion/migration, LAQ also modulates the coagulation pathway, contributes to fibrinolysis (by effective fibrin removal) and thereby reduces neuronal damage. The majority of changes described in this report could be explained by considerable suppression TGFB1 mechanism.

### Conclusion

Laquinimod suppresses inflammation as shown by down-regulation of genes of pro-inflammatory cytokines, TGFb and NFkB pathways.
Laquinimod suppresses the entire set of genes associated in the multistep paradigm of leukocyte extravasation suggesting its capability to inhibit infiltration of inflammatory cells to the CNS.
These effects on inflammation and cell movement occurred either directly or via TGFb suppression were observed after one month and strengthened after six months of Laquinimod treatment.
The down-regulation of TGFb, NFkB and cellular movement components by Laquinimod strongly suggests diminished CNS infiltration and subsequent reduction in axonal damage which may contribute to the therapeutic benefits of laquinimod in amelioration of MS clinical symptoms.

### Example 2

### The Role of Laquinimod in Modulation of the Immune Response in Relapsing-Remitting Multiple Sclerosis: Lessons from Gene Expression Signature

### Abstract

The inventors analyzed the molecular pathways induced by LAQ treatment in patients that participated in the ALLEGRO trial using gene expression microarray analysis. Blood transcriptional changes after one and six months of treatment were compared to baseline to identify LAQ induced MIGs (p<0.01) and operating pathways.

The inventors identified 354 MIGs at one month and 1562 MIGs at six months of treatment. LAQ treatment effects were enhanced by duration of treatment and characterized by down-regulation of inflammatory responses via TGFb and NFkB signaling in combination with suppression of genes associated with cellular movement including adhesion, migration and leukocyte extravasation signaling like integrins, chemokines and metalloproteinases with further down-regulation of genes encoding pro-inflammatory cytokines.

These results demonstrate that LAQ acts via suppression of inflammation mainly through arrest of leukocytes extravasation and thereby could contribute to amelioration of disease activity in RRMS patients.

### 1. Introduction

LAQ was demonstrated to inhibit the development of acute experimental autoimmune encephalomyelitis (EAE) and to reduce EAE clinical score in mice treated after disease onset (Brück and Wegner, 2011; Brunmark et al., 2002; Jolivel et al., 2013; Ruffini et al, 2013; Schulze-Topphoff et al., 2012; Wegner et al., 2010). Clinically, LAQ demonstrated about 40% reduction in the cumulative number of gadolinium enhanced lesions in brain MRI in 106 RRMS patients as compared to 102 placebo treated RRMS patients (Comi et al., 2008). Recently, the Assessment of Oral Laquinimod in Preventing Progression in Multiple Sclerosis (Filippi et al., 2014) study demonstrated that LAQ treatment modestly decreased annualized relapse rate, slowed progression of disability and prevented white and gray matter atrophy in RRMS patients treated for 24 months (Comi et al., 2008; Filippi et al., 2014).

The mechanisms by which LAQ suppresses the development of EAE involve modulation of Th1/Th2 response, interference with the migration capacity of T cells (Bruck and Vollmer, 2013; Brück and Wegner, 2011; Wegner et al., 2010; Yang et al., 2004; Zou et al., 2002), and prevention of inflammation-induced synaptic alterations occurring in EAE (Ruffini et al., 2013). In addition, in MS patients, it has been reported that LAQ modulates B cells and their regulatory effects on T cells (Toubi et al., 2012), and down-regulates immunogenicity of dendritic cell (Jolivel et al., 2013).

In a previous study (Gurevich et at, 2010), the inventors characterized the molecular effects of LAQ in-vitro in separated immune cells subtypes obtained from RRMS patients using gene expression microarrays. The inventors demonstrated that LAQ induced suppression of genes related to antigen presentation and corresponding inflammatory pathways involving NFkB signaling, pleiotrophin-induced inflammatory cytokines, chemokine and toll like receptor signaling, down-regulation of Th2 response in CD14+ macrophages and CD4+ T cells, suppression of proliferation in CD8+ T cells, and suppression of antigen presentation and adhesion in CD19+ B cells via suppression of NFkB pathway.

To further elucidate the molecular mechanisms underlying the therapeutic effects of LAQ in RRMS, the inventors performed high throughput gene expression microarray analysis of PBMCs from RRMS patients that participated in the ALLEGRO trial.

### 2. Methods

Peripheral blood samples were obtained from RRMS patients treated with LAQ 0.6 mg/day or placebo as an ancillary study to the Assessment of Oral Laquinimod in Preventing Progression in Multiple Sclerosis trial (Filippi et al., 2014). Blood samples were obtained at baseline and after one and six months of treatment.

### 2.1. RNA Isolation and Hybridization

PBMC were extracted from 15 ml peripheral blood, separated by Ficoll-Hypaque gradient. Total RNA was extracted using both Trizol including a DNase digestion step. RNA integrity was assessed by RNA Experion automated electrophoresis system. Probe synthesis using 3 μg total RNA, hybridization, detection, and scanning was performed according to the standard Affymetrix, Inc. USA protocols; cDNA was synthesized using the Two-Cycle cDNA Synthesis Kit (Affymetrix, Inc., USA), and in-vitro transcription performed with the GeneChip IVT Labeling Kit (Affymetrix, Inc., USA). The biotin-labeled IVT-RNA was hybridized to HG-U133A-2 arrays (Affymetrix, Inc., USA) containing 14,500 well-annotated human genes, washed in a GeneChip Fluidics Station 450 and scanned according to the manufacturer's protocol using GeneArray-™ scanner G2500A (Hewlett Packard, USA).

### 2.2. Data Analysis

Data analysis was performed using Partek Genomics Solution software (www.partek.com). Expression values were computed from raw CEL files by applying the Robust Multi-Chip Average (RMA) background correction algorithm. RMA correction included: 1) values background correction; 2) quintile normalization; 3) log 2 transformation; and 4) median polish summarization. ANOVA analysis was applied to compare PBMC gene expression after one and six months of LAQ/placebo treatment as compared with baseline. Age, gender and batch effects were regarded as confounders in the ANOVA model. Genes that significantly changed in the placebo treated patients as compared to baseline were excluded from further analysis to yield only genes exclusively associated with LAQ effects. MIGs were defined as those that differentiated between groups with p<0.01. Gene functional annotation, enrichment and pathway analyses to identify the involved biological pathways were performed by Ingenuity Pathways Analysis software (www.ingenuity.com). All p values were applied for multiple testing corrections using False Discovery Rate (FDR) method with a cut off at p*0.05.

### 2.3. Verification by Western Blot

Protein fractions were purified from PBMC of 5 patients at baseline and after six months of LAQ treatment. Proteins were extracted from TRIZOL fractions and solubilized following the method reported by Hummon et al., 2007 (Hummon et al., 2007). Equal amounts of proteins were resolved on 10% SDS-PAGE and transferred onto nitrocellulose membranes (Invitrogen kit) for subsequent immune-blotting with antibodies specific for TGFb, ITGB1, CXCR1 and alpha Tubulin (Santa Cruz Biotechnology, Inc Santa Cruz, Calif., USA). Blots were analyzed by standard chemi-luminescence (Supersignal Kit, Pierce, Rockford, Ill., USA) and visualization was done by ChemiDoc™ XRS System (Bio-Rad).

### 3. Results

Samples were obtained from 25 RRMS patients, age 38.0±2.0 years, female/male ratio 16/9. The LAQ treatment arm consists of 13 patients, female/male ratio 8/5, age 38.8±2.3 years and the placebo arm consists of 12 patients, female/male ratio 8/4, age 37.2±3.4 years.

LAQ induced a differential gene expression of 354 MIGs after one month of treatment and the number of MIGs increased to 1562 after six months (Table 6 and 7).

The majority of genes that significantly changed expression under LAQ treatment at one and six months of treatment were down regulated (98% and 99%, respectively).

3.1. Biological Pathways Associated with LAQ Treatment: Down-Regulation of TGFb and NFkB Signaling and Pro Inflammatory Cytokines

Functional enrichment analysis of 354 MIGs after one month of LAQ treatment disclosed the suppression of molecules associated with different mechanisms of inflammatory response and cellular movement presented in Table 5. Indeed, analysis of the 1562 MIGs after six months showed growing number of genes involved with these mechanisms. Of the significantly suppressed pathways, the TGFb superfamily signaling (Table 5, p=3.2*10⁻³) was suppressed after one as well as after six months of LAQ treatment (p=4.32*10⁻²).

TABLE 5
Major biological pathways and functions affected by LAQ treatment

| After one month of treatment n = 345 | | | | After six months of treatment n = 1562 | | | |
|---|---|---|---|---|---|---|---|
| | | No. of | | | | No. of | |
| Function | Pathway | genes | p-value | Function | Pathway | genes | p-value |
| Inflammatory | TGFb | 10 | 3.2 × 10⁻³ | Inflammatory | TGFb | 14 | 4.3 × 10⁻² |
| response | signaling | | | response | signaling | | |
| | | | | | IL-12 | 11 | 3.2 × 10⁻³ |
| | | | | | signaling | | |
| Cellular | Adhesion & | 9 | 1.2 × 10⁻³ | Cellular | Invasion of | 120 | 5.6 × 10⁻⁵ |
| movement | migration of | | | movement | cells | | |
| | phagocytes | | | | | | |
| | Chemotaxis of | 4 | 6.0 × 10⁻³ | | Adhesion | 119 | 2.4 × 10⁻⁵ |
| | Neutrophils | | | | of cells | | |
| | Transmigration | 8 | 1.9 × 10⁻³ | | Leukocyte | 29 | 9.4 × 10⁻³ |
| | of leukocytes | | | | extravasation | | |
| | | | | | signaling | | |

Downregulation of the TGFb signaling pathway after one month of LAQ treatment was evident by suppression of TGFb and LTBP1 genes, the latter regulates secretion and activation of TGFb and thus promoting a feedback mechanism. After six months, down-regulation of additional TGFb superfamily related genes like BMP2/4/7, MIS, Type II BMP receptor, Smad14/5/6/8, TCF20, TCF2, Runx2 and the downstream ITGB1 was demonstrated (FIG. 9B).

Also, LAQ induced down-regulation of LFA-1 and VLA-4 expression that act with ITGB1 in adhesion of immune cells. The suppression of TGFb pathway after six months of LAQ treatment was accompanied by down regulation of IL-12 signaling pathway (p=6.2*10⁻³) and a wide range of other pro-inflammatory cytokines such as IL-9/11/12/20/36, TNFRSF11A/B, IFNA4/8/10/17, and also the receptors for IL-5/13/20/22 (p=3*10⁻³ to 9*10⁻³).

The molecular signature of LAQ after 6 months was also characterized by suppression of NFkB signaling as demonstrated by down regulation of members of the NFkB signaling that play a role in inflammation including IL-1, IL-1R and IKKg (FIG. 9B).

Altogether, these findings figure out a comprehensive suppression of pro-inflammatory cytokines and the key TGFb and NFkB pathways following six months of LAQ treatment. In view of the early down-regulation of TGFb at one month that precede the down-regulation of genes of pro-inflammatory cytokines, the inventors suggest that TGFb signaling precedes the suppression of inflammatory cytokines and that LAQ down regulates cytokine expression via suppression of TGFb.

Only five LAQ responsive MIGs were upregulated with the common three genes SASH1, FUCA1 and XYLT1 at one and six months. Although none of them integrated in firmed canonical pathway, overexpression of SASH1 and FUCA1 is associated with the inhibition of growth, proliferation, and invasion of cells (Meng et al., 2013).

### 3.2. LAQ Down-Regulates Expression of Migration, Adhesion and Leukocyte Extravasations Genes

Differential expression of cellular movement and migration were observed already after one month of LAQ treatment (p=3.49*10⁻⁴). These included down-regulation of genes associated with adhesion and migration of phagocytes (p=1.2*10⁻³), chemotaxis of neutrophils (p=6*10⁻³) and transmigration of leukocytes (p=1.9*10⁻³). Genes associated with cell movement and suppressed by LAQ were P selectin that is involved in the initial stage of adhesion and the integrin family members like ITGB1/3/5/6/8 and ITGA8 involved in later steps of adhesion and locomotion during leukocytes extravasation (p-value 1.72*10⁻³ to 5.5*10⁻³).

The suppressing effects of LAQ on cell adhesion and integrin expression were further enhanced after six months of treatment as was evident by down regulation of genes associated with cellular movement mechanisms (p value 3.15*10⁻⁶ to 3.79*10⁻³) including cell invasion (p=5.6*10⁻⁵), adhesion (p=2.4*10⁻⁵) and leukocyte extravasation (p=9.4*10⁻³), (Table 5, supra).

Similar to the observed effects of suppressed expression of the integrin family members after one month of treatment, suppression was even more evident after six months of LAQ treatment including integrin genes like ITGB/5/6/8, ITGA8, ITGB8, and ITGA2B (p value 9.84*10⁻⁴ to 1.1*10⁻³). In addition, suppression of inflammatory related chemokines like CCL19 and chemokine receptor CXCR1/2 was also demonstrated (p=6.79*10⁻³). Moreover, LAQ down-regulated a range of metalloproteinase family members such as MMP16/24/26/28, and ADAM12/18/22 that play a role during extravasation (p=4.95*10⁻⁴ to 1.26*10⁻³).

3.3. Verification of Key Genes Associated with LAQ Induced Molecular Pathways

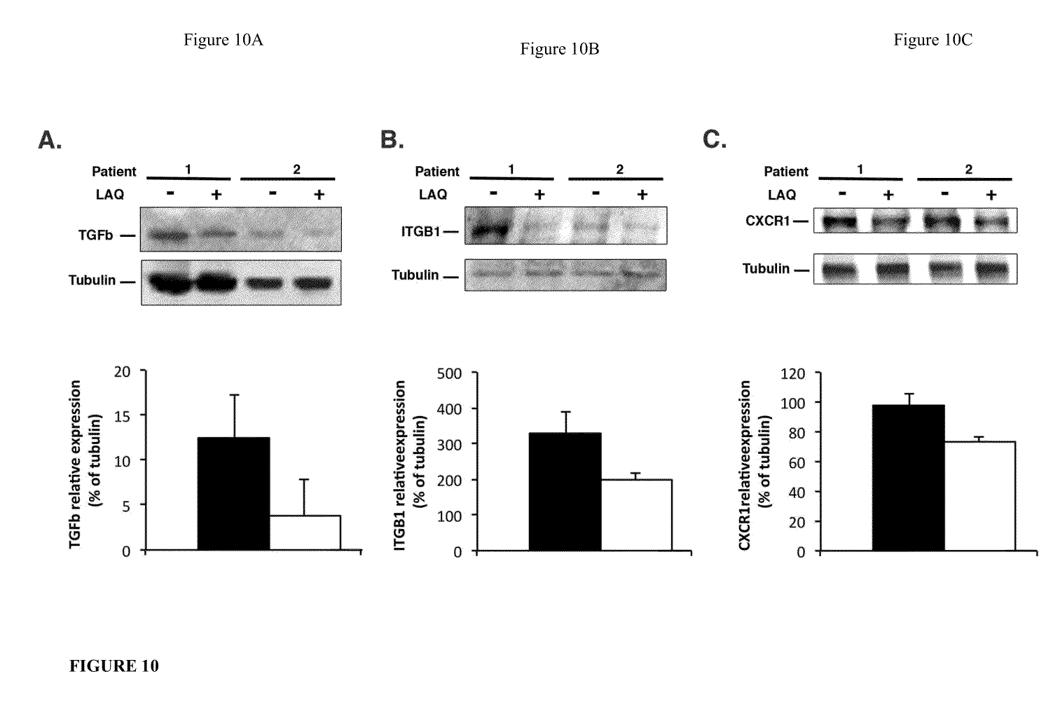

The verification experiments performed by Western Blot analysis show significant down-regulation of key genes associated with most significantly affected biological mechanisms of LAQ. The TGFb protein following six months of LAQ treatment was suppressed by 69.0% (p=0.009) as could be seen from quantification of bands intensities (FIG. 10A). Accordingly, FIG. 10B shows down regulation of ITGB1, a common subunit of different integrin receptors by 40% (p=0.03) and of CXCR1 by 24.7% (p=0.014) (FIG. 10C).

### 4. Discussion

The results demonstrate that the most significant effect of LAQ is induction of suppression of inflammatory response via TGFb and NFkB pathways, as well as decrease in cell movement processes including adhesion, migration and leukocyte extravasation.

The inventors observed down-regulation of signaling pathways involving integrins, chemokines and metalloproteinases accompanied by repression of pro-inflammatory cytokines. These effects were observed in RRMS patients treated over six months-period as compared with baseline. Notably, the suppressive effects of LAQ are already detected as early as one month after initiation of treatment although to a lesser extent, suggesting a time-dependent treatment effect.

The pivotal function of TGFb in the immune system is anti-inflammatory, to maintain tolerance via regulation of lymphocyte proliferation, differentiation and survival. However, in MS it has been shown that in addition to its anti-inflammatory role, TGFb paradoxically can act as pro-inflammatory factor involved in the genesis of the pathogenic EAE-inducing TH17 cells. Thus, deletion of the TGFb gene from activated T cells, is known to abrogate Th17 cell differentiation, resulting in almost complete protection from EAE, confirming TGFb proinflammatory potential (Oh and Li, 2013). In the same process of events, TGFb is involved in stimulation of inflammatory cells adhesion, migration and extravasation, and could promote penetration of auto-aggressive lymphocytes to the central nervous system (CNS) (Bartolome et al., 2003; Brill et al., 2001). TGFb is also known to regulate the expression of IL-9 (Takami et al, 2012) and IL-22 (Sanjabi et al., 2009), thereby enhancing the expression of molecules associated with inflammation. TGFb itself can be activated by IL-1 (Luo et al., 2009), however IL-1 was also found to be suppressed in LAQ gene expression signature.

In accordance with observations linking TGFb with inflammatory process, the suppression of TGFb and members of the TGFb pathway by LAQ could result in beneficial reduction of active inflammation in MS. The suppression of TGFb signaling by LAQ corroborates with previous publications in which LAQ suppresses MAP3K7 (TAK1) that is strong positive regulator of cellular proliferation mediated by TGFb activation in CD14+ cells (Gurevich et al., 2010; Wan et al., 2006).

The inventors have demonstrated the suppressed expression of large number of cell adhesion and cell movement molecules involved in different stages of leukocytes extravasation under LAQ treatment. The ability of inflammatory cells to move from the periphery to the CNS is a crucial multistep process in MS with the following components down regulated by LAQ: a) Selectin P and IL-8R (CXCR1/2), that mediate rolling and the initial leukocyte-endothelial interactions; b) VLA-4, LFA-1, ITGA2/8, and ITGB1-6 integrins that mediate leukocyte adhesion and transmigration; c) chemokines and chemokine receptors for integrin activation like CCL19 that is responsible for leukocyte arrest and transmigration, and IL-8 receptor (CXCR1/2). These genes are well fitted with the steps of rolling, activation, adhesion, locomotion protrusion and transmigration of immune cells during extravasation to the CNS (as shown in FIG. 8C). Taken together, findings of the present study suggest that LAQ acts through inhibition of immune cells movement, adhesion and transmigration, thereby reducing the migratory capacity of active inflammatory cells trough the blood brain barrier (BBB). The supression of these cell migration functions corroborate with previously reported effects of LAQ- to induce down regulation of various cytokines and integrins such as IL-12, IL-13, IL-17, IFN-γ, TNF-α and VLA-4-mediated adhesiveness resulting in interference with migratory capacity of T cells in EAE (Brück and Wegner, 2011; Jadidi-Niaragh et al., 2011; Wegner et al., 2010).

Similarly, in MS patients, the inventors have demonstrated that LAQ down-regulates IL-1, IL-1R, IL12 and IKKg genes associated with pro-inflammatory NFkB pathway. The suppression of NFkB mechanism by LAQ was also demonstrated in an in-vitro study on PBMC obtained from MS patients (Gurevich et al., 2010) and in astrocytes following LAQ treatment in cuprizone-induced demyelination model (Bruck et al., 2012). NFkB signaling mediates IL-12 activation in macrophages (Murphy et al., 1995). The inventors have determined that LAQ may suppress both IL1 and IL2 dependent inflammation via down regulation of NFkB signaling.

These inflammation counteracting effects of LAQ could be the molecular basis of the positive imaging effect of LAQ in the ALLEGRO trial (Comi et al., 2012; Filippi et al., 2014).

Only 5 genes were up-regulated by LAQ; three of these up regulated genes were up-regulated already after one month of treatment with sustained effect at 6 months; Sash1 and FUCA1 are involved in suppression of proliferation while XYLT catalyzes the biosynthesis of glycosaminoglycan and its high activity was reported in patients with impaired BBB (Ponighaus et al., 2007). After six months of treatment, another growth inhibitor gene PID1 was overexpressed, confirming the suppression of proliferation of CD8+ cells by LAQ (Gurevich et al., 2010).

The inventors believe this to be the first study that characterizes LAQ induced transcriptional profile of RRMS patients demonstrating LAQ suppression of inflammatory cytokines and leukocytes extravasation either directly or via suppression of TGFb superfamily and NFkB signaling, thereby contributing to amelioration of the disease process of MS.

TABLE 6

| | | | p-value | Fold-Change |
|---|---|---|---|---|
| Column ID | Gene Symbol | Gene Title | (1.0 vs. 0.0) | (1.0 vs. 0.0) |
| 202380_s_at | NKTR | natural killer-tumor | 3.20E−05 | −1.12256 |
| | | recognition sequence | | |
| 215673_at | TEF | thyrotrophic embryonic | 4.00E−05 | −1.16076 |
| | | factor | | |
| 219414_at | CLSTN2 | calsyntenin 2 | 4.51E−05 | −1.12845 |
| 220099_s_at | LUC7L2 | LUC7-like 2 | 9.60E−05 | −1.15527 |
| | | (S. cerevisiae) | | |
| 215492_x_at | PTCRA | pre T-cell antigen | 0.000172567 | −1.52614 |
| | | receptor alpha | | |
| 207426_s_at | TNFSF4 | tumor necrosis factor | 0.000172751 | −1.7061 |
| | | (ligand) superfamily, | | |
| | | member 4 | | |
| 205030_at | FABP7 | fatty acid binding | 0.000176697 | −1.17613 |
| | | protein 7, brain | | |
| 220205_at | TPTE | transmembrane | 0.000181472 | −1.15822 |
| | | phosphatase with tensin | | |
| | | homology | | |
| 208782_at | FSTL1 | follistatin-like 1 | 0.00019072 | −1.69352 |
| 201071_x_at | SF3B1 | splicing factor 3b, | 0.000259586 | −1.0879 |
| | | subunit 1, 155 kDa | | |
| 207198_s_at | LIMS1 | LIM and senescent cell | 0.000294426 | −1.44487 |
| | | antigen-like domains 1 | | |
| 206757_at | PDE5A | phosphodiesterase 5A, | 0.000306957 | −1.213 |
| | | cGMP-specific | | |
| 209045_at | XPNPEP1 | X-prolyl aminopeptidase | 0.000348404 | −1.19661 |
| | | (aminopeptidase P) 1, | | |
| | | soluble | | |
| 48031_r_at | C5orf4 | chromosome 5 open | 0.000363815 | −1.51923 |
| | | reading frame 4 | | |
| 220921_at | SPANXB1 /// | SPANX family, member | 0.000369349 | −1.16871 |
| | SPANXB2 /// | B1 /// SPANX family, | | |
| | SPANXF1 | member B2 /// SPANX | | |
| | | family, member F1 | | |
| 202729_s_at | LTBP1 | latent transforming | 0.000383136 | −1.71014 |
| | | growth factor beta | | |
| | | binding protein 1 | | |
| 213953_at | KRT20 | keratin 20 | 0.000398517 | −1.13707 |
| 214146_s_at | PPBP | pro-platelet basic | 0.000468646 | −1.45959 |
| | | protein (chemokine (C- | | |
| | | X-C motif) ligand 7) | | |
| 214013_s_at | TBC1D1 | TBC1 (tre-2/USP6, | 0.000514065 | −1.21818 |
| | | BUB2, cdc16) domain | | |
| | | family, member 1 | | |
| 219388_at | GRHL2 | grainyhead-like 2 | 0.000551908 | −1.13133 |
| | | (Drosophila) | | |
| 220751_s_at | C5orf4 | chromosome 5 open | 0.000640518 | −1.96738 |
| | | reading frame 4 | | |
| 211252_x_at | PTCRA | pre T-cell antigen | 0.000650924 | −1.38911 |
| | | receptor alpha | | |
| 206390_x_at | PF4 | platelet factor 4 | 0.00069054 | −1.76355 |
| 213666_at | SEPT6 | septin 6 | 0.000693884 | −1.13383 |
| 212942_s_at | KIAA1199 | KIAA1199 | 0.000714369 | −1.12102 |
| 203016_s_at | SSX2IP | synovial sarcoma, X | 0.000739497 | −1.36137 |
| | | breakpoint 2 interacting | | |
| | | protein | | |
| 206116_s_at | TPM1 | tropomyosin 1 (alpha) | 0.00075228 | −1.49626 |
| 221556_at | CDC14B | CDC14 cell division | 0.000762595 | −1.48795 |
| | | cycle 14 homolog B | | |
| | | (S. cerevisiae) | | |
| 221518_s_at | USP47 | ubiquitin specific | 0.000785063 | −1.07574 |
| | | peptidase 47 | | |
| 205612_at | MMRN1 | multimerin 1 | 0.000786871 | −1.37634 |
| 202468_s_at | CTNNAL1 | catenin (cadherin- | 0.000828338 | −1.41611 |
| | | associated protein), | | |
| | | alpha-like 1 | | |
| 210357_s_at | SMOX | spermine oxidase | 0.000848049 | −1.40727 |
| 207206_s_at | ALOX12 | arachidonate 12- | 0.000911895 | −1.83581 |
| | | lipoxygenase | | |
| 210661_at | GLRA3 | glycine receptor, alpha 3 | 0.000946566 | −1.15569 |
| 209301_at | CA2 | carbonic anhydrase II | 0.000964786 | −1.68995 |
| 211555_s_at | GUCY1B3 | guanylate cyclase 1, | 0.00100045 | −1.61803 |
| | | soluble, beta 3 | | |
| 207934_at | RFPL1 | ret finger protein-like 1 | 0.00102558 | −1.14458 |
| 220496_at | CLEC1B | C-type lectin domain | 0.00102643 | −2.04495 |
| | | family 1, member B | | |
| 204115_at | GNG11 | guanine nucleotide | 0.00102931 | −1.67885 |
| | | binding protein (G | | |
| | | protein), gamma 11 | | |
| 220558_x_at | TSPAN32 | tetraspanin 32 | 0.00108103 | −1.15615 |
| 204319_s_at | RGS10 | regulator of G-protein | 0.00108604 | −1.27188 |
| | | signaling 10 | | |
| 201615_x_at | CALD1 | caldesmon 1 | 0.00112603 | −1.44221 |
| 203680_at | PRKAR2B | protein kinase, cAMP- | 0.00119671 | −1.87579 |
| | | dependent, regulatory, | | |
| | | type II, beta | | |
| 206153_at | CYP4F11 | cytochrome P450, | 0.00121638 | −1.09486 |
| | | family 4, subfamily F, | | |
| | | polypeptide 11 | | |
| 220810_at | CLCA3P | chloride channel | 0.00123986 | −1.14123 |
| | | accessory 3 | | |
| | | (pseudogene) | | |
| 40020_at | CELSR3 | cadherin, EGF LAG | 0.00127162 | −1.12078 |
| | | seven-pass G-type | | |
| | | receptor 3 (flamingo | | |
| | | homolog, Drosophila) | | |
| 208022_s_at | CDC14B | CDC14 cell division | 0.00133236 | −1.4133 |
| | | cycle 14 homolog B | | |
| | | (S. cerevisiae) | | |
| 210987_x_at | TPM1 | tropomyosin 1 (alpha) | 0.00135846 | −1.44653 |
| 214298_x_at | SEPT6 | septin 6 | 0.00137756 | −1.13328 |
| 207119_at | PRKG1 | protein kinase, cGMP- | 0.00141929 | −1.18474 |
| | | dependent, type I | | |
| 210734_x_at | MAX | MYC associated factor X | 0.00142961 | −1.28358 |
| 209689_at | CCDC93 | coiled-coil domain | 0.0014704 | −1.13336 |
| | | containing 93 | | |
| 214749_s_at | ARMCX6 /// | armadillo repeat | 0.00149785 | −1.17832 |
| | LOC653354 | containing, X-linked 6 | | |
| | | /// similar to armadillo | | |
| | | repeat containi | | |
| 214023_x_at | TUBB2B | tubulin, beta 2B | 0.00153776 | −1.15934 |
| 208583_x_at | HIST1H2AJ | histone cluster 1, H2aj | 0.00154449 | −1.27798 |
| 205442_at | MFAP3L | microfibrillar-associated | 0.0015663 | −1.85392 |
| | | protein 3-like | | |
| 212687_at | LIMS1 | LIM and senescent cell | 0.00160765 | −1.3332 |
| | | antigen-like domains 1 | | |
| 211871_x_at | GNB5 | guanine nucleotide | 0.00163175 | −1.15867 |
| | | binding protein (G | | |
| | | protein), beta 5 | | |
| 204793_at | GPRASP1 | G protein-coupled | 0.00165477 | −1.16021 |
| | | receptor associated | | |
| | | sorting protein 1 | | |
| 201680_x_at | SRRT | serrate RNA, effector | 0.00172271 | −1.11791 |
| | | molecule homolog | | |
| | | (Arabidopsis) | | |
| 211837_s_at | PTCRA | pre T-cell antigen | 0.00177798 | −1.27392 |
| | | receptor alpha | | |
| 219476_at | C1orf116 | chromosome 1 open | 0.00181581 | −1.22156 |
| | | reading frame 116 | | |
| 201178_at | FBXO7 | F-box protein 7 | 0.00186196 | −1.13506 |
| 203966_s_at | PPM1A | protein phosphatase 1A | 0.00188506 | −1.18222 |
| | | (formerly 2C), | | |
| | | magnesium-dependent, | | |
| | | alpha isoform | | |
| 203817_at | GUCY1B3 | guanylate cyclase 1, | 0.00189066 | −1.72974 |
| | | soluble, beta 3 | | |
| 201125_s_at | ITGB5 | integrin, beta 5 | 0.00191776 | −1.71341 |
| 201905_s_at | CTDSPL | CTD (carboxy-terminal | 0.00197783 | −1.46779 |
| | | domain, RNA | | |
| | | polymerase II, | | |
| | | polypeptide A) small | | |
| | | phosphatas | | |
| 200780_x_at | GNAS | GNAS complex locus | 0.00198667 | −1.15838 |
| 203819_s_at | IGF2BP3 | insulin-like growth | 0.0019919 | −1.71954 |
| | | factor 2 mRNA binding | | |
| | | protein 3 | | |
| 210986_s_at | TPM1 | tropomyosin 1 (alpha) | 0.00199934 | −1.55544 |
| 209806_at | HIST1H2BK | histone cluster 1, H2bk | 0.00202655 | −1.41838 |
| 210684_s_at | DLG4 | discs, large homolog 4 | 0.00202851 | −1.18659 |
| | | (Drosophila) | | |
| 222157_s_at | WDR48 | WD repeat domain 48 | 0.00207579 | −1.10297 |
| 212077_at | CALD1 | caldesmon 1 | 0.00217742 | −1.89576 |
| 214839_at | LOC157627 | hypothetical | 0.00223956 | 1.32598 |
| | | LOC157627 | | |
| 207124_s_at | GNB5 | guanine nucleotide | 0.00230176 | −1.1663 |
| | | binding protein (G | | |
| | | protein), beta 5 | | |
| 205514_at | ZNF415 | zinc finger protein 415 | 0.00231529 | −1.15423 |
| 206049_at | SELP | selectin P (granule | 0.00232343 | −1.64193 |
| | | membrane protein | | |
| | | 140 kDa, antigen CD62) | | |
| 206414_s_at | ASAP2 | ArfGAP with SH3 | 0.00233503 | −1.77303 |
| | | domain, ankyrin repeat | | |
| | | and PH domain 2 | | |
| 218613_at | PSD3 | pleckstrin and Sec7 | 0.00234479 | −1.25828 |
| | | domain containing 3 | | |
| 200981_x_at | GNAS | GNAS complex locus | 0.00238211 | −1.1585 |
| 211945_s_at | ITGB1 | integrin, beta 1 | 0.00241794 | −1.17026 |
| | | (fibronectin receptor, | | |
| | | beta polypeptide, | | |
| | | antigen CD29 includes | | |
| 219926_at | POPDC3 | popeye domain | 0.00243065 | −1.1575 |
| | | containing 3 | | |
| 204081_at | NRGN | neurogranin (protein | 0.00243424 | −1.60366 |
| | | kinase C substrate, RC3) | | |
| 205730_s_at | ABLIM3 | actin binding LIM | 0.00246133 | −1.54178 |
| | | protein family, member 3 | | |
| 213725_x_at | XYLT1 | xylosyltransferase I | 0.00253677 | 1.31402 |
| 210702_s_at | PTGIS | prostaglandin I2 | 0.00258067 | −1.09522 |
| | | (prostacyclin) synthase | | |
| 215139_at | ARHGEF10 | Rho guanine nucleotide | 0.00258297 | −1.17563 |
| | | exchange factor (GEF) 10 | | |
| 205463_s_at | PDGFA | platelet-derived growth | 0.00261003 | −1.53627 |
| | | factor alpha polypeptide | | |
| 204628_s_at | ITGB3 | integrin, beta 3 (platelet | 0.00264187 | −1.57906 |
| | | glycoprotein IIIa, | | |
| | | antigen CD61) | | |
| 201121_s_at | PGRMC1 | progesterone receptor | 0.00266076 | −1.44314 |
| | | membrane component 1 | | |
| 215071_s_at | HIST1H2AC | histone cluster 1, H2ac | 0.00271978 | −1.62836 |
| 212273_x_at | GNAS | GNAS complex locus | 0.00273624 | −1.15606 |
| 204482_at | CLDN5 | claudin 5 | 0.00276964 | −1.36386 |
| 210493_s_at | MFAP3L | microfibrillar-associated | 0.0027754 | −1.46358 |
| | | protein 3-like | | |
| 201120_s_at | PGRMC1 | progesterone receptor | 0.0027801 | −1.43219 |
| | | membrane component 1 | | |
| 202423_at | MYST3 | MYST histone | 0.00280673 | −1.07974 |
| | | acetyltransferase | | |
| | | (monocytic leukemia) 3 | | |
| 200722_s_at | CAPRIN1 | cell cycle associated | 0.00288696 | −1.15665 |
| | | protein 1 | | |
| 201617_x_at | CALD1 | caldesmon 1 | 0.0028931 | −1.32568 |
| 218751_s_at | FBXW7 | F-box and WD repeat | 0.00289921 | −1.11226 |
| | | domain containing 7 | | |
| 209839_at | DNM3 | dynamin 3 | 0.00294256 | −1.79997 |
| 211190_x_at | CD84 | CD84 molecule | 0.0029693 | −1.16616 |
| 202127_at | PRPF4B | PRP4 pre-mRNA | 0.00299632 | −1.13501 |
| | | processing factor 4 | | |
| | | homolog B (yeast) | | |
| 202280_at | — | — | 0.00302852 | −1.15673 |
| 212031_at | RBM25 | RNA binding motif | 0.00302972 | −1.10451 |
| | | protein 25 | | |
| 204042_at | WASF3 | WAS protein family, | 0.00304453 | −1.60611 |
| | | member 3 | | |
| 208406_s_at | GRAP2 | GRB2-related adaptor | 0.0030481 | −1.39443 |
| | | protein 2 | | |
| 200665_s_at | SPARC | secreted protein, acidic, | 0.00304843 | −1.77594 |
| | | cysteine-rich | | |
| | | (osteonectin) | | |
| 206283_s_at | TAL1 | T-cell acute | 0.00307052 | −1.75245 |
| | | lymphocytic leukemia 1 | | |
| 218407_x_at | NENF | neuron derived | 0.0030954 | −1.14125 |
| | | neurotrophic factor | | |
| 206698_at | XK | X-linked Kx blood | 0.00309645 | −1.88028 |
| | | group (McLeod | | |
| | | syndrome) | | |
| 207389_at | GP1BA | glycoprotein Ib | 0.00310255 | −1.54848 |
| | | (platelet), alpha | | |
| | | polypeptide | | |
| 215240_at | ITGB3 | integrin, beta 3 (platelet | 0.00313548 | −1.58871 |
| | | glycoprotein IIIa, | | |
| | | antigen CD61) | | |
| 217456_x_at | HLA-E | major histocompatibility | 0.00314152 | −1.06184 |
| | | complex, class I, E | | |
| 207421_at | CA5A | carbonic anhydrase VA, | 0.00315794 | −1.24281 |
| | | mitochondrial | | |
| 220037_s_at | LYVE1 | lymphatic vessel | 0.00316412 | −1.13941 |
| | | endothelial hyaluronan | | |
| | | receptor 1 | | |
| 203085_s_at | TGFB1 | transforming growth | 0.00316654 | −1.24041 |
| | | factor, beta 1 | | |
| 201736_s_at | MARCH6 | membrane-associated | 0.00323685 | −1.13929 |
| | | ring finger (C3HC4) 6 | | |
| 206964_at | NAT8B | N-acetyltransferase 8B | 0.00333385 | −1.74593 |
| | | (GCN5-related, putative, | | |
| | | gene/pseudogene) | | |
| 215047_at | TRIM58 | tripartite motif- | 0.00338565 | −1.77665 |
| | | containing 58 | | |
| 211421_s_at | RET | ret proto-oncogene | 0.00341726 | −1.22099 |
| 218711_s_at | SDPR | serum deprivation | 0.00342263 | −1.66157 |
| | | response | | |
| | | (phosphatidylserine | | |
| | | binding protein) | | |
| 336_at | TBXA2R | thromboxane A2 | 0.00343156 | −1.43266 |
| | | receptor | | |
| 200929_at | TMED10 | transmembrane emp24- | 0.00344518 | −1.09881 |
| | | like trafficking protein | | |
| | | 10 (yeast) | | |
| 209871_s_at | APBA2 | amyloid beta (A4) | 0.00349415 | −1.16326 |
| | | precursor protein- | | |
| | | binding, family A, | | |
| | | member 2 | | |
| 201058_s_at | MYL9 | myosin, light chain 9, | 0.003504 | −1.74013 |
| | | regulatory | | |
| 207846_at | POU1F1 | POU class 1 homeobox 1 | 0.00351318 | −1.09086 |
| 208579_x_at | H2BFS /// | H2B histone family, | 0.00351435 | −1.48066 |
| | HIST1H2BK | member S /// histone | | |
| | | cluster 1, H2bk | | |
| 220759_at | FAM12B | family with sequence | 0.0035357 | −1.1468 |
| | | similarity 12, member B | | |
| | | (epididymal) | | |
| 200931_s_at | VCL | vinculin | 0.00355492 | −1.40602 |
| 217595_at | GSPT1 | G1 to S phase transition 1 | 0.00359115 | −1.11645 |
| 204704_s_at | ALDOB | aldolase B, fructose- | 0.00362444 | −1.11912 |
| | | bisphosphate | | |
| 207856_s_at | LOC150776 /// | sphingomyelin | 0.00363765 | −1.0672 |
| | SMPD4 | phosphodiesterase 4, | | |
| | | neutral membrane | | |
| | | pseudogene /// | | |
| | | sphingomyelin | | |
| 218928_s_at | SLC37A1 | solute carrier family 37 | 0.00364173 | −1.12611 |
| | | (glycerol-3-phosphate | | |
| | | transporter), member 1 | | |
| 212667_at | SPARC | secreted protein, acidic, | 0.00365945 | −1.72956 |
| | | cysteine-rich | | |
| | | (osteonectin) | | |
| 214548_x_at | GNAS | GNAS complex locus | 0.00366147 | −1.15948 |
| 221392_at | TAS2R4 | taste receptor, type 2, | 0.00366806 | −1.20308 |
| | | member 4 | | |
| 200622_x_at | CALM3 | calmodulin 3 | 0.00368294 | −1.2967 |
| | | (phosphorylase kinase, | | |
| | | delta) | | |
| 212178_s_at | POM121 /// | POM121 membrane | 0.00369813 | −1.09132 |
| | POM121C | glycoprotein (rat) /// | | |
| | | POM121 membrane | | |
| | | glycoprotein C | | |
| 215993_at | — | — | 0.00369964 | −1.07521 |
| 215655_at | GRIK2 | Glutamate receptor, | 0.00371565 | −1.12171 |
| | | ionotropic, kainate 2 | | |
| 218468_s_at | GREM1 | gremlin 1, cysteine knot | 0.00374196 | −1.11604 |
| | | superfamily, homolog | | |
| | | (Xenopus laevis) | | |
| 205388_at | TNNC2 | troponin C type 2 (fast) | 0.00376489 | −1.21183 |
| 207750_at | EPS15L2 | epidermal growth factor | 0.00376694 | −1.1441 |
| | | receptor pathway | | |
| | | substrate 15-like 2 | | |
| 212573_at | ENDOD1 | endonuclease domain | 0.00376788 | −1.29339 |
| | | containing 1 | | |
| 210270_at | RGS6 | regulator of G-protein | 0.00377039 | −1.25086 |
| | | signaling 6 | | |
| 211185_s_at | SF3B1 | splicing factor 3b, | 0.00378516 | −1.0809 |
| | | subunit 1, 155 kDa | | |
| 205347_s_at | TMSB15A | thymosin beta 15a | 0.0038153 | −1.11916 |
| 205383_s_at | ZBTB20 | zinc finger and BTB | 0.00387924 | −1.13702 |
| | | domain containing 20 | | |
| 214046_at | FUT9 | fucosyltransferase 9 | 0.00390947 | −1.12688 |
| | | (alpha (1,3) | | |
| | | fucosyltransferase) | | |
| 212062_at | ATP9A | ATPase, class II, type 9A | 0.00394326 | −1.44071 |
| 214974_x_at | CXCL5 | chemokine (C-X-C | 0.00401473 | −1.73103 |
| | | motif) ligand 5 | | |
| 209331_s_at | MAX | MYC associated factor X | 0.00402443 | −1.24493 |
| 215306_at | — | — | 0.00405048 | −1.22628 |
| 214542_x_at | HIST1H2AI | histone cluster 1, H2ai | 0.0040542 | −1.2842 |
| 211948_x_at | BAT2D1 | BAT2 domain | 0.00405795 | −1.08274 |
| | | containing 1 | | |
| 202123_s_at | ABL1 | c-abl oncogene 1, | 0.00406538 | −1.10234 |
| | | receptor tyrosine kinase | | |
| 211546_x_at | SNCA | synuclein, alpha (non | 0.00409573 | −1.35092 |
| | | A4 component of | | |
| | | amyloid precursor) | | |
| 208501_at | GFI1B | growth factor | 0.00411391 | −1.55771 |
| | | independent 1B | | |
| | | transcription repressor | | |
| 200661_at | CTSA | cathepsin A | 0.00411941 | −1.29244 |
| 215820_x_at | SNX13 | sorting nexin 13 | 0.00412146 | −1.18742 |
| 204486_at | — | — | 0.00413941 | −1.18519 |
| 201529_s_at | RPA1 | replication protein A1, | 0.00422086 | −1.26569 |
| | | 70 kDa | | |
| 214752_x_at | FLNA | filamin A, alpha | 0.00426746 | −1.14045 |
| 208453_s_at | XPNPEP1 | X-prolyl aminopeptidase | 0.00430113 | −1.19059 |
| | | (aminopeptidase P) 1, | | |
| | | soluble | | |
| 203086_at | KIF2A | kinesin heavy chain | 0.00434355 | −1.27703 |
| | | member 2A | | |
| 214631_at | ZBTB33 | zinc finger and BTB | 0.0043722 | −1.13452 |
| | | domain containing 33 | | |
| 202728_s_at | LTBP1 | latent transforming | 0.00437355 | −1.47988 |
| | | growth factor beta | | |
| | | binding protein 1 | | |
| 208776_at | PSMD11 | proteasome (prosome, | 0.00439519 | −1.12238 |
| | | macropain) 26S subunit, | | |
| | | non-ATPase, 11 | | |
| 212751_at | UBE2N | ubiquitin-conjugating | 0.00441391 | −1.10218 |
| | | enzyme E2N (UBC13 | | |
| | | homolog, yeast) | | |
| 204437_s_at | FOLR1 | folate receptor 1 (adult) | 0.0044397 | −1.21956 |
| 215111_s_at | TSC22D1 | TSC22 domain family, | 0.00446803 | −1.61432 |
| | | member 1 | | |
| 217816_s_at | PCNP | PEST proteolytic signal | 0.00447658 | −1.13528 |
| | | containing nuclear | | |
| | | protein | | |
| 205165_at | CELSR3 | cadherin, EGF LAG | 0.00452773 | −1.16533 |
| | | seven-pass G-type | | |
| | | receptor 3 (flamingo | | |
| | | homolog, Drosophila) | | |
| 206465_at | ACSBG1 | acyl-CoA synthetase | 0.004567 | −1.54878 |
| | | bubblegum family | | |
| | | member 1 | | |
| 208924_at | RNF11 | ring finger protein 11 | 0.00458077 | −1.38056 |
| 206941_x_at | SEMA3E | sema domain, | 0.00459381 | −1.14106 |
| | | immunoglobulin domain | | |
| | | (Ig), short basic domain, | | |
| | | secreted, (semaphor | | |
| 210075_at | MARCH2 | membrane-associated | 0.00459416 | −1.3917 |
| | | ring finger (C3HC4) 2 | | |
| 220186_s_at | PCDH24 | protocadherin 24 | 0.00460173 | −1.12071 |
| 201480_s_at | SUPT5H | suppressor of Ty 5 | 0.00461915 | −1.10301 |
| | | homolog (S. cerevisiae) | | |
| 200904_at | HLA-E | major histocompatibility | 0.00463895 | −1.12592 |
| | | complex, class I, E | | |
| 206254_at | EGF | epidermal growth factor | 0.00468322 | −1.73397 |
| | | (beta-urogastrone) | | |
| 214459_x_at | HLA-C | major histocompatibility | 0.00473524 | −1.06284 |
| | | complex, class I, C | | |
| 200859_x_at | FLNA | filamin A, alpha | 0.00474228 | −1.15462 |
| 201938_at | CDK2AP1 | cyclin-dependent kinase | 0.0047647 | −1.36598 |
| | | 2 associated protein 1 | | |
| 202378_s_at | LEPROT | leptin receptor | 0.00479261 | −1.26088 |
| | | overlapping transcript | | |
| 219710_at | SH3TC2 | SH3 domain and | 0.00480083 | −1.22839 |
| | | tetratricopeptide repeats 2 | | |
| 212242_at | TUBA4A | tubulin, alpha 4a | 0.00489677 | −1.25565 |
| 213511_s_at | MTMR1 | myotubularin related | 0.004902 | −1.09827 |
| | | protein 1 | | |
| 220109_at | TF | transferrin | 0.00492572 | −1.12186 |
| 217705_at | PRKD1 | protein kinase D1 | 0.00494361 | −1.08153 |
| 208753_s_at | NAP1L1 | nucleosome assembly | 0.00495688 | −1.22128 |
| | | protein 1-like 1 | | |
| 201280_s_at | DAB2 | disabled homolog 2, | 0.00497063 | −1.64395 |
| | | mitogen-responsive | | |
| | | phosphoprotein | | |
| | | (Drosophila) | | |
| 202838_at | FUCA1 | fucosidase, alpha-L-1, | 0.00499711 | 1.56419 |
| | | tissue | | |
| 205426_s_at | HIP1 | huntingtin interacting | 0.00499868 | −1.14213 |
| | | protein 1 | | |
| 211154_at | THPO | thrombopoietin | 0.00502652 | −1.11697 |
| 214577_at | MAP1B | micro tubule-associated | 0.00512459 | −1.14783 |
| | | protein 1B | | |
| 37966_at | PARVB | parvin, beta | 0.00513617 | −1.45109 |
| 209767_s_at | GP1BB /// | glycoprotein Ib | 0.00515773 | −1.47137 |
| | SEPT5 | (platelet), beta | | |
| | | polypeptide /// septin 5 | | |
| 40687_at | GJA4 | gap junction protein, | 0.00516689 | −1.14585 |
| | | alpha 4, 37 kDa | | |
| 216463_at | — | — | 0.00517514 | −1.14524 |
| 205128_x_at | PTGS1 | prostaglandin- | 0.00517864 | −1.54268 |
| | | endoperoxide synthase 1 | | |
| | | (prostaglandin G/H | | |
| | | synthase and | | |
| | | cyclooxyge | | |
| 221942_s_at | GUCY1A3 | guanylate cyclase 1, | 0.00526751 | −1.70831 |
| | | soluble, alpha 3 | | |
| 207156_at | HIST1H2AG | histone cluster 1, H2ag | 0.0053042 | −1.67358 |
| 211858_x_at | GNAS | GNAS complex locus | 0.00533874 | −1.13613 |
| 212692_s_at | LRBA | LPS-responsive vesicle | 0.00540408 | −1.1862 |
| | | trafficking, beach and | | |
| | | anchor containing | | |
| 211728_s_at | HYAL3 | hyaluronoglucosaminidase 3 | 0.00545859 | −1.16784 |
| 220336_s_at | GP6 | glycoprotein VI | 0.00546958 | −1.61325 |
| | | (platelet) | | |
| 217083_at | IGHG1 | Immunoglobulin heavy | 0.00548613 | −1.16387 |
| | | constant gamma 1 (G1m | | |
| | | marker) | | |
| 208327_at | CYP2A13 | cytochrome P450, | 0.00550711 | −1.14862 |
| | | family 2, subfamily A, | | |
| | | polypeptide 13 | | |
| 221555_x_at | CDC14B | CDC 14 cell division | 0.0055286 | −1.35261 |
| | | cycle 14 homolog B | | |
| | | (S. cerevisiae) | | |
| 211567_at | — | — | 0.00553946 | −1.12571 |
| 208403_x_at | MAX | MYC associated factor X | 0.00557349 | −1.29399 |
| 208989_s_at | KDM2A | lysine (K)-specific | 0.00557809 | −1.10556 |
| | | demethylase 2A | | |
| 201616_s_at | CALD1 | caldesmon 1 | 0.00558092 | −1.48431 |
| 204993_at | GNAZ | guanine nucleotide | 0.00558421 | −1.47787 |
| | | binding protein (G | | |
| | | protein), alpha z | | |
| | | polypeptide | | |
| 221764_at | C19orf22 | chromosome 19 open | 0.00558498 | −1.1412 |
| | | reading frame 22 | | |
| 206167_s_at | ARHGAP6 | Rho GTPase activating | 0.0055881 | −1.76822 |
| | | protein 6 | | |
| 204627_s_at | ITGB3 | integrin, beta 3 (platelet | 0.00559095 | −1.9911 |
| | | glycoprotein IIIa, | | |
| | | antigen CD61) | | |
| 200885_at | RHOC | ras homolog gene | 0.00563494 | −1.28435 |
| | | family, member C | | |
| 218117_at | RBX1 | ring-box 1 | 0.0056402 | −1.1728 |
| 206655_s_at | GP1BB /// | glycoprotein Ib | 0.00564505 | −1.81428 |
| | SEPT5 | (platelet), beta | | |
| | | polypeptide /// septin 5 | | |
| 200844_s_at | PRDX6 | peroxiredoxin 6 | 0.00565286 | −1.14511 |
| 216881_x_at | PRB4 | proline-rich protein | 0.00566372 | −1.11675 |
| | | BstNI subfamily 4 | | |
| 213746_s_at | FLNA | filamin A, alpha | 0.00567458 | −1.16364 |
| 208490_x_at | HIST1H2BF | histone cluster 1, H2bf | 0.00567635 | −1.43467 |
| 219202_at | RHBDF2 | rhomboid 5 homolog 2 | 0.00568725 | −1.12168 |
| | | (Drosophila) | | |
| 222382_x_at | NUP205 | nucleoporin 205 kDa | 0.00571901 | −1.14196 |
| 203998_s_at | SYT1 | synaptotagmin I | 0.00575584 | −1.13457 |
| 209826_at | EGFL8 /// | EGF-like-domain, | 0.0058323 | −1.1054 |
| | PPT2 | multiple 8 /// palmitoyl- | | |
| | | protein thioesterase 2 | | |
| 208601_s_at | TUBB1 | tubulin, beta 1 | 0.00591408 | −1.84224 |
| 214958_s_at | TMC6 | transmembrane channel- | 0.00592428 | −1.12494 |
| | | like 6 | | |
| 217335_at | FLJ11292 | hypothetical protein | 0.00594468 | −1.15949 |
| | | FLJ11292 | | |
| 213864_s_at | NAP1L1 | nucleosome assembly | 0.00597059 | −1.13884 |
| | | protein 1-like 1 | | |
| 203180_at | ALDH1A3 | aldehyde dehydrogenase | 0.00600686 | −1.16713 |
| | | 1 family, member A3 | | |
| 202332_at | CSNK1E | casein kinase 1, epsilon | 0.00600712 | −1.08867 |
| 210988_s_at | PRUNE | prune homolog | 0.00603465 | −1.30051 |
| | | (Drosophila) | | |
| 216896_at | COL4A3 | collagen, type IV, alpha | 0.00603529 | −1.14088 |
| | | 3 (Goodpasture antigen) | | |
| 220847_x_at | ZNF221 | zinc finger protein 221 | 0.00606358 | 1.15477 |
| 208931_s_at | ILF3 | interleukin enhancer | 0.00609592 | −1.14798 |
| | | binding factor 3, 90 kDa | | |
| 221160_s_at | CABP5 | calcium binding protein 5 | 0.00612012 | −1.56239 |
| 201528_at | RPA1 | replication protein A1, | 0.00612054 | −1.2309 |
| | | 70 kDa | | |
| 208750_s_at | ARF1 | ADP-ribosylation factor 1 | 0.00615213 | −1.10243 |
| 208523_x_at | HIST1H2BI | histone cluster 1, H2bi | 0.00617675 | −1.47477 |
| 215813_s_at | PTGS1 | prostaglandin- | 0.00620004 | −1.55575 |
| | | endoperoxide synthase 1 | | |
| | | (prostaglandin G/H | | |
| | | synthase and | | |
| | | cyclooxyge | | |
| 214917_at | PRKAA1 | protein kinase, AMP- | 0.00621432 | −1.17283 |
| | | activated, alpha 1 | | |
| | | catalytic subunit | | |
| 204000_at | GNB5 | guanine nucleotide | 0.00623899 | −1.19485 |
| | | binding protein (G | | |
| | | protein), beta 5 | | |
| 207046_at | HIST2H4A /// | histone cluster 2, H4a /// | 0.00626313 | −1.23715 |
| | HIST2H4B | histone cluster 2, H4b | | |
| 201885_s_at | CYB5R3 | cytochrome b5 | 0.0062861 | −1.15347 |
| | | reductase 3 | | |
| 221748_s_at | TNS1 | tensin 1 | 0.0062959 | −1.4944 |
| 216513_at | DCT | dopachrome | 0.00633287 | −1.16456 |
| | | tautomerase | | |
| | | (dopachrome delta- | | |
| | | isomerase, tyrosine- | | |
| | | related protein 2) | | |
| 204187_at | GMPR | guanosine | 0.00636191 | −1.47328 |
| | | monophosphate | | |
| | | reductase | | |
| 220518_at | ABI3BP | ABI family, member 3 | 0.00645579 | −1.13443 |
| | | (NESH) binding protein | | |
| 217673_x_at | GNAS | GNAS complex locus | 0.00646359 | −1.13184 |
| 213236_at | SASH1 | SAM and SH3 domain | 0.0064869 | 1.90481 |
| | | containing 1 | | |
| 205434_s_at | AAK1 | AP2 associated kinase 1 | 0.00656002 | −1.0984 |
| 211982_x_at | XPO6 | exportin 6 | 0.00657111 | −1.07265 |
| 210074_at | CTSL2 | cathepsin L2 | 0.00658045 | −1.24334 |
| 219705_at | QSER1 | glutamine and serine | 0.00662733 | −1.17822 |
| | | rich 1 | | |
| 208786_s_at | MAP1LC3B | microtubule-associated | 0.00665976 | −1.14821 |
| | | protein 1 light chain 3 | | |
| | | beta | | |
| 209651_at | TGFB1I1 | transforming growth | 0.00668902 | −1.76594 |
| | | factor beta 1 induced | | |
| | | transcript 1 | | |
| 215122_at | TBX6 | T-box 6 | 0.00679541 | −1.14526 |
| 206110_at | — | — | 0.00681334 | −1.79605 |
| 207745_at | CABP2 | calcium binding protein 2 | 0.00682426 | −1.13079 |
| 211334_at | MRE11A | MRE11 meiotic | 0.00687602 | −1.12163 |
| | | recombination 11 | | |
| | | homolog A | | |
| | | (S. cerevisiae) | | |
| 202501_at | MAPRE2 | microtubule-associated | 0.00688368 | −1.12271 |
| | | protein, RP/EB family, | | |
| | | member 2 | | |
| 204328_at | TMC6 | transmembrane channel- | 0.00689317 | −1.08397 |
| | | like 6 | | |
| 213598_at | — | — | 0.00699509 | −1.22087 |
| 205870_at | BDKRB2 | bradykinin receptor B2 | 0.00701629 | −1.14085 |
| 211026_s_at | MGLL | monoglyceride lipase | 0.00710106 | −1.60334 |
| 219983_at | HRASLS | HRAS-like suppressor | 0.00719792 | −1.52976 |
| 213908_at | WHAMML1 /// | WAS protein homolog | 0.00719896 | −1.37039 |
| | WHAMML2 | associated with actin, | | |
| | | golgi membranes and | | |
| | | microtubules-like | | |
| 208792_s_at | CLU | clusterin | 0.00721848 | −1.79473 |
| 204597_x_at | STC1 | stanniocalcin 1 | 0.00723265 | −1.14445 |
| 207963_at | C6orf54 | chromosome 6 open | 0.00723289 | −1.15074 |
| | | reading frame 54 | | |
| 213046_at | PABPN1 | poly(A) binding protein, | 0.00723382 | −1.1431 |
| | | nuclear 1 | | |
| 208690_s_at | PDLIM1 | PDZ and LIM domain 1 | 0.00723389 | −1.321 |
| 208791_at | CLU | clusterin | 0.00725265 | −1.77614 |
| 209423_s_at | PHF20 | PHD finger protein 20 | 0.00725781 | −1.14876 |
| 221746_at | UBL4A | ubiquitin-like 4A | 0.00726803 | −1.2092 |
| 212742_at | RNF115 | ring finger protein 115 | 0.00727752 | −1.09141 |
| 205221_at | HGD | homogentisate 1,2- | 0.00729169 | −1.52962 |
| | | dioxygenase | | |
| | | (homogentisate oxidase) | | |
| 214369_s_at | RASGRP2 | RAS guanyl releasing | 0.00738935 | −1.10618 |
| | | protein 2 (calcium and | | |
| | | DAG-regulated) | | |
| 210183_x_at | PNN | pinin, desmosome | 0.00742741 | −1.10331 |
| | | associated protein | | |
| 207799_x_at | — | — | 0.00743344 | −1.20384 |
| 217928_s_at | SAPS3 | SAPS domain family, | 0.00749046 | −1.10225 |
| | | member 3 | | |
| 213431_x_at | SFI1 | Sfi1 homolog, spindle | 0.00750358 | −1.06935 |
| | | assembly associated | | |
| | | (yeast) | | |
| 211059_s_at | GOLGA2 | golgi autoantigen, | 0.00750762 | −1.14379 |
| | | golgin subfamily a, 2 | | |
| 202708_s_at | HIST2H2BE | histone cluster 2, H2be | 0.00757968 | −1.54991 |
| 222121_at | SGEF | Src homology 3 domain- | 0.00760512 | −1.1164 |
| | | containing guanine | | |
| | | nucleotide exchange | | |
| | | factor | | |
| 215653_at | — | — | 0.00760953 | −1.11511 |
| 201124_at | ITGB5 | integrin, beta 5 | 0.0076181 | −1.25169 |
| 214308_s_at | HGD | homogentisate 1,2- | 0.00764686 | −1.64821 |
| | | dioxygenase | | |
| | | (homogentisate oxidase) | | |
| 217912_at | DUS1L | dihydrouridine synthase | 0.00767399 | −1.08336 |
| | | 1-like (S. cerevisiae) | | |
| 202974_at | MPP1 | membrane protein, | 0.00775127 | −1.36968 |
| | | palmitoylated 1, 55 kDa | | |
| 200905_x_at | HLA-E | major histocompatibility | 0.00775135 | −1.07063 |
| | | complex, class I, E | | |
| 216261_at | ITGB3 | integrin, beta 3 (platelet | 0.0077566 | −1.37764 |
| | | glycoprotein IIIa, | | |
| | | antigen CD61) | | |
| 206204_at | GRB14 | growth factor receptor- | 0.00781703 | −1.79964 |
| | | bound protein 14 | | |
| 203414_at | MMD | monocyte to | 0.00782001 | −1.41852 |
| | | macrophage | | |
| | | differentiation- | | |
| | | associated | | |
| 219779_at | ZFHX4 | zinc finger homeobox 4 | 0.00782075 | −1.16711 |
| 202573_at | CSNK1G2 | casein kinase 1, gamma 2 | 0.00784114 | −1.11116 |
| 208527_x_at | HIST1H2BE | histone cluster 1, H2be | 0.00788446 | −1.41486 |
| 213306_at | MPDZ | multiple PDZ domain | 0.00799342 | −1.10219 |
| | | protein | | |
| 216231_s_at | B2M | beta-2-microglobulin | 0.00809127 | −1.05409 |
| 207554_x_at | TBXA2R | thromboxane A2 | 0.00813716 | −1.28702 |
| | | receptor | | |
| 217963_s_at | NGFRAP1 | nerve growth factor | 0.00815053 | −1.41005 |
| | | receptor (TNFRSF16) | | |
| | | associated protein 1 | | |
| 201904_s_at | CTDSPL | CTD (carboxy-terminal | 0.00815064 | −1.51442 |
| | | domain, RNA | | |
| | | polymerase II, | | |
| | | polypeptide A) small | | |
| | | phosphatas | | |
| 205754_at | F2 | coagulation factor II | 0.00816234 | −1.14373 |
| | | (thrombin) | | |
| 204466_s_at | SNCA | synuclein, alpha (non | 0.0081789 | −1.56209 |
| | | A4 component of | | |
| | | amyloid precursor) | | |
| 201029_s_at | CD99 | CD99 molecule | 0.00820335 | −1.12753 |
| 202466_at | POLS | polymerase (DNA | 0.00822683 | −1.10075 |
| | | directed) sigma | | |
| 207550_at | MPL | myeloproliferative | 0.00828883 | −1.89086 |
| | | leukemia virus | | |
| | | oncogene | | |
| 208506_at | HIST1H3F | histone cluster 1, H3f | 0.0083864 | −1.13083 |
| 202774_s_at | SFRS8 | splicing factor, | 0.00842382 | −1.09754 |
| | | arginine/serine-rich 8 | | |
| | | (suppressor-of-white- | | |
| | | apricot homolog, Dr | | |
| 208343_s_at | NR5A2 | nuclear receptor | 0.00845048 | −1.11703 |
| | | subfamily 5, group A, | | |
| | | member 2 | | |
| 202778_s_at | ZMYM2 | zinc finger, MYM-type 2 | 0.00850235 | −1.12722 |
| 207523_at | C6orf10 | chromosome 6 open | 0.00859283 | −1.11067 |
| | | reading frame 10 | | |
| 219503_s_at | TMEM40 | transmembrane protein 40 | 0.00859971 | −1.45661 |
| 218704_at | RNF43 | ring finger protein 43 | 0.00873092 | −1.14893 |
| 209586_s_at | PRUNE | prune homolog | 0.00873312 | −1.23705 |
| | | (Drosophila) | | |
| 211449_at | MSH6 | mutS homolog 6 | 0.00875942 | −1.11398 |
| | | (E. coli) | | |
| 203896_s_at | PLCB4 | phospholipase C, beta 4 | 0.00876295 | −1.13939 |
| 204629_at | PARVB | parvin, beta | 0.00884733 | −1.27543 |
| 216623_x_at | TOX3 | TOX high mobility | 0.00885472 | −1.09839 |
| | | group box Family | | |
| | | member 3 | | |
| 204196_x_at | PKNOX1 | PBX/knotted 1 | 0.00892223 | −1.11739 |
| | | homeobox 1 | | |
| 215101_s_at | CXCL5 | chemokine (C-X-C | 0.00893221 | −1.66669 |
| | | motif) ligand 5 | | |
| 218243_at | RUFY1 | RUN and FYVE | 0.00894778 | −1.43684 |
| | | containing 1 | | |
| 204467_s_at | SNCA | A4 component of | 0.00896008 | −1.47629 |
| | | amyloid precursor) | | |
| 219857_at | C10orf81 | chromosome 10 open | 0.00897475 | −1.18546 |
| | | reading frame 81 | | |
| 216867_s_at | PDGFA | platelet-derived growth | 0.00897825 | −1.3116 |
| | | factor alpha polypeptide | | |
| 206779_s_at | ASMT | acetylserotonin O- | 0.00905468 | −1.13282 |
| | | methyltransferase | | |
| 214938_x_at | HMGB1 | high-mobility group box 1 | 0.00906415 | −1.08357 |
| 220094_s_at | CCDC90A | coiled-coil domain | 0.00907588 | −1.32015 |
| | | containing 90A | | |
| 207808_s_at | PROS1 | protein S (alpha) | 0.00911187 | −1.96407 |
| 200833_s_at | hCG_1757335 /// | RAP1B, member of | 0.00912774 | −1.12905 |
| | RAP1B | RAS oncogene family | | |
| | | pseudogene /// RAP1B, | | |
| | | member of RAS | | |
| | | oncogen | | |
| 206176_at | BMP6 | bone morphogenetic | 0.00916216 | −1.52992 |
| | | protein 6 | | |
| 210360_s_at | MTSS1 | metastasis suppressor 1 | 0.00917743 | −1.16565 |
| 211522_s_at | GNRHR | gonadotropin-releasing | 0.00918361 | −1.12079 |
| | | hormone receptor | | |
| 209840_s_at | LRRN3 | leucine rich repeat | 0.00922884 | 1.86067 |
| | | neuronal 3 | | |
| 214514_at | MCM3AP | minichromosome | 0.00924948 | −1.13224 |
| | | maintenance complex | | |
| | | component 3 associated | | |
| | | protein | | |
| 202620_s_at | PLOD2 | procollagen-lysine, 2- | 0.00936232 | −1.26035 |
| | | oxoglutarate 5- | | |
| | | dioxygenase 2 | | |
| 208752_x_at | NAP1L1 | nucleosome assembly | 0.00940735 | −1.13425 |
| | | protein 1-like 1 | | |
| 202619_s_at | PLOD2 | procollagen-lysine, 2- | 0.00942205 | −1.28918 |
| | | oxoglutarate 5- | | |
| | | dioxygenase 2 | | |
| 207397_s_at | HOXD13 | homeobox D13 | 0.00958266 | −1.11525 |
| 61297_at | CASKIN2 | CASK interacting | 0.00961064 | −1.11013 |
| | | protein 2 | | |
| 213765_at | MFAP5 | microfibrillar associated | 0.00964722 | −1.09199 |
| | | protein 5 | | |
| 207558_s_at | PITX2 | paired-like | 0.00964956 | −1.10328 |
| | | homeodomain 2 | | |
| 207827_x_at | SNCA | synuclein, alpha (non | 0.00976329 | −1.35299 |
| | | A4 component of | | |
| | | amyloid precursor) | | |
| 202555_s_at | MYLK | myosin light chain | 0.00978872 | −1.59189 |
| | | kinase | | |
| 212151_at | PBX1 | pre-B-cell leukemia | 0.00982704 | −1.5597 |
| | | homeobox 1 | | |
| 200845_s_at | PRDX6 | peroxiredoxin 6 | 0.00986511 | −1.2308 |
| 217736_s_at | EIF2AK1 | eukaryotic translation | 0.00989935 | −1.23452 |
| | | initiation factor 2-alpha | | |
| | | kinase 1 | | |
| 213828_x_at | H3F3A /// | H3 histone, family 3A /// | 0.00991103 | −1.09735 |
| | H3F3B /// | H3 histone, family 3B | | |
| | LOC440926 | (H3.3B) /// H3 histone, | | |
| | | family 3 | | |
| 216625_at | — | — | 0.00997586 | −1.10058 |

TABLE 7

| | | p-value | Fold-Change |
|---|---|---|---|
| Gene Symbol | Gene Title | (4.0 vs. 0.0) | (4.0 vs. 0.0) |
| TMEM158 | transmembrane protein 158 | 0.001631 | −1.88182 |
| TRIM58 | tripartite motif-containing 58 | 0.004607 | −1.73605 |
| FSTL1 | follistatin-like 1 | 0.001763 | −1.66003 |
| SNCA | synuclein, alpha (non A4 | 0.006379 | −1.59767 |
| | component of amyloid | | |
| | precursor) | | |
| ITGB5 | Integrin, beta 5 | 0.00485 | −1.5805 |
| TNS1 | tensin 1 | 0.003402 | −1.53358 |
| ATP1B1 | ATPase, Na+/K+ transporting, | 0.008818 | −1.51106 |
| | beta 1 polypeptide | | |
| C5orf4 | chromosome 5 open reading | 0.005208 | −1.46004 |
| | frame 4 | | |
| LRP12 | low density lipoprotein-related | 0.002832 | −1.42261 |
| | protein 12 | | |
| CTNNAL1 | catenin (cadherin-associated | 0.009018 | −1.40804 |
| | protein), alpha-like 1 | | |
| GEM | GTP binding protein | 0.002764 | −1.40178 |
| | overexpressed in skeletal | | |
| | muscle | | |
| KIAA1466 | KIAA1466 gene | 0.002973 | −1.39035 |
| ALDH1A2 | aldehyde dehydrogenase 1 | 0.000677 | −1.38981 |
| | family, member A2 | | |
| MAP4K3 | mitogen-activated protein | 0.007221 | −1.37714 |
| | kinase kinase kinase kinase 3 | | |
| SNCA | synuclein, alpha (non A4 | 0.007255 | −1.37607 |
| | component of amyloid | | |
| | precursor) | | |
| RAB6B | RAB6B, member RAS | 0.007576 | −1.37568 |
| | oncogene family | | |
| PSD3 | pleckstrin and Sec7 domain | 0.000178 | −1.37423 |
| | containing 3 | | |
| RIPK2 | receptor-interacting serine- | 0.008392 | −1.36879 |
| | threonine kinase 2 | | |
| RAMP3 | receptor (G protein-coupled) | 0.002203 | −1.36845 |
| | activity modifying protein 3 | | |
| PTCRA | pre T-cell antigen receptor | 0.003563 | −1.35879 |
| | alpha | | |
| CALD1 | caldesmon 1 | 0.002914 | −1.35604 |
| CYP2E1 | cytochrome P450, family 2, | 0.001334 | −1.35372 |
| | subfamily E, polypeptide 1 | | |
| PSD3 | pleckstrin and Sec7 domain | 0.000673 | −1.35294 |
| | containing 3 | | |
| PDLIM7 | PDZ and LIM domain 7 | 0.003532 | −1.34658 |
| | (enigma) | | |
| COBLL1 | COBL-like 1 | 0.002662 | −1.34562 |
| FUT3 | fucosyltransferase 3 | 2.63E−05 | −1.34512 |
| | (galactoside 3(4)-L- | | |
| | fucosyltransferase, Lewis blood | | |
| | group) | | |
| SMOX | spermine oxidase | 0.006872 | −1.34018 |
| TGM2 | transglutaminase 2 (C | 0.002055 | −1.33815 |
| | polypeptide, protein-glutamine- | | |
| | gamma-glutamyltransferase) | | |
| LRRC50 | leucine rich repeat containing 50 | 0.004871 | −1.33114 |
| CST6 | cystatin E/M | 0.001427 | −1.33016 |
| OR7A17 | olfactory receptor, family 7, | 0.000118 | −1.32853 |
| | subfamily A, member 17 | | |
| C6orf145 | chromosome 6 open reading | 0.00109 | −1.32816 |
| | frame 145 | | |
| DLEU2 /// | deleted in lymphocytic | 0.009215 | −1.32582 |
| DLEU2L | leukemia 2 (non-protein | | |
| | coding) /// deleted in | | |
| | lymphocytic leuke | | |
| CPT2 | carnitine palmitoyltransferase 2 | 0.002605 | −1.32033 |
| HGF | hepatocyte growth factor | 0.007517 | −1.31941 |
| | (hepapoietin A; scatter factor) | | |
| TNS1 | tensin 1 | 0.002806 | −1.31579 |
| SPRY1 | sprouty homolog 1, antagonist | 0.004614 | −1.30993 |
| | of FGF signaling (Drosophila) | | |
| PLOD2 | procollagen-lysine, 2- | 0.007309 | −1.30719 |
| | oxoglutarate 5-dioxygenase 2 | | |
| CD80 | CD80 molecule | 0.008637 | −1.30572 |
| KYNU | kynureninase (L-kynurenine | 0.009541 | −1.30549 |
| | hydrolase) | | |
| BCAT1 | branched chain | 0.009502 | −1.30486 |
| | aminotransferase 1, cytosolic | | |
| NHLH1 | nescient helix loop helix 1 | 0.00122 | −1.30451 |
| AHCTF1 | AT hook containing | 0.006984 | −1.30418 |
| | transcription factor 1 | | |
| HOXA10 | homeobox A10 | 0.007051 | −1.30259 |
| MTMR3 | myotubularin related protein 3 | 0.001598 | −1.30189 |
| — | — | 0.000939 | −1.30069 |
| VAC14 | Vac14 homolog (S. cerevisiae) | 2.51E−05 | −1.29695 |
| CLCF1 | cardiotrophin-like cytokine | 0.003153 | −1.2966 |
| | factor 1 | | |
| FGF5 | fibroblast growth factor 5 | 0.001 | −1.29505 |
| TAL1 | T-cell acute lymphocytic | 0.000808 | −1.29347 |
| | leukemia 1 | | |
| SAMD14 | sterile alpha motif domain | 0.00157 | −1.29276 |
| | containing 14 | | |
| ELL2 | elongation factor, RNA | 0.006259 | −1.29209 |
| | polymerase II, 2 | | |
| CHN1 | chimerin (chimaerin) 1 | 0.006634 | −1.2914 |
| SLC7A1 | solute carrier family 7 (cationic | 0.009963 | −1.28978 |
| | amino acid transporter, y+ | | |
| | system), member 1 | | |
| GRK5 | G protein-coupled receptor | 0.000218 | −1.28944 |
| | kinase 5 | | |
| PARD3 | par-3 partitioning defective 3 | 0.000992 | −1.28781 |
| | homolog (C. elegans) | | |
| VPS37B | vacuolar protein sorting 37 | 0.004355 | −1.28765 |
| | homolog B (S. cerevisiae) | | |
| CYP2B6 /// | cytochrome P450, family 2, | 0.001079 | −1.2873 |
| CYP2B7P1 | subfamily B, polypeptide 6 /// | | |
| | cytochrome P450, family 2, su | | |
| MALL | mal, T-cell differentiation | 0.000476 | −1.28554 |
| | protein-like | | |
| ALX4 | ALX homeobox 4 | 1.18E−05 | −1.28536 |
| SOX15 | SRY (sex determining region | 0.000755 | −1.28501 |
| | Y)-box 15 | | |
| KRT5 | keratin 5 | 0.000738 | −1.28477 |
| ESPL1 | extra spindle pole bodies | 0.003676 | −1.28424 |
| | homolog 1 (S. cerevisiae) | | |
| STARD8 | StAR-related lipid transfer | 0.00219 | −1.28408 |
| | (START) domain containing 8 | | |
| PSD3 | pleckstrin and Sec7 domain | 0.003653 | −1.28307 |
| | containing 3 | | |
| KIAA0195 | KIAA0195 | 3.69E−05 | −1.28154 |
| MYO9B | myosin IXB | 0.000252 | −1.27944 |
| HIP1R /// | huntingtin interacting protein 1 | 0.006353 | −1.2794 |
| LOC100294412 | related /// similar to KIAA0655 | | |
| | protein | | |
| EFNB1 | ephrin-B1 | 0.000145 | −1.27858 |
| ERN1 | endoplasmic reticulum to | 0.001593 | −1.27656 |
| | nucleus signaling 1 | | |
| RHD | Rh blood group, D antigen | 0.005698 | −1.27635 |
| MFAP3L | microfibrillar-associated protein | 0.002875 | −1.27538 |
| | 3-like | | |
| PLA1A | phospholipase A1 member A | 0.005885 | −1.27427 |
| POFUT2 | protein O-fucosyltransferase 2 | 0.004736 | −1.27411 |
| C8orf39 | chromosome 8 open reading | 0.002547 | −1.27348 |
| | frame 39 | | |
| CRYBB2 | crystallin, beta B2 | 0.000156 | −1.27288 |
| CYP4A11 | cytochrome P450, family 4, | 0.000381 | −1.27285 |
| | subfamily A, polypeptide 11 | | |
| PVRL2 | poliovirus receptor-related 2 | 0.007308 | −1.27216 |
| | (herpesvirus entry mediator B) | | |
| CLCNKB | chloride channel Kb | 0.001537 | −1.27136 |
| MRAS | muscle RAS oncogene homolog | 0.002321 | −1.27101 |
| NFIB | nuclear factor I/B | 0.000362 | −1.2706 |
| FKSG2 | apoptosis inhibitor | 0.003687 | −1.27027 |
| SLC11A2 | solute carrier family 11 (proton- | 0.008176 | −1.26987 |
| | coupled divalent metal ion | | |
| | transporters), member 2 | | |
| FZR1 | fizzy/cell division cycle 20 | 0.006166 | −1.26883 |
| | related 1 (Drosophila) | | |
| ZNF550 | zinc finger protein 550 | 0.00302 | −1.26876 |
| GLP1R | glucagon-like peptide 1 receptor | 0.001684 | −1.26854 |
| SLC19A1 | solute carrier family 19 (folate | 0.003885 | −1.26843 |
| | transporter), member 1 | | |
| RTN2 | reticulon 2 | 0.008304 | −1.26775 |
| PAPOLA | poly(A) polymerase alpha | 0.009359 | −1.2676 |
| STC1 | stanniocalcin 1 | 0.001341 | −1.26734 |
| GK | glycerol kinase | 0.004541 | −1.26678 |
| EXOSC6 | exosome component 6 | 0.00268 | −1.26637 |
| — | — | 4.96E−05 | −1.26602 |
| RAPSN | receptor-associated protein of | 0.003697 | −1.26598 |
| | the synapse | | |
| HFE | hemochromatosis | 0.000648 | −1.26583 |
| EHD2 | EH-domain containing 2 | 0.001249 | −1.26575 |
| RIOK3 | RIO kinase 3 (yeast) | 0.004132 | −1.26516 |
| UBE2I | Ubiquitin-conjugating enzyme | 0.00062 | −1.26466 |
| | E2I (UBC9 homolog, yeast) | | |
| C15orf2 | chromosome 15 open reading | 0.002573 | −1.26354 |
| | frame 2 | | |
| DMD | dystrophin | 0.006011 | −1.26327 |
| PRLH | prolactin releasing hormone | 0.001657 | −1.26177 |
| MAP2K2 | Mitogen-activated protein | 0.001555 | −1.26176 |
| | kinase kinase 2 | | |
| TP63 | tumor protein p63 | 0.001463 | −1.26066 |
| DACH1 | dachshund homolog 1 | 0.002299 | −1.26061 |
| | (Drosophila) | | |
| PPP5C | protein phosphatase 5, catalytic | 0.002092 | −1.26051 |
| | subunit | | |
| SLC26A1 | solute carrier family 26 (sulfate | 0.000553 | −1.26034 |
| | transporter), member 1 | | |
| NUDT7 | nudix (nucleoside diphosphate | 0.004276 | −1.25953 |
| | linked moiety X)-type motif 7 | | |
| KCNJ12 | potassium inwardly-rectifying | 0.000307 | −1.25907 |
| | channel, subfamily J, member 12 | | |
| ENTPD7 | ectonucleoside triphosphate | 0.00881 | −1.25885 |
| | diphosphohydrolase 7 | | |
| SLC26A1 | solute carrier family 26 (sulfate | 0.000894 | −1.25847 |
| | transporter), member 1 | | |
| PRRG3 | proline rich Gla (G- | 0.001239 | −1.25847 |
| | carboxyglutamic acid) 3 | | |
| | (transmembrane) | | |
| RGS6 | regulator of G-protein signaling 6 | 0.007795 | −1.25638 |
| ZBED2 | zinc finger, BED-type | 0.000482 | −1.25597 |
| | containing 2 | | |
| — | — | 1.57E−05 | −1.25554 |
| FICD | FIC domain containing | 0.005002 | −1.25533 |
| ARHGAP1 | Rho GTPase activating protein 1 | 0.002967 | −1.25434 |
| ARHGDIA | Rho GDP dissociation inhibitor | 0.00427 | −1.25429 |
| | (GDI) alpha | | |
| SDHB | succinate dehydrogenase | 0.003554 | −1.25315 |
| | complex, subunit B, iron sulfur Ip) | | |
| AMHR2 | anti-Mullerian hormone | 0.000653 | −1.25279 |
| | receptor, type II | | |
| ABCA4 | ATP-binding cassette, sub- | 0.001332 | −1.25263 |
| | family A (ABC1), member 4 | | |
| TCF20 | transcription factor 20 (AR1) | 0.005851 | −1.2525 |
| BGN | biglycan | 0.00473 | −1.25217 |
| CASP7 | caspase 7, apoptosis-related | 0.003516 | −1.25129 |
| | cysteine peptidase | | |
| LPAR4 | lysophosphatidic acid receptor 4 | 0.005372 | −1.25127 |
| GNA12 | guanine nucleotide binding | 0.009051 | −1.2511 |
| | protein (G protein) alpha 12 | | |
| CYP2W1 | cytochrome P450, family 2, | 0.00037 | −1.25048 |
| | subfamily W, polypeptide 1 | | |
| — | — | 0.005763 | −1.25006 |
| RAX | retina and anterior neural fold | 0.002983 | −1.24963 |
| | homeobox | | |
| C4A /// C4B /// | complement component 4A | 0.002229 | −1.24845 |
| LOC100292046 /// | (Rodgers blood group) /// | | |
| LOC100294156 | complement component 4B | | |
| | (Chido blood | | |
| ELAVL4 | ELAV (embryonic lethal, | 0.005864 | −1.24796 |
| | abnormal vision, Drosophila)- | | |
| | like 4 (Hu antigen D) | | |
| PXN | paxillin | 0.00025 | −1.24781 |
| ESR2 | estrogen receptor 2 (ER beta) | 0.000571 | −1.24778 |
| MYL10 | myosin, light chain 10, | 0.002715 | −1.24748 |
| | regulatory | | |
| EFS | embryonal Fyn-associated | 0.004955 | −1.24747 |
| | substrate | | |
| TFF3 | trefoil factor 3 (intestinal) | 0.000444 | −1.24739 |
| ADAM22 | ADAM metallopeptidase | 0.000495 | −1.24728 |
| | domain 22 | | |
| SRPK1 | SFRS protein kinase 1 | 0.008451 | −1.24704 |
| LOC441601 | septin 7 pseudogene | 8.14E−05 | −1.24632 |
| BIRC5 | baculoviral IAP repeat- | 0.000591 | −1.24548 |
| | containing 5 | | |
| CCT8L2 | chaperonin containing TCP1, | 0.0033 | −1.24521 |
| | subunit 8 (theta)-like 2 | | |
| PPAP2B | phosphatidic acid phosphatase | 0.008026 | −1.2452 |
| | type 2B | | |
| CMA1 | chymase 1, mast cell | 0.000993 | −1.245 |
| APOA2 | apolipoprotein A-II | 0.000594 | −1.24371 |
| KDELR2 | KDEL (Lys-Asp-Glu-Leu) | 0.007788 | −1.24358 |
| | endoplasmic reticulum protein | | |
| | retention receptor 2 | | |
| ASCL3 | achaete-scute complex homolog | 0.00054 | −1.24293 |
| | 3 (Drosophila) | | |
| RLINX1 | runt-related transcription | 0.0054 | −1.24289 |
| | factor 1 | | |
| BUB1 | budding uninhibited by | 0.000294 | −1.24284 |
| | benzimidazoles 1 homolog | | |
| | (yeast) | | |
| — | — | 0.003969 | −1.24241 |
| SLC6A8 | solute carrier family 6 | 0.000656 | −1.24067 |
| | (neurotransmitter transporter, | | |
| | creatine), member 8 | | |
| HNRNPC /// | heterogeneous nuclear | 0.008367 | −1.24043 |
| HNRNPCL1 /// | ribonucleoprotein C (C1/C2) /// | | |
| LOC440563 /// | heterogeneous nuclear | | |
| LOC649330 | ribonucleop | | |
| RIBC2 | RIB43A domain with coiled- | 4.24E−05 | −1.24036 |
| | coils 2 | | |
| CLIC4 | chloride intracellular channel 4 | 0.005848 | −1.24019 |
| RAB17 | RAB17, member RAS | 0.001346 | −1.24001 |
| | oncogene family | | |
| SCML2 | sex comb on midleg-like 2 | 0.008595 | −1.23921 |
| | (Drosophila) | | |
| SPINLW1 | serine peptidase inhibitor-like, | 9.13E−05 | −1.23909 |
| | with Kunitz and WAP domains | | |
| | 1 (eppin) | | |
| ANK1 | ankyrin 1, erythrocytic | 0.006497 | −1.23867 |
| EDA2R | ectodysplasin A2 receptor | 0.004698 | −1.2385 |
| — | — | 0.003041 | −1.23803 |
| — | — | 0.000661 | −1.23797 |
| HTR4 | 5-hydroxytryptamine | 1.84E−05 | −1.2378 |
| | (serotonin) receptor 4 | | |
| CDC42EP4 | CDC42 effector protein (Rho | 0.001214 | −1.23768 |
| | GTPase binding) 4 | | |
| KANK2 | KN motif and ankyrin repeat | 0.000895 | −1.23765 |
| | domains 2 | | |
| ANK1 | ankyrin 1, erythrocytic | 0.009625 | −1.2373 |
| ITGB3 | integrin, beta 3 (platelet | 0.001114 | −1.23728 |
| | glycoprotein IIIa, antigen | | |
| | CD61) | | |
| SYN1 | synapsin I | 0.005147 | −1.23728 |
| DUX3 /// | double homeobox, 3 /// double | 0.007355 | −1.23705 |
| DUX4 /// | homeobox, 4 /// FSHD region | | |
| FRG2C /// | gene 2 family, member C /// s | | |
| HPX-2 /// | | | |
| LOC100134409 /// | | | |
| LOC652119 /// | | | |
| LOC653543 /// | | | |
| LOC653544 /// | | | |
| LOC653545 /// | | | |
| LOC728410 | | | |
| PKNOX2 | PBX/knotted 1 homeobox 2 | 0.005082 | −1.23701 |
| MLLT4 | myeloid/lymphoid or mixed- | 0.002526 | −1.23601 |
| | lineage leukemia (trithorax | | |
| | homolog, Drosophila); | | |
| | translocate | | |
| APOA2 | apolipoprotein A-II | 0.004185 | −1.23591 |
| PENK | proenkephalin | 0.000174 | −1.23569 |
| GNAT1 | guanine nucleotide binding | 0.00958 | −1.23545 |
| | protein (G protein), alpha | | |
| | transducing activity polypeptide | | |
| FURIN | furin (paired basic amino acid | 0.006444 | −1.23543 |
| | cleaving enzyme) | | |
| SEMA6A | sema domain, transmembrane | 0.000683 | −1.23507 |
| | domain (TM), and cytoplasmic | | |
| | domain, (semaphorin) 6A | | |
| EGFL6 | EGF-like-domain, multiple 6 | 0.000502 | −1.23478 |
| HRH1 | histamine receptor H1 | 0.008279 | −1.23466 |
| TSPAN1 | tetraspanin 1 | 0.002802 | −1.23452 |
| DBC1 | deleted in bladder cancer 1 | 0.001766 | −1.23445 |
| TRPC7 | transient receptor potential | 2.45E−07 | −1.23402 |
| | cation channel, subfamily C, | | |
| | member 7 | | |
| MDM2 | Mdm2 p53 binding protein | 0.008092 | −1.23388 |
| | homolog (mouse) | | |
| GPR52 | G protein-coupled receptor 52 | 0.000198 | −1.23387 |
| HAMP | hepcidin antimicrobial peptide | 0.006054 | −1.2333 |
| PRSS2 | protease, serine, 2 (trypsin 2) | 0.001936 | −1.2322 |
| GPR107 | G protein-coupled receptor 107 | 0.008739 | −1.23212 |
| FLJ11292 | hypothetical protein FLJ11292 | 5.57E−05 | −1.23211 |
| FLJ20184 | hypothetical protein FLJ20184 | 0.005162 | −1.23203 |
| B4GALT1 | UDP-Gal: betaGlcNAc beta 1,4- | 0.000192 | −1.23117 |
| | galactosyltransferase, | | |
| | polypeptide 1 | | |
| NKX3-1 | NK3 homeobox 1 | 0.009204 | −1.23108 |
| ASIP | agouti signaling protein, | 0.002916 | −1.23063 |
| | nonagouti homolog (mouse) | | |
| SMAD4 | SMAD family member 4 | 0.004268 | −1.2306 |
| EFCAB6 | EF-hand calcium binding | 0.000165 | −1.23058 |
| | domain 6 | | |
| GPR20 | G protein-coupled receptor 20 | 0.008518 | −1.23016 |
| CA5A | carbonic anhydrase VA, | 0.004021 | −1.22996 |
| | mitochondrial | | |
| PLK4 | polo-like 4 (Drosophila) | 0.004056 | −1.22981 |
| TAAR5 | trace amine associated | 0.00273 | −1.22947 |
| | receptor 5 | | |
| SRPX2 | sushi-repeat-containing | 0.000298 | −1.22939 |
| | protein, X-linked 2 | | |
| CNTD2 | cyclin N-terminal domain | 1.28E−05 | −1.22932 |
| | containing 2 | | |
| AZGP1 | alpha-2-glycoprotein 1, zinc- | 0.004331 | −1.22925 |
| | binding | | |
| TIMP3 | TIMP metallopeptidase | 0.002046 | −1.22923 |
| | inhibitor 3 | | |
| RGS6 | regulator of G-protein | 0.006087 | −1.22916 |
| | signaling 6 | | |
| ADARB1 | adenosine deaminase, RNA- | 0.00212 | −1.22908 |
| | specific, B1 (RED1 homolog | | |
| | rat) | | |
| DYNC1I1 | dynein, cytoplasmic 1, | 0.000291 | −1.22872 |
| | intermediate chain 1 | | |
| C10orf10 | chromosome 10 open reading | 0.001942 | −1.22872 |
| | frame 10 | | |
| PDIA2 | protein disulfide isomerase | 0.001498 | −1.22865 |
| | family A, member 2 | | |
| PITX3 | paired-like homeodomain 3 | 0.009246 | −1.22861 |
| HOXC13 | homeobox C13 | 8.28E−05 | −1.22836 |
| LPAR3 | lysophosphatidic acid receptor 3 | 0.001583 | −1.22805 |
| CTRC | chymotrypsin C (caldecrin) | 0.008361 | −1.22773 |
| CTSL2 | cathepsin L2 | 0.005554 | −1.2276 |
| MUC8 | mucin 8 | 0.005519 | −1.22759 |
| AQP5 | aquaporin 5 | 0.000994 | −1.22755 |
| UGT1A1 /// | UDP glucuronosyltransferase 1 | 0.001167 | −1.22729 |
| UGT1A10 /// | family, polypeptide A1 /// UDP | | |
| UGT1A4 /// | glucuronosyltransferase 1 | | |
| UGT1A6 /// | | | |
| UGT1A8 /// | | | |
| UGT1A9 | | | |
| KCNQ2 | potassium voltage-gated | 0.001293 | −1.22727 |
| | channel, KQT-like subfamily, | | |
| | member 2 | | |
| CYP2A13 | cytochrome P450, family 2, | 0.00551 | −1.22653 |
| | subfamily A, polypeptide 13 | | |
| ZNF155 | zinc finger protein 155 | 0.005718 | −1.22653 |
| KIAA0892 | KIAA0892 | 0.000223 | −1.22645 |
| ATP2A2 | ATPase, Ca++ transporting, | 0.008882 | −1.22601 |
| | cardiac muscle, slow twitch 2 | | |
| MMP26 | matrix metallopeptidase 26 | 0.001265 | −1.22581 |
| FGF5 | fibroblast growth factor 5 | 0.003695 | −1.22569 |
| FGF18 | fibroblast growth factor 18 | 0.003001 | −1.22556 |
| FUT2 | fucosyltransferase 2 (secretor | 0.003882 | −1.22538 |
| | status included) | | |
| SHROOM2 | shroom family member 2 | 0.000419 | −1.22534 |
| PRSS3 | protease, serine, 3 | 0.006779 | −1.22529 |
| CREB3L1 | cAMP responsive element | 0.002111 | −1.22516 |
| | binding protein 3-like 1 | | |
| — | — | 0.008631 | −1.22511 |
| MGAT2 | mannosyl (alpha-1,6-)- | 0.006509 | −1.2251 |
| | glycoprotein beta-1,2-N- | | |
| | acetylglucosaminyltransferase | | |
| — | — | 0.000415 | −1.2249 |
| CSF1 | colony stimulating factor 1 | 0.001088 | −1.22487 |
| | (macrophage) | | |
| SMAD3 | SMAD family member 3 | 0.007701 | −1.22479 |
| PLCE1 | Phospholipase C, epsilon 1 | 0.005157 | −1.22464 |
| MLXIPL | MLX interacting protein-like | 0.004864 | −1.22443 |
| OR10H3 | olfactory receptor, family 10, | 0.001893 | −1.2243 |
| | subfamily H, member 3 | | |
| — | — | 0.000353 | −1.22418 |
| ABCB11 | ATP-binding cassette, sub- | 0.004152 | −1.224 |
| | family B (MDR/TAP), member 11 | | |
| CD84 | CD84 molecule | 0.009088 | −1.22398 |
| ARHGEF4 | Rho guanine nucleotide | 0.005157 | −1.22395 |
| | exchange factor (GEF) 4 | | |
| ORC1L | origin recognition complex, | 0.003618 | −1.22366 |
| | subunit 1-like (yeast) | | |
| PCIF1 | PDX1 C-terminal inhibiting | 0.007109 | −1.22348 |
| | factor 1 | | |
| CD177 | CD177 molecule | 0.000868 | −1.22342 |
| — | — | 0.000414 | −1.22314 |
| C1orf116 | chromosome 1 open reading | 0.000626 | −1.22307 |
| | frame 116 | | |
| — | — | 0.000385 | −1.2228 |
| IFT122 | intraflagellar transport 122 | 0.000359 | −1.22277 |
| | homolog (Chlamydomonas) | | |
| — | — | 0.000968 | −1.22273 |
| C11orf20 | chromosome 11 open reading | 0.002516 | −1.2225 |
| | frame 20 | | |
| DUSP13 | dual specificity phosphatase 13 | 0.000847 | −1.22179 |
| C6orf208 | chromosome 6 open reading | 0.001257 | −1.22163 |
| | frame 208 | | |
| PLA2G5 | phospholipase A2, group V | 5.46E−05 | −1.22142 |
| PRAMEF1 /// | PRAME family member 1 /// | 0.001073 | −1.22136 |
| PRAMEF2 | PRAME family member 2 | | |
| CYP4F8 | cytochrome P450, family 4, | 0.001494 | −1.22114 |
| | subfamily F, polypeptide 8 | | |
| KCNA1 | potassium voltage-gated | 0.00046 | −1.22105 |
| | channel, shaker-related | | |
| | subfamily, member 1 (episodic | | |
| | ataxia wi | | |
| MFAP4 | microfibrillar-associated | 0.000166 | −1.2209 |
| | protein 4 | | |
| C6 | complement component 6 | 0.006533 | −1.22081 |
| SLC4A3 | solute carrier family 4, anion | 0.009715 | −1.22068 |
| | exchanger, member 3 | | |
| IL1RAPL1 | interleukin 1 receptor accessory | 0.000271 | −1.22049 |
| | protein-like 1 | | |
| SERPINE1 | serpin peptidase inhibitor, clade | 0.001839 | −1.22049 |
| | E (nexin, plasminogen activator | | |
| | inhibitor type 1), me | | |
| ZCCHC14 | zinc finger, CCHC domain | 0.004618 | −1.22042 |
| | containing 14 | | |
| POLR3G | polymerase (RNA) III (DNA | 0.001007 | −1.22028 |
| | directed) polypeptide G (32 kD) | | |
| C16orf68 | chromosome 16 open reading | 0.006601 | −1.22026 |
| | frame 68 | | |
| FLJ14100 | hypothetical protein FLJ14100 | 0.003745 | −1.22017 |
| SMCHD1 | structural maintenance of | 0.008572 | −1.2201 |
| | chromosomes flexible hinge | | |
| | domain containing 1 | | |
| ASCL1 | achaete-scute complex homolog | 0.002304 | −1.21998 |
| | 1 (Drosophila) | | |
| FOXA2 | forkhead box A2 | 0.00025 | −1.2197 |
| SLC23A2 | solute carrier family 23 | 0.005914 | −1.21969 |
| | (nucleobase transporters), | | |
| | member 2 | | |
| KLK13 | kallikrein-related peptidase 13 | 0.000211 | −1.21966 |
| MTSS1L | metastasis suppressor 1-like | 0.001589 | −1.21956 |
| DNMT3L | DNA (cytosine-5-)- | 0.000936 | −1.21952 |
| | methyltransferase 3-like | | |
| RREB1 | ras responsive element binding | 0.006278 | −1.21948 |
| | protein 1 | | |
| DNMBP | dynamin binding protein | 0.007794 | −1.21943 |
| PKLR | pyruvate kinase, liver and RBC | 0.000571 | −1.21918 |
| C1orf106 | chromosome 1 open reading | 0.005004 | −1.21911 |
| | frame 106 | | |
| CCDC134 | coiled-coil domain containing 134 | 0.000478 | −1.21888 |
| MTSS1 | metastasis suppressor 1 | 0.002441 | −1.21878 |
| CCDC40 | coiled-coil domain containing 40 | 0.000701 | −1.21869 |
| HOXB1 | homeobox B1 | 0.006406 | −1.21825 |
| SCNN1B | sodium channel, nonvoltage- | 0.001488 | −1.2182 |
| | gated 1, beta | | |
| SEMA4G | sema domain, immunoglobulin | 0.002662 | −1.2182 |
| | domain (Ig), transmembrane | | |
| | domain (TM) and short | | |
| | cytoplasmi | | |
| RAPGEFL1 | Rap guanine nucleotide | 0.000162 | −1.21787 |
| | exchange factor (GEF)-like 1 | | |
| MAGEL2 | MAGE-like 2 | 0.000123 | −1.21777 |
| — | — | 0.000234 | −1.21771 |
| PLSCR2 | phospholipid scramblase 2 | 0.000386 | −1.21727 |
| CHD2 | chromodomain helicase DNA | 0.000841 | −1.21722 |
| | binding protein 2 | | |
| PLCD1 | phospholipase C, delta 1 | 0.005374 | −1.2171 |
| C1orf116 | chromosome 1 open reading | 0.006 | −1.21704 |
| | frame 116 | | |
| CHRNA2 | cholinergic receptor, nicotinic, | 0.008482 | −1.21702 |
| | alpha 2 (neuronal) | | |
| MBP | myelin basic protein | 0.008574 | −1.21675 |
| CDC42BPA | CDC42 binding protein kinase | 0.000334 | −1.21665 |
| | alpha (DMPK-like) | | |
| TNFRSF11A | tumor necrosis factor receptor | 0.007883 | −1.21627 |
| | superfamily, member 11a, | | |
| | NFKB activator | | |
| MYF6 | myogenic factor 6 (herculin) | 0.003356 | −1.21615 |
| PI15 | peptidase inhibitor 15 | 0.004832 | −1.21612 |
| LOC440895 | LIM and senescent cell antigen- | 0.003588 | −1.21578 |
| | like domains 3-like | | |
| SBF1 | SET binding factor 1 | 0.002572 | −1.21568 |
| MAST1 | microtubule associated | 0.001899 | −1.21565 |
| | serine/threonine kinase 1 | | |
| GLT8D2 | glycosyltransferase 8 domain | 0.000458 | −1.21564 |
| | containing 2 | | |
| ERBB3 | v-erb-b2 erythroblastic | 0.000806 | −1.21564 |
| | leukemia viral oncogene | | |
| | homolog 3 (avian) | | |
| LOH3CR2A | loss of heterozygosity, 3, | 0.004412 | −1.21562 |
| | chromosomal region 2, gene A | | |
| AMH | anti-Mullerian hormone | 0.000237 | −1.21552 |
| HR | hairless homolog (mouse) | 0.005332 | −1.21547 |
| RDH8 | retinol dehydrogenase 8 (all- | 0.000487 | −1.21536 |
| | trans) | | |
| PAWR | PRKC, apoptosis, WT1, | 0.005543 | −1.2152 |
| | regulator | | |
| DRD3 | dopamine receptor D3 | 0.000203 | −1.21493 |
| CCT8 | chaperonin containing TCP1, | 0.009015 | −1.21463 |
| | subunit 8 (theta) | | |
| PRELP | proline/arginine-rich end | 0.007385 | −1.21443 |
| | leucine-rich repeat protein | | |
| SPOCK3 | sparc/osteonectin, cwcv and | 0.000394 | −1.21434 |
| | kazal-like domains | | |
| | proteoglycan (testican) 3 | | |
| EPS8L3 | EPS8-like 3 | 0.007312 | −1.21407 |
| NXN | nucleoredoxin | 0.003294 | −1.21404 |
| SEMA4G | sema domain, immunoglobulin | 0.001706 | −1.21395 |
| | domain (Ig), transmembrane | | |
| | domain (TM) and short | | |
| | cytoplasmi | | |
| P2RY1 | purinergic receptor P2Y, G- | 0.002207 | −1.21385 |
| | protein coupled, 1 | | |
| AVL9 | AVL9 homolog (S. cerevisiase) | 0.002166 | −1.21376 |
| TEK | TEK tyrosine kinase, | 0.000493 | −1.21369 |
| | endothelial | | |
| MOGAT2 | monoacylglycerol O- | 0.002638 | −1.21358 |
| | acyltransferase 2 | | |
| KLK7 | kallikrein-related peptidase 7 | 0.007089 | −1.21357 |
| MT1E /// | metallothionein 1E /// | 0.008728 | −1.21355 |
| MT1H /// | metallothionein 1H /// | | |
| MT1M | metallothionein 1M | | |
| CLDN18 | claudin 18 | 0.002968 | −1.21353 |
| RHBDF2 | rhomboid 5 homolog 2 | 0.007107 | −1.21331 |
| | (Drosophila) | | |
| SIX1 | SIX homeobox 1 | 0.006149 | −1.21304 |
| INPP5A | inositol polyphosphate-5- | 0.00971 | −1.21301 |
| | phosphatase, 40 kDa | | |
| KCNMB3 | potassium large conductance | 0.007976 | −1.213 |
| | calcium-activated channel, | | |
| | subfamily M beta member 3 | | |
| MAP2K5 | mitogen-activated protein | 0.00099 | −1.21293 |
| | kinase kinase 5 | | |
| GPD1 | glycerol-3-phosphate | 0.003338 | −1.21278 |
| | dehydrogenase 1 (soluble) | | |
| LPO | lactoperoxidase | 0.001326 | −1.21277 |
| LOC729143 /// | similar to Myosin phosphatase | 0.007077 | −1.21259 |
| MPRIP | Rho-interacting protein (Rho- | | |
| | interacting protein 3) (M-RI | | |
| WNT7A | wingless-type MMTV | 0.004044 | −1.21249 |
| | integration site family, | | |
| | member 7A | | |
| — | — | 0.000279 | −1.21223 |
| RARG | retinoic acid receptor, gamma | 0.002589 | −1.21222 |
| CDH7 | cadherin 7, type 2 | 0.004733 | −1.2116 |
| MBNL2 | muscleblind-like 2 (Drosophila) | 0.006252 | −1.21154 |
| RASGRP2 | RAS guanyl releasing protein 2 | 0.007323 | −1.21144 |
| | (calcium and DAG-regulated) | | |
| RBMY2FP | RNA binding motif protein, Y- | 2.59E−05 | −1.21141 |
| | linked, family 2, member F | | |
| | pseudogene | | |
| MASP1 | mannan-binding lectin serine | 0.009232 | −1.2109 |
| | peptidase 1 (C4/C2 activating | | |
| | component of Ra-reactive fac | | |
| CASR | calcium-sensing receptor | 0.004273 | −1.21088 |
| EGR4 | early growth response 4 | 0.001108 | −1.21043 |
| APOC2 | apolipoprotein C-II | 0.002122 | −1.21042 |
| HECW1 | HECT, C2 and WW domain | 0.005258 | −1.2103 |
| | containing E3 ubiquitin protein | | |
| | ligase 1 | | |
| HOXB3 | homeobox B3 | 0.003953 | −1.21029 |
| IRF5 | interferon regulatory factor 5 | 0.009858 | −1.21029 |
| NNMT | nicotinamide N- | 0.000406 | −1.21028 |
| | methyltransferase | | |
| AOC2 | amine oxidase, copper | 0.004428 | −1.21023 |
| | containing 2 (retina-specific) | | |
| ESRRG | estrogen-related receptor | 0.001335 | −1.20993 |
| | gamma | | |
| LPIN1 | lipin 1 | 0.009736 | −1.20987 |
| ACOT11 | acyl-CoA thioesterase 11 | 0.000945 | −1.20973 |
| CCDC33 | coiled-coil domain containing 33 | 0.007172 | −1.20945 |
| MBD2 | methyl-CpG binding domain | 0.003023 | −1.20941 |
| | protein 2 | | |
| ZNF323 | zinc finger protein 323 | 0.009551 | −1.20931 |
| NTRK2 | neurotrophic tyrosine kinase, | 0.000251 | −1.20921 |
| | receptor, type 2 | | |
| TMEM151B | transmembrane protein 151B | 0.009983 | −1.20898 |
| GPLD1 | glycosylphosphatidylinositol | 0.006394 | −1.20848 |
| | specific phospholipase D1 | | |
| LENEP | lens epithelial protein | 0.000284 | −1.20832 |
| HNF1B | HNF1 homeobox B | 0.001386 | −1.20824 |
| NXPH3 | neurexophilin 3 | 0.001589 | −1.20798 |
| — | — | 0.006641 | −1.20793 |
| ALDH1A3 | aldehyde dehydrogenase 1 | 0.000392 | −1.20788 |
| | family, member A3 | | |
| PHF20L1 | PHD finger protein 20-like 1 | 0.002957 | −1.20781 |
| CKM | creatine kinase, muscle | 0.0008 | −1.20774 |
| — | — | 0.001361 | −1.20746 |
| PARD6B | par-6 partitioning defective 6 | 0.000827 | −1.20711 |
| | homolog beta (C. elegans) | | |
| CRYGB | crystallin, gamma B | 0.005502 | −1.20704 |
| HAB1 | B1 for mucin | 0.001879 | −1.20699 |
| LARGE | like-glycosyltransferase | 0.009606 | −1.20682 |
| RAB40C | RAB40C, member RAS | 0.00324 | −1.20676 |
| | oncogene family | | |
| MPL | myeloproliferative leukemia | 0.007992 | −1.20668 |
| | virus oncogene | | |
| CHIT1 | chitinase 1 (chitotriosidase) | 0.003357 | −1.20667 |
| METTL10 | methyltransferase like 10 | 0.003511 | −1.20663 |
| DUS4L | dihydrouridine synthase 4-like | 0.00298 | −1.20661 |
| | (S. cerevisiae) | | |
| PNLIPRP1 | pancreatic lipase-related | 0.000459 | −1.20659 |
| | protein 1 | | |
| ELL | elongation factor RNA | 0.001662 | −1.20651 |
| | polymerase II | | |
| ST8SIA5 | ST8 alpha-N-acetyl- | 0.000615 | −1.20633 |
| | neuraminide alpha-2,8- | | |
| | sialyltransferase 5 | | |
| ITGA8 | integrin, alpha 8 | 0.009387 | −1.20629 |
| GRIN2B | glutamate receptor, ionotropic, | 0.000406 | −1.20603 |
| | N-methyl D-aspartate 2B | | |
| MC4R | melanocortin 4 receptor | 0.00036 | −1.20584 |
| RTDR1 | rhabdoid tumor deletion region | 0.000275 | −1.20581 |
| | gene 1 | | |
| HDAC6 | histone deacetylase 6 | 0.001545 | −1.2058 |
| KCNJ13 | potassium inwardly-rectifying | 0.001433 | −1.20567 |
| | channel, subfamily J, member 13 | | |
| CPSF1 | cleavage and polyadenylation | 1.67E−05 | −1.20546 |
| | specific factor 1, 160 kDa | | |
| SPANXC | SPANX family, member C | 0.001064 | −1.2054 |
| CNOT4 | CCR4-NOT transcription | 0.007152 | −1.20522 |
| | complex, subunit 4 | | |
| LAMA2 | Laminin, alpha 2 | 7.89E−05 | −1.20506 |
| SLC1A6 | solute carrier family 1 (high | 0.00372 | −1.205 |
| | affinity aspartate/glutamate | | |
| | transporter), member 6 | | |
| ABCA2 | ATP-binding cassette, sub- | 0.002267 | −1.20494 |
| | family A (ABC1), member 2 | | |
| KLK11 | kallikrein-related peptidase 11 | 0.000758 | −1.20493 |
| GFRA3 | GDNF family receptor alpha 3 | 0.002967 | −1.2047 |
| CYP3A4 | cytochrome P450, family 3, | 0.002771 | −1.20468 |
| | subfamily A, polypeptide 4 | | |
| SLC1A3 | solute carrier family 1 (glial | 0.004552 | −1.20467 |
| | high affinity glutamate | | |
| | transporter), member 3 | | |
| ATP2B2 | ATPase, Ca++ transporting, | 0.000594 | −1.20453 |
| | plasma membrane 2 | | |
| APBB2 | amyloid beta (A4) precursor | 0.005968 | −1.20439 |
| | protein-binding, family B, | | |
| | member 2 | | |
| VPS45 | vacuolar protein sorting 45 | 0.000839 | −1.20431 |
| | homolog (S. cerevisiae) | | |
| GHRHR | growth hormone releasing | 0.003426 | −1.20425 |
| | hormone receptor | | |
| HOXD4 | homeobox D4 | 0.004276 | −1.20421 |
| PRPH | peripherin | 4.94E−05 | −1.20416 |
| ADCY2 | adenylate cyclase 2 (brain) | 0.006778 | −1.20412 |
| LEFTY2 | left-right determination factor 2 | 0.00084 | −1.20391 |
| CYP1B1 | cytochrome P450, family 1, | 0.002715 | −1.20353 |
| | subfamily B, polypeptide 1 | | |
| PCP4 | Purkinje cell protein 4 | 2.27E−05 | −1.20337 |
| C8B | complement component 8, beta | 0.0017 | −1.2033 |
| | polypeptide | | |
| RANBP3 | RAN binding protein 3 | 0.001832 | −1.2033 |
| PDE6H | phosphodiesterase 6H, cGMP- | 0.002496 | −1.20303 |
| | specific, cone, gamma | | |
| TRIM15 | tripartite motif-containing 15 | 0.00027 | −1.20261 |
| VGLL1 | vestigial like 1 (Drosophila) | 0.001092 | −1.20257 |
| TRIM3 | tripartite motif-containing 3 | 0.000537 | −1.20249 |
| LTBP4 | latent transforming growth | 0.000462 | −1.20238 |
| | factor beta binding protein 4 | | |
| CRKL | v-crk sarcoma virus CT10 | 0.008611 | −1.20236 |
| | oncogene homolog (avian)-like | | |
| ADH7 | alcohol dehydrogenase 7 (class | 0.000166 | −1.20227 |
| | IV), mu or sigma polypeptide | | |
| PSG3 | pregnancy specific beta-1- | 0.00053 | −1.20227 |
| | glycoprotein 3 | | |
| GPR153 | G protein-coupled receptor 153 | 0.008656 | −1.20222 |
| MFAP2 | microfibrillar-associated | 0.003428 | −1.20216 |
| | protein 2 | | |
| FGF13 | fibroblast growth factor 13 | 0.002263 | −1.20212 |
| — | — | 0.007125 | −1.202 |
| NAPA | N-ethylmaleimide-sensitive | 0.006488 | −1.20191 |
| | factor attachment protein, alpha | | |
| ALDH3A1 | aldehyde dehydrogenase 3 | 0.000897 | −1.20175 |
| | family, member A1 | | |
| MCM10 | minichromosome maintenance | 0.005216 | −1.20168 |
| | complex component 10 | | |
| TLE4 | transducin-like enhancer of split | 0.006143 | −1.20166 |
| | 4 (E(sp1) homolog, Drosophila) | | |
| ITPR3 | inositol 1,4,5-triphosphate | 0.006944 | −1.20157 |
| | receptor, type 3 | | |
| CCDC87 | coiled-coil domain containing 87 | 0.001771 | −1.20124 |
| C9orf7 | chromosome 9 open reading | 0.009273 | −1.2011 |
| | frame 7 | | |
| ACTC1 | actin, alpha, cardiac muscle 1 | 0.00076 | −1.20109 |
| OBSL1 | obscurin-like 1 | 0.002861 | −1.20096 |
| — | — | 0.000232 | −1.20095 |
| MAP2 | microtubule-associated | 0.003324 | −1.20084 |
| | protein 2 | | |
| CRYM | crystallin, mu | 0.005793 | −1.20073 |
| RNF122 | ring finger protein 122 | 0.003704 | −1.20071 |
| SST | somatostatin | 0.003629 | −1.2007 |
| HLA-DRB6 | major histocompatibility | 0.009489 | −1.20021 |
| | complex, class II, DR beta 6 | | |
| | (pseudogene) | | |
| SLC22A17 | solute carrier family 22, | 0.00219 | −1.20018 |
| | member 17 | | |
| HSPG2 | heparan sulfate proteoglycan 2 | 0.000654 | −1.20017 |
| HIP1 | huntingtin interacting protein 1 | 4.28E−05 | −1.20004 |
| GRIK2 | glutamate receptor, ionotropic, | 3.13E−06 | −1.19991 |
| | kainate 2 | | |
| — | — | 0.008492 | −1.19976 |
| UNKL | unkempt homolog | 0.005034 | −1.19954 |
| | (Drosophila)-like | | |
| GPR144 | G protein-coupled receptor 144 | 0.007186 | −1.19948 |
| KIR3DX1 | killer cell immunoglobulin-like | 0.003825 | −1.1993 |
| | receptor, three domains, X1 | | |
| — | — | 0.007994 | −1.1993 |
| NARFL | nuclear prelamin A recognition | 0.003052 | −1.19926 |
| | factor-like | | |
| — | — | 0.000127 | −1.19903 |
| UCP3 | uncoupling protein 3 | 0.009564 | −1.19903 |
| | (mitochondrial, proton carrier) | | |
| — | — | 0.001429 | −1.19877 |
| PLXNA2 | plexin A2 | 0.001989 | −1.19862 |
| BTN1A1 | butyrophilin, subfamily 1, | 0.003656 | −1.19858 |
| | member A1 | | |
| ERCC4 | excision repair cross- | 0.007138 | −1.19837 |
| | complementing rodent repair | | |
| | deficiency, complementation | | |
| | group 4 | | |
| CIITA | class II, major | 0.008237 | −1.1982 |
| | histocompatibility complex, | | |
| | transactivator | | |
| EGFR | epidermal growth factor | 0.008886 | −1.19797 |
| | receptor (erythroblastic | | |
| | leukemia viral (v-erb-b) | | |
| | oncogene homo | | |
| — | — | 0.005458 | −1.19781 |
| KRT33A | keratin 33A | 0.006693 | −1.19769 |
| CLTB | Clathrin, light chain (Lcb) | 0.008512 | −1.19768 |
| B3GALT5 | UDP-Gal: betaGlcNAc beta 1,3- | 0.001241 | −1.19754 |
| | galactosyltransferase, | | |
| | polypeptide 5 | | |
| — | — | 0.009616 | −1.19751 |
| AP3M2 | adaptor-related protein complex | 0.002731 | −1.19749 |
| | 3, mu 2 subunit | | |
| GJC1 | gap junction protein, gamma 1, | 0.009693 | −1.19749 |
| | 45 kDa | | |
| MYO3A | myosin IIIA | 0.000406 | −1.19726 |
| ADAM12 | ADAM metallopeptidase | 0.000608 | −1.19713 |
| | domain 12 | | |
| ARHGAP1 | Rho GTPase activating protein 1 | 0.001148 | −1.19713 |
| PPP2R3A | protein phosphatase 2 (formerly | 0.002 | −1.19703 |
| | 2A), regulatory subunit B″, | | |
| | alpha | | |
| CLIC4 | chloride intracellular channel 4 | 0.00595 | −1.19699 |
| C20orf195 | chromosome 20 open reading | 0.005195 | −1.19672 |
| | frame 195 | | |
| SIGLEC8 | sialic acid binding Ig-like | 0.000256 | −1.19653 |
| | lectin 8 | | |
| GPRC5A | G protein-coupled receptor, | 0.002762 | −1.19624 |
| | family C, group 5, member A | | |
| CACNB1 | calcium channel, voltage- | 0.003107 | −1.19613 |
| | dependent, beta 1 subunit | | |
| MYL10 | myosin, light chain 10, | 0.009335 | −1.19609 |
| | regulatory | | |
| PRLR | prolactin receptor | 0.000985 | −1.19602 |
| OR2S2 | olfactory receptor, family 2, | 0.003564 | −1.19593 |
| | subfamily S, member 2 | | |
| NCR2 | Natural cytotoxicity triggering | 0.005113 | −1.19575 |
| | receptor 2 | | |
| CHAF1B | chromatin assembly factor 1, | 8.04E−05 | −1.19574 |
| | subunit B (p60) | | |
| EYA3 | eyes absent homolog 3 | 0.005876 | −1.19566 |
| | (Drosophila) | | |
| CDS1 | CDP-diacylglycerol synthase | 0.006301 | −1.19565 |
| | (phosphatidate | | |
| | cytidylyltransferase) 1 | | |
| FBXL18 | F-box and leucine-rich repeat | 6.72E−06 | −1.1956 |
| | protein 18 | | |
| — | — | 3.49E−05 | −1.19547 |
| — | — | 0.006093 | −1.19544 |
| ADAM22 | ADAM metallopeptidase | 0.000119 | −1.19543 |
| | domain 22 | | |
| ACTL6B | actin-like 6B | 0.001385 | −1.19543 |
| ZNF821 | zinc finger protein 821 | 0.002862 | −1.19538 |
| C16orf71 | chromosome 16 open reading | 0.006501 | −1.19537 |
| | frame 71 | | |
| HBBP1 | hemoglobin, beta pseudogene 1 | 0.006504 | −1.19525 |
| PLXNA1 | plexin A1 | 0.003653 | −1.1951 |
| CDC45L | CDC45 cell division cycle 45- | 0.00364 | −1.19488 |
| | like (S. cerevisiae) | | |
| MTCP1 | mature T-cell proliferation 1 | 0.002145 | −1.19479 |
| PLCB4 | phospholipase C, beta 4 | 0.006205 | −1.19469 |
| PLVAP | plasmalemma vesicle associated | 0.007844 | −1.19456 |
| | protein | | |
| PROX1 | prospero homeobox 1 | 0.003286 | −1.19447 |
| CYP3A43 | cytochrome P450, family 3, | 0.004232 | −1.19391 |
| | subfamily A, polypeptide 43 | | |
| ICHG1 | Immunoglobulin heavy constant | 0.000798 | −1.1939 |
| | gamma 1 (G1m marker) | | |
| RECQL5 | RecQ protein-like 5 | 0.00231 | −1.19387 |
| IDUA | Iduronidase, alpha-L- | 0.007734 | −1.19383 |
| DLGAP4 | discs, large (Drosophila) | 0.009247 | −1.19341 |
| | homolog-associated protein 4 | | |
| PLXNB1 | plexin B1 | 0.007795 | −1.19307 |
| HSD17B14 | hydroxysteroid (17-beta) | 0.002049 | −1.19271 |
| | dehydrogenase 14 | | |
| FOXP3 | forkhead box P3 | 0.007901 | −1.19261 |
| C19orf26 | chromosome 19 open reading | 0.00256 | −1.19219 |
| | frame 26 | | |
| EPB41L1 | erythrocyte membrane protein | 0.000528 | −1.19208 |
| | band 4.1-like 1 | | |
| RBBP9 | retinoblastoma binding | 0.003886 | −1.19197 |
| | protein 9 | | |
| GJB4 | gap junction protein, beta 4, | 0.005636 | −1.19173 |
| | 30.3 kDa | | |
| UPK1B | uroplakin 1B | 0.001588 | −1.19168 |
| CYP19A1 | cytochrome P450, family 19, | 0.002082 | −1.1916 |
| | subfamily A, polypeptide 1 | | |
| LOC55908 | hepatocellular carcinoma- | 0.002937 | −1.1916 |
| | associated gene TD26 | | |
| CLDN18 | claudin 18 | 0.003193 | −1.1916 |
| C2orf72 | chromosome 2 open reading | 0.002873 | −1.19147 |
| | frame 72 | | |
| NTRK3 | neurotrophic tyrosine kinase, | 3.09E−05 | −1.19142 |
| | receptor, type 3 | | |
| NRXN2 | neurexin 2 | 0.000836 | −1.1914 |
| SPDEF | SAM pointed domain | 0.000244 | −1.19138 |
| | containing ets transcription | | |
| | factor | | |
| IGH@ /// | immunoglobulin heavy locus /// | 0.001803 | −1.19135 |
| IGHD /// | immunoglobulin heavy constant | | |
| IGHG1 /// | delta /// immunoglobulin h | | |
| IGHM /// | | | |
| LOC100289944 /// | | | |
| VSIG6 | | | |
| ACRV1 | acrosomal vesicle protein 1 | 0.003333 | −1.19132 |
| PHLDB1 | pleckstrin homology-like | 0.001717 | −1.1913 |
| | domain, family B, member 1 | | |
| SORBS1 | sorbin and SH3 domain | 0.00522 | −1.19127 |
| | containing 1 | | |
| — | — | 6.67E−05 | −1.19122 |
| HAPLN2 | hyaluronan and proteoglycan | 0.001502 | −1.19118 |
| | link protein 2 | | |
| FABP3 | fatty acid binding protein 3, | 0.003523 | −1.19097 |
| | muscle and heart (mammary- | | |
| | derived growth inhibitor) | | |
| EFS | embryonal Fyn-associated | 0.001768 | −1.19081 |
| | substrate | | |
| ACVR1B | activin A receptor, type IB | 0.00457 | −1.19081 |
| CHST3 | carbohydrate (chondroitin 6) | 0.001252 | −1.19075 |
| | sulfotransferase 3 | | |
| UGT2A1 /// | UDP glucuronosyltransferase 2 | 0.000599 | −1.19065 |
| UGT2A2 | family, polypeptide A1 /// UDP | | |
| | glucuronosyltransferase 2 | | |
| TAF1 | TAF1 RNA polymerase II, | 0.007846 | −1.1905 |
| | TATA box binding protein | | |
| | (TBP)-associated factor, | | |
| | 250 kDa | | |
| MT4 | metallothionen 4 | 0.002292 | −1.19047 |
| MFAP3 | microfibrillar-associated | 0.008836 | −1.19025 |
| | protein 3 | | |
| ETV5 | ets variant 5 | 0.002412 | −1.19021 |
| UBQLN3 | ubiquilin 3 | 0.001961 | −1.1902 |
| TBX10 | T-box 10 | 0.001032 | −1.19013 |
| — | — | 0.00191 | −1.18979 |
| GJB1 | gap junction protein, beta 1, | 0.008453 | −1.18979 |
| | 32 kDa | | |
| ABO | ABO blood group (transferase | 0.007208 | −1.18959 |
| | A, alpha 1-3-N- | | |
| | acetylgalactosaminyltransferase; | | |
| | transferas | | |
| SPINK5 | serine peptidase inhibitor, Kazal | 0.001357 | −1.18917 |
| | type 5 | | |
| ATAD4 | ATPase family, AAA domain | 0.000327 | −1.18914 |
| | containing 4 | | |
| CDH11 | cadherin 11, type 2, OB- | 0.000198 | −1.18913 |
| | cadherin (osteoblast) | | |
| CARD14 | caspase recruitment domain | 0.002462 | −1.18906 |
| | family, member 14 | | |
| ALPP /// | alkaline phosphatase, placental | 0.001709 | −1.18902 |
| ALPPL2 | (Regan isozyme) /// alkaline | | |
| | phosphatase, placental-lik | | |
| CBL | Cas-Br-M (murine) ecotropic | 0.009088 | −1.18899 |
| | retroviral transforming | | |
| | sequence | | |
| LRP4 | low density lipoprotein | 0.005919 | −1.18889 |
| | receptor-related protein 4 | | |
| CDKL2 | cyclin-dependent kinase-like 2 | 0.00225 | −1.18883 |
| | (CDC2-related kinase) | | |
| SSX3 | synovial sarcoma, X breakpoint 3 | 0.002688 | −1.18867 |
| DSG2 | desmoglein 2 | 0.006638 | −1.18848 |
| SLC45A2 | solute carrier family 45, | 0.001818 | −1.18847 |
| | member 2 | | |
| LAMA4 | laminin, alpha 4 | 0.00392 | −1.18846 |
| WFDC8 | WAP four-disulfide core | 0.001163 | −1.18843 |
| | domain 8 | | |
| HTR7 | 5-hydroxytryptamine | 0.001731 | −1.18841 |
| | (serotonin) receptor 7 | | |
| | (adenylate cyclase-coupled) | | |
| EFNB3 | ephrin-B3 | 0.005729 | −1.18838 |
| TUBB2B | tubulin, beta 2B | 0.000497 | −1.18837 |
| OR7E19P | olfactory receptor, family 7, | 0.001943 | −1.18834 |
| | subfamily E, member 19 | | |
| | pseudogene | | |
| PMS2L4 | postmeiotic segregation | 0.006959 | −1.1883 |
| | increased 2-like 4 pseudogene | | |
| ASAP3 | ArfGAP with SH3 domain, | 0.000269 | −1.18819 |
| | ankyrin repeat and PH domain 3 | | |
| FRZB | frizzled-related protein | 0.001369 | −1.1881 |
| PDLIM4 | PDZ and LIM domain 4 | 0.003582 | −1.18805 |
| PVT1 | Pvt1 oncogene (non-protein | 0.001967 | −1.18803 |
| | coding) | | |
| TFR2 | transferrin receptor 2 | 0.00593 | −1.18802 |
| AHI1 | Abelson helper integration | 0.008274 | −1.18798 |
| | site 1 | | |
| — | — | 0.001985 | −1.18788 |
| TAF4 | TAF4 RNA polymerase II, | 0.000919 | −1.18784 |
| | TATA box binding protein | | |
| | (TBP)-associated factor, | | |
| | 135 kDa | | |
| ADAMTSL2 | ADAMTS-like 2 | 0.008834 | −1.18783 |
| CLDN4 | claudin 4 | 0.000164 | −1.1878 |
| KIR2DL1 /// | killer cell immunoglobulin-like | 0.000231 | −1.1878 |
| KIR2DL2 /// | receptor, two domains, long | | |
| KIR2DL3 /// | cytoplasmic tail, 1 /// kil | | |
| KIR2DL5A /// | | | |
| KIR2DL5B /// | | | |
| KIR2DS1 /// | | | |
| KIR2DS2 /// | | | |
| KIR2DS3 /// | | | |
| KIR2DS4 /// | | | |
| KIR2DS5 /// | | | |
| KIR3DL2 /// | | | |
| KIR3DL3 /// | | | |
| KIR3DP1 /// | | | |
| LOC727787 | | | |
| RAPGEF5 | Rap guanine nucleotide | 0.002052 | −1.18774 |
| | exchange factor (GEF) 5 | | |
| CRMP1 | collapsin response mediator | 0.008402 | −1.18763 |
| | protein 1 | | |
| LDB3 | LIM domain binding 3 | 0.000824 | −1.18759 |
| — | — | 0.001275 | −1.18749 |
| F11 | coagulation factor XI | 0.004401 | −1.18745 |
| USP46 | ubiquitin specific peptidase 46 | 0.009226 | −1.18742 |
| PTN | pleiotrophin | 0.00012 | −1.18707 |
| IBSP | integrin-binding sialoprotein | 0.000822 | −1.18706 |
| SLC9A3 | solute carrier family 9 | 0.003578 | −1.18695 |
| | (sodium/hydrogen exchanger), | | |
| | member 3 | | |
| FLRT3 | fibronectin leucine rich | 0.002107 | −1.18691 |
| | transmembrane protein 3 | | |
| TRIM17 | tripartite motif-containing 17 | 0.002821 | −1.18688 |
| FGF17 | fibroblast growth factor 17 | 0.005417 | −1.18682 |
| CAMK1G | calcium/calmodulin-dependent | 0.003767 | −1.18654 |
| | protein kinase IG | | |
| GLYR1 | glyoxylate reductase 1 homolog | 0.001708 | −1.18625 |
| | (Arabidopsis) | | |
| CSH1 | chorionic somatomammotropin | 0.000163 | −1.18612 |
| | hormone 1 (placental lactogen) | | |
| NTF3 | neurotrophin 3 | 0.002903 | −1.18611 |
| ABHD6 | abhydrolase domain containing 6 | 0.000573 | −1.18608 |
| TRIM15 | tripartite motif-containing 15 | 0.002896 | −1.18596 |
| OR52A1 | olfactory receptor, family 52, | 0.002896 | −1.18579 |
| | subfamily A, member 1 | | |
| FGFR2 | fibroblast growth factor | 0.000178 | −1.18567 |
| | receptor 2 | | |
| ORAI2 | ORAI calcium release-activated | 0.007127 | −1.18563 |
| | calcium modulator 2 | | |
| — | — | 0.002783 | −1.18518 |
| — | — | 0.002321 | −1.18507 |
| C17orf53 | chromosome 17 open reading | 0.001902 | −1.18505 |
| | frame 53 | | |
| GLP1R | glucagon-like peptide 1 receptor | 0.003491 | −1.1849 |
| SL1T1 | slit homolog 1 (Drosophila) | 0.002042 | −1.18475 |
| TP63 | tumor protein p63 | 0.006308 | −1.18464 |
| DDR1 | discoidin domain receptor | 0.007775 | −1.18462 |
| | tyrosine kinase 1 | | |
| CFTR | cystic fibrosis transmembrane | 0.000534 | −1.18451 |
| | conductance regulator (ATP- | | |
| | binding cassette sub-family C, | | |
| DIO2 | deiodinase, iodothyronine, | 0.003653 | −1.18445 |
| | type II | | |
| LETM1 | leucine zipper-EF-hand | 0.005131 | −1.18438 |
| | containing transmembrane | | |
| | protein 1 | | |
| ACSM5 | acyl-CoA synthetase medium- | 0.000303 | −1.18437 |
| | chain family member 5 | | |
| — | — | 0.001738 | −1.18434 |
| ACTA1 | actin, alpha 1, skeletal muscle | 0.003411 | −1.18432 |
| NPR1 | natriuretic peptide receptor | 0.004745 | −1.1842 |
| | A/guanylate cyclase A | | |
| | (atrionatriuretic peptide | | |
| | receptor A | | |
| KCND3 | potassium voltage-gated | 0.008592 | −1.18418 |
| | channel, ShaI-related subfamily, | | |
| | member 3 | | |
| POPDC3 | popeye domain containing 3 | 0.002195 | −1.18411 |
| DNAH3 | dynein, axonemal, heavy chain 3 | 0.008169 | −1.18403 |
| SPDEF | SAM pointed domain | 0.007526 | −1.18397 |
| | containing ets transcription | | |
| | factor | | |
| CLEC4M | C-type lectin domain family 4, | 0.000696 | −1.18389 |
| | member M | | |
| — | — | 0.004001 | −1.18375 |
| SLC30A3 | solute carrier family 30 (zinc | 0.00669 | −1.18367 |
| | transporter), member 3 | | |
| NAGLU | N-acetylglucosaminidase, | 0.002574 | −1.18361 |
| | alpha- | | |
| AAK1 | AP2 associated kinase 1 | 0.004007 | −1.18358 |
| DHX34 | DEAH (Asp-Glu-Ala-His) box | 0.002492 | −1.18357 |
| | polypeptide 34 | | |
| NNAT | neuronatin | 0.008336 | −1.18355 |
| — | — | 0.007629 | −1.18337 |
| AKAP9 | A kinase (PRKA) anchor | 0.00231 | −1.18329 |
| | protein (yotiao) 9 | | |
| ICMT | isoprenylcysteine carboxyl | 0.007841 | −1.18329 |
| | methyltransferase | | |
| FAM189A1 | family with sequence similarity | 0.007897 | −1.18319 |
| | 189, member A1 | | |
| C10orf81 | chromosome 10 open reading | 0.009408 | −1.18318 |
| | frame 81 | | |
| MYOZ1 | myozenin 1 | 0.008907 | −1.18309 |
| PKNOX2 | PBX/knotted 1 homeobox 2 | 8.10E−05 | −1.18298 |
| MGC31957 | hypothetical protein | 0.001407 | −1.18284 |
| | MGC31957 | | |
| PRDM11 | PR domain containing 11 | 0.004128 | −1.18266 |
| RET | ret proto-oncogene | 0.004396 | −1.18265 |
| IGHG1 | Immunoglobulin heavy constant | 0.002101 | −1.18263 |
| | gamma 1 (G1m marker) | | |
| XPNPEP2 | X-prolyl aminopeptidase | 0.004951 | −1.18263 |
| | (aminopeptidase P) 2, | | |
| | membrane-bound | | |
| NTRK2 | neurotrophic tyrosine kinase, | 7.26E-05 | −1.18262 |
| | receptor, type 2 | | |
| — | — | 0.009809 | −1.1826 |
| — | — | 0.004011 | −1.18253 |
| SLC25A10 | solute carrier family 25 | 0.000841 | −1.18243 |
| | (mitochondrial carrier; | | |
| | dicarboxylate transporter), | | |
| | member 10 | | |
| NR1I2 | nuclear receptor subfamily 1, | 0.004273 | −1.18219 |
| | group I, member 2 | | |
| — | — | 0.0068 | −1.18217 |
| GRM8 | glutamate receptor, | 0.002678 | −1.18202 |
| | metabotropic 8 | | |
| OR3A3 | olfactory receptor, family 3, | 0.001803 | −1.18201 |
| | subfamily A, member 3 | | |
| GIPR | gastric inhibitory polypeptide | 0.001874 | −1.1819 |
| | receptor | | |
| PAH | phenylalanine hydroxylase | 0.001658 | −1.18186 |
| PACRG | PARK2 co-regulated | 0.007415 | −1.18175 |
| — | — | 0.003881 | −1.18173 |
| CLN8 | ceroid-lipofuscinosis, neuronal | 0.001987 | −1.18166 |
| | 8 (epilepsy, progressive with | | |
| | mental retardation) | | |
| ZNF215 | zinc finger protein 215 | 0.000173 | −1.18165 |
| TRIO | Triple functional domain | 0.003634 | −1.1816 |
| | (PTPRF interacting) | | |
| TTLL5 | tubulin tyrosine ligase-like | 0.008239 | −1.18155 |
| | family, member 5 | | |
| GRM1 | glutamate receptor, | 0.004897 | −1.18148 |
| | metabotropic 1 | | |
| PRKG1 | protein kinase, cGMP- | 0.002024 | −1.18147 |
| | dependent, type I | | |
| HHLA1 | HERV-H LTR-associating 1 | 0.008614 | −1.18137 |
| LAMA3 | laminin, alpha 3 | 0.002922 | −1.18134 |
| PTN | pleiotrophin | 0.002345 | −1.18131 |
| SLC37A4 | solute carrier family 37 | 0.006933 | −1.18114 |
| | (glucose-6-phosphate | | |
| | transporter), member 4 | | |
| HOXC11 | homeobox C11 | 0.000624 | −1.18111 |
| SLCO5A1 | solute carrier organic anion | 6.88E−05 | −1.18102 |
| | transporter family, member 5A1 | | |
| CA10 | carbonic anhydrase X | 0.001818 | −1.18102 |
| — | — | 0.005863 | −1.18094 |
| RRBP1 | ribosome binding protein 1 | 0.000657 | −1.1809 |
| | homolog 180 kDa (dog) | | |
| SOD3 | superoxide dismutase 3, | 0.00355 | −1.18082 |
| | extracellular | | |
| NTRK3 | neurotrophic tyrosine kinase, | 0.003705 | −1.18081 |
| | receptor, type 3 | | |
| CYR61 | cysteine-rich, angiogenic | 0.003919 | −1.18079 |
| | inducer, 61 | | |
| STRA6 | stimulated by retinoic acid gene | 0.005735 | −1.18068 |
| | 6 homolog (mouse) | | |
| SLC6A11 | solute carrier family 6 | 0.007789 | −1.18065 |
| | (neurotransmitter transporter, | | |
| | GABA), member 11 | | |
| CNOT4 | CCR4-NOT transcription | 0.004365 | −1.18064 |
| | complex, subunit 4 | | |
| ATN1 | Atrophin 1 | 0.004412 | −1.18059 |
| ITGB4 | integrin, beta 4 | 0.001879 | −1.18054 |
| BCAP29 | B-cell receptor-associated | 0.005292 | −1.18045 |
| | protein 29 | | |
| — | — | 0.004726 | −1.18036 |
| NOVA2 | neuro-oncological ventral | 0.00162 | −1.18035 |
| | antigen 2 | | |
| — | — | 0.005358 | −1.18035 |
| RELN | reelin | 0.003425 | −1.18034 |
| LAMC2 | laminin, gamma 2 | 0.006538 | −1.18034 |
| — | — | 0.003782 | −1.18031 |
| RAD51 | RAD51 homolog (RecA | 0.008493 | −1.18024 |
| | homolog, E. coli) (S. cerevisiae) | | |
| — | — | 0.000913 | −1.18016 |
| PRSS7 | protease, serine, 7 | 0.005123 | −1.18016 |
| | (enterokinase) | | |
| DCBLD2 | discoidin, CUB and LCCL | 0.000493 | −1.18007 |
| | domain containing 2 | | |
| TACR2 | tachykinin receptor 2 | 0.002078 | −1.18003 |
| RAB11B | RAB11B, member RAS | 0.004596 | −1.17994 |
| | oncogene family | | |
| OR2J2 | olfactory receptor, family 2, | 0.000236 | −1.17993 |
| | subfamily J, member 2 | | |
| VSNL1 | visinin-like 1 | 0.001379 | −1.17992 |
| IFNA17 | interferon, alpha 17 | 0.003586 | −1.17985 |
| DPYSL4 | dihydropyrimidinase-like 4 | 0.00248 | −1.17961 |
| — | — | 0.009056 | −1.17959 |
| MGC2889 | hypothetical protein MGC2889 | 0.001552 | −1.17951 |
| RRBP1 | Ribosome binding protein 1 | 0.007965 | −1.17935 |
| | homolog 180 kDa (dog) | | |
| POLQ | polymerase (DNA directed), | 0.002209 | −1.17934 |
| | theta | | |
| OR1A2 | olfactory receptor, family 1, | 7.49E−06 | −1.17927 |
| | subfamily A, member 2 | | |
| PURA | Purine-rich element binding | 0.00771 | −1.17918 |
| | protein A | | |
| AIF1 | allograft inflammatory factor 1 | 0.00406 | −1.17917 |
| CBS | cystathionine-beta-synthase | 0.008348 | −1.17902 |
| NECAB2 | N-terminal EF-hand calcium | 0.003146 | −1.17901 |
| | binding protein 2 | | |
| PRKCE | protein kinase C, epsilon | 0.003727 | −1.17899 |
| NOX1 | NADPH oxidase 1 | 0.003303 | −1.17898 |
| IHH | Indian hedgehog homolog | 0.001392 | −1.17891 |
| | (Drosophila) | | |
| EXO1 | exonuclease 1 | 0.002234 | −1.17891 |
| GPRIN2 | G protein regulated inducer of | 0.005827 | −1.17888 |
| | neurite outgrowth 2 | | |
| PDX1 | pancreatic and duodenal | 0.003138 | −1.17881 |
| | homeobox 1 | | |
| GPR12 | G protein-coupled receptor 12 | 0.007938 | −1.17835 |
| — | — | 0.004616 | −1.17827 |
| FAM188A | family with sequence similarity | 0.005191 | −1.17827 |
| | 188, member A | | |
| HS3ST3B1 | heparan sulfate (glucosamine) | 0.003282 | −1.17824 |
| | 3-O-sulfotransferase 3B1 | | |
| ASCL1 | achaete-scute complex homolog | 0.000169 | −1.17813 |
| | 1 (Drosophila) | | |
| ZNF484 | zinc finger protein 484 | 0.000728 | −1.1781 |
| SERPINB3 | serpin peptidase inhibitor, clade | 5.85E−05 | −1.17802 |
| | B (ovalbumin), member 3 | | |
| CSH1 | chorionic somatomammotropin | 0.000315 | −1.178 |
| | hormone 1 (placental lactogen) | | |
| BCAN | brevican | 0.006433 | −1.17796 |
| DDN | dendrin | 0.005892 | −1.17792 |
| DUOX2 | dual oxidase 2 | 0.002385 | −1.17761 |
| MORN1 | MORN repeat containing 1 | 0.004195 | −1.17751 |
| SLC39A2 | solute carrier family 39 (zinc | 0.006145 | −1.17751 |
| | transporter), member 2 | | |
| CLCN7 | chloride channel 7 | 0.00054 | −1.17749 |
| RUNX2 | runt-related transcription factor 2 | 0.000734 | −1.17741 |
| TTYH1 | tweety homolog 1 (Drosophila) | 0.001039 | −1.17723 |
| ZNF280B | zinc finger protein 280B | 0.008339 | −1.17716 |
| PAX3 | paired box 3 | 0.000716 | −1.17714 |
| LZTS1 | leucine zipper, putative tumor | 0.009862 | −1.17712 |
| | suppressor 1 | | |
| SLC8A2 | solute carrier family 8 | 0.003583 | −1.17706 |
| | (sodium/calcium exchanger), | | |
| | member 2 | | |
| HAB1 | B1 for mucin | 0.00946 | −1.17705 |
| KIF1A | kinesin family member 1A | 0.002068 | −1.17694 |
| ARL4D | ADP-ribosylation factor-like 4D | 0.002302 | −1.17694 |
| UGT2B15 | UDP glucuronosyltransferase 2 | 0.007983 | −1.17694 |
| | family, polypeptide B15 | | |
| NACA2 | nascent polypeptide-associated | 0.00631 | −1.17693 |
| | complex alpha subunit 2 | | |
| THRB | thyroid hormone receptor, beta | 0.000259 | −1.17685 |
| | (erythroblastic leukemia viral | | |
| | (v-erb-a) oncogene homolo | | |
| C6orf15 | chromosome 6 open reading | 0.004187 | −1.17685 |
| | frame 15 | | |
| — | — | 0.008907 | −1.17685 |
| GPR176 | G protein-coupled receptor 176 | 0.00317 | −1.17651 |
| WSCD1 | WSC domain containing 1 | 0.005206 | −1.17645 |
| PLXNB3 | plexin B3 | 0.002725 | −1.17642 |
| CADM3 | cell adhesion molecule 3 | 0.008183 | −1.17636 |
| HAP1 | huntingtin-associated protein 1 | 2.19E−05 | −1.17629 |
| CYP1A2 | cytochrome P450, family 1, | 0.003159 | −1.17629 |
| | subfamily A, polypeptide 2 | | |
| SPAM1 | sperm adhesion molecule 1 | 0.000727 | −1.17625 |
| | (PH-20 hyaluronidase, zona | | |
| | pellucida binding) | | |
| IL22RA1 | interleukin 22 receptor, alpha 1 | 0.001309 | −1.17617 |
| CDC2L5 | cell division cycle 2-like 5 | 0.007821 | −1.17609 |
| | (cholinesterase-related cell | | |
| | division controller) | | |
| IRX5 | iroquois homeobox 5 | 0.000291 | −1.17596 |
| PPFIA2 | protein tyrosine phosphatase, | 0.001152 | −1.17588 |
| | receptor type, f polypeptide | | |
| | (PTPRF), interacting protein | | |
| — | — | 0.004585 | −1.17587 |
| KDELR3 | KDEL (Lys-Asp-Glu-Leu) | 0.000471 | −1.17559 |
| | endoplasmic reticulum protein | | |
| | retention receptor 3 | | |
| CEACAM7 | carcinoembryonic antigen- | 0.005552 | −1.17556 |
| | related cell adhesion molecule 7 | | |
| KCMF1 | potassium channel modulatory | 0.009164 | −1.17553 |
| | factor 1 | | |
| DUOX1 | dual oxidase 1 | 0.008808 | −1.17546 |
| — | — | 0.000615 | −1.17528 |
| CDC27 | cell division cycle 27 homolog | 0.009706 | −1.17522 |
| | (S. cerevisiae) | | |
| HIST2H2AA3 | histone cluster 2, H2aa3 | 0.002777 | −1.17519 |
| CAV3 | caveolin 3 | 0.008482 | −1.17519 |
| APOA4 | apolipoprotein A-IV | 0.006002 | −1.17518 |
| — | — | 0.001198 | −1.17511 |
| NPR3 | natriuretic peptide receptor | 0.004663 | −1.1751 |
| | C/guanylate cyclase C | | |
| | (atrionatriuretic peptide | | |
| | receptor C | | |
| PRG3 | proteoglycan 3 | 3.39E−05 | −1.17507 |
| TBC1D22B | TBC1 domain family, member 22B | 0.004838 | −1.17506 |
| TUSC3 | tumor suppressor candidate 3 | 0.000348 | −1.175 |
| RIMS2 | regulating synaptic membrane | 0.005824 | −1.175 |
| | exocytosis 2 | | |
| CYP4F12 | cytochrome P450, family 4, | 0.007756 | −1.1748 |
| | subfamily F, polypeptide 12 | | |
| TBXA2R | thromboxane A2 receptor | 0.000835 | −1.17478 |
| HBEGF | Heparin-binding EGF-like | 0.001173 | −1.17476 |
| | growth factor | | |
| PSG9 | pregnancy specific beta-1- | 0.000597 | −1.17461 |
| | glycoprotein 9 | | |
| PYGO1 | pygopus homolog 1 | 0.000119 | −1.17423 |
| | (Drosophila) | | |
| RASGRF1 | Ras protein-specific guanine | 0.007711 | −1.17412 |
| | nucleotide-releasing factor 1 | | |
| SCN2A | sodium channel, voltage-gated, | 0.005444 | −1.17405 |
| | type II, alpha subunit | | |
| KLHL1 | kelch-like 1 (Drosophila) | 0.003584 | −1.17404 |
| DTNB | dystrobrevin, beta | 0.005577 | −1.17402 |
| GREM1 | gremlin 1, cysteine knot | 0.008798 | −1.17396 |
| | superfamily, homolog | | |
| | (Xenopus laevis) | | |
| SNCG | synuclein, gamma (breast | 0.005937 | −1.17388 |
| | cancer-specific protein 1) | | |
| C22orf24 | chromosome 22 open reading | 0.000444 | −1.17382 |
| | frame 24 | | |
| PALM | paralemmin | 0.006745 | −1.17378 |
| COBLL1 | COBL-like 1 | 0.003288 | −1.17374 |
| DNPEP | aspartyl aminopeptidase | 0.008863 | −1.17361 |
| MNS1 | meiosis-specific nuclear | 0.009321 | −1.1735 |
| | structural 1 | | |
| NFATC4 | nuclear factor of activated T- | 0.003566 | −1.17336 |
| | cells, cytoplasmic, calcineurin- | | |
| | dependent 4 | | |
| — | — | 0.001222 | −1.1733 |
| DLC1 | deleted in liver cancer 1 | 0.009225 | −1.17318 |
| — | — | 0.002702 | −1.17316 |
| HSPC072 | hypothetical LOC29075 | 0.003774 | −1.17306 |
| MCAM | melanoma cell adhesion | 0.00272 | −1.17289 |
| | molecule | | |
| CA12 | carbonic anhydrase XII | 0.006108 | −1.17285 |
| CSHL1 | chorionic somatomammotropin | 0.000243 | −1.17282 |
| | hormone-like 1 | | |
| RPAIN | RPA interacting protein | 0.000483 | −1.17274 |
| COL5A2 | collagen, type V, alpha 2 | 0.004487 | −1.1727 |
| UGT1A8 /// | UDP glucuronosyltransferase 1 | 3.79E−05 | −1.17265 |
| UGT1A9 | family, polypeptide A8 /// UDP | | |
| | glucuronosyltransferase 1 | | |
| IGH@ /// | immunoglobulin heavy locus /// | 0.002422 | −1.17249 |
| IGHA1 /// | immunoglobulin heavy constant | | |
| IGHG1 /// | alpha 1 /// immunoglobulin | | |
| IGHG2 /// | | | |
| IGHG3 /// | | | |
| IGHM /// | | | |
| LOC100126583 /// | | | |
| LOC100290036 /// | | | |
| LOC100290320 /// | | | |
| LOC100293211 /// | | | |
| LOC652494 | | | |
| ITGB1 | integrin, beta 1 (fibronectin | 0.001351 | −1.17248 |
| | receptor, beta polypeptide, | | |
| | antigen CD29 includes MDF2, M | | |
| TGFB2 | transforming growth factor, | 0.003578 | −1.17248 |
| | beta 2 | | |
| ACSM5 | acyl-CoA synthetase medium- | 0.00119 | −1.17244 |
| | chain family member 5 | | |
| — | — | 0.000204 | −1.17236 |
| ALOX12P2 | arachidonate 12-lipoxygenase | 0.00767 | −1.17234 |
| | pseudogene 2 | | |
| ERBB4 | v-erb-a erythroblastic leukemia | 0.006521 | −1.17232 |
| | viral oncogene homolog 4 | | |
| | (avian) | | |
| CLDN16 | claudin 16 | 0.008608 | −1.17225 |
| CIB2 | calcium and integrin binding | 0.006423 | −1.17213 |
| | family member 2 | | |
| GALR3 | galanin receptor 3 | 0.001999 | −1.1721 |
| MSMB | microseminoprotein, beta- | 0.000282 | −1.17208 |
| FABP7 | fatty acid binding protein 7, | 0.009982 | −1.17199 |
| | brain | | |
| ATXN3 | ataxin 3 | 0.009922 | −1.17197 |
| KCNJ5 | potassium inwardly-rectifying | 0.00027 | −1.17188 |
| | channel, subfamily J, member 5 | | |
| TRDN | triadin | 0.005982 | −1.1718 |
| CYP3A43 | cytochrome P450, family 3, | 0.000729 | −1.17176 |
| | subfamily A, polypeptide 43 | | |
| BAZ2A | bromodomain adjacent to zinc | 0.000788 | −1.17174 |
| | finger domain, 2A | | |
| ACCN4 | amiloride-sensitive cation | 0.006157 | −1.17166 |
| | channel 4, pituitary | | |
| SILV | silver homolog (mouse) | 0.001891 | −1.17163 |
| DGCR14 | DiGeorge syndrome critical | 0.008083 | −1.17146 |
| | region gene 14 | | |
| SEMA6C | sema domain, transmembrane | 0.003714 | −1.17139 |
| | domain (TM), and cytoplasmic | | |
| | domain, (semaphorin) 6C | | |
| DIO2 | deiodinase, iodothyronine, | 0.001589 | −1.17126 |
| | type II | | |
| PTHLH | parathyroid hormone-like | 0.000476 | −1.17108 |
| | hormone | | |
| CSF3 | colony stimulating factor 3 | 0.003909 | −1.17105 |
| | (granulocyte) | | |
| — | — | 0.002628 | −1.17103 |
| LEP | leptin | 0.006607 | −1.17102 |
| PDZRN3 | PDZ domain containing ring | 0.006658 | −1.171 |
| | finger 3 | | |
| RGSL1 | regulator of G-protein signaling | 0.000118 | −1.17097 |
| | like 1 | | |
| GJA4 | gap junction protein, alpha 4, | 0.002623 | −1.17081 |
| | 37 kDa | | |
| F2 | coagulation factor II (thrombin) | 0.00539 | −1.17065 |
| SLC22A6 | solute carrier family 22 (organic | 0.002803 | −1.17063 |
| | anion transporter), member 6 | | |
| RASGRF1 | Ras protein-specific guanine | 0.000634 | −1.17056 |
| | nucleotide-releasing factor 1 | | |
| MAPRE2 | microtubule-associated protein, | 0.000948 | −1.17055 |
| | RP/EB family, member 2 | | |
| PVRL1 | poliovirus receptor-related 1 | 0.008624 | −1.17042 |
| | (herpesvirus entry mediator C) | | |
| AKAP1 | A kinase (PRKA) anchor | 0.002224 | −1.17036 |
| | protein 1 | | |
| — | — | 0.001632 | −1.17035 |
| POMP | proteasome maturation protein | 0.00605 | −1.17031 |
| SOX21 | SRY (sex determining region | 0.003094 | −1.17029 |
| | Y)-box 21 | | |
| DNAH9 | dynein, axonemal, heavy chain 9 | 0.001951 | −1.1701 |
| HOXC5 | homeobox C5 | 0.005033 | −1.17002 |
| SERHL2 | serine hydrolase-like 2 | 0.007046 | −1.17001 |
| KIAA0485 | hypothetical LOC57235 | 0.005249 | −1.16992 |
| ITSN1 | intersectin 1 (SH3 domain | 0.004533 | −1.16989 |
| | protein) | | |
| B4GALT1 | UDP-Gal: betaGlcNAc beta 1,4- | 0.008844 | −1.16988 |
| | galactosyltransferase, | | |
| | polypeptide 1 | | |
| NEK2 | NIMA (never in mitosis gene | 0.002232 | −1.16958 |
| | a)-related kinase 2 | | |
| NUPR1 | nuclear protein, transcriptional | 0.007281 | −1.16954 |
| | regulator, 1 | | |
| CCDC93 | coiled-coil domain containing 93 | 0.009039 | −1.16948 |
| EPO | erythropoietin | 0.00697 | −1.16943 |
| CRABP2 | cellular retinoic acid binding | 0.008297 | −1.16942 |
| | protein 2 | | |
| TYRO3 | TYRO3 protein tyrosine kinase | 0.002608 | −1.16924 |
| GOLGA2 | golgi autoantigen, golgin | 0.00754 | −1.16892 |
| | subfamily a, 2 | | |
| SEMA3F | sema domain, immunoglobulin | 0.008138 | −1.1688 |
| | domain (Ig), short basic | | |
| | domain, secreted, (semaphorin) 3F | | |
| BFSP2 | beaded filament structural | 0.009323 | −1.16867 |
| | protein 2, phakinin | | |
| NCAM1 | neural cell adhesion molecule 1 | 0.001786 | −1.16866 |
| FOLH1 | folate hydrolase (prostate- | 0.002392 | −1.16854 |
| | specific membrane antigen) 1 | | |
| SSX2 | synovial sarcoma, X breakpoint 2 | 0.001495 | −1.16849 |
| TMPRSS4 | transmembrane protease, serine 4 | 0.002865 | −1.16833 |
| DCN | decorin | 0.007122 | −1.16824 |
| LPHN3 | latrophilin 3 | 0.000384 | −1.16821 |
| POU4F3 | POU class 4 homeobox 3 | 0.008224 | −1.1682 |
| CEACAM5 | carcinoembryonic antigen- | 0.007102 | −1.16817 |
| | related cell adhesion molecule 5 | | |
| BCL3 | B-cell CLL/lymphoma 3 | 0.006056 | −1.16816 |
| — | — | 0.001654 | −1.16813 |
| EXTL3 | exostoses (multiple)-like 3 | 0.007597 | −1.16811 |
| CCNA1 | cyclin A1 | 0.00771 | −1.16794 |
| DDR2 | discoidin domain receptor | 0.002146 | −1.16784 |
| | tyrosine kinase 2 | | |
| PAX8 | paired box 8 | 0.001053 | −1.16778 |
| SOX5 | SRY (sex determining region | 0.003283 | −1.16769 |
| | Y)-box 5 | | |
| POU3F1 | POU class 3 homeobox 1 | 0.002775 | −1.16762 |
| PEX16 | peroxisomal biogenesis factor 16 | 0.002334 | −1.16754 |
| IL4I1 /// | interleukin 4 induced 1 /// | 0.005035 | −1.16752 |
| NUP62 /// | nucleoporin 62 kDa /// sialic | | |
| SIGLEC11 | acid binding Ig-like lectin 11 | | |
| ALDOB | aldolase B, fructose- | 0.000319 | −1.16747 |
| | bisphosphate | | |
| GPC3 | glypican 3 | 0.001612 | −1.1674 |
| IGFALS | insulin-like growth factor | 0.000261 | −1.16732 |
| | binding protein, acid labile | | |
| | subunit | | |
| WDR25 | WD repeat domain 25 | 0.004535 | −1.16731 |
| FGF1 | fibroblast growth factor 1 | 0.003604 | −1.1673 |
| | (acidic) | | |
| OSR2 | odd-skipped related 2 | 0.005103 | −1.1673 |
| | (Drosophila) | | |
| ARID1A | AT rich interactive domain 1A | 0.007435 | −1.16727 |
| | (SWI-like) | | |
| GYPA | glycophorin A (MNS blood | 0.009414 | −1.16715 |
| | group) | | |
| KLK13 | kallikrein-related peptidase 13 | 0.008814 | −1.16712 |
| PARVB | parvin, beta | 0.000462 | −1.16709 |
| LILRB5 | leukocyte immunoglobulin-like | 0.006486 | −1.16709 |
| | receptor, subfamily B (with TM | | |
| | and ITIM domains), member | | |
| RIMS2 | regulating synaptic membrane | 0.003506 | −1.16705 |
| | exocytosis 2 | | |
| C19orf21 | chromosome 19 open reading | 0.003213 | −1.16704 |
| | frame 21 | | |
| HOXD1 | homeobox D1 | 0.00567 | −1.16704 |
| PRSS3 | protease, serine, 3 | 0.007816 | −1.167 |
| FLT1 | fms-related tyrosine kinase 1 | 0.002491 | −1.16699 |
| | (vascular endothelial growth | | |
| | factor/vascular permeability | | |
| ATP6V1C1 | ATPase, H+ transporting, | 0.00431 | −1.16699 |
| | lysosomal 42 kDa, VI subunit C1 | | |
| LOX | lysyl oxidase | 0.000711 | −1.16681 |
| CRYBB3 | crystallin, beta B3 | 0.001902 | −1.16676 |
| CA12 | carbonic anhydrase XII | 0.006921 | −1.16662 |
| PRKG2 | protein kinase, cGMP- | 0.006891 | −1.16659 |
| | dependent, type II | | |
| MASP1 | mannan-binding lectin serine | 0.003795 | −1.16655 |
| | peptidase 1 (C4/C2 activating | | |
| | component of Ra-reactive fac | | |
| LOC728395 /// | testis specific protein, Y-linked | 9.49E−05 | −1.16641 |
| LOC728403 /// | 1-like /// similar to Testis- | | |
| TSPY1 | specific Y-encoded prote | | |
| PDCD1 | programmed cell death 1 | 0.004701 | −1.16634 |
| GGTLC1 | gamma-glutamyltransferase | 0.004441 | −1.16622 |
| | light chain 1 | | |
| AQP8 | aquaporin 8 | 0.004705 | −1.16618 |
| IL1F9 | interleukin 1 family, member 9 | 0.00516 | −1.16614 |
| KRT16 | keratin 16 | 0.0054 | −1.16604 |
| AICDA | activation-induced cytidine | 0.002152 | −1.16602 |
| | deaminase | | |
| BRD8 | bromodomain containing 8 | 0.005311 | −1.16593 |
| C1orf95 | Chromosome 1 open reading | 0.003655 | −1.16587 |
| | frame 95 | | |
| OR3A2 | olfactory receptor, family 3, | 0.006942 | −1.16583 |
| | subfamily A, member 2 | | |
| — | — | 0.002314 | −1.1656 |
| PFKFB2 | 6-phosphofructo-2- | 0.001095 | −1.16553 |
| | kinase/fructose-2,6- | | |
| | biphosphatase 2 | | |
| — | — | 0.007371 | −1.16546 |
| FRZB | frizzled-related protein | 0.004073 | −1.16541 |
| PAK3 | p21 protein (Cdc42/Rac)- | 0.001322 | −1.16538 |
| | activated kinase 3 | | |
| MEIS2 | Meis homeobox 2 | 0.005478 | −1.16537 |
| ZSCAN2 | zinc finger and SCAN domain | 0.007216 | −1.16537 |
| | containing 2 | | |
| MYH7 | myosin, heavy chain 7, cardiac | 0.00763 | −1.16506 |
| | muscle, beta | | |
| VWA1 | von Willebrand factor A | 0.005843 | −1.165 |
| | domain containing 1 | | |
| LSAMP | limbic system-associated | 0.00683 | −1.16484 |
| | membrane protein | | |
| SRC | v-src sarcoma (Schmidt-Ruppin | 0.000259 | −1.16471 |
| | A-2) viral oncogene homolog | | |
| | (avian) | | |
| UGT1A1 /// | UDP glucuronosyltransferase 1 | 0.002476 | −1.16454 |
| UGT1A10 /// | family, polypeptide A1 /// UDP | | |
| UGT1A3 /// | glucuronosyltransferase 1 | | |
| UGT1A4 /// | | | |
| UGT1A5 /// | | | |
| UGT1A6 /// | | | |
| UGT1A7 /// | | | |
| UGT1A8 /// | | | |
| UGT1A9 | | | |
| DIO1 | deiodinase, iodothyronine, type I | 0.002692 | −1.16452 |
| — | — | 0.009224 | −1.16449 |
| TADA3L | transcriptional adaptor 3 | 0.00694 | −1.16443 |
| | (NGG1 homolog, yeast)-like | | |
| F10 | coagulation factor X | 0.004459 | −1.16432 |
| NFASC | neurofascin homolog (chicken) | 0.001454 | −1.16431 |
| CALCRL | calcitonin receptor-like | 0.001263 | −1.16423 |
| NBLA00301 | Nbla00301 | 0.008572 | −1.16423 |
| MAB21L1 | mab-21-like 1 (C. elegans) | 0.006335 | −1.16412 |
| FBXO42 | F-box protein 42 | 0.002917 | −1.16408 |
| COL10A1 | collagen, type X, alpha 1 | 0.003871 | −1.16407 |
| CFB | complement factor B | 0.003696 | −1.16404 |
| SNX7 | sorting nexin 7 | 0.007996 | −1.16401 |
| FOXN1 | forkhead box N1 | 0.007972 | −1.16365 |
| SRY | sex determining region Y | 0.007481 | −1.16363 |
| HLF | hepatic leukemia factor | 0.009185 | −1.16361 |
| CLCA3P | chloride channel accessory 3 | 0.00306 | −1.16343 |
| | (pseudogene) | | |
| DAZ1 /// | deleted in azoospermia 1 /// | 0.009345 | −1.16343 |
| DAZ2 /// | deleted in azoospermia 2 /// | | |
| DAZ3 /// | deleted in azoospermia 3 /// | | |
| DAZ4 | | | |
| GPR3 | G protein-coupled receptor 3 | 0.001459 | −1.16341 |
| TMPRSS11E | transmembrane protease, serine 11E | 0.000617 | −1.1634 |
| EMID1 | EMI domain containing 1 | 0.009937 | −1.1634 |
| KCNMB2 | potassium large conductance | 0.003215 | −1.16335 |
| | calcium-activated channel, | | |
| | subfamily M, beta member 2 | | |
| MUC5AC | mucin 5AC, oligomeric | 0.003908 | −1.16324 |
| | mucus/gel-forming | | |
| SORT1 | sortilin 1 | 0.004071 | −1.16318 |
| HIF3A | hypoxia inducible factor 3, | 0.001905 | −1.16316 |
| | alpha subunit | | |
| — | — | 0.005107 | −1.16312 |
| MAPK4 | mitogen-activated protein | 0.005343 | −1.16312 |
| | kinase 4 | | |
| TCP11L1 | t-complex 11 (mouse)-like 1 | 0.006965 | −1.16308 |
| ZZEF1 | zinc finger, ZZ-type with EF- | 0.009197 | −1.16307 |
| | hand domain 1 | | |
| DCAF7 | DDB1 and CUL4 associated | 0.00843 | −1.16305 |
| | factor 7 | | |
| DMWD | dystrophia myotonica, WD | 0.005744 | −1.16304 |
| | repeat containing | | |
| CLCA2 | chloride channel accessory 2 | 0.000363 | −1.16297 |
| VAC14 | Vac14 homolog (S. cerevisiae) | 0.004469 | −1.16297 |
| CSPG5 | chondroitin sulfate | 8.48E−05 | −1.16282 |
| | proteoglycan 5 (neuroglycan C) | | |
| — | — | 0.005222 | −1.16268 |
| STMN2 | stathmin-like 2 | 0.006817 | −1.16268 |
| MLLT4 | myeloid/lymphoid or mixed- | 0.003474 | −1.16262 |
| | lineage leukemia (trithorax | | |
| | homolog, Drosophila); | | |
| | translocate | | |
| GALNT14 | UDP-N-acetyl-alpha-D- | 0.008433 | −1.16262 |
| | galactosamine: polypeptide N- | | |
| | acetylgalactosaminyltransferase | | |
| | 14 (Ga | | |
| FGF12 | fibroblast growth factor 12 | 0.000459 | −1.16254 |
| MFAP5 | microfibrillar associated | 0.008062 | −1.16239 |
| | protein 5 | | |
| SUMO3 | SMT3 suppressor of mif two 3 | 0.006747 | −1.16225 |
| | homolog 3 (S. cerevisiae) | | |
| HTR3A | 5-hydroxytryptamine | 0.002037 | −1.16224 |
| | (serotonin) receptor 3A | | |
| GDF5 | growth differentiation factor 5 | 0.006583 | −1.16222 |
| — | — | 0.008115 | −1.16217 |
| MMP24 | matrix metallopeptidase 24 | 0.0086 | −1.16211 |
| | (membrane-inserted) | | |
| TSSK1B | testis-specific serine kinase 1B | 0.001104 | −1.16208 |
| CYP2A7P1 | cytochrome P450, family 2, | 0.002137 | −1.16208 |
| | subfamily A, polypeptide 7 | | |
| | pseudogene 1 | | |
| MARK1 | MAP/microtubule affinity- | 0.00508 | −1.16207 |
| | regulating kinase 1 | | |
| ATP1B2 | ATPase, Na+/K+ transporting, | 0.001769 | −1.16204 |
| | beta 2 polypeptide | | |
| TBX6 | T-box 6 | 0.005223 | −1.162 |
| PAX8 | paired box 8 | 0.001211 | −1.16199 |
| IL1R1 | interleukin 1 receptor, type I | 0.002307 | −1.16176 |
| RALYL | RALY RNA binding protein- | 0.004265 | −1.16172 |
| | like | | |
| OR2B2 | olfactory receptor, family 2, | 0.00135 | −1.16157 |
| | subfamily B, member 2 | | |
| TAAR3 | trace amine associated receptor | 0.00469 | −1.16152 |
| | 3 (gene/pseudogene) | | |
| C12orf32 /// | Chromosome 12 open reading | 0.006624 | −1.1615 |
| IGHG1 /// | frame 32 /// Immunoglobulin | | |
| LOC642131 | heavy constant gamma 1 (G1m | | |
| | mark | | |
| DICER1 | dicer 1, ribonuclease type III | 0.003322 | −1.16138 |
| MMP28 | matrix metallopeptidase 28 | 0.00533 | −1.16138 |
| GLRA3 | glycine receptor, alpha 3 | 0.00029 | −1.16137 |
| PPARD | peroxisome proliferator- | 0.007231 | −1.1612 |
| | activated receptor delta | | |
| HSPA4L | heat shock 70 kDa protein 4-like | 0.001573 | −1.16116 |
| WNT2 | wingless-type MMTV | 0.009189 | −1.16115 |
| | integration site family member 2 | | |
| VIPR2 | vasoactive intestinal peptide | 0.005059 | −1.16112 |
| | receptor 2 | | |
| CYP2C9 | Cytochrome P450, family 2, | 0.001861 | −1.16111 |
| | subfamily C, polypeptide 9 | | |
| SRPX2 | sushi-repeat-containing protein, | 0.000138 | −1.16109 |
| | X-linked 2 | | |
| IGSF1 | immunoglobulin superfamily, | 0.001192 | −1.16104 |
| | member 1 | | |
| ALPK3 | alpha-kinase 3 | 0.00608 | −1.16101 |
| TFPI | tissue factor pathway inhibitor | 0.006389 | −1.16092 |
| | (lipoprotein-associated | | |
| | coagulation inhibitor) | | |
| KCNS3 | potassium voltage-gated | 0.003038 | −1.16085 |
| | channel, delayed-rectifier, | | |
| | subfamily S, member 3 | | |
| MARCH8 | membrane-associated ring | 0.006576 | −1.16083 |
| | finger (C3HC4) 8 | | |
| FRMD4B | FERM domain containing 4B | 0.000444 | −1.1607 |
| TACR3 | tachykinin receptor 3 | 0.00896 | −1.16066 |
| FIGF | c-fos induced growth factor | 0.005183 | −1.16058 |
| | (vascular endothelial growth | | |
| | factor D) | | |
| PDCD6 | Programmed cell death 6 | 0.006092 | −1.16044 |
| TNN | tenascin N | 0.006607 | −1.16044 |
| SPANXB1 /// | SPANX family, member B1 /// | 0.001174 | −1.16041 |
| SPANXB2 /// | SPANX family, member B2 /// | | |
| SPANXF1 | SPANX family, member F1 | | |
| RHBDD3 | rhomboid domain containing 3 | 0.000508 | −1.16033 |
| SPP2 | secreted phosphoprotein 2, | 0.005711 | −1.16031 |
| | 24 kDa | | |
| PDE10A | phosphodiesterase 10A | 0.005387 | −1.16026 |
| ZNF224 | zinc finger protein 224 | 0.009425 | −1.16025 |
| FGL1 | fibrinogen-like 1 | 0.004849 | −1.1602 |
| PGAM2 | phosphoglycerate mutase 2 | 0.003588 | −1.16019 |
| | (muscle) | | |
| CADM4 | cell adhesion molecule 4 | 0.004403 | −1.1601 |
| APOBEC2 | apolipoprotein B mRNA editing | 0.007177 | −1.15988 |
| | enzyme, catalytic polypeptide- | | |
| | like 2 | | |
| SLC9A5 | solute carrier family 9 | 0.003795 | −1.15983 |
| | (sodium/hydrogen exchanger), | | |
| | member 5 | | |
| SERPINA3 | serpin peptidase inhibitor, clade | 0.001263 | −1.15982 |
| | A (alpha-1 antiproteinase, | | |
| | antitrypsin), member 3 | | |
| GNAT1 | guanine nucleotide binding | 1.43E−05 | −1.15969 |
| | protein (G protein), alpha | | |
| | transducing activity polypeptide | | |
| — | — | 0.003568 | −1.15968 |
| ARHGEF16 | Rho guanine exchange factor | 0.004721 | −1.15963 |
| | (GEF) 16 | | |
| SMARCA2 | SWI/SNF related, matrix | 0.00734 | −1.15962 |
| | associated, actin dependent | | |
| | regulator of chromatin, | | |
| | subfamily a | | |
| DNAH9 | dynein, axonemal, heavy chain 9 | 0.009008 | −1.15957 |
| RBM26 | RNA binding motif protein 26 | 0.00116 | −1.15955 |
| WNT2B | wingless-type MMTV | 0.004131 | −1.1595 |
| | integration site family, | | |
| | member 2B | | |
| KCNK2 | potassium channel, subfamily | 0.00217 | −1.15945 |
| | K, member 2 | | |
| NPBWR2 | neuropeptides B/W receptor 2 | 0.007611 | −1.15945 |
| SP2 | Sp2 transcription factor | 0.002898 | −1.15944 |
| — | — | 0.001126 | −1.1594 |
| TMPRSS11D | transmembrane protease, | 0.00681 | −1.15937 |
| | serine 11D | | |
| DENND2A | DENN/MADD domain | 0.003959 | −1.15926 |
| | containing 2A | | |
| TNIP3 | TNFAIP3 interacting protein 3 | 0.006903 | −1.15918 |
| — | — | 0.009743 | −1.15905 |
| STC1 | stanniocalcin 1 | 0.009242 | −1.15902 |
| DOCK6 | dedicator of cytokinesis 6 | 0.007919 | −1.15896 |
| ADAM5P | ADAM metallopeptidase | 0.004414 | −1.1589 |
| | domain 5 pseudogene | | |
| SYDE1 | synapse defective 1, Rho | 0.002852 | −1.15886 |
| | GTPase, homolog 1 | | |
| | (C. elegans) | | |
| TNPO2 | transportin 2 | 0.002767 | −1.15883 |
| LRTM1 | leucine-rich repeats and | 0.002691 | −1.15877 |
| | transmembrane domains 1 | | |
| USH1C | Usher syndrome 1C (autosomal | 0.006427 | −1.15873 |
| | recessive, severe) | | |
| PDE12 | Phosphodiesterase 12 | 0.007267 | −1.15873 |
| SRCAP | Snf2-related CREBBP activator | 0.004676 | −1.15866 |
| | protein | | |
| OR10J1 | olfactory receptor, family 10, | 0.005572 | −1.15866 |
| | subfamily J, member 1 | | |
| OR2H2 | olfactory receptor, family 2, | 0.000927 | −1.15859 |
| | subfamily H, member 2 | | |
| KCNJ8 | potassium inwardly-rectifying | 0.008335 | −1.15855 |
| | channel, subfamily J, member 8 | | |
| — | — | 0.005451 | −1.15854 |
| RP11-257K9.7 | hypothetical protein | 0.002187 | −1.15851 |
| | LOC100129128 | | |
| DOCK5 | dedicator of cytokinesis 5 | 0.000433 | −1.1585 |
| TPD52L1 | tumor protein D52-like 1 | 0.005443 | −1.15839 |
| PAEP | progestagen-associated | 0.005702 | −1.15836 |
| | endometrial protein | | |
| — | — | 0.008347 | −1.15836 |
| GGA2 | golgi associated, gamma | 0.005179 | −1.15822 |
| | adaptin ear containing, ARF | | |
| | binding protein 2 | | |
| PHLDA3 | pleckstrin homology-like | 0.008165 | −1.15821 |
| | domain, family A, member 3 | | |
| HES2 | hairy and enhancer of split 2 | 0.00726 | −1.15816 |
| | (Drosophila) | | |
| MLL | myeloid/lymphoid or mixed- | 0.00632 | −1.15814 |
| | lineage leukemia (trithorax | | |
| | homolog, Drosophila) | | |
| PTN | pleiotrophin | 0.001451 | −1.15812 |
| ITGB6 | integrin, beta 6 | 0.000984 | −1.1581 |
| CHRNA6 | cholinergic receptor, nicotinic, | 0.008293 | −1.15806 |
| | alpha 6 | | |
| CIB2 | calcium and integrin binding | 0.004346 | −1.15803 |
| | family member 2 | | |
| — | — | 0.008761 | −1.15788 |
| PTPRF | protein tyrosine phosphatase, | 0.009748 | −1.15777 |
| | receptor type, F | | |
| TM7SF4 | transmembrane 7 superfamily | 0.007817 | −1.15761 |
| | member 4 | | |
| DAZ1 /// | deleted in azoospermia 1 /// | 0.001893 | −1.15743 |
| DAZ2 /// | deleted in azoospermia 2 /// | | |
| DAZ3 /// | deleted in azoospermia 3 /// | | |
| DAZ4 | | | |
| ALX1 | ALX homeobox 1 | 0.000867 | −1.15731 |
| OR2F1 /// | olfactory receptor, family 2, | 0.009833 | −1.15725 |
| OR2F2 | subfamily F, member 1 /// | | |
| | olfactory receptor, family 2, s | | |
| — | — | 0.003275 | −1.15723 |
| PLAT | plasminogen activator, tissue | 0.005329 | −1.15717 |
| — | — | 0.00262 | −1.15716 |
| HGC6.3 | similar to HGC6.3 | 0.00088 | −1.15709 |
| — | — | 0.002596 | −1.15707 |
| WNT11 | wingless-type MMTV | 0.003994 | −1.15705 |
| | integration site family, | | |
| | member 11 | | |
| PGK2 | phosphoglycerate kinase 2 | 0.001775 | −1.15699 |
| SNAI2 | snail homolog 2 (Drosophila) | 0.001463 | −1.15694 |
| — | — | 0.001954 | −1.15682 |
| IL11 | interleukin 11 | 0.005023 | −1.15682 |
| COL4A6 | collagen, type IV, alpha 6 | 0.00986 | −1.15677 |
| PRUNE2 | prune homolog 2 (Drosophila) | 0.003014 | −1.15675 |
| — | — | 0.004631 | −1.15658 |
| ANKS1B | ankyrin repeat and sterile alpha | 0.000784 | −1.15638 |
| | motif domain containing 1B | | |
| — | — | 0.008146 | −1.1563 |
| LOC81691 | exonuclease NEF-sp | 0.008969 | −1.15628 |
| FERMT2 | fermitin family homolog 2 | 0.006573 | −1.15622 |
| | (Drosophila) | | |
| TIMP3 | TIMP metallopeptidase | 0.005686 | −1.15618 |
| | inhibitor 3 | | |
| CST8 | cystatin 8 (cystatin-related | 0.006664 | −1.15617 |
| | epididymal specific) | | |
| CAPN6 | calpain 6 | 0.006576 | −1.15614 |
| IDUA | iduronidase, alpha-L- | 0.002113 | −1.15597 |
| GPR32 | G protein-coupled receptor 32 | 0.000773 | −1.15585 |
| AKR1B10 | aldo-keto reductase family 1, | 0.007442 | −1.15571 |
| | member B10 (aldose reductase) | | |
| GRHL2 | grainyhead-like 2 (Drosophila) | 5.24E−07 | −1.15561 |
| FBXO24 | F-box protein 24 | 0.003095 | −1.1555 |
| HSF4 | heat shock transcription factor 4 | 0.007043 | −1.15548 |
| IGHG1 | Immunoglobulin heavy constant | 0.00779 | −1.15512 |
| | gamma 1 (G1m marker) | | |
| HCN2 | hyperpolarization activated | 0.006206 | −1.15509 |
| | cyclic nucleotide-gated | | |
| | potassium channel 2 | | |
| LRP12 | low density lipoprotein-related | 0.007931 | −1.15508 |
| | protein 12 | | |
| ADAM 18 | ADAM metallopeptidase | 0.006422 | −1.15501 |
| | domain 18 | | |
| ITGA2 | integrin, alpha 2 (CD49B, alpha | 0.005786 | −1.15485 |
| | 2 subunit of VLA-2 receptor) | | |
| ARHGEF15 | Rho guanine nucleotide | 0.003722 | −1.15475 |
| | exchange factor (GEF) 15 | | |
| UGT1A1 /// | UDP glucuronosyltransferase 1 | 0.004274 | −1.1547 |
| UGT1A10 /// | family, polypeptide A1 /// UDP | | |
| UGT1A7 /// | glucuronosyltransferase 1 | | |
| UGT1A8 | | | |
| GUCA2A | guanylate cyclase activator 2A | 0.003837 | −1.15459 |
| | (guanylin) | | |
| MDK | midkine (neurite growth- | 0.003419 | −1.15451 |
| | promoting factor 2) | | |
| ITIH1 | inter-alpha (globulin) | 0.003175 | −1.15449 |
| | inhibitor H1 | | |
| EGFR | epidermal growth factor | 0.004643 | −1.15435 |
| | receptor (erythroblastic | | |
| | leukemia viral (v-erb-b) | | |
| | oncogene homo | | |
| UGT1A1 /// | UDP glucuronosyltransferase 1 | 0.008973 | −1.15435 |
| UGT1A10 /// | family, polypeptide A1 /// UDP | | |
| UGT1A3 /// | glucuronosyltransferase 1 | | |
| UGT1A4 /// | | | |
| UGT1A5 /// | | | |
| UGT1A6 /// | | | |
| UGT1A7 /// | | | |
| UGT1A8 /// | | | |
| UGT1A9 | | | |
| MYOG | myogenin (myogenic factor 4) | 0.001644 | −1.15431 |
| TMSB15A | thymosin beta 15a | 0.001935 | −1.15428 |
| TLX1 | T-cell leukemia homeobox 1 | 0.005999 | −1.15425 |
| EDNRA | endothelin receptor type A | 0.002498 | −1.15397 |
| LOC100289791 | hypothetical protein | 0.00917 | −1.1539 |
| | LOC100289791 | | |
| MDFI | MyoD family inhibitor | 0.008115 | −1.15389 |
| ZER1 | zer-1 homolog (C. elegans) | 0.003119 | −1.15387 |
| MYH15 | myosin, heavy chain 15 | 0.00261 | −1.15381 |
| CDH20 | cadherin 20, type 2 | 0.004804 | −1.15374 |
| GPR63 | G protein-coupled receptor 63 | 0.003041 | −1.15367 |
| — | — | 0.009921 | −1.15354 |
| LOC440345 /// | hypothetical protein | 0.002618 | −1.15347 |
| LOC440354 /// | LOC440345 /// PI-3-kinase- | | |
| LOC595101 /// | related kinase SMG-1 | | |
| LOC641298 /// | pseudogene /// PI-3 | | |
| SMG1 | | | |
| HOXC10 | homeobox C10 | 0.000495 | −1.15345 |
| KRTAP1-1 | keratin associated protein 1-1 | 8.96E−06 | −1.15325 |
| ARSD | arylsulfatase D | 0.004513 | −1.15315 |
| CPLX3 /// | complexin 3 /// lectin, mannose- | 0.004705 | −1.15295 |
| LMAN1L | binding, 1 like | | |
| IFNA4 | interferon, alpha 4 | 0.004607 | −1.15289 |
| ABCC1 | ATP-binding cassette, sub- | 0.006325 | −1.15283 |
| | family C (CFTR/MRP), | | |
| | member 1 | | |
| SEMA3E | sema domain, immunoglobulin | 0.004174 | −1.15277 |
| | domain (Ig), short basic | | |
| | domain, secreted, (semaphorin) 3E | | |
| MRE11A | MRE11 meiotic recombination | 0.003835 | −1.15276 |
| | 11 homolog A (S. cerevisiae) | | |
| C1QL1 | Complement component 1, q | 0.004524 | −1.15267 |
| | subcomponent-like 1 | | |
| LIPF | lipase, gastric | 0.00095 | −1.15265 |
| TRIM9 | tripartite motif-containing 9 | 0.008678 | −1.15258 |
| BBOX1 | butyrobetaine (gamma), 2- | 0.001994 | −1.15252 |
| | oxoglutarate dioxygenase | | |
| | (gamma-butyrobetaine | | |
| | hydroxylase) 1 | | |
| LRRC17 | leucine rich repeat containing 17 | 0.005074 | −1.15235 |
| WNT2B | wingless-type MMTV | 0.006181 | −1.15231 |
| | integration site family, member 2B | | |
| CYP3A4 | cytochrome P450, family 3, | 0.007633 | −1.15227 |
| | subfamily A, polypeptide 4 | | |
| SI | sucrase-isomaltase (alpha- | 0.001001 | −1.1522 |
| | glucosidase) | | |
| ANO3 | anoctamin 3 | 0.00341 | −1.15219 |
| OBSL1 | obscurin-like 1 | 0.003099 | −1.15215 |
| — | — | 0.007073 | −1.152 |
| CHRD | chordin | 0.004608 | −1.15192 |
| MSX2 | msh homeobox 2 | 0.009125 | −1.15179 |
| PSG1 | pregnancy specific beta-1- | 0.00029 | −1.15178 |
| | glycoprotein 1 | | |
| FAM107A | family with sequence similarity | 0.001347 | −1.15167 |
| | 107, member A | | |
| LRRC37B2 | leucine rich repeat containing | 0.001053 | −1.15156 |
| | 37, member B2 | | |
| ANKLE2 | Ankyrin repeat and LEM | 0.004674 | −1.15155 |
| | domain containing 2 | | |
| PAX2 | paired box 2 | 0.004533 | −1.15149 |
| UNC5B | Unc-5 homolog B (C. elegans) | 0.001994 | −1.15124 |
| ADCYAP1R1 | adenylate cyclase activating | 0.001818 | −1.15119 |
| | polypeptide 1 (pituitary) | | |
| | receptor type I | | |
| HFE | hemochromatosis | 0.0019 | −1.15119 |
| — | — | 0.006488 | −1.15106 |
| SYT1 | synaptotagmin 1 | 0.002216 | −1.15088 |
| GJC2 | gap junction protein, gamma 2, | 0.006151 | −1.15082 |
| | 47 kDa | | |
| LOC100293871 | similar to PRO2325 | 0.005525 | −1.15064 |
| FGF8 | fibroblast growth factor 8 | 0.008777 | −1.15061 |
| | (androgen-induced) | | |
| ACRV1 | acrosomal vesicle protein 1 | 0.005022 | −1.15056 |
| NRXN1 | neurexin 1 | 0.004617 | −1.15047 |
| GDPD2 | glycerophosphodiester | 0.004533 | −1.15039 |
| | phosphodiesterase domain | | |
| | containing 2 | | |
| RGS4 | regulator of G-protein | 0.003963 | −1.15033 |
| | signaling 4 | | |
| CELA2A | chymotrypsin-like elastase | 7.18E−05 | −1.15022 |
| | family, member 2A | | |
| IFNW1 | interferon, omega 1 | 0.001727 | −1.15017 |
| MLNR | motilin receptor | 0.000122 | −1.15014 |
| RNF17 | ring finger protein 17 | 0.003651 | −1.15006 |
| LAD1 | ladinin 1 | 0.001937 | −1.15 |
| GLRA2 | glycine receptor, alpha 2 | 0.006763 | −1.14955 |
| RASL12 | RAS-like, family 12 | 0.000306 | −1.14945 |
| MAGOH2 | mago-nashi homolog 2, | 0.003414 | −1.14942 |
| | proliferation-associated | | |
| | (Drosophila) | | |
| C6orf54 | chromosome 6 open reading | 0.007125 | −1.14931 |
| | frame 54 | | |
| — | — | 0.001197 | −1.14922 |
| ZNF214 | zinc finger protein 214 | 0.000481 | −1.1492 |
| IKBKG | inhibitor of kappa light | 0.005732 | −1.14913 |
| | polypeptide gene enhancer in | | |
| | B-cells, kinase gamma | | |
| AP4E1 | adaptor-related protein complex | 0.004158 | −1.14904 |
| | 4, epsilon 1 subunit | | |
| ZNRF4 | zinc and ring finger 4 | 0.000445 | −1.14875 |
| OSBPL10 | oxysterol binding protein-like 10 | 0.008717 | −1.14862 |
| C1orf175 /// | chromosome 1 open reading | 0.002146 | −1.14841 |
| TTC4 | frame 175 /// tetratricopeptide | | |
| | repeat domain 4 | | |
| PCDHB3 | protocadherin beta 3 | 0.006249 | −1.14837 |
| ADRBK1 | adrenergic, beta, receptor | 0.009822 | −1.14828 |
| | kinase 1 | | |
| ITSN1 | intersectin 1 (SH3 domain | 0.002166 | −1.14826 |
| | protein) | | |
| XAGE1A /// | X antigen family, member 1A /// | 0.00656 | −1.1482 |
| XAGE1B /// | X antigen family, member | | |
| XAGE1C /// | 1B /// X antigen family, membe | | |
| XAGE1D /// | | | |
| XAGE1E | | | |
| CDH22 | cadherin-like 22 | 0.008849 | −1.14819 |
| FARP2 | FERM, RhoGEF and pleckstrin | 0.002273 | −1.14813 |
| | domain protein 2 | | |
| MYT1 | myelin transcription factor 1 | 0.003875 | −1.14809 |
| TNC | Tenascin C | 0.004194 | −1.14799 |
| MUC5AC | mucin 5AC, oligomeric | 0.009053 | −1.14791 |
| | mucus/gel-forming | | |
| SLC6A15 | solute carrier family 6 (neutral | 0.008601 | −1.1479 |
| | amino acid transporter), | | |
| | member 15 | | |
| PP14571 | similar to hCG1777210 | 0.004733 | −1.14789 |
| SMR3A /// | submaxillary gland androgen | 0.002495 | −1.14788 |
| SMR3B | regulated protein 3A /// | | |
| | submaxillary gland androgen | | |
| | regula | | |
| RXRG | retinoid X receptor, gamma | 0.006609 | −1.14772 |
| SNX1 | sorting nexin 1 | 0.004678 | −1.14771 |
| GLP1R | glucagon-like peptide 1 receptor | 0.00094 | −1.14751 |
| C6orf155 | chromosome 6 open reading | 0.000855 | −1.14743 |
| | frame 155 | | |
| ATP1A2 | ATPase, Na+/K+ transporting, | 0.005511 | −1.14737 |
| | alpha 2 (+) polypeptide | | |
| TFAP4 | transcription factor AP-4 | 0.006392 | −1.14734 |
| | (activating enhancer binding | | |
| | protein 4) | | |
| PNPLA2 | Patatin-like phospholipase | 0.005616 | −1.14727 |
| | domain containing 2 | | |
| DIRAS3 | DIRAS family, GTP-binding | 0.008453 | −1.14717 |
| | RAS-like 3 | | |
| ANO2 | anoctamin 2 | 0.000478 | −1.14709 |
| TACSTD2 | tumor-associated calcium signal | 0.003737 | −1.14682 |
| | transducer 2 | | |
| MCM3AP | minichromosome maintenance | 0.000118 | −1.14681 |
| | complex component 3 | | |
| | associated protein | | |
| IL13RA2 | interleukin 13 receptor, alpha 2 | 0.002253 | −1.14678 |
| TRIM10 | tripartite motif-containing 10 | 0.005191 | −1.14676 |
| RTEL1 | regulator of telomere elongation | 0.008986 | −1.14672 |
| | helicase 1 | | |
| PRRX2 | paired related homeobox 2 | 0.006073 | −1.14659 |
| TSHB | thyroid stimulating hormone, | 0.009948 | −1.14656 |
| | beta | | |
| TIMELESS | timeless homolog (Drosophila) | 0.005683 | −1.14649 |
| FMO1 | flavin containing | 0.006505 | −1.14614 |
| | monooxygenase 1 | | |
| KIF18A | kinesin family member 18A | 0.009581 | −1.14614 |
| KIAA1199 | KIAA1199 | 0.002884 | −1.14612 |
| CALB2 | calbindin 2 | 0.005686 | −1.14598 |
| MFAP3L | microfibrillar-associated protein | 0.00354 | −1.14575 |
| | 3-like | | |
| PTGER3 | prostaglandin E receptor 3 | 0.006984 | −1.14569 |
| | (subtype EP3) | | |
| EPAS1 | endothelial PAS domain protein 1 | 0.005676 | −1.14564 |
| — | — | 0.000263 | −1.14559 |
| SQSTM1 | sequestosome 1 | 0.009341 | −1.14558 |
| TSPY1 | testis specific protein, Y-linked 1 | 0.002158 | −1.14553 |
| CPM | Carboxypeptidase M | 0.001695 | −1.14545 |
| DLGAP1 | discs, large (Drosophila) | 0.000345 | −1.14542 |
| | homolog-associated protein 1 | | |
| CYP4F11 | cytochrome P450, family 4, | 0.0029 | −1.14536 |
| | subfamily F, polypeptide 11 | | |
| TLX3 | T-cell leukemia homeobox 3 | 0.003278 | −1.14527 |
| PCDHA10 | protocadherin alpha 10 | 0.006222 | −1.14513 |
| TAOK2 | TAO kinase 2 | 0.001305 | −1.14511 |
| ERC1 | ELKS/RAB6-interacting/CAST | 0.002053 | −1.14511 |
| | family member 1 | | |
| TBX2 | T-box 2 | 0.005151 | −1.14502 |
| KALRN | kalirin, RhoGEF kinase | 0.008586 | −1.14478 |
| DICER1 | dicer 1, ribonuclease type III | 0.000825 | −1.14473 |
| PAPPA | pregnancy-associated plasma | 0.004805 | −1.14473 |
| | protein A, pappalysin 1 | | |
| — | — | 0.003479 | −1.14468 |
| — | — | 0.008082 | −1.14467 |
| KIF5A | kinesin family member 5A | 0.003962 | −1.14458 |
| DNAJC22 | DnaJ (Hsp40) homolog, | 0.007069 | −1.14453 |
| | subfamily C, member 22 | | |
| — | — | 0.006388 | −1.14452 |
| OTUB1 | OTU domain, ubiquitin | 0.007521 | −1.14451 |
| | aldehyde binding 1 | | |
| ITGBL1 | integrin, beta-like 1 (with EGF- | 0.005427 | −1.14445 |
| | like repeat domains) | | |
| KIAA1644 | KIAA1644 | 0.009294 | −1.14444 |
| SEZ6L2 | seizure related 6 homolog | 0.005712 | −1.14434 |
| | (mouse)-like 2 | | |
| PCNXL2 | pecanex-like 2 (Drosophila) | 0.008015 | −1.14434 |
| HMHB1 | histocompatibility (minor) HB-1 | 0.003775 | −1.14427 |
| ERG | v-ets erythroblastosis virus E26 | 0.008735 | −1.14427 |
| | oncogene homolog (avian) | | |
| SNTB2 | syntrophin, beta 2 (dystrophin- | 0.004153 | −1.14426 |
| | associated protein A1, 59 kDa, | | |
| | basic component 2) | | |
| GJA5 | gap junction protein, alpha 5, | 0.003562 | −1.14404 |
| | 40 kDa | | |
| AGTR2 | angiotensin II receptor, type 2 | 0.007867 | −1.14384 |
| GJA3 | gap junction protein, alpha 3, | 0.000595 | −1.14377 |
| | 46 kDa | | |
| GCK | glucokinase (hexokinase 4) | 0.005137 | −1.14365 |
| LRRC61 | leucine rich repeat containing 61 | 0.008969 | −1.14358 |
| CNTF /// | ciliary neurotrophic factor /// | 0.000997 | −1.14357 |
| ZFP91 /// | zinc finger protein 91 homolog | | |
| ZFP91-CNTF | (mouse) /// ZFP91-CNTF r | | |
| PDLIM4 | PDZ and LIM domain 4 | 0.008589 | −1.14351 |
| MPPED2 | metallophosphoesterase domain | 1.95E−05 | −1.1435 |
| | containing 2 | | |
| IFNA10 | interferon, alpha 10 | 0.003286 | −1.14346 |
| ACTN2 | actinin, alpha 2 | 0.005077 | −1.14343 |
| VGLL1 | vestigial like 1 (Drosophila) | 0.00531 | −1.14337 |
| GJA9 | gap junction protein, alpha 9, | 0.003777 | −1.14331 |
| | 59 kDa | | |
| LDLR | low density lipoprotein receptor | 0.003143 | −1.1433 |
| ANK2 | ankyrin 2, neuronal | 0.001761 | −1.14329 |
| COL1A1 | collagen, type I, alpha 1 | 0.004543 | −1.14329 |
| TIMP3 | TIMP metallopeptidase | 0.00282 | −1.14323 |
| | inhibitor 3 | | |
| OTOF | otoferlin | 0.006622 | −1.14322 |
| AGXT | alanine-glyoxylate | 0.005384 | −1.14318 |
| | aminotransferase | | |
| GLI2 | GLI family zinc finger 2 | 0.008292 | −1.14291 |
| TRMT61A | tRNA methyltransferase 61 | 0.00203 | −1.1428 |
| | homolog A (S. cerevisiae) | | |
| FOXD2 | forkhead box D2 | 0.003543 | −1.14275 |
| TMEM212 | transmembrane protein 212 | 0.003253 | −1.14258 |
| DENND2A | DENN/MADD domain | 0.007115 | −1.14257 |
| | containing 2A | | |
| B3GALT1 | UDP-Gal: betaGlcNAc beta 1,3- | 0.003568 | −1.14256 |
| | galactosyltransferase, | | |
| | polypeptide 1 | | |
| SPAG11A | sperm associated antigen 11A | 0.006235 | −1.14249 |
| MMP16 | matrix metallopeptidase 16 | 0.008228 | −1.1424 |
| | (membrane-inserted) | | |
| PRDM4 | PR domain containing 4 | 0.0049 | −1.14239 |
| TF | transferrin | 0.004293 | −1.14233 |
| ELF5 | E74-like factor 5 (ets domain | 0.004394 | −1.14201 |
| | transcription factor) | | |
| GSC2 | goosecoid homeobox 2 | 0.000273 | −1.14181 |
| EPB41L4B | erythrocyte membrane protein | 0.003468 | −1.14166 |
| | band 4.1 like 4B | | |
| GYG2 | glycogenin 2 | 0.009413 | −1.14164 |
| LYZL6 | lysozyme-like 6 | 0.006035 | −1.14149 |
| DCHS2 | dachsous 2 (Drosophila) | 0.006341 | −1.14146 |
| OBP2A /// | odorant binding protein 2A /// | 0.00776 | −1.14144 |
| OBP2B | odorant binding protein 2B | | |
| ANGPTL3 | angiopoietin-like 3 | 0.007619 | −1.1414 |
| MYH11 | myosin, heavy chain 11, smooth | 0.002648 | −1.14138 |
| | muscle | | |
| — | — | 0.00271 | −1.1412 |
| NES | nestin | 0.002731 | −1.14119 |
| SLC17A1 | solute carrier family 17 (sodium | 0.004803 | −1.14111 |
| | phosphate), member 1 | | |
| RBM15B | RNA binding motif protein 15B | 0.004537 | −1.14109 |
| CSH1 | chorionic somatomammotropin | 0.009332 | −1.14094 |
| | hormone 1 (placental lactogen) | | |
| HTR5A | 5-hydroxytryptamine | 0.000162 | −1.1409 |
| | (serotonin) receptor 5A | | |
| CYP3A7 | cytochrome P450, family 3, | 0.00199 | −1.1409 |
| | subfamily A, polypeptide 7 | | |
| HTR2A | 5-hydroxytryptamine | 0.004894 | −1.14084 |
| | (serotonin) receptor 2A | | |
| KCNV2 | potassium channel, subfamily | 0.005931 | −1.14082 |
| | V, member 2 | | |
| TOX3 | TOX high mobility group box | 0.001764 | −1.14064 |
| | family member 3 | | |
| CLOCK | clock homolog (mouse) | 0.006632 | −1.14057 |
| — | — | 0.006807 | −1.14053 |
| MAGEA6 | melanoma antigen family A, 6 | 0.00046 | −1.14048 |
| — | — | 0.000518 | −1.1404 |
| FAM12A | family with sequence similarity | 0.001548 | −1.14036 |
| | 12, member A | | |
| COL4A3 | collagen, type IV, alpha 3 | 0.007446 | −1.14035 |
| | (Goodpasture antigen) | | |
| S1PR2 | sphingosine-1-phosphate | 0.008163 | −1.14034 |
| | receptor 2 | | |
| NAT8 | N-acetyltransferase 8 (GCN5- | 0.002654 | −1.14031 |
| | related, putative) | | |
| ACE2 | angiotensin 1 converting | 0.007666 | −1.14031 |
| | enzyme (peptidyl-dipeptidase | | |
| | A) 2 | | |
| SLC22A6 | solute carrier family 22 (organic | 0.00816 | −1.14031 |
| | anion transporter), member 6 | | |
| SLC13A2 | solute carrier family 13 | 0.005043 | −1.14029 |
| | (sodium-dependent | | |
| | dicarboxylate transporter), | | |
| | member 2 | | |
| MYH4 | myosin, heavy chain 4, skeletal | 0.009604 | −1.14029 |
| | muscle | | |
| APBB2 | amyloid beta (A4) precursor | 0.009135 | −1.14026 |
| | protein-binding, family B, | | |
| | member 2 | | |
| RAP1GAP | RAP1 GTPase activating | 0.007618 | −1.14025 |
| | protein | | |
| SHOX2 | short stature homeobox 2 | 0.004273 | −1.14022 |
| SLCO1A2 | solute carrier organic anion | 0.003589 | −1.14006 |
| | transporter family, member 1A2 | | |
| ETV1 | ets variant 1 | 0.00888 | −1.14001 |
| MAGEA12 | melanoma antigen family A, 12 | 0.003805 | −1.13997 |
| PLA2G6 | phospholipase A2, group VI | 0.006425 | −1.13996 |
| | (cytosolic, calcium- | | |
| | independent) | | |
| ADRA1A | adrenergic, alpha-1A-, receptor | 0.007465 | −1.13995 |
| — | — | 0.008637 | −1.13992 |
| SYT5 | synaptotagmin V | 0.004699 | −1.1399 |
| GPR161 | G protein-coupled receptor 161 | 0.004773 | −1.13989 |
| SEMA3F | sema domain, immunoglobulin | 0.006736 | −1.13975 |
| | domain (Ig), short basic | | |
| | domain, secreted, (semaphorin) 3F | | |
| CYP3A43 | cytochrome P450, family 3, | 0.003382 | −1.13964 |
| | subfamily A, polypeptide 43 | | |
| HOMER2 | homer homolog 2 (Drosophila) | 0.003535 | −1.13961 |
| KCNJ5 | potassium inwardly-rectifying | 0.005942 | −1.13913 |
| | channel, subfamily J, member 5 | | |
| PPL | periplakin | 0.00221 | −1.13899 |
| COL17A1 | collagen, type XVII, alpha 1 | 0.003115 | −1.13894 |
| CSHL1 | chorionic somatomammotropin | 0.004285 | −1.13887 |
| | hormone-like 1 | | |
| C9orf116 | chromosome 9 open reading | 0.004915 | −1.13886 |
| | frame 116 | | |
| PARK2 | Parkinson disease (autosomal | 0.00541 | −1.13885 |
| | recessive, juvenile) 2, parkin | | |
| UGT2B15 | UDP glucuronosyltransferase 2 | 0.002586 | −1.13876 |
| | family, polypeptide B15 | | |
| — | — | 0.002677 | −1.13855 |
| CDK6 | cyclin-dependent kinase 6 | 0.005041 | −1.13851 |
| FAM174B | family with sequence similarity | 0.00769 | −1.13845 |
| | 174, member B | | |
| — | — | 6.02E−05 | −1.13837 |
| CELA2A /// | chymotrypsin-like elastase | 0.008746 | −1.13825 |
| CELA2B | family, member 2A /// | | |
| | chymotrypsin-like elastase | | |
| | family, mem | | |
| SPDEF | SAM pointed domain | 0.006039 | −1.13824 |
| | containing ets transcription | | |
| | factor | | |
| EPB41 | erythrocyte membrane protein | 0.00381 | −1.13816 |
| | band 4.1 (elliptocytosis 1, RH- | | |
| | linked) | | |
| GAB1 | GRB2-associated binding | 0.008578 | −1.13811 |
| | protein 1 | | |
| SMAD6 | SMAD family member 6 | 0.002361 | −1.13807 |
| SMR3A | submaxillary gland androgen | 0.004041 | −1.13806 |
| | regulated protein 3 A | | |
| PDE6G | phosphodiesterase 6G, cGMP- | 0.009564 | −1.13803 |
| | specific, rod, gamma | | |
| COL5A1 | collagen, type V, alpha 1 | 0.003287 | −1.13787 |
| ABCA6 | ATP-binding cassette, sub- | 0.008623 | −1.13787 |
| | family A (ABC1), member 6 | | |
| DMD | dystrophin | 0.000248 | −1.13783 |
| CYLC2 | cylicin, basic protein of sperm | 0.008464 | −1.13771 |
| | head cytoskeleton 2 | | |
| CIDEA | cell death-inducing DFFA-like | 0.007254 | −1.13768 |
| | effector a | | |
| RAG2 | recombination activating gene 2 | 0.002984 | −1.13757 |
| HIST1H2BN | histone cluster 1, H2bn | 0.007319 | −1.13751 |
| FMO6P | flavin containing | 0.007564 | −1.13747 |
| | monooxygenase 6 pseudogene | | |
| — | — | 0.009453 | −1.13738 |
| MAOA | monoamine oxidase A | 0.004806 | −1.13729 |
| ANKRD53 | ankyrin repeat domain 53 | 0.00488 | −1.13725 |
| HAPLN1 | hyaluronan and proteoglycan | 0.00865 | −1.13719 |
| | link protein 1 | | |
| MT1M | metallothionein 1M | 0.009364 | −1.13718 |
| EHD2 | EH-domain containing 2 | 0.006795 | −1.13713 |
| GAD2 | glutamate decarboxylase 2 | 0.007785 | −1.13709 |
| | (pancreatic islets and brain, | | |
| | 65 kDa) | | |
| CRISP2 | cysteine-rich secretory protein 2 | 0.009143 | −1.13708 |
| CSN2 | casein beta | 0.006005 | −1.13695 |
| — | — | 0.006648 | −1.13691 |
| SULT1C2 | sulfotransferase family, | 0.001313 | −1.13685 |
| | cytosolic, 1C, member 2 | | |
| PCDHGA3 | protocadherin gamma | 0.002114 | −1.13681 |
| | subfamily A, 3 | | |
| SSX3 | synovial sarcoma, X breakpoint 3 | 0.001106 | −1.13668 |
| FGFR2 | fibroblast growth factor | 0.006105 | −1.13666 |
| | receptor 2 | | |
| GPR161 | G protein-coupled receptor 161 | 0.00957 | −1.13664 |
| ATN1 | atrophin 1 | 0.009835 | −1.13654 |
| CHD5 | chromodomain helicase DNA | 0.007274 | −1.13651 |
| | binding protein 5 | | |
| A4GALT | alpha 1,4-galactosyltransferase | 0.001062 | −1.13647 |
| MYBPH | myosin binding protein H | 0.007527 | −1.13634 |
| CSHL1 | chorionic somatomammotropin | 0.002335 | −1.1363 |
| | hormone-like 1 | | |
| — | — | 0.004169 | −1.13618 |
| NCAPH2 | non-SMC condensin II | 0.0079 | −1.13612 |
| | complex, subunit H2 | | |
| CAPN9 | calpain 9 | 0.003071 | −1.13597 |
| CNGB1 | cyclic nucleotide gated channel | 0.007125 | −1.13588 |
| | beta 1 | | |
| BCAM | basal cell adhesion molecule | 0.00753 | −1.13578 |
| | (Lutheran blood group) | | |
| DRD5 | dopamine receptor D5 | 0.00281 | −1.13557 |
| NR5A2 | nuclear receptor subfamily 5, | 0.000913 | −1.13547 |
| | group A, member 2 | | |
| TEF | thyrotrophic embryonic factor | 0.004588 | −1.13544 |
| ELAVL2 | ELAV (embryonic lethal, | 0.002205 | −1.13541 |
| | abnormal vision, Drosophila)- | | |
| | like 2 (Hu antigen B) | | |
| DGKB | diacylglycerol kinase, beta | 0.00918 | −1.13537 |
| | 90 kDa | | |
| HTR7P | 5-hydroxytryptamine | 0.00209 | −1.13524 |
| | (serotonin) receptor 7 | | |
| | pseudogene | | |
| RHAG | Rh-associated glycoprotein | 0.005396 | −1.1352 |
| GH2 | growth hormone 2 | 0.006321 | −1.13519 |
| — | — | 0.000833 | −1.13517 |
| — | — | 0.00238 | −1.13477 |
| COL4A6 | collagen, type IV, alpha 6 | 0.004113 | −1.13473 |
| BMP7 | bone morphogenetic protein 7 | 0.005776 | −1.13444 |
| — | — | 0.008419 | −1.13443 |
| — | — | 0.007353 | −1.13442 |
| SOSTDC1 | sclerostin domain containing 1 | 0.001856 | −1.13439 |
| SOX14 | SRY (sex determining region | 0.001541 | −1.13438 |
| | Y)-box 14 | | |
| TAS2R9 | taste receptor, type 2, member 9 | 0.002889 | −1.13437 |
| LPHN2 | latrophilin 2 | 0.009349 | −1.13418 |
| — | — | 0.0099 | −1.13418 |
| — | — | 0.007172 | −1.13409 |
| MAP1A | microtubule-associated protein 1A | 0.004384 | −1.13404 |
| OSGIN2 | Oxidative stress induced growth | 0.009721 | −1.13398 |
| | inhibitor family member 2 | | |
| SLC10A2 | solute carrier family 10 | 0.006712 | −1.13395 |
| | (sodium/bile acid cotransporter | | |
| | family), member 2 | | |
| FAM13C | family with sequence similarity | 0.005974 | −1.13385 |
| | 13, member C | | |
| EMX1 | empty spiracles homeobox 1 | 0.005643 | −1.13366 |
| FLJ40330 | hypothetical LOC645784 | 0.005033 | −1.13365 |
| CHI3L1 | chitinase 3-like 1 (cartilage | 0.002657 | −1.13357 |
| | glycoprotein-39) | | |
| — | — | 0.002091 | −1.1335 |
| CDH16 | cadherin 16, KSP-cadherin | 0.004017 | −1.13345 |
| SPRR1A | small proline-rich protein 1A | 0.00177 | −1.13344 |
| LOX | lysyl oxidase | 0.008602 | −1.13331 |
| — | — | 0.009241 | −1.13331 |
| CALCB | calcitonin-related polypeptide | 0.005526 | −1.13322 |
| | beta | | |
| GABBR2 | gamma-aminobutyric acid | 0.003986 | −1.1332 |
| | (GABA) B receptor, 2 | | |
| CPB2 | carboxypeptidase B2 (plasma) | 0.004188 | −1.13308 |
| RASL11B | RAS-like, family 11, member B | 0.007069 | −1.1329 |
| CCDC81 | coiled-coil domain containing | 0.009508 | −1.13288 |
| | 81 | | |
| RUNX1 | runt-related transcription | 0.006825 | −1.13281 |
| | factor 1 | | |
| CPA1 | carboxypeptidase A1 | 0.002754 | −1.13221 |
| | (pancreatic) | | |
| CLCNKA /// | chloride channel Ka /// chloride | 0.001569 | −1.13213 |
| CLCNKB | channel Kb | | |
| FHL5 | four and a half LIM domains 5 | 0.008016 | −1.13211 |
| THSD7A | thrombospondin, type I, domain | 0.001208 | −1.1321 |
| | containing 7A | | |
| TFAP2C | transcription factor AP-2 | 0.004298 | −1.13184 |
| | binding protein 2 gamma) | | |
| SPAG11B | sperm associated antigen 11B | 0.005305 | −1.1317 |
| CAP2 | CAP, adenylate cyclase- | 0.002715 | −1.13166 |
| | associated protein, 2 (yeast) | | |
| PODNL1 | podocan-like 1 | 0.00498 | −1.13165 |
| SSX4 /// | synovial sarcoma, X breakpoint | 0.009142 | −1.13163 |
| SSX4B | 4 /// synovial sarcoma, X | | |
| | breakpoint 4B | | |
| — | — | 0.008421 | −1.13155 |
| G6PC | glucose-6-phosphatase, | 0.006788 | −1.13144 |
| | catalytic subunit | | |
| RPE65 | retinal pigment epithelium- | 0.00168 | −1.13112 |
| | specific protein 65 kDa | | |
| TMEM222 | transmembrane protein 222 | 0.003611 | −1.13108 |
| — | — | 0.0082 | −1.13098 |
| — | — | 0.000935 | −1.13097 |
| KDR | kinase insert domain receptor (a | 0.002995 | −1.13074 |
| | type III receptor tyrosine | | |
| | kinase) | | |
| — | — | 0.004838 | −1.13055 |
| CHP2 | calcineurin B homologous | 0.00539 | −1.13026 |
| | protein 2 | | |
| GPR64 | G protein-coupled 64 | 0.001932 | −1.13023 |
| TPM2 | tropomyosin 2 (beta) | 0.008933 | −1.13017 |
| TCEB3B | transcription elongation factor | 0.006753 | −1.13005 |
| | B polypeptide 3B (elongin A2) | | |
| E2F5 | E2F transcription factor 5, | 0.001129 | −1.13001 |
| | p130-binding | | |
| IL5RA | interleukin 5 receptor, alpha | 0.003392 | −1.12982 |
| AOC3 | amine oxidase, copper | 0.000247 | −1.12972 |
| | containing 3 (vascular adhesion | | |
| | protein 1) | | |
| ABCF3 | ATP-binding cassette, sub- | 0.004992 | −1.12966 |
| | family F (GCN20), member 3 | | |
| CPN2 | carboxypeptidase N, | 0.006009 | −1.12966 |
| | polypeptide 2 | | |
| ACE | angiotensin 1 converting | 0.00879 | −1.12948 |
| | enzyme (peptidyl-dipeptidase A) 1 | | |
| NRP2 | neuropilin 2 | 0.008745 | −1.12938 |
| INPP5J | inositol polyphosphate-5- | 0.007608 | −1.12935 |
| | phosphatase J | | |
| SMAD9 | SMAD family member 9 | 0.006141 | −1.1293 |
| FAM155A | family with sequence similarity | 0.005057 | −1.12928 |
| | 155, member A | | |
| GART | phosphoribosylglycinamide | 0.00165 | −1.12917 |
| | formyltransterase, | | |
| | phosphoribosylglycinamide | | |
| | synthetase, phos | | |
| PIR | pirin (iron-binding nuclear | 0.004128 | −1.12911 |
| | protein) | | |
| ZNF467 | Zinc finger protein 467 | 0.006009 | −1.12907 |
| ITSN2 | intersectin 2 | 0.001473 | −1.12903 |
| NR1D1 /// | nuclear receptor subfamily 1, | 0.001964 | −1.12896 |
| THRA | group D, member 1 /// thyroid | | |
| | hormone receptor, alpha (er | | |
| RP11-35N6.1 | plasticity related gene 3 | 0.005452 | −1.12885 |
| LAMB1 | laminin, beta 1 | 0.006026 | −1.12864 |
| EPHB3 | EPH receptor B3 | 0.003783 | −1.12857 |
| — | — | 0.008156 | −1.12856 |
| PLA2R1 | phospholipase A2 receptor 1, | 0.005834 | −1.12854 |
| | 180 kDa | | |
| RAPGEF4 | Rap guanine nucleotide | 0.008542 | −1.12853 |
| | exchange factor (GEF) 4 | | |
| DNAJC8 | DnaJ (Hsp40) homolog, | 0.007393 | −1.12851 |
| | (subfamily C, member 8 | | |
| ARSJ | arylsulfatase family, member J | 0.002634 | −1.12845 |
| TRIM49 | tripartite motif-containing 49 | 0.002053 | −1.12832 |
| IL9 | interleukin 9 | 0.003046 | −1.12812 |
| GC | group-specific component | 0.001817 | −1.12797 |
| | (vitamin D binding protein) | | |
| IL12B | interleukin 12B (natural killer | 0.006181 | −1.12764 |
| | cell stimulatory factor 2, | | |
| | cytotoxic lymphocyte maturat | | |
| CDH2 | cadherin 2, type 1, N-cadherin | 0.00855 | −1.12758 |
| | (neuronal) | | |
| ATXN3L | ataxin 3-like | 0.006981 | −1.12737 |
| BTF3L1 | basic transcription factor 3, | 0.008187 | −1.12701 |
| | like 1 pseudogene | | |
| CCL19 | chemokine (C-C motif) ligand 19 | 0.006791 | −1.12696 |
| BICC1 | bicaudal C homolog 1 | 0.007006 | −1.12695 |
| | (Drosophila) | | |
| FAM186A | family with sequence similarity | 0.00559 | −1.1268 |
| | 186, member A | | |
| PTPRF | protein tyrosine phosphatase, | 0.004195 | −1.12674 |
| | receptor type, F | | |
| TRPC4 | transient receptor potential | 0.009479 | −1.12666 |
| | cation channel, subfamily C, | | |
| | member 4 | | |
| TCL6 | T-cell leukemia/lymphoma 6 | 0.009932 | −1.12663 |
| CYP4A22 | cytochrome P450, family 4, | 0.004269 | −1.12654 |
| | subfamily A, polypeptide 22 | | |
| FUT6 | fucosyltransferase 6 (alpha (1,3) | 0.001302 | −1.12636 |
| | fucosyltransferase) | | |
| MUC1 | mucin 1, cell surface associated | 0.008714 | −1.12624 |
| DKFZP434B2016 /// | similar to hypothetical protein | 0.002252 | −1.12623 |
| LOC643313 | LOC284701 /// similar to | | |
| | hypothetical protein | | |
| | LOC284701 | | |
| — | — | 0.005094 | −1.12618 |
| LDHA | lactate dehydrogenase A | 0.009545 | −1.12609 |
| — | — | 0.007472 | −1.12582 |
| — | — | 0.003741 | −1.12572 |
| LOC100131613 | PRO1454 | 0.00409 | −1.12556 |
| TRIM3 | tripartite motif-containing 3 | 0.004335 | −1.12551 |
| MLLT10 | myeloid/lymphoid or mixed- | 0.006971 | −1.12505 |
| | lineage leukemia (trithorax | | |
| | homolog, Drosophila); | | |
| | translocate | | |
| DZIP1 | DAZ interacting protein 1 | 0.003223 | −1.12501 |
| ANKRD34C | ankyrin repeat domain 34C | 0.000954 | −1.12484 |
| BUB1 | budding uninhibited by | 0.002759 | −1.12479 |
| | benzimidazoles 1 homolog | | |
| | (yeast) | | |
| CSPG5 | chondroitin sulfate | 0.000873 | −1.12474 |
| | proteoglycan 5 (neuroglycan C) | | |
| FBLN1 | fibulin 1 | 0.002979 | −1.12464 |
| GAD2 | glutamate decarboxylase 2 | 0.007671 | −1.12463 |
| | (pancreatic islets and brain, | | |
| | 65 kDa) | | |
| — | — | 0.000944 | −1.12451 |
| CLDN1 | claudin 1 | 0.002407 | −1.12447 |
| CHRNA3 | cholinergic receptor, nicotinic, | 0.006458 | −1.12436 |
| | alpha 3 | | |
| SCN11A | sodium channel, voltage-gated, | 0.003796 | −1.12417 |
| | type XI, alpha subunit | | |
| TEX11 | testis expressed 11 | 0.006239 | −1.12409 |
| IL20RA | interleukin 20 receptor, alpha | 0.004863 | −1.124 |
| AKAP5 | A kinase (PRKA) anchor | 0.006577 | −1.12361 |
| | protein 5 | | |
| KBTBD10 | kelch repeat and BTB (POZ) | 0.005771 | −1.12343 |
| | domain containing 10 | | |
| MSTN | myostatin | 0.001873 | −1.1234 |
| TLL2 | tolloid-like 2 | 0.009968 | −1.12321 |
| NACAD | NAC alpha domain containing | 0.000782 | −1.12294 |
| UNC93A | unc-93 homolog A (C. elegans) | 0.006006 | −1.12294 |
| PTGER1 | prostaglandin E receptor 1 | 0.007003 | −1.12294 |
| | (subtype EP1), 42 kDa | | |
| OLAH | oleoyl-ACP hydrolase | 0.00896 | −1.12289 |
| NHLH2 | nescient helix loop helix 2 | 0.001059 | −1.12286 |
| SERPINA6 | serpin peptidase inhibitor, clade | 0.002411 | −1.12272 |
| | A (alpha-1 antiproteinase, | | |
| | antitrypsin), member 6 | | |
| — | — | 0.006655 | −1.12253 |
| KRT17 | keratin 17 | 0.003553 | −1.12241 |
| — | — | 0.006777 | −1.12239 |
| KCNMA1 | potassium large conductance | 0.0067 | −1.12228 |
| | calcium-activated channel, | | |
| | subfamily M, alpha member 1 | | |
| PRKCA | protein kinase C, alpha | 0.006437 | −1.12198 |
| STS | steroid sulfatase (microsomal), | 0.005597 | −1.12182 |
| | isozyme S | | |
| LAMA1 | laminin, alpha 1 | 0.004674 | −1.1216 |
| GPR88 | G protein-coupled receptor 88 | 0.008737 | −1.1216 |
| ACTN2 | actinin, alpha 2 | 0.003627 | −1.12157 |
| TREH | trehalase (brush-border | 0.005379 | −1.12152 |
| | membrane glycoprotein) | | |
| AKAP4 | A kinase (PRKA) anchor | 0.004629 | −1.12147 |
| | protein 4 | | |
| DKX4 | dickkopf homolog 4 | 0.005161 | −1.1214 |
| | (Xenopus laevis) | | |
| — | — | 0.007472 | −1.12129 |
| PRICKLE3 | prickle homolog 3 (Drosophila) | 0.007288 | −1.12117 |
| IRS4 | insulin receptor substrate 4 | 0.004715 | −1.12108 |
| TRPV4 | transient receptor potential | 0.008105 | −1.12103 |
| | cation channel, subfamily V, | | |
| | member 4 | | |
| PCDH11Y | protocadherin 11 Y-linked | 0.002241 | −1.121 |
| — | — | 0.009044 | −1.12095 |
| APBB2 | amyloid beta (A4) precursor | 0.002596 | −1.12093 |
| | protein-binding, family B, | | |
| | member 2 | | |
| SLCO2A1 | solute carrier organic anion | 0.004733 | −1.12079 |
| | transporter family, member 2A1 | | |
| — | — | 0.00447 | −1.12075 |
| DRD2 | dopamine receptor D2 | 0.002588 | −1.12059 |
| MTMR7 | myotubularin related protein 7 | 0.009209 | −1.12027 |
| ZNF471 | zinc finger protein 471 | 0.004677 | −1.12005 |
| TF | transferrin | 0.007274 | −1.11993 |
| NRIP2 | nuclear receptor interacting | 0.005607 | −1.11966 |
| | protein 2 | | |
| ST6GALNAC5 | ST6 (alpha-N-acetyl- | 0.005579 | −1.11932 |
| | neuraminyl-2,3-beta-galactosyl- | | |
| | 1,3)-N-acetylgalactosaminide | | |
| | alpha-2 | | |
| COMT | Catechol-O-methyltransterase | 0.007202 | −1.11926 |
| — | — | 0.008063 | −1.11915 |
| — | — | 0.005963 | −1.11891 |
| — | — | 0.001254 | −1.11889 |
| PAH | phenylalanine hydroxylase | 0.002401 | −1.11852 |
| LRRC19 | leucine rich repeat containing 19 | 0.003683 | −1.11836 |
| PRKAR1B | protein kinase, cAMP- | 0.007626 | −1.11835 |
| | dependent, regulatory, type I, | | |
| | beta | | |
| HPR | haptoglobin-related protein | 0.005044 | −1.11829 |
| PRDM5 | PR domain containing 5 | 0.006648 | −1.11821 |
| — | — | 0.008206 | −1.11819 |
| NCRNA00120 | non-protein coding RNA 120 | 0.004933 | −1.11814 |
| LOC79999 | hypothetical LOC79999 | 0.008297 | −1.11814 |
| ITSN2 | intersectin 2 | 0.004428 | −1.118 |
| CACNB2 | calcium channel, voltage- | 0.008677 | −1.11798 |
| | dependent, beta 2 subunit | | |
| GPR98 | G protein-coupled receptor 98 | 0.003265 | −1.11788 |
| PREX2 | phosphatidylinositol-3,4,5- | 0.00758 | −1.11779 |
| | trisphosphate-dependent Rac | | |
| | exchange factor 2 | | |
| FAM182B | family with sequence similarity | 0.008506 | −1.11758 |
| | 182, member B | | |
| LAMA4 | laminin, alpha 4 | 0.001859 | −1.11742 |
| — | — | 0.008182 | −1.117 |
| ARVCF | armadillo repeat gene deletes in | 0.004203 | −1.11699 |
| | velocardiofacial syndrome | | |
| HAS2 | hyaluronan synthase 2 | 0.005988 | −1.11699 |
| YOD1 | YOD1 OTU deubiquinating | 0.007323 | −1.11668 |
| | enzyme 1 homolog | | |
| | (S. cerevisiae) | | |
| — | — | 0.003067 | −1.11636 |
| PPP2R3A | protein phosphatase 2 (formerly | 0.00874 | −1.11635 |
| | 2A), regulatory subunit B″, | | |
| | alpha | | |
| COL4A1 | collagen, type IV, alpha 1 | 0.002703 | −1.11621 |
| — | — | 0.005114 | −1.11613 |
| RBM12B | RNA binding motif protein 12B | 0.004522 | −1.11612 |
| TNFRSF11B | tumor necrosis factor receptor | 0.009363 | −1.11609 |
| | superfamily, member 11b | | |
| GSTA3 | glutathione S-transferase alpha 3 | 0.009885 | −1.11598 |
| FAM66D | family with sequence similarity | 0.007508 | −1.11588 |
| | 66, member D | | |
| OR10H2 | olfactory receptor, family 10, | 0.001279 | −1.11576 |
| | subfamily H, member 2 | | |
| PTHLH | parathyroid hormone-like | 0.003579 | −1.1157 |
| | hormone | | |
| ZNF674 | zinc finger family member 674 | 0.007146 | −1.11565 |
| — | — | 0.004948 | −1.11555 |
| KRT19 | keratin 19 | 0.005928 | −1.11545 |
| — | — | 0.006606 | −1.11543 |
| ACCN2 | amiloride-sensitive cation | 0.006168 | −1.11533 |
| | channel 2, neuronal | | |
| COL6A1 | Collagen, type VI, alpha 1 | 0.006453 | −1.11496 |
| — | — | 0.007286 | −1.11496 |
| LOC100288442 /// | hypothetical LOC100288442 /// | 0.008735 | −1.11485 |
| LOC100289169 /// | hypothetical protein | | |
| LOC728888 /// | LOC100289169 /// similar to | | |
| LOC729602 /// | acyl-CoA | | |
| NPIPL2 /// | | | |
| NPIPL3 /// | | | |
| PDXDC2 | | | |
| SLC37A1 | solute carrier family 37 | 0.00935 | −1.11481 |
| | (glycerol-3-phosphate | | |
| | transporter), member 1 | | |
| ATP6V1B1 | ATPase, H+ transporting, | 0.006862 | −1.11475 |
| | lysosomal 56/58 kDa, V1 | | |
| | subunit B1 | | |
| PTN | pleiotrophin | 0.009862 | −1.11456 |
| ABI3BP | ABI family, member 3 (NESH) | 0.004323 | −1.11452 |
| | binding protein | | |
| HR44 | Hr44 antigen | 0.002042 | −1.11417 |
| ZNF324B /// | zinc finger protein 324B /// zinc | 0.00485 | −1.11417 |
| ZNF584 | finger protein 584 | | |
| HOXD13 | homeobox D13 | 0.005052 | −1.11415 |
| ADH6 | alcohol dehydrogenase 6 (class V) | 0.00011 | −1.11405 |
| IFNA8 | interferon, alpha 8 | 0.004318 | −1.1136 |
| — | — | 0.001609 | −1.1135 |
| — | — | 0.002312 | −1.11325 |
| MYOZ2 | myozenin 2 | 0.009469 | −1.11325 |
| NFATC4 | nuclear factor of activated T- | 0.008681 | −1.11245 |
| | cells, cytoplasmic, calcineurin- | | |
| | dependent 4 | | |
| ADAMTS7 | ADAM metallopeptidase with | 0.007469 | −1.11244 |
| | thrombospondin type 1 motif, 7 | | |
| FOXL1 | forkhead box L1 | 0.009415 | −1.11184 |
| GPR17 | G protein-coupled receptor 17 | 0.009308 | −1.11183 |
| SLC18A3 | solute carrier family 18 | 0.006308 | −1.11131 |
| | (vesicular acetylcholine), | | |
| | member 3 | | |
| MYH6 | myosin, heavy chain 6, cardiac | 0.005188 | −1.11125 |
| | muscle, alpha | | |
| BOK | BCL2-related ovarian killer | 0.008744 | −1.111 |
| FGA | fibrinogen alpha chain | 0.006323 | −1.11088 |
| TEAD4 | TEA domain family member 4 | 0.008474 | −1.11075 |
| — | — | 0.005611 | −1.11074 |
| GRM1 | glutamate receptor, | 0.003649 | −1.11073 |
| | metabotropic 1 | | |
| — | — | 0.008379 | −1.11041 |
| EDNRA | endothelin receptor type A | 0.00586 | −1.11035 |
| C8orf79 | chromosome 8 open reading | 0.008561 | −1.11003 |
| | frame 79 | | |
| METTL7A | methyltransferase like 7A | 0.004255 | −1.10991 |
| FOLH1 | folate hydrolase (prostate- | 0.002431 | −1.10932 |
| | specific membrane antigen) 1 | | |
| RAD54L | RAD54-like (S. cerevisiae) | 0.007758 | −1.10898 |
| — | — | 0.003086 | −1.10888 |
| — | — | 0.006615 | −1.10851 |
| SOX11 | SRY (sex determining region | 0.007605 | −1.10851 |
| | Y)-box 11 | | |
| CNOT3 | CCR4-NOT transcription | 0.006753 | −1.10843 |
| | complex, subunit 3 | | |
| NTS | neurotensin | 0.008975 | −1.10791 |
| MAPK12 | mitogen-activated protein | 0.001821 | −1.10765 |
| | kinase 12 | | |
| DOCK6 | dedicator of cytokinesis 6 | 0.004973 | −1.10674 |
| DNAJC6 | DnaJ (Hsp40) homolog, | 0.008411 | −1.10661 |
| | subfamily C, member 6 | | |
| HS3ST3A1 | heparan sulfate (glucosamine) | 0.002138 | −1.10659 |
| | 3-O-sulfotransferase 3A1 | | |
| — | — | 0.009792 | −1.10575 |
| LOC728395 /// | testis specific protein, Y-linked | 0.005872 | −1.10572 |
| TSPY1 /// | 1-like /// testis specific protein, | | |
| TSPY3 | Y-linked 1 /// te | | |
| PTH | parathyroid hormone | 0.00392 | −1.10538 |
| — | — | 0.004436 | −1.10502 |
| LAMB4 | laminin, beta 4 | 0.008445 | −1.10472 |
| ALDOB | aldolase B, fructose- | 0.00586 | −1.10469 |
| | bisphosphate | | |
| FLG | filaggrin | 0.007077 | −1.10448 |
| MLANA | melan-A | 0.007622 | −1.10421 |
| UBE2D4 | ubiquitin-conjugating enzyme | 0.005409 | −1.10409 |
| | E2D 4 (putative) | | |
| LOC100287483 | transcription elongation factor | 0.007954 | −1.10403 |
| | B (SIII), polypeptide I | | |
| | pseudogene | | |
| KRT20 | keratin 20 | 0.00756 | −1.10399 |
| — | — | 0.009136 | −1.10295 |
| POU1F1 | POU class 1 homeobox 1 | 0.009389 | −1.10165 |
| SLCO1B3 | solute carrier organic anion | 0.003628 | −1.10159 |
| | transporter family, member 1B3 | | |
| CLTA | Clathrin, light chain (Lca) | 0.009895 | −1.09903 |
| MECOM | MDS1 and EVI1 complex locus | 0.009639 | −1.09815 |
| C8orf71 | chromosome 8 open reading | 0.005004 | −1.09779 |
| | frame 71 | | |
| SULT2A1 | sulfotransferase, family, | 0.006809 | −1.09766 |
| | cytosolic, 2A, | | |
| | dehydroepiandrosterone | | |
| | (DHEA)-preferring, membe | | |
| C6orf10 | chromosome 6 open reading | 0.007231 | −1.09608 |
| | frame 10 | | |
| — | — | 0.00835 | −1.09349 |
| SLC27A6 | solute carrier family 27 (fatty | 0.007873 | −1.09267 |
| | transporter), member 6 | | |
| PRKD1 | protein kinase D1 | 0.00074 | −1.09164 |
| SYNPO2L | synaptopodin 2-like | 0.003229 | −1.0912 |
| THPO | thrombopoietin | 0.008285 | −1.09086 |
| GABRR1 | gamma-aminobutyric acid | 0.008683 | −1.09024 |
| | (GABA) receptor, rho 1 | | |
| CFTR | cystic fibrosis transmembrane | 0.003801 | −1.0898 |
| | conductance regulator (ATP- | | |
| | binding cassette sub-family C, | | |
| PPP2R3A | protein phosphatase 2 (formerly | 0.005056 | −1.08882 |
| | 2A), regulatory subunit B″, | | |
| | alpha | | |
| DCBLD2 | discoidin, CUB and LCCL | 0.008379 | −1.08262 |
| | domain containing 2 | | |
| — | — | 0.008713 | −1.08123 |
| ANP32A /// | acidic (leucine-rich) nuclear | 0.007633 | −1.07373 |
| ANP32C /// | phosphoprotein 32 family, | | |
| ANP32D /// | member A /// acidic (leucine-ri | | |
| LOC723972 | | | |
| XYLT1 | xylosyltransferase I | 0.002463 | 1.33229 |
| STAB1 | stabilin 1 | 0.002614 | 1.46208 |
| STAB1 | stabilin 1 | 0.004388 | 1.52209 |
| SASH1 | SAM and SH3 domain | 0.000209 | 1.64433 |
| | containing 1 | | |
| PID1 | phosphotyrosine interaction | 0.008082 | 1.65163 |
| | domain containing 1 | | |
| FUCA1 | fucosidase, alpha-L-1, tissue | 0.00028 | 1.67342 |
| SASH1 | SAM and SH3 domain | 0.000586 | 2.01341 |
| | containing 1 | | |
| LRRN3 | leucine rich repeat neuronal 3 | 0.00066 | 2.52567 |
| LRRN3 | leucine rich repeat neuronal 3 | 0.000927 | 2.54705 |

### REFERENCES

1. PCT international Application Publication No. WO 2007/047863, published Apr. 26, 2007, international filing date Oct. 18, 2006.
2. PCT International Application Publication No. WO 2007/146248, published Dec. 21, 2007, international filing date Jun. 12, 2007.
3. Barkhof, F. (1999) “MRI in Multiple Sclerosis: Correlation with Expanded Disability Status Scale (EDSS)”, Multiple Sclerosis. 5(4):283-286 (Abstract).
4. Bartolome, et al. (2003) “Rapid up-regulation of alpha4 integrin-mediated leukocyte adhesion by transforming growth factor-beta1”, Mol Biol Cell 14, 54-66.
5. Bjartmar and Fox (2002) “Pathological mechanisms and disease progression of multiple sclerosis: therapeutic implication”, Drugs of Today. 38:7-29.
6. Brex et al. (2002) “A longitudinal study of abnormalities on MRI and disability from multiple sclerosis”, N Eng J Med. Jan. 17, 2002 346(3):158-64.
7. Brill et al. (2001) “Regulation of T-cell interaction with fibronectin by transforming growth factor-beta is associated with altered Pyk2 phosphorylation”, Immunology 104, 149-156.
8. Brück et al. (2011) “Insight into the mechanism of laquinimod action”, J. Neurol Sci. 306:173-179.
9. Bruck et al. (2012) “Reduced astrocytic NF-kappaB activation by laquinimod protects from cuprizone-induced demyelination”, Acta Neuropathol 124, 411-424.
10. Bruck and Vollmer (2013) “Multiple sclerosis: Oral laquinimod for MS-bringing the brain into focus”, Nat Rev Neurol 9, 664-665.
11. Brunmark et al. (2002) “The new orally active immunoregulator laquinimod (ABR-215062) effectively inhibits development and relapses of experimental autoimmune encephalomyelitis”, J Neuroimmunology. 130:163-172.
12. Cohen et al. (2010) “Oral fingolimod or intramuscular interferon for relapsing multiple sclerosis”. N Eng J Med; 362:402-415.
13. Comi et al. (2007) LAQ/5062 Study Group. “The Effect of Two Doses of Laquinimod on MRI-Monitored Disease Activity in Patients with Relapsing-Remitting Multiple Sclerosis: A Multi-Center, Randomized, Double-Blind, Placebo-Controlled Study”, Presented at: 59th Annual Meeting of the American Academy of Neurology: Apr. 28-May 5, 2007; Boston, Mass.
14. Comi et al. (2008) “Effect of laquinimod on MRI-monitored disease activity in patients with relapsing-remitting multiple sclerosis: a multicentre, randomised, double-blind, placebo-controlled phase IIb study”, Lancet. 371:2085-2092.
15. Comi et al. (2009) for the LAQ/5062 Clinical Advisory Board and Study Group. Long-term open extension of oral laquinimod in patients with relapsing multiple sclerosis shows favorable safety and sustained low relapse rate and MRI activity. [Ectrims abstract P443]. Mult Scler. 15(Suppl 2):S127.
16. Comi et al. (2010) for the LAQ/5062 Clinical Advisory Board and Study Group. The effect of laquinimod on MRI-monitored disease activity in patients with relapsing-remitting multiple sclerosis: a double-blind active extension of the multicentre, randomised, double-blind, parallel-group placebo-controlled study. Mult Scler. 16:1360-1366.
17. Comi et al. (2012) “Placebo-controlled trial of oral laquinimod for multiple sclerosis”, N Engl J Med 366, 1000-1009.
18. Cutter et al. (1999) “Development of a multiple sclerosis functional composite as a clinical trial outcome measure”, Brain. 122:871-882.
19. De Stefano et al. (1999) “Evidence of early axonal damage in patients with multiple sclerosis”, Neurology. 52(Suppl 2):A378.
20. Dunitz. M. (1999) Multiple sclerosis therapeutics, Ed. Rudick and Goodkin. London: Taylor & Francis, 1999.
21. Durelli et al. and the Independent Comparison of Interferon (INCOMIN) Trial Study Group. (2002) “Every-other-day interferon beta-1b versus once-weekly interferon beta-1a for multiple sclerosis: results of a 2-year prospective randomised multicentre study (INCOMIN)”, Lancet. 359:1453-60.
22. East et al., (2008) “Chronic relapsing experimental allergic encephalomyelitis (CREAE) in plasminogen activator inhibitor-1 knockout mice: the effect of fibrinolysis during neuroinflammation”, Neuropathol Appl Neurobiol. 34:16-30
23. EMEA Guideline on Clinical Investigation of Medicinal Products for the Treatment of Multiple Sclerosis (CPMP/EWP/561/98 Rev. 1, Nov. 2006).
24. EPAR, Rebif®, Scientific Discussion.
25. Filippi et al. (2014) “Placebo-controlled trial of oral laquinimod in multiple sclerosis: MRI evidence of an effect on brain tissue damage”, J Neurol Neurosurg Psychiatry 85, 851-858.
26. Fischer et al. (1999) “The Multiple Sclerosis Functional Composite measure (MSFC): an integrated approach to MS clinical outcome assessment” Multiple Sclerosis. 5(4):244-250.
27. Fisk et al. (1994) “Measuring the Functional Impact of Fatigue: Initial Validation of Fatigue Impact Scale”, Clin Inf Dis. 18 Suppl 1:S79-83.
28. Fisk et al. (1994) “The Impact of Fatigue on Patients with Multiple Sclerosis”, Can J Neurol S. 21:9-14.
29. Frohman et al. (2003) “The utility of MRI in suspected MS: report of the Therapeutics and Technology Assessment Subcommittee of the American Academy of Neurology”, Neurology. Sep. 9, 2003, 61(5):602-11.
30. Giovannoni et al. (2010) “A placebo-controlled trial of oral cladribine for relapsing multiple sclerosis”, N Eng J Mod. 362:416-426.
31. Golder W. (2007) “Magnetic resonance spectroscopy in clinical oncology”, Onkologie. 27(3): 304-9.
32. Grossman et al. (1994) Magnetization transfer: theory and clinical applications in neuroradiology”, RadioGraphics. 14:279-290.
33. Gurevich et al., (2010) “Laquinimod suppress antigen presentation in relapsing-remitting multiple sclerosis: in-vitro high-throughput gene expression study”, J. Neuroimmunol 221:87-94
34. Gveric et al., (2003) “Impaired fibrinolysis in multiple sclerosis: a role for tissue plasminogen activator inhibitors”, Brain 126: 1590-8
35. Hartung et al. (2005) “Significance of neutralizing antibodies to interferon beta during treatment of multiple sclerosis: expert opinions based on the Proceedings of an International Consensus Conference”, Eur J Neurol. 12:588-601.
36. Hauser et al. (1983) “Intensive immunosuppression in progressive multiple sclerosis”, E Eng J Med. 308:173-180.
37. Hohlfeld et al. (2000) “The neuroprotective effect of inflammation: implications for the therapy of multiple sclerosis”, J Neuroimmunol. 107:161-166.
38. Hummon et al. (2007) “Isolation and solubilization of proteins after TRIzo1 extraction of RNA and DNA from patient material following prolonged storage”, Biotechniques 42, 467-470, 472.
39. Jacobs et al. (1996) “Intramuscular interferon beta-1a for disease progression in relapsing multiple sclerosis”, Ann Neurol. 39:285-294.
40. Jadidi-Niaragh et al., (2011) “Th17 cell, the new player of neuroinflammatory process in multiple sclerosis”, Scand J Immunol 74:1-13.
41. Jolivel et al. (2013) “Modulation of dendritic cell properties by laquinimod as a mechanism for modulating multiple sclerosis”, Brain 136, 1048-1066.
42. Kappos et al. (2010) “A placebo-controlled trial of oral fingolimod in relapsing multiple sclerosis”, N Eng J Md. 362:387-401.
43. Kurtzke J F. (1983) “Rating neurologic impairment in multiple sclerosis: an expanded disability status scale (EDSS)”, Neurology 33(11):1444-1452.
44. Li et al., (2011) “IL-9 is important for T-cell activation and differentiation in autoimmune inflammation of the central nervous system”, Eur J ImmunoL 41:2197-2206
45. Lublin and Reingold (1996) “Defining the clinical course of multiple sclerosis”, Neurol. 46:907-911.
46. Luo et al. (2009) “Interleukin-1 beta regulates proximal tubular cell transforming growth factor beta-1 signalling”, Nerol Dial Transplant 24, 2655-2665.
47. McDonald, (2001) “Guidelines from the International Panel on the Diagnosis of Multiple Sclerosis” Ann. Neurol. 50:121-127.
48. Mehta et al. (1996) “Magnetization transfer magnetic resonance imaging: a clinical review”, Topics in Magnetic Resonance Imaging 8(4):214-30.
49. Meng et al. (2013) “SASH1 regulates proliferation, apoptosis, and invasion of osteosarcoma cell”, Mol Cell Biochem 373, 201-210.
50. Miki et al. (1999) “Relapsing-Remitting Multiple Sclerosis: Longitudinal Analysis of MR Images—Lack of Correlation between Changes in T2 Lesion Volume and Clinical Findings”, Radiology. 213:395-399.
51. Miller et al. (2007) “MRI outcomes in a placebo-controlled trial of natalizumab in relapsing MS”, Neurology. 68:1390-1401.
52. Mirshafiey et al., (2009) “TGF-beta as a promising option in the treatment of multiple sclerosis”, Neuropharmacology 56:929-936
53. Murphy et al. (1995) “Regulation of interleukin 12 p40 expression through an NF-kappa B half-site”, Mol Cell Biol 15, 5258-5267.
54. Neuhaus et al. (2003) “Immunomodulation in multiple sclerosis: from immunosuppression to neuprotection”, Trends Pharmacol Sci. 24:131-138.
55. Noseworthy et al. (2000) “Multiple sclerosis”, N Engl J Med. 343:938-952.
56. Noseworthy et al. (2000) “Linomide in relapsing and secondary progressive MS. Part 1: Trial Design and clinical results”, Neurology. 54:1726-1733.
57. Oh et al. (2013) “TGF-beta: guardian of T cell function”, J Immunol 191, 3973-3979.
58. Panitch H, Goodin D S, Francis G, Chang P, Coyle P K, O'Connor P, Monaghan E, Li D, Weinsjenker B, for the EVIDENCE (Evidence of Interferon Dose-response: European North American Comparative Efficacy) Study Group and the University of British Columbia MS/MRI Research Group. (2002) “Randomized comparative study of interferon β-1a treatment regiments in MS”, The EVIDENCE Trial. Neurology. 59:1496-1506.
59. Polman et al. (2005) “Diagnostic criteria for multiple sclerosis: 2005 revisions to the McDonald Criteria”, Annals of Neurology, Volume 58 Issue 6, Pages 840-846.
60. Polman et al. (2005) “Treatment with laquinimod reduces development of active MRI lesions in relapsing MS”, Neurology. 64:987-991.
61. Polman et al. (2006) “A randomized, placebo-controlled trial of natalizumab for relapsing multiple sclerosis”, N Eng J Med. 354:899-910.
62. Ponighaus et al. (2007) “Human xylosyltransferase II is involved in the biosynthesis of the uniform tetrasaccharide linkage region in chondroitin sulfate and heparan sulfate proteoglycans”, J Biol Chem 282, 5201-5206.
63. Poser et al. (1983) “New Diagnostic Criteria for Multiple Sclerosis: Guidelines for Research Protocols”, Annals of Neurology March 1983, 13(3):227-230.
64. Preiningerova J. (2009) “Oral laquinimod therapy in relapsing multiple sclerosis”, Expert Opin Investig Drugs. 18:985-989.
65. PRISMS Study Group. Randomized double-blind placebo-controlled study of interferon β-1a in relapsing/remitting multiple sclerosis. Lancet 1998; 352:1498-1506.
66. Rosen Y. (2007) “The Recent advances in magnetic resonance neurospectroscopy”, Neurotherapeutics. 27(3): 330-45.
67. Rudick et al. (1999) “Use of the brain parenchymal fraction to measure whole brain atrophy in relapsing-remitting MS: Multiple Sclerosis Collaborative Research Group”. Neurology. 53:1698-1704.
68. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
69. Ruffini et al. (2013) “Laquinimod prevents inflammation-induced synaptic alterations occurring in experimental autoimmune encephalomyelitis”, Mult Scler 19, 1084-1094.
70. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
71. Sandberg-Wollheim et al. (2005) “48-week open safety study with high-dose oral laquinimod in patients”, Mult Scler. 11:S154 (Abstract).
72. Sanjabi et al. (2009) “Anti-inflammatory and pro-inflammatory roles of TGF-beta, IL-10, and IL-22 in immunity and autoimmunity”, Curr Opin Pharmacol 9, 447-453.
73. Schulze-Topphoff et al. (2012) “Laquinimod, a quinoline-3-carboxamide, induces type II myeloid cells that modulate central nervous system autoimmunity”, PLoS One 7, e33797.
74. SIENA and SIENAX available from the FMRIB Software Library, Oxford University. Oxford, UK; http//www.fmnrib.ox.ac.uk/analysis/research/siena/siena.
75. Sorenson P S. (2006) “Neutralising antibodies to interferon-β—measurement, clinical relevance, and management”, J Neurol. 253[Suppl 6]:VI/6-VI/22.
76. Sormani et al. (2004) “Measurement error of two different techniques for brain atrophy assessment in multiple sclerosis”, Neurology. 62:1432-1434.
77. Takami et al. (2012) “TGF-beta converts apoptotic stimuli into the signal for Th9 differentiation”, J Immunol 188, 4369-4375.
78. Temple R. (2006) “Hy's law: predicting serious hepatoxicity”, Pharmacoepdemiol Drug Saf. 15(4):241-3.
79. The IFNB Multiple Sclerosis Study Group. (1993) Interferon beta-1b is effective in relapsing-remitting multiple sclerosis. I. Clinical results of a multicenter, randomized, double-bind, placebo-controlled trial. Neurology; 43:655-661.
80. The IFNB Multiple Sclerosis Study Group. (1993) Interferon beta-1b is effective in relapsing-remitting multiple sclerosis. II. MRI analysis results of a multicenter, randomized, double-blind, placebo-controlled trial. Neurology; 43:662-667.
81. The National MS Society (USA), The Disease Modifying Drug Brochure, Oct. 19, 2006.
82. Thane and Gold (2011) “Laquinimod: a promising oral medication for the treatment of relapsing-remitting multiple sclerosis”, Expert Opin Drug Metab Toxicol. 2011 March; 7(3): 365-70.
83. Toubi et al. (2012) “Laquinimod modulates B cells and their regulatory effects on T cells in Multiple Sclerosis”, J Neuroimmunol 251, 45-54.
84. US Food and Drug Administration, Center for Drug Evaluation and Research. Peripheral and Central Nervous System (PCNS) Advisory Committee. US Department of Health and Human Services 2006. Briefing Document. Biogen Idec Biologics Marketing Application STN 125104/15. Natalizumab (Tysabri) for Multiple Sclerosis. Dated Feb. 9, 2006. Pages 45-48.
85. Wan et al. (2006) “The kinase TAK1 integrates antigen and cytokine receptor signaling for T cell development, survival and function”, Nat Immunol 7, 851-858.
86. Wegner et al., (2010) “Laquinimod interferes with migratory capacity of T cells and reduces IL-17 levels, inflammatory demyelination and acute axonal damage in mice with experimental autoimmune encephalomyelitis”, J. Neuroimmunol 227:133-143.
87. Yang et al., (2004) “Laquinimod (ABR-215062) suppresses the development of experimental autoimmune encephalomyelitis, modulates the Th1/Th2 balance and induces the Th3 cytokine TGF-0 in Lewis rats”, J. Neuroimmunol. 156:3-9.
88. Zou et al. (2002) “Suppression of experimental autoimmune neuritis by ABR-215062 is associated with altered Th1/Th2 balance and inhibited migration of inflammatory cells into the peripheral nerve tissue”, Neuropharmacology. 42:731.

What is claimed is:

1. A method of predicting clinical responsiveness to laquinimod therapy in a subject afflicted with multiple sclerosis or presenting a clinically isolated syndrome, the method comprising evaluating expression of a biomarker in the subject, so as to thereby predict clinical responsiveness to laquinimod, wherein the biomarker is a gene associated with inflammatory response, a gene associated with cellular movement, a gene associated with cell signaling, a gene associated with cell development, a gene associated with hematological system, or a combination thereof.

2. The method of claim 1, further comprising predicting positive clinical responsiveness to laquinimod if the biomarker is up-regulated in the subject.

3. The method of claim 2, wherein the subject is naïve to laquinimod.

4. The method of claim 1, further comprising predicting positive clinical responsiveness to laquinimod if the biomarker suppressed in the subject.

5. The method of claim 4, wherein the subject has previously received periodic laquinimod administration.

6. The method of claim 5, wherein the subject has received periodic laquinimod administration for at least one month, for at least 6 months, for at least 12 months, or for at least 24 months.

7. The method of any one of claims 1-6, wherein the gene associated with inflammatory response is a gene associated with or involved in TGFb signaling, IL-12 signaling, the pathway of adhesion of phagocytes, chemotaxis of neutrophils, transmigration of leukocytes, cavcolar mediated endocytosis, clathrin mediated endocytosis, and/or leukocyte extravasation signaling.

8. The method of any one of claims 1-7, wherein the gene associated with cellular movement is a gene associated with or involved in adhesion and migration of phagocytes, chemotaxis of neutrophils, transmigration of leukocytes, invasion of cells, adhesion of cells, and/or leukocyte extravasation signaling.

9. The method of any one of claims 1-8, wherein the gene associated with cell signaling is a gene associated with or involved in the pathway of adhesion of cells and/or neurotransmission.

10. The method of any one of claims 1-9, wherein the gene associated with cell development is a gene associated with or involved in the pathway of G protein coupled receptor signaling, arachidonic acid metabolism and/or TGFβ signaling.

11. The method of any one of claims 1-10, wherein the gene associated with hematological system is a gene associated with or involved in the pathway of aggregation of blood platelets, activation of blood platelets, aggregation of blood cells, coagulation of blood, intrinsic prothrombin activation pathway and/or coagulation system.

12. The method of any one of claims 1-11, wherein the gene is TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1 or a combination thereof.

13. The method of any one of claims 1-11, wherein the gene is ITGB1/3/5, CXCL5/7, BMP6, ITGA2/8, ITGB1/3/4/5/6, ITGBL1, MMP6/24/26/28, ADAM12/18/22, IL-5/20/22, IL-9/36, TNFRSF11A/B, TGβ, LTBP4, MEK1/2, Smad2/3/4, PAI-1, SELP, ITFA8, ITGB1/3/5, CXCL5/7, BMP6, ITGA2/8, ITGB1/3/4/5/6, ITGBL1, MMP16/24/26/28, ADAM12/18/22, IL-5/13/20/22, IL-9/11/36, TNFRSF11A/B, TGβ, LTBP4, MEK1/2, Smad1/2/3/4/5/6/8, PAI-1, CCL19, IKKg, LTBP1, Alpha tubulin, BMP4/7, MIS, TCF2, IL5R, IL13R, IL20R, ITGB2, NKTR, TEF, CLSTN2, LUC7L2, FABP7, TPTE, FSTL1, SF3B1, LIMS1, PDE5A, XPNPEP1, C5orf4, SPANXB1, SPANXB2, SPANXF1, KRT20, TBC1D1, GRHL2, C5orf4, SEPT6, KIAA1199, SSX21P, TPM1, CDC14B, USP47, MMRN1, CTNNAL1, SMOX, ALOX12, GLRA3, CA2, GUCY1B3, RFPL1, CLEC1B, GNG11, TSPAN32, RGS10, CALD1, PRKAR2B, CYP4F11, CLCA3P, CELSR3, CDC14B, TPM1, SEPT6, PRKG1, MAX, CCDC93, ARMCX6, LOC653354, TUBB2B, HIST1H2AJ, MFAP3L, LIMS1, GNB5, GPRASP1, SRRT, C1orf116, FBXO7, PPM1A, GUCY1B3, CTDSPL, GNAS, IGF2BP3, TPM1, HIST1H2BK, DLG4, WDR48, CALD1, LOC157627, GNB5, ZNF415, ASAP2, PSD3, GNAS, POPDC3, NRGN, ABL1M3, XYLT1, PTGIS, ARHGEF10, PDGFA, PGRMC1, HIST1H2AC, GNAS, CLDN5, MFAP3L, PGRMC1, MYST3, CAPRIN1, CALD1, FBXW7, DNM3, CD84, PRPF4B, RBM25, WASF3, GRAP2, SPARC, TAL1, NENF, XK, GP1BA, HLA-E, CA5A, LYVE1, MARCH6, NAT8B, TRIM58, RET, SDPR, TBXA2R, TMED10, APBA2, MYL9, POU1F1, H2BFS, HIST1H2BK, FAM12B, VCL, GSPT1, ALDOB, LOC150776, SMPD4, SLC37A1, SPARC, GNAS, TAS2R4, CALM3, POM121, POM121C, GRIK2, GREM1, TNNC2, EPS15L2, ENDOD1, RGS6, SF3B1, TMSB15A, ZBTB20, FUT9, ATP9A, MAX, HIST1H2AI, BAT2D1, ABL1, SNCA, GFI1B, CTSA, SNX13, RPA1, FLNA, XPNPEP1, KIF2A, ZBTB33, PSMD11, UBE2N, FOLR1, TSC22D1, PCNP, CELSR3, ACSBG1, RNF11, SEMA3E, MARCH2, PCDH24, SUPT5H, HLA-E, EGF, HLA-C, FLNA, CDK2AP1, LEPROT, SH3TC2, TUBA4A, MTMR1, TF, PRKD1, NAP1L1, DAB2, FUCA1, HIP1, THPO, MAP1B, PARVB, GP1BB, SEPT5, GJA4, PTGS1, GUCY1A3, HIST1H2AG, GNAS, LRBA, HYAL3, GP6, IGHG1, CYP2A13, CDC14B, MAX, KDM2A, CALD1, GNAZ, C19orf22, ARHGAP6, RHOC, RBX1, GP1BB, SEPT5, PRDX6, PRB4, FLNA, HIST1H2BF, RHBDF2, NUP205, SYT1, EGFL8, PPT2, TUBB1, TMC6, FLJ11292, NAP1L1, ALDH1A3, CSNK1E, PRUNE, COL4A3, ZNF221, ILF3, CABP5, RPA1, ARF1, HIST1H2BI, PTGS1, PRKAA1, GNB5, HIST2H4A, HIST2H4B, CYB5R3, TNS1, DCT, GMPR, ABI3BP, GNAS, SASH1, AAK1, XPO6, CTSL2, QSER1, MAP1LC3B, TBX6, CABP2, MRE11A, MAPRE2, TMC6, BDKRB2, MGLL, HRASLS, WHAMML1, WHAMML2, CLU, STC1, C6orf54, PABPN1, PDLIM1, CLU, PHF20, UBL4A, RNF115, HGD, RASGRP2, PNN, SAPS3, SFI1, GOLGA2, HIST2H2BE, SGEF, HGD, DUS1L, MPP1, HLA-E, GRB14, MMD, ZFHX4, CSNK1G2, HIST1H2BE, MPDZ, B2M, TBXA2R, CTDSPL, SNCA, CD99, POLS, MPL, HIST1H3F, SFRS8, NR5A2, ZMYM2, C6orf10, TMEM40, RNF43, PRUNE, MSH6, PLCB4, PARVB, TOX3, PKNOX1, RUFY1, SNCA, C10orf81, PDGFA, ASMT, HMGB1, CCDC90A, PROS1, hCG_1757335, RAP1B, MTSS1, GNRHR, LRRN3, MCM3AP, PLOD2, NAP1L1, PLOD2, HOXD13 CASKIN2, MFAP5, PITX2, SNCA, MYLK, PBX1, PRDX6, H3F3A, H3F3B, LOC440926, TMEM158, TRIM58, FSTL1, SNCA, TNS1, ATP1B1, C5orf4, LRP12, CTNNAL1, GEM, KIAA1466, ALDH1A2, MAP4K3, SNCA, RAB6B, PSD3, RIPK2, RAMP3, CALD1, CYP2E1, PSD3, PDLIM7, COBLL1, FUT3, SMOX, TGM2, LRRC50, CST6, OR7A17, C6orf145, DLEU2, DLEU2L, CPT2, HGF, TNS1, SPRY1, PLOD2, CD80, KYNU, BCAT1, NHLH1, AHCTF1, HOXA10, MTMR3, VAC14, CLCF1, FGF5, TAL1, SAMD14, ELL2, CHN1, SLC7A1, GRK5, PARD3, VPS37B, CYP2B6, CYP2B7P1, MALL, ALX4, SOX15, KRT5, ESPL1, STARD8, PSD3, KIAA0195, MYO9B, HIP1R, LOC100294412, EFNB1, ERN1, RHD, MFAP3L, PLA1A, POFUT2, C8orf39, CRYBB2, CYP4A1, PVRL2, CLCNKB, MRAS, NFIB, FKSG2, SLC11A2, FZR1, ZNF550, GLP1R, SLC19A1, RTN2, PAPOLA, STC1, GK, EXOSC6, RAPSN, HFE, EHD2, RIOK3, UBE2I, C15orf2, DMD, PRLH, MAP2K2, TP63, DACH1, PPP5C, SLC26A1, NUDT7, KCNJ12, ENTPD7, SLC26A, PRRG3, RGS6, ZBED2, FICD, ARHGAP1, ARHGDIA, SDHB, AMHR2, ABCA4, TCF20, BGN, CASP7, LPAR4, GNA12, CYP2W1, RAX, C4A, C4B, LOC100292046, LOC100294156, PXN, ESR2, MYL10, EFS, TFF3, SRPK1, LOC441601, BIRC5, CCT8L2, PPAP2B, CMA1, APOA2, KDELR2, ASCL3, RUNX1, BUB1, SLC6A8, HNRNPC, HNRNPCL1, LOC440563, LOC649330, RIBC2, CLIC4, RAB17, SCML2, SPINLW1, ANK1, EDA2R, HTR4, CDC42EP4, KANK2, ANK1, SYN1, DUX3, DUX4, FRG2C, HPX-2, LOC100134409, LOC652119, LOC653543, LOC653544, LOC653545, LOC728410, PKNOX2, MLLT4, APOA2, PENK, GNAT1, FURIN, SEMA6A, EGFL6, HRH1, TSPAN1, DBC1, TRPC7, GPR52, HAMP, PRSS2, GPR107, FLJ11292, FLJ20184, B34GALT1, NKX3-1, ASIP, EFCAB6, GPR20, CA5A, PLK4, TAAR5, SRPX2, CNTD2, AZGP1, TIMP3, RGS6, ADARB1, DYNC1I1, C10orf10, PDIA2, PITX3, HOXC13, LPAR3, CTRC, CTSL2, MUC8, AQP5, UGT1A1, UGT1A10, UGT1A4, UGT1A6, UGT1A8, UGT1A9, KCNQ2, CYP2A13, ZNF155, KIAA0892, ATP2A2, FGF5, FGF18, FUT2, SHROOM2, PRSS3, CREB3L1, MGAT2, PLCE1, MLXIPL, OR10H3, ABCB11, CD84, ARHGEF4, ORC1L, PCIF1, CD177, C1orf116, IFT122, C11orf20, DUSP13, C6orf208, PLA2G5, PRAMEF1, PRAMEF2, CYP4F8, KCNA1, MFAP4, SLC4A3, IL1RAPL1, SERPINE1, ZCCHC14, POLR3G, C16orf68, FLJ14100, SMCHD1, ASCL1, FOXA2, SLC23A2, KLK13, MTSS1L, DNMT3L, RREB1, DNMBP, PKLR, C1orf106, CCDC134, MTSS1, CCDC40, HOXB1, SCNN1B, SEMA4G, RAPGEFL1, MAGEL2, PLSCR2, CHD2, PLCD1, C1orf116, CHRNA2, MBP, CDC42BPA, MYF6, PI15, LOC440895, SBF1, MAST1, GLT8D2, ERBB3, LOH3CR2A, AMH, HR, RDH8, PAWR, DRD3, CCT8, PRELP, SPOCK3, EPS8L3, NXN, SEMA4G, P2RY1, AVL9, TEK, MOGAT2, KLK7, MT1E, MT1H, MT1M, CLDN18, RHBDF2, SIX1, INPP5A, KCNMB3, MAP2K5, GPD1, LPO, LOC729143, MPRIP, WNT7A, RARG, CDH7, MBNL2, RASGRP2, RBMY2FP, MASP1, CASR, EGR4, APOC2, HECW1, HOXB3, IRF5, NNMT, AOC2, ESRRG, LPIN1, ACOT11, CCDC33, MBD2, ZNF323, NTRK2, TMEM151B3, GPLD1, LENEP, HNF1B, NXPH3, ALDH1A3, PHF20L1, CKM, PARD6B, CRYGB, HAB1, LARGE, RAB40C, MPL, CHIT1, METTL10, DUS4L, PNLIPRP1, ELL, ST8SIA5, GRIN2B, MC4R, RTDR1, HDAC6, KCNJ13, CPSF1, SPANXC, CNOT4, LAMA2, SLC1A6, ABCA2, KLK11, GFRA3, CYP3A4, SLC1A3, ATP2B2, APBB2, VPS45, GHRHR, HOXD4, PRPH, ADCY2, LEFTY2, CYP1B1, PCP4, C8B, RANBP3, PDE6H, TRIM15, VGLL1, TRIM3, CRKL, ADH7, PSG3, GPR153, MFAP2, FGF13, NAPA, ALDH3A1, MCM10, TLE4, ITPR3, CCDC87, C9orf7, ACTC1, OBSL1, MAP2, CRYM, RNF122, SST, HLA-DRB6, SLC22A17, HSPG2, HIP1, GRIK2, UNKL, GPR144, KIR3DX1, NARFL, UCP3, PLXNA2, BTN1A1, ERCC4, CIITA, EGFR, KRT33A, CLTB, B3GALT5, AP3M2, GJC1, MYO3A, ARHGAP1, PPP2R3A, CLIC4, C20orf195, SIGLEC8, GPRC5A, CACNB1, MYL10, PRLR, OR2S2, NCR2, CHAF1B, EYA3, CDS1, FBXL18, ACTL6B, ZNF821, C16orf71, HBBP1, PLXNA1, CDC45L, MTCP1, PLCB34, PLVAP, PROX1, CYP3A43, IGHG1, RECQL5, IDUA, DLGAP4, PLXNB1, HSD17B14, FOXP3, C19orf26, EPB41L1, RBBP9, GJB4, UPK1B, CYP19A1, LOC55908, CLDN18, C2orf72, NTRK3, NRXN2, SPDEF, IGH@, IGHD, IGHG1, IGHM, LOC100289944, VSIG6, ACRV1, PHLDB1, SORBS1, HAPLN2, FABP3, EFS, ACVR1B, CHST3, UGT2A1, UGT2A2, TAF1, MT4, MFAP3, ETV5, UBQLN3, TBX10, GJB1, ABO, SPINK5, ATAD4, CDH11, CARD14, ALPP, ALPPL2, CBL, LRP4, CDKL2, SSX3, DSG2, SLC45A2, LAMA4, WFDC8, HTR7, EFNB3, TUBB2B, OR7E19P, PMS2L4, ASAP3, FRZB, PDLIM4, PVT1, TFR2, AHI1, TAF4, ADAMTSL2, CLDN4, KIR2DL1, KIR2DL2, KIR2DL3, KIR2DL5A, KIR2DL5B, KIR2DS1, KIR2DS2, KIR2DS4, KIR2DS5, KIR3DL2, KIR3DL3, KIR3DP1, LOC727787, RAPGEF5, CRMP1, LDB3, F11, USP46, IBSP, SLC9A3, FLRT3, TRIM17, FGF17, CAMK1G, GLYR1, CSH1, NTF3, ABHD6, TRIM15, OR52A1, FGFR2, ORAI2, C17orf53, GLP1R, SLIT1, TP63, DDR1, CFTR, DIO2, LETM1, ACSM5, ACTA1, NPR1, KCND3, POPDC3, DNAH3, SPDEF, CLEC4M, SLC30A3, NAGLU, AAK1, DHX34, NNAT, AKAP9, ICMT, FAM189A1, C10orf81, MYOZ1, PKNOX2, MGC31957, PRDM11, RET, IGHG1, XPNPEP2, NTRK2, SLC25A10, NR1I2, GRM8, OR3A3, GIPR, PAH, PACRG, CLN8, ZNF2I5, TRIO, TTLL5, GRM1, PRKG1, HHLA1, LAMA3, SLC37A4, HOXC11, SLCO5A1, CA10, RRBP1, SOD3, NTRK3, CYR61, STRA6, SLC6A11, CNOT4, ATN1, BCAP29, NOVA2, RELN, LAMC2, RAD51, PRSS7, DCBLD2, TACR2, RAB11B, OR2J2, VSNL1, IFNA17, DPYSL4, MGC2889, RRBP1, POLQ, OR1A2, PURA, AIF1, CBS, NECAB2, PRKCE, NOX1, IHH, EXO1, GPRIN2, PDX1, GPR12, FAM188A, HS3ST3B1, ASCL1, ZNF484, CSH1, BCAN, DDN, DUOX2, MORN1, SLC39A2, CLCN7, RUNX2, TTYH1, ZNF280B, PAX3, LZTS1, SLC8A2, HAB1, KIF1A, ARL4D, UGT2B15, NACA2, THRB, C6orf15, GPR176, WSCD1, PLXNB3, CADM3, HAP1, CYP1A2, SPAM1, IL22RA1, CDC2L5, IRX5, PPFIA2, KDELR3, CEACAM7, KCMF1, DUOX1, CDC27, HIST2H2AA3, CAV3, APOA4, NPR3, PRG3, TBC1D22B, TUSC3, RIMS2, CYP4F12, TBXA2R, HBEGF, PSG9, PYGO1, RASGRF1, SCN2A, KLHL1, DTNB, GREM1, SNCG, C22orf24, PALM, COBLL1, DNPEP, MNS1, NFATC4, DLC1, HSPC072, MCAM, CA12, CSHL1, RPA1N, COL5A2, UGT1A8, UGT1A9, IGH@, IGHA1, IGHG1, IGHG2, IGHG3, IGHM, LOC100126583, LOC100290036, LOC100290320, LOC100293211, LOC652494, ACSM5, ALOX12P2, ERBB4, CLDN16, CIB2, GALR3, MSMB, FABP7, ATXN3, KCNJ5, TRDN, CYP3A43, BAZ2A, ACCN4, SILV, DGCR14, SEMA6C, DIO2, PTHLH, LEP, PDZRN3, RGSL1, GJA4, SLC22A6, RASGRF1, MAPRE2, PVRL1, AKAP1, POMP, SOX21, DNAH9, HOXC5, SERHL2, KIAA0485, ITSN1, B4GALT1, NEK2, NUPR1, CCDC93, EPO, CRABP2, TYRO3, GOLGA2, SEMA3F, BFSP2, NCAM1, FOLH1, SSX2, TMPRSS4, DCN, LPHN3, POU4F3, CEACAM5, BCL3, EXTL3, CCNA1, DDR2, PAX8, SOX5, POU3F1, PEX16, NUP62, SIGLEC11, ALDOB, GPC3, IGFALS, WDR25, FGF1, OSR2, ARID1A, GYPA, KLK13, PAR VB, LILRB5, RIMS2, C19orf21, HOXD1, PRSS3, FLT1, ATP6V1C1, LOX, CRYBB3, CA12, PRKG2, MASP1, LOC728395, LOC728403, TSPY1, PDCD1, GGTLC1, AQP8, KRT16, AICDA, BRD8, C1orf95, OR3A2, PFKFB2, FRZB, PAK3, MEIS2, ZSCAN2, MYH7, VWA1, LSAMP, SRC, UGT1A1, UGT1A10, UGT1A3, UGT1A4, UGT1A5, UGT1A6, UGT1A7, UGT1A8, UGT1A9, DIO1, TADA3L, NFASC, CALCRL, NBLA00301, MAB2 L1, FBXO42, COL10A1, CFB, SNX7, FOXN1, SRY, HLF, CLCA3P, DAZ1, DAZ2, DAZ3, DAZ4, GPR3, TMPRSS11E, EMID1, KCNMB2, MUC5AC, SORT1, HIF3A, MAPK4, TCP11L1, ZZEF1, DCAF7, DMWD, CLCA2, VAC14, CSPG5, STMN2, MLLT4, GALNT14, FGF12, MFAP5, SUMO3, HTR3A, GDF5, TSSK1B, CYP2A7P1, MARK1, ATP1B2, TBX6, PAX8, IL1R1, RALYL, OR2B2, TAAR3, C12orf32, IGHG1, LOC642131, DICER1, GLRA3, PPARD, HSPA4L, WNT2, VIPR2, CYP2C9, SRPX2, IGSF1, ALPK3, TFPI, KCNS3, MARCH8, FRMD4B, TACR3, FIGF, PDCD6, TNN, SPANXB1, SPANXB2, SPANXF1, RHBDD3, SPP2, PDE10A, ZNF224, FGL1, PGAM2, CADM4, APOBEC2, SLC9A5, GNAT1, ARHGEF16, SMARCA2, DNAH9, RBM26, WNT2B, KCNK2, NPBWR2, SP2, TMPRSS11D, DENND2A, TNIP3, STC1, DOCK6, ADAM5P, SYDE1, TNPO2, LRTM1, USH1C, PDE12, SRCAP, OR10J1, OR2H2, KCNJ8, RP11-257K9.7, DOCK5, TPD52L1, PAEP, GGA2, PHLDA3, HES2, MLL, CHRNA6, CIB2, PTPRF, TM7SF4, DAZ1, DAZ2, DAZ3, DAZ4, ALX1, OR2F1, OR2F2, PLAT, HGC6.3, WNT11, PGK2, SNAI2, COL4A6, PRUNE2, ANKS1B, LOC81691, FERMT2, TIMP3, CST8, CAPN6, IDUA, GPR32, AKR1B10, GRHL2, FBXO24, HSF4, IGHG1, HCN2, LRP12, ARHGEF15, UGT1A1, UGT1A10, UGT1A7, UGT1A8, GUCA2A, ITIH1, EGFR, UGT1A1, UGT1A10, UGT1A3, UGT1A4, UGT1A5, UGT1A6, UGT1A7, UGT1A8, UGT1A9, MYOG, TMSB15A, TLX1, EDNRA, LOC100289791, MDFI, ZER1, MYH15, CDH20, GPR63, LOC440345, LOC440354, LOC595101, LOC641298, SMG1, HOXC10, KRTAP1-1, ARSD, CPLX3, LMAN1L, IFNA4, ABCC1, SEMA3E, MRE11A, C1QL1, LIPF, TRIM9, BBOX1, LRRC17, WNT2B, CYP3A4, SI, ANO3, OBSL1, CHRD, MSX2, PSG1, FAM107A, LRRC37B2, ANKLE2, PAX2, UNC5B, ADCYAP1R1, HFE, SYT1, GJC2, LOC100293871, FGF8, ACRV1, NRXN1, GDPD2, RGS4, CELA2A, IFNW1, MLNR, RNF17, LAD1, GLRA2, RASL12, MAGOH2, C6orf54, ZNF214, IKBKG, AP4E1, ZNRF4, OSBPL10, C1orf175, TTC4, PCDHB3, ADRBK1, ITSN1, XAGE1A, XAGE1B, XAGE1C, XAGE1D, XAGE1E, CDH22, FARP2, MYT1, TNC, MUC5AC, SLC6A15, PP14571, SMR3A, SMR3B, RXRG, SNX1, GLP1R, C6orf155, ATP1A2, TFAP4, PNPLA2, DIRAS3, ANO2, TACSTD2, MCM3AP, IL13RA2, TRIM10, RTEL1, PRRX2, TSHB, TIMELESS, FMO1, KIF18A, KIAA1199, CALB2, MFAP3L, PTGER3, EPAS1, SQSTM1, TSPY1, CPM, DLGAP1, CYP4F11, TLX3, PCDHA10, TAOK2, ERC1, TBX2, KALRN, DICER1, PAPPA, KIF5A, DNAJC22, OTUB1, KIAA1644, SEZ6L2, PCNXL2, HMHB1, ERG, SNTB2, GJA5, AGTR2, GJA3, GCK, LRRC61, CNTF, ZFP91, ZFP91-CNTF, PDLIM4, MPPED2, IFNA10, ACTN2, VGLL1, GJA9, LDLR, ANK2, COL1A1, TIMP3, OTOF, AGXT, GLI2, TRMT61A, FOXD2, TMEM212, DENND2A, B3GALT1, SPAG11A, PRDM4, TF, ELF5, GSC2, EPB41L4B, GYG2, LYZL6, DCHS2, OBP2A, OBP2B, ANGPTL3, MYH11, NES, SLC17A1, RBM15B, CSH1, HTR5A, CYP3A7, HTR2A, KCNV2, TOX3, CLOCK, MAGEA6, FAM12A, COL4A3, S1PR2, NAT8, ACE2, SLC22A6, SLC13A2, MYH4, APBB2, RAP1GAP, SHOX2, SLCO1A2, ETV1, MAGEA12, PLA2G6, ADRA1A, SYT5, GPR161, SEMA3F, CYP3A43, HOMER2, KCNJ5, PPL, COL17A1, CSHL1, C9orf116, PARK2, UGT2B15, CDK6, FAM174B, CELA2A, CELA2B, SPDEF, EPB41, GAB1, SMR3A, PDE6G, COL5A1, ABCA6, DMD, CYLC2, CIDEA, RAG2, HIST1H2BN, FMO6P, MAOA, ANKRD53, HAPLN1, MT1M, EHD2, GAD2, CRISP2, CSN2, SULT1C2, PCDHGA3, SSX3, FGFR2, GPR161, ATN1, CHD5, A4GALT, MYBPH, CSHL1, NCAPH2, CAPN9, CNGB1, BCAM, DRD5, NR5A2, TEF, ELAVL2, DGKB, HTR7P, RHAG, GH2, COL4A6, BMP7, SOSTDC1, SOX14, TAS2R9, LPHN2, MAP1A, OSGIN2, SLC10A2, FAM13C, EMX1, FLJ40330, CHI3L1, CDH16, SPRR1A, LOX, CALCB, GABBR2, CPB2, RASL11B, CCDC81, RUNX1, CPA1, CLCNKA, CLCNKB, FHL5, THSD7A, TFAP2C, SPAG11B, CAP2, PODNL1, SSX4, SSX4B, G6PC, RPE65, TMEM222, KDR, CHP2, GPR64, TPM2, TCEB3B, E2F5, IL5RA, AOC3, ABCF3, CPN2, ACE, NRP2, INPP5J, SMAD9, FAM155A, GART, PIR, ZNF467, ITSN2, NR1D1, THRA, RP11-35N6.1, LAMB1, EPHB3, PLA2R1, RAPGEF4, DNAJC8, ARSJ, TRIM49, GC, CDH2, ATXN3L, BTF3L1, BICC1, FAM186A, PTPRF, TRPC4, TCL6, CYP4A22, FUT6, MUC1, DKFZP434B2016, LOC643313, LDHA, LOC100131613, TRIM3, MLLT10, DZIP1, ANKRD34C, BUB1, CSPG5, FBLN1, GAD2, CLDN1, CHRNA3, SCN11A, TEX11, IL20RA, AKAP5, KBTBD10, MSTN, TLL2, NACAD, UNC93A, PTGER1, OLAH, NHLH2, SERPINA6, KRT117, KCNMA1, PRKCA, STS, LAMA1, GPR88, ACTN2, TREH, AKAP4, DKK4, PRICKLE3, IRS4, TRPV4, PCDH11Y, APBB2, SLCO2A1, DRD2, MTMR7, ZNF471, TF, NRIP2, ST6GALNAC5, COMT, PAH, LRRC19, PRKAR1B, HPR, PRDM5, NCRNA00120, LOC79999, ITSN2, CACNB2, GPR98, PREX2, FAM182B, LAMA4, ARVCF, HAS2, YOD1, PPP2R3A, COL4A1, RBM12B, GSTA3, FAM66D, OR10H2, PTHLH, ZNF674, KRT19, ACCN2, COL6A1, LOC100288442, LOC100289169, LOC728888, LOC729602, NPIPL2, NPIPL3, PDXDC2, SLC37A1, ATP6V1B1, ABI3BP, HR44, ZNF324B, ZNF584, HOXD13, ADH6, IFNA8, MYOZ2, NFATC4, ADAMTS7, FOXL1, GPR17, SLC18A3, MYH6, BOK, FGA, TEAD4, GRM1, EDNRA, C8orf79, METTL7A, FOLH1, RAD54L, SOX11, CNOT3, NTS, MAPK12, DOCK6, DNAJC6, HS3ST3A1, LOC728395, TSPY1, TSPY3, PTH, LAMB4, ALDOB, FLG, MLANA, UBE2D4, LOC100287483, KRT20, POU1F1, SLCO1B3, CLTA, MECOM, C8orf11, SULT2A1, C6orf10, SLC27A6, PRKD1, SYNPO2L, THPO, GABRR1, CFTR, PPP2R3A, DCBLD2, ANP32A, ANP32C, ANP32D, LOC723972, XYLT1, STAB1, STAB1, SASH1, PID1, FUCA1, SASH1, LRRN3, LRRN3 or a combination thereof.

14. A method of treating a subject afflicted with multiple sclerosis or presenting a clinically isolated syndrome with laquinimod, comprising the steps of:
a) determining whether the subject is a laquinimod responder by evaluating expression of a biomarker in the subject, and
b) administering to the subject an amount of laquinimod effective to treat the subject only if the subject is identified as a laquinimod responder, so as to thereby treat the subject,
wherein the biomarker is a gene associated with inflammatory response, a gene associated with cellular movement, a gene associated with cell signaling, a gene associated with cell development, a gene associated with hematological system, or a combination thereof.

15. A method for treating a subject afflicted with multiple sclerosis or presenting a clinically isolated syndrome comprising the steps of:
a) administering to the subject a therapeutically effective amount of laquinimod,
b) determining whether the subject is a laquinimod responder by evaluating expression of a biomarker in the subject; and
c) administering to the subject an amount of laquinimod effective to treat the subject only if the subject is identified as a laquinimod responder, or modifying the administration of laquinimod to the subject if the subject is not identified as a laquinimod responder, so as to thereby treat the subject,
wherein the biomarker is a gene associated with inflammatory response, a gene associated with cellular movement, a gene associated with cell signaling, a gene associated with cell development, a gene associated with hematological system, or a combination thereof.

16. The method of claims 14 and 15, wherein the subject is identified as a laquinimod responder if the biomarker is up-regulated in the subject.

17. The method of claims 14 and 15, wherein the subject is identified as a laquinimod responder if the biomarker is suppressed in the subject.

18. The method of any one of claims 14-17, wherein the gene associated with inflammatory response is a gene associated with or involved in TGFb signaling, IL-12 signaling, the pathway of adhesion of phagocytes, chemotaxis of neutrophils, transmigration of leukocytes, caveolar mediated endocytosis, clathrin mediated endocytosis, and/or leukocyte extravasation signaling.

19. The method of any one of claims 14-18, wherein the gene associated with cellular movement is a gene associated with or involved in adhesion and migration of phagocytes, chemotaxis of neutrophils, transmigration of leukocytes, invasion of cells, adhesion of cells, and/or leukocyte extravasation signaling.

20. The method of any one of claims 14-19, wherein the gene associated with cell signaling is a gene associated with or involved in the pathway of adhesion of cells and/or neurotransmission.

21. The method of any one of claims 14-20, wherein the gene associated with cell development is a gene associated with or involved in the pathway of G protein coupled receptor signaling, arachidonic acid metabolism and/or TGFβ signaling.

22. The method of any one of claims 14-21, wherein the gene associated with hematological system is a gene associated with or involved in the pathway of aggregation of blood platelets, activation of blood platelets, aggregation of blood cells, coagulation of blood, intrinsic prothrombin activation pathway and/or coagulation system.

23. The method of any one of claims 14-22, wherein the gene is TNFSF4, SELP, ITFA8, ITGB1/3/5, CXCL5/7, a BMP6 gene, ITGA2/8, ITGβ1/3/4/5/6, ITGBL1, MMP16/24/26/28, ADAM12/18/22, IL-1/1R/5/8/13/20/22R, IL-9/11/12/36, TNFRSF11A/B, IFNA4/8/10/17, TGβ, LTBP4, MEK1/2, TGFβ type 1 receptor, type II BMPR, smad1/2/3/4/5/6/8, PAI-1, CCL19, IKKg, LTBP1 or a combination thereof.

24. The method of any one of claims 14-22, wherein the gene is ITGB1/3/5, CXCL5/7, BMP6, ITGA2/8, ITGB1/3/4/5/6, ITGBL1, MMP16/24/26/28, ADAM12/18/22, IL-5/20/22, IL-9/36, TNFRSF11A/B, TGβ, LTBP4, MEK1/2, Smad2/3/4, PAI-1, SELP, ITFA8, ITGB1/3/5, CXCL5/7, BMP6, ITGA2/8, ITGB1/3/4/5/6, ITGBL1, MMP16/24/26/28, ADAM12/18/22, IL-5/13/20/22, IL-9/11/36, TNFRSF11A/B, TGβ, LTBP4, MEK1/2, Smad1/2/3/4/5/6/8, PAI-1, CCL19, IKKg, LTBP1, Alpha tubulin, BMP4/7, MIS, TCF2, IL5R, IL13R, IL20R, ITGB2, NKTR, TEF, CLSTN2, LUC7L2, FABP7, TPTE, FSTL1, SF3B1, LIMS1, PDE5A, XPNPEP1, C5orf4, SPANXB1, SPANXB2, SPANXF1, KRT20, TBC1D1, GRHL2, C5orf4, SEPT6, KIAA1199, SSX21P, TPM1, CDC14B, USP47, MMRN1, CTNNAL1, SMOX, ALOX12, GLRA3, CA2, GUCY1B3, RFPL1, CLEC1B, GNG11, TSPAN32, RGS10, CALD1, PRKAR2B, CYP4F11, CLCA3P, CELSR3, CDC14B, TPM1, SEPT6, PRKG1, MAX, CCDC93, ARMCX6, LOC653354, TUBB2B, HIST1H2AJ, MFAP3L, LIMS1, GNB5, GPRASP1, SRRT, C1orf116, FBXO7, PPM1A, GUCY1B3, CTDSPL, GNAS, IGF2BP3, TPM1, HIST1H2BK, DLG4, WDR48, CALD1, LOC157627, GNB5, ZNF415, ASAP2, PSD3, GNAS, POPDC3, NRGN, ABL1M3, XYLT1, PTGIS, ARHGEF10, PDGFA, PGRMC1, HIST1H2AC, GNAS, CLDN5, MFAP3L, PGRMC1, MYST3, CAPRIN1, CALD1, FBXW7, DNM3, CD84, PRPF4B, RBM25, WASF3, GRAP2, SPARC, TAL1, NENF, XK, GP1BA, HLA-E, CA5A, LYVE1, MARCH6, NAT8B, TRIM58, RET, SDPR, TBXA2R, TMED10, APBA2, MYL9, POU1F1, H2BFS, HIST1H2BK, FAM12B, VCL, GSPT1, ALDOB, LOC150776, SMPD4, SLC37A1, SPARC, GNAS, TAS2R4, CALM3, POM121, POM121C, GRIK2, GREM1, TNNC2, EPS15L2, ENDOD1, RGS6, SF3B1, TMSB15A, ZBTB20, FUT9, ATP9A, MAX, HIST1H2AI, BAT2D1, ABL1, SNCA, GFI1B, CTSA, SNX13, RPA1, FLNA, XPNPEP1, KIF2A, ZBTB33, PSMD11, UBE2N, FOLR1, TSC22D1, PCNP, CELSR3, ACSBG1, RNF11, SEMA3E, MARCH2, PCDH24, SUPT5H, HLA-E, EGF, HLA-C, FLNA, CDK2AP1, LEPROT, SH3TC2, TUBA4A, MTMR1, TF, PRKD1, NAP1L1, DAB2, FUCA1, HIP1, THPO, MAP1B, PARVB, GP1BB, SEPT5, GJA4, PTGS1, GUCY1A3, HIST1H2AG, GNAS, LRBA, HYAL3, GP6, IGHG1, CYP2A13, CDC14B, MAX, KDM2A, CALD1, GNAZ, C19orf22, ARHGAP6, RHOC, RBX1, GP1BB, SEPT5, PRDX6, PRB4, FLNA, HIST1H2BF, RHBDF2, NUP205, SYT1, EGFL8, PPT2, TUBB1, TMC6, FLJ11292, NAP1L1, ALDH1A3, CSNK1E, PRUNE, COL4A3, ZNF221, ILF3, CABP5, RPA1, ARF1, HIST1H2BI, PTGS1, PRKAA1, GNB5, HIST2H4A, HIST2H4B, CYB5R3, TNS1, DCT, GMPR, ABI3BP, GNAS, SASH1, AAK1, XPO6, CTSL2, QSER1, MAP1LC3B, TBX6, CABP2, MRE11A, MAPRE2, TMC6, BDKRB2, MGLL, HRASLS, WHAMML1, WHAMML2, CLU, STC1, C6orf54, PABPN1, PDLIM1, CLU, PHF20, UBL4A, RNF115, HGD, RASGRP2, PNN, SAPS3, SFI1, GOLGA2, HIST2H2BE, SGEF, HGD, DUS1L, MPP1, HLA-E, GRB14, MMD, ZFHX4, CSNK1G2, HIST1H2BE, MPDZ, B2M, TBXA2R, CTDSPL, SNCA, CD99, POLS, MPL, HIST1H3F, SFRS8, NR5A2, ZMYM2, C6orf10, TMEM40, RNF43, PRUNE, MSH6, PLCB4, PARVB, TOX3, PKNOX1, RUFY1, SNCA, C10orf81, PDGFA, ASMT, HMGB1, CCDC90A, PROS1, hCG_1757335, RAP1B, MTSS1, GNRHR, LRRN3, MCM3AP, PLOD2, NAP1L1, PLOD2, HOXD13 CASKIN2, MFAP5, PITX2, SNCA, MYLK, PBX1, PRDX6, H3F3A, H3F3B, LOC440926, TMEM158, TRIM58, FSTL1, SNCA, TNS1, ATP1B1, C5orf4, LRP12, CTNNAL1, GEM, KIAA1466, ALDH1A2, MAP4K3, SNCA, RAB6B, PSD3, RIPK2, RAMP3, CALD1, CYP2E1, PSD3, PDLIM7, COBLL1, FUT3, SMOX, TGM2, LRRC50, CST6, OR7A17, C6orf145, DLEU2, DLEU2L, CPT2, HGF, TNS11, SPRY1, PLOD2, CD80, KYNU, BCAT1, NHLH1, AHCTF1, HOXA10, MTMR3, VAC14, CLCF1, FGF5, TAL1, SAMD14, ELL2, CHN1, SLC7A1, GRK5, PARD3, VPS37B, CYP2B6, CYP2B7P1, MALL, ALX4, SOX15, KRT5, ESPL1, STARD8, PSD3, KIAA0195, MYO9B, HIP1R, LOC100294412, EFNB1, ERN1, RHD, MFAP3L, PLA1A, POFUT2, C8orf39, CRYBB2, CYP4A11, PVRL2, CLCNKB, MRAS, NFIB, FKSG2, SLC11A2, FZR1, ZNF550, GLP1R, SLC19A1, RTN2, PAPOLA, STC1, GK, EXOSC6, RAPSN, HFE, EHD2, RIOK3, UBE2I, C15orf2, DMD, PRLH, MAP2K2, TP63, DACH1, PPP5C, SLC26A1, NUDT7, KCNJ12, ENTPD7, SLC26A1, PRRG3, RGS6, ZBED2, FICD, ARHGAP1, ARHGDIA, SDHB, AMHR2, ABCA4, TCF20, BGN, CASP7, LPAR4, GNA12, CYP2W1, RAX, C4A, C4B, LOC100292046, LOC100294156, PXN, ESR2, MYL10, EFS, TFF3, SRPK1, LOC441601, BIRC5, CCT8L2, PPAP2B, CMA1, APOA2, KDELR2, ASCL3, RUNX1, BUB1, SLC6A8, HNRNPC, HNRNPCL1, LOC440563, LOC649330, RIBC2, CLIC4, RAB17, SCML2, SPINLW1, ANK1, EDA2R, HTR4, CDC42EP4, KANK2, ANK1, SYN1, DUX3, DUX4, FRG2C, HPX-2, LOC100134409, LOC652119, LOC653543, LOC653544, LOC653545, LOC728410, PKNOX2, MLLT4, APOA2, PENK, GNAT1, FURIN, SEMA6A, EGFL6, HRH1, TSPAN1, DBC1, TRPC7, GPR52, HAMP, PRSS2, GPR107, FLJ11292, FLJ20184, B4GALT1, NKX3-1, ASIP, EFCAB6, GPR20, CA5A, PLK4, TAAR5, SRPX2, CNTD2, AZGP1, TIMP3, RGS6, ADARB1, DYNC1I1, C10orf10, PDIA2, PITX3, HOXC13, LPAR3, CTRC, CTSL2, MUC8, AQP5, UGT1A1, UGT1A10, UGT1A4, UGT1A6, UGT1A8, UGT1A9, KCNQ2, CYP2A13, ZNF155, KIAA0892, ATP2A2, FGF5, FGF18, FUT2, SHROOM2, PRSS3, CREB3L1, MGAT2, PLCE1, MLXIPL, OR10113, ABCB11, CD84, ARHGEF4, ORC1L, PCIF1, CD177, C1orf116, IFT122, C11orf20, DUSP13, C6orf208, PLA2G5, PRAMEF1, PRAMEF2, CYP4F8, KCNA1, MFAP4, SLC4A3, IL1RAPL1, SERPINE1, ZCCHC14, POLR3G, C16orf68, FLJ14100, SMCHD1, ASCL1, FOXA2, SLC23A2, KLK13, MTSS1L, DNMT3L, RREB1, DNMBP, PKLR, C1orf106, CCDC134, MTSS1, CCDC40, HOXB1, SCNN1B, SEMA4G, RAPGEFL1, MAGEL2, PLSCR2, CHD2, PLCD1, C1orf116, CHRNA2, MBP, CDC42BPA, MYF6, PI15, LOC440895, SBF1, MAST1, GLT8D2, ERBB3, LOH3CR2A, AMH, HR, RDH8, PAWR, DRD3, CCT8, PRELP, SPOCK3, EPS8L3, NXN, SEMA4G, P2RY1, AVL9, TEK, MOGAT2, KLK7, MT1E, MT1H, MT1M, CLDN18, RHBDF2, SIX1, INPP5A, KCNMB3, MAP2K5, GPD1, LPO, LOC729143, MPRIP, WNT7A, RARG, CDH7, MBNL2, RASGRP2, RBMY2FP, MASP1, CASR, EGR4, APOC2, HECW1, HOXB3, IRF5, NNMT, AOC2, ESRRG, LPIN1, ACOT11, CCDC33, MBD2, ZNF323, NTRK2, TMEM151B, GPLD1, LENEP, HNF1B, NXPH3, ALDH1A3, PHF20L1, CKM, PARD6B, CRYGB, HAB1, LARGE, RAB40C, MPL, CHIT1, METTL10, DUS4L, PNLIPRP1, ELL, ST8SIA5, GRIN2B, MC4R, RTDR1, HDAC6, KCNJ13, CPSF1, SPANXC, CNOT4, LAMA2, SLC1A6, ABCA2, KLK11, GFRA3, CYP3A4, SLC1A3, ATP2B2, APBB2, VPS45, GHRHR, HOXD4, PRPH, ADCY2, LEFTY2, CYP1B1, PCP4, C8B, RANBP3, PDE6H, TRIM15, VGLL1, TRIM3, CRKL, ADH7, PSG3, GPR153, MFAP2, FGF13, NAPA, ALDH3A1, MCM10, TLE4, ITPR3, CCDC87, C9orf7, ACTC1, OBSL1, MAP2, CRYM, RNF122, SST, HLA-DRB6, SLC22A17, HSPG2, HIP1, GRIK2, UNKL, GPR144, KIR3DX1, NARFL, UCP3, PLXNA2, BTN1A1, ERCC4, CIITA, EGFR, KRT33A, CLTB, B3GALT5, AP3M2, GJC1, MYO3A, ARHGAP1, PPP2R3A, CLIC4, C20orf195, SIGLEC8, GPRC5A, CACNB1, MYL10, PRLR, OR2S2, NCR2, CHAF1B, EYA3, CDS1, FBXL18, ACTL6B, ZNF821, C16orf71, HBBP1, PLXNA1, CDC45L, MTCP1, PLCB4, PLVAP, PROX1, CYP3A43, IGHG1, RECQL5, IDUA, DLGAP4, PLXNB1, HSD17B14, FOXP3, C19orf26, EPB4 L1, RBBP9, GJB4, UPK1B, CYP19A1, LOC55908, CLDN18, C2orf72, NTRK3, NRXN2, SPDEF, IGH@, IGHD, IGHG1, IGHM, LOC100289944, VSIG6, ACRV1, PHLDB1, SORBS1, HAPLN2, FABP3, EFS, ACVR1B, CHST3, UGT2A1, UGT2A2, TAF1, MT4, MFAP3, ETV5, UBQLN3, TBX10, GJB1, ABO, SPINK5, ATAD4, CDH11, CARD14, ALPP, ALPPL2, CBL, LRP4, CDKL2, SSX3, DSG2, SLC45A2, LAMA4, WFDC8, HTR7, EFNB3, TUBB2B, OR7E19P, PMS2L4, ASAP3, FRZB, PDLIM4, PVT1, TFR2, AHI1, TAF4, ADAMTSL2, CLDN4, KIR2DL1, KIR2DL2, KIR2DL3, KIR2DL5A, KIR2DL5B, KIR2DS1, KIR2DS2, KIR2DS4, KIR2DS5, KIR3DL2, KIR3DL3, KIR3 DP1, LOC727787, RAPGEF5, CRMP1, LDB3, F11, USP46, IBSP, SLC9A3, FLRT3, TRIM17, FGF17, CAMK1G, GLYR1, CSH1, NTF3, ABHD6, TRIM15, OR52A1, FGFR2, ORAI2, C17orf53, GLP1R, SLIT1, TP63, DDR1, CFTR, DIO2, LETM1, ACSM5, ACTA1, NPR1, KCND3, POPDC3, DNAH3, SPDEF, CLEC4M, SLC30A3, NAGLU, AAK1, DHX34, NNAT, AKAP9, ICMT, FAM189A1, C10orf81, MYOZ1, PKNOX2, MGC31957, PRDM11, RET, IGHG1, XPNPEP2, NTRK2, SLC25A10, NR1I2, GRM8, OR3A3, GIPR, PAH, PACRG, CLN8, ZNF215, TRIO, TTLL5, GRM1, PRKG1, HHLA1, LAMA3, SLC37A4, HOXC11, SLCO5A1, CA10, RRBP1, SOD3, NTRK3, CYR61, STRA6, SLC6A11, CNOT4, ATN1, BCAP29, NOVA2, RELN, LAMC2, RAD51, PRSS7, DCBLD2, TACR2, RAB11B, OR2J2, VSNL1, IFNA17, DPYSL4, MGC2889, RRBP1, POLQ, OR1A2, PURA, AIF1, CBS, NECAB2, PRKCE, NOX1, IHH, EXO1, GPRIN2, PDX1, GPR12, FAM188A, HS3ST3B1, ASCL1, ZNF484, CSH1, BCAN, DDN, DUOX2, MORN1, SLC39A2, CLCN7, RUNX2, TTYH1, ZNF280B, PAX3, LZTS1, SLC8A2, HAB1, KIF1A, ARL4D, UGT2B15, NACA2, THRB, C6orf15, GPR176, WSCD1, PLXNB3, CADM3, HAP1, CYP1A2, SPAM1, IL22RA1, CDC2L5, IRX5, PPFIA2, KDELR3, CEACAM7, KCMF1, DUOX1, CDC27, HIST2H2AA3, CAV3, APOA4, NPR3, PRG3, TBC1D22B, TUSC3, RIMS2, CYP4F12, TBXA2R, HBEGF, PSG9, PYGO1, RASGRF1, SCN2A, KLHL1, DTNB, GREM1, SNCG, C22orf24, PALM, COBLL1, DNPEP, MNS1, NFATC4, DLC1, HSPC072, MCAM, CA12, CSHL1, RPA1N, COL5A2, UGT1A8, UGT1A9, IGH@, IGHA1, IGHG1, IGHG2, IGHG3, IGHM, LOC100126583, LOC100290036, LOC100290320, LOC100293211, LOC652494, ACSM5, ALOX12P2, ERBB4, CLDN16, CIB2, GALR3, MSMB, FABP7, ATXN3, KCNJ5, TRDN, CYP3A43, BAZ2A, ACCN4, SILV, DGCR14, SEMA6C, DIO2, PTHLH, LEP, PDZRN3, RGSL1, GJA4, SLC22A6, RASGRF1, MAPRE2, PVRL1, AKAP1, POMP, SOX21, DNAH9, HOXC5, SERHL2, KIAA0485, ITSN1, B4GALT1, NEK2, NUPR1, CCDC93, EPO, CRABP2, TYRO3, GOLGA2, SEMA3F, BFSP2, NCAM1, FOLH1, SSX2, TMPRSS4, DCN, LPHN3, POU4F3, CEACAM5, BCL3, EXTL3, CCNA1, DDR2, PAX8, SOX5, POU3F1, PEX16, NUP62, SIGLEC11, ALDOB, GPC3, IGFALS, WDR25, FGF1, OSR2, ARID1A, GYPA, KLK13, PARVB, LILRB5, RIMS2, C19orf21, HOXD1, PRSS3, FLT1, ATP6V1C1, LOX, CRYBB3, CA112, PRKG2, MASP1, LOC728395, LOC728403, TSPY11, PDCD1, GGTLC1, AQP8, KRT16, AICDA, BRD8, C1orf95, OR3A2, PFKFB2, FRZB, PAK3, MEIS2, ZSCAN2, MYH7, VWA1, LSAMP, SRC, UGT1A1, UGT1A10, UGT1A3, UGT1A4, UGT1A5, UGT1A6, UGT1A7, UGT1A8, UGT1A9, DIO1, TADA3L, NFASC, CALCRL, NBLA00301, MAB21L1, FBXO42, COL10A1, CFB, SNX7, FOXN1, SRY, HLF, CLCA3P, DAZ1, DAZ2, DAZ3, DAZ4, GPR3, TMPRSS11E, EMID1, KCNMB2, MUC5AC, SORT1, HIF3A, MAPK4, TCP11L1, ZZEF1, DCAF7, DMWD, CLCA2, VAC14, CSPG5, STMN2, MLLT4, GALNT14, FGF12, MFAP5, SUMO3, HTR3A, GDF5, TSSK1B, CYP2A7P1, MARK1, ATP1B2, TBX6, PAX8, IL1R1, RALYL, OR2B2, TAAR3, C12orf32, IGHG1, LOC642131, DICER1, GLRA3, PPARD, HSPA4L, WNT2, VIPR2, CYP2C9, SRPX2, IGSF1, ALPK3, TFPI, KCNS3, MARCH8, FRMD4B, TACR3, FIGF, PDCD6, TNN, SPANXB1, SPANXB2, SPANXF1, RHBDD3, SPP2, PDE10A, ZNF224, FGL1, PGAM2, CADM4, APOBEC2, SLC9A5, GNAT1, ARHGEF16, SMARCA2, DNAH9, RBM26, WNT2B, KCNK2, NPBWR2, SP2, TMPRSS11D, DENND2A, TNIP3, STC1, DOCK6, ADAM5P, SYDE1, TNPO2, LRTM1, USH1C, PDE12, SRCAP, OR10J1, OR2H2, KCNJ8, RP11-257K9.7, DOCK5, TPD52L1, PAEP, GGA2, PHLDA3, HES2, MLL, CHRNA6, CIB2, PTPRF, TM7SF4, DAZ1, DAZ2, DAZ3, DAZ4, ALX1, OR2F1, OR2F2, PLAT, HGC6.3, WNT11, PGK2, SNAI2, COL4A6, PRUNE2, ANKS1B, LOC81691, FERMT2, TIMP3, CST8, CAPN6, IDUA, GPR32, AKR1B10, GRHL2, FBXO24, HSF4, IGHG1, HCN2, LRP12, ARHGEF15, UGT1A1, UGT1A10, UGT1A7, UGT1A8, GUCA2A, ITIH1, EGFR, UGT1A1, UGT1A10, UGT1A3, UGT1A4, UGT1A5, UGT1A6, UGT1A7, UGT1A8, UGT1A9, MYOG, TMSB15A, TLX1, EDNRA, LOC100289791, MDFI, ZER1, MYH15, CDH20, GPR63, LOC440345, LOC440354, LOC595101, LOC641298, SMG1, HOXC10, KRTAP1-1, ARSD, CPLX3, LMAN1L, IFNA4, ABCC11, SEMA3E, MRE11A, C1QL1, LIPF, TRIM9, BBOX1, LRRC17, WNT2B, CYP3A4, SI, ANO3, OBSL1, CHRD, MSX2, PSG1, FAM107A, LRRC37B2, ANKLE2, PAX2, UNC5B, ADCYAP1R1, HFE, SYT1, GJC2, LOC100293871, FGF8, ACRV1, NRXN1, GDPD2, RGS4, CELA2A, IFNW1, MLNR, RNF17, LAD1, GLRA2, RASL12, MAGOH2, C6orf54, ZNF214, IKBKG, AP4E1, ZNRF4, OSBPL10, C1orf175, TTC4, PCDHB3, ADRBK1, ITSN1, XAGE1A, XAGE1B, XAGE1C, XAGE1D, XAGE1E, CDH22, FARP2, MYT1, TNC, MUC5AC, SLC6A15, PP14571, SMR3A, SMR3B, RXRG, SNX1, GLP1R, C6orf155, ATP1A2, TFAP4, PNPLA2, DIRAS3, ANO2, TACSTD2, MCM3AP, IL13RA2, TRIM10, RTEL1, PRRX2, TSHB, TIMELESS, FMO1, KIF18A, KIAA1199, CALB2, MFAP3L, PTGER3, EPAS1, SQSTM1, TSPY1, CPM, DLGAP1, CYP4F11, TLX3, PCDHA10, TAOK2, ERC1, TBX2, KALRN, DICER1, PAPPA, KIF5A, DNAJC22, OTUB1, KIAA1644, SEZ6L2, PCNXL2, HMHB1, ERG, SNTB2, GJA5, AGTR2, GJA3, GCK, LRRC61, CNTF, ZFP91, ZFP91-CNTF, PDLIM4, MPPED2, IFNA10, ACTN2, VGLL1, GJA9, LDLR, ANK2, COL1A1, TIMP3, OTOF, AGXT, GLI2, TRMT61A, FOXD2, TMEM2I2, DENND2A, B3GALT1, SPAG11A, PRDM4, TF, ELF5, GSC2, EPB41 L4B, GYG2, LYZL6, DCHS2, OBP2A, OBP2B, ANGPTL3, MYH11, NES, SLC17A1, RBM15B, CSH1, HTR5A, CYP3A7, HTR2A, KCNV2, TOX3, CLOCK, MAGEA6, FAM12A, COL4A3, S1PR2, NAT8, ACE2, SLC22A6, SLC13A2, MYH4, APBB2, RAP1GAP, SHOX2, SLCO1A2, ETV1, MAGEA12, PLA2G6, ADRA1A, SYT5, GPR161, SEMA3F, CYP3A43, HOMER2, KCNJ5, PPL, COL17A1, CSHL1, C9orf116, PARK2, UGT2B15, CDK6, FAM174B3, CELA2A, CELA2B, SPDEF, EPB41, GAB1, SMR3A, PDE6G, COL5A1, ABCA6, DMD, CYLC2, CIDEA, RAG2, HIST1H2BN, FMO6P, MAOA, ANKRD53, HAPLN1, MT1M, EHD2, GAD2, CRISP2, CSN2, SULT1C2, PCDHGA3, SSX3, FGFR2, GPR161, ATN1, CHD5, A4GALT, MYBPH, CSHL1, NCAPH2, CAPN9, CNGB1, BCAM, DRD5, NR5A2, TEF, ELAVL2, DGKB, HTR7P, RHAG, GH2, COL4A6, BMP7, SOSTDC1, SOX14, TAS2R9, LPHN2, MAP1A, OSGIN2, SLC10A2, FAM13C, EMX1, FLJ40330, CHI3L1, CDH16, SPRR1A, LOX, CALCB, GABBR2, CPB2, RASL11B, CCDC81, RUNX1, CPA1, CLCNKA, CLCNKB, FHL5, THSD7A, TFAP2C, SPAG11B, CAP2, PODNL1, SSX4, SSX4B, G6PC, RPE65, TMEM222, KDR, CHP2, GPR64, TPM2, TCEB3B, E2F5, IL5RA, AOC3, ABCF3, CPN2, ACE, NRP2, INPP5J, SMAD9, FAM155A, GART, PIR, ZNF467, ITSN2, NR1D1, THRA, RP11-35N6.1, LAMB1, EPHB3, PLA2R1, RAPGEF4, DNAJC8, ARSJ, TRIM49, GC, CDH2, ATXN3L, BTF3L1, BICC1, FAM186A, PTPRF, TRPC4, TCL6, CYP4A22, FUT6, MUC1, DKFZP434B2016, LOC643313, LDHA, LOC100131613, TRIM3, MLLT10, DZIP1, ANKRD34C, BUB1, CSPG5, FBLN1, GAD2, CLDN1, CHRNA3, SCN11A, TEX11, IL20RA, AKAP5, KBTBD10, MSTN, TLL2, NACAD, UNC93A, PTGER1, OLAH, NHLH2, SERPINA6, KRT17, KCNMA1, PRKCA, STS, LAMA1, GPR88, ACTN2, TREH, AKAP4, DKK4, PRICKLE3, IRS4, TRPV4, PCDH11Y, APBB2, SLCO2A1, DRD2, MTMR7, ZNF471, TF, NRIP2, ST6GALNAC5, COMT, PAH, LRRC19, PRKAR1B, HPR, PRDM5, NCRNA00120, LOC79999, ITSN2, CACNB2, GPR98, PREX2, FAM182B, LAMA4, ARVCF, HAS2, YOD1, PPP2R3A, COL4A1, RBM12B, GSTA3, FAM66D, OR10H2, PTHLH, ZNF674, KRT119, ACCN2, COL6A1, LOC100288442, LOC100289169, LOC728888, LOC729602, NPIPL2, NPIPL3, PDXDC2, SLC37A1, ATP6V1B1, ABI3BP, HR44, ZNF324B, ZNF584, HOXD13, ADH6, IFNA8, MYOZ2, NFATC4, ADAMTS7, FOXL1, GPR17, SLC18A3, MYH6, BOK, FGA, TEAD4, GRM1, EDNRA, C8orf79, METTL7A, FOLH1, RAD54L, SOX11, CNOT3, NTS, MAPK12, DOCK6, DNAJC6, HS3ST3A1, LOC728395, TSPY1, TSPY3, PTH, LAMB4, ALDOB, FLG, MLANA, UBE2D4, LOC100287483, KRT20, POU1F1, SLCO1B3, CLTA, MECOM, C8orf71, SULT2A1, C6orf10, SLC27A6, PRKD1, SYNPO2L, THPO, GABRR1, CFTR, PPP2R3A, DCBLD2, ANP32A, ANP32C, ANP32D, LOC723972, XYLT1, STAB1, STAB1, SASH1, PID1, FUCA1, SASH1, LRRN3, LRRN3 or a combination thereof.

25. The method of any one of claims 14-24, wherein laquinimod is administered orally.

26. The method of any one of claims 14-25, wherein laquinimod is administered daily.

27. The method of any one of claims 14-26, wherein laquinimod is administered at a dose of less than 0.6 mg/day, of 0.1-40.0 mg/day, 0.1-2.5 mg/day, 0.25-2.0 mg/day, 0.5-1.2 mg/day, 0.25 mg/day, 0.3 mg/day, 0.5 mg/day, 0.6 mg/day, 1.0 mg/day, 1.2 mg/day, 1.5 mg/day or 2.0 mg/day.

28. The method of any one of claims 14-27, wherein the subject is naïve to laquinimod.

29. The method of any one of claims 14-27, wherein the subject has been previously administered laquinimod.

30. The method of any one of claims 14-27, wherein the subject has been previously administered a multiple sclerosis drug other than laquinimod.

31. The method of any one of claims 14-30, wherein the step of evaluating expression of the biomarker comprises normalization of the subject's gene expression.

32. The method of any one of claims 14-31, wherein the step of evaluating expression of the biomarker comprises comparing expression level in the subject relative to a reference value.

33. The method of claim 32, wherein the reference value is based on the level of expression of the biomarker in a laquinimod Non-Responder population.

34. The method of claim 32, wherein the reference value is based on the level of expression of the biomarker in a healthy control population.

35. The method of any one of claims 32-34, wherein the subject is identified as a laquinimod responder if expression of the biomarker is higher than a reference value.

36. The method of any one of claims 32-34, wherein the subject is identified as a laquinimod responder if expression level of the biomarker is lower than a reference value.

37. The method of any one of claims 1-36, wherein expression of the biomarker is evaluated in the blood of the subject.

38. The method of claim 37, wherein expression of the biomarker is evaluated in the peripheral blood mononuclear cells (PBMCs) of the subject.

39. The method of any one of claims 14-38, wherein expression of the biomarker is evaluated prior to treatment with laquinimod.

40. The method of any one of claims 14-38, wherein expression of the biomarker is evaluated after beginning treatment with laquinimod.

41. The method of claim 40, wherein expression of the biomarker is evaluated one month, 6 months, 12 months or 24 months after beginning treatment with laquinimod.

42. The method of any one of claims 14-41, wherein if the subject is identified as a laquinimod responder, the subject is thereafter administered a pharmaceutical composition comprising laquinimod and a pharmaceutically acceptable carrier as monotherapy.

43. The method of any one of claims 14-41, wherein if the subject is identified as a laquinimod responder, the subject is thereafter administered a pharmaceutical composition comprising laquinimod and a pharmaceutically acceptable carrier in combination with another multiple sclerosis drug.

44. The method of any one of claims 14-43, wherein if the subject is identified as a laquinimod non-responder, the subject is thereafter administered a multiple sclerosis drug which is not laquinimod.

45. The method of any one of claims 1-44, wherein the subject is a human patient.

46. Laquinimod for use in treating a subject afflicted with multiple sclerosis or presenting a clinically isolated syndrome, wherein the subject has been identified as a laquinimod responder.

47. A pharmaceutical composition comprising an amount of laquinimod for use in treating a subject afflicted with multiple sclerosis or presenting a clinically isolated syndrome, wherein the subject has been identified as a laquinimod responder.

48. Laquinimod for use in treating a subject afflicted with multiple sclerosis or presenting a clinically isolated syndrome, wherein expression of a biomarker in the subject is up-regulated and the biomarker is a gene associated with inflammatory response, a gene associated with cellular movement, a gene associated with cell signaling, a gene associated with cell development, a gene associated with hematological system, or a combination thereof.

49. A pharmaceutical composition comprising an amount of laquinimod for use in treating a subject afflicted with multiple sclerosis or presenting a clinically isolated syndrome, wherein expression of a biomarker in the subject is up-regulated and the biomarker is a gene associated with inflammatory response, a gene associated with cellular movement, a gene associated with cell signaling, a gene associated with cell development, a gene associated with hematological system, or a combination thereof.

50. Laquinimod for use in treating a subject afflicted with multiple sclerosis or presenting a clinically isolated syndrome, wherein expression of a biomarker in the subject is suppressed and the biomarker is a gene associated with inflammatory response, a gene associated with cellular movement, a gene associated with cell signaling, a gene associated with cell development, a gene associated with hematological system, or a combination thereof.

51. A pharmaceutical composition comprising an amount of laquinimod for use in treating a subject afflicted with multiple sclerosis or presenting a clinically isolated syndrome, wherein expression of a biomarker in the subject is suppressed and the biomarker is a gene associated with inflammatory response, a gene associated with cellular movement, a gene associated with cell signaling, a gene associated with cell development, a gene associated with hematological system, or a combination thereof.

52. A therapeutic package for dispensing to, or for use in dispensing to, a subject identified as a laquinimod responder afflicted with multiple sclerosis or presenting a clinically isolated syndrome, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject.

53. A therapeutic package for dispensing to, or for use in dispensing to, a subject afflicted with multiple sclerosis or presenting a clinically isolated syndrome, which comprises:
a) one or more unit doses, each such unit dose comprising an amount of laquinimod, and
b) a finished pharmaceutical container therefor, said container containing said unit dose or unit doses, said container further containing or comprising labeling directing the use of said package in the treatment of said subject,
wherein expression of a biomarker in the subject is suppressed or up-regulated and the biomarker is a gene associated with inflammatory response, a gene associated with cellular movement, a gene associated with cell signaling, a gene associated with cell development, a gene associated with hematological system, or a combination thereof.
